SEQUENCE LISTING (38 sequences)

SEQ ID NO: 1 - protein, 5 aa, synthetic construct
RFGVH

SEQ ID NO: 2 - protein, 16 aa, synthetic construct
VIWRGGSTDYNAAFVS

SEQ ID NO: 3 - protein, 12 aa, synthetic construct
HYYGSSDYALDN

SEQ ID NO: 4 - protein, 11 aa, synthetic construct
KASQDVRNLVV

SEQ ID NO: 5 - protein, 7 aa, synthetic construct
SGSYRYS

SEQ ID NO: 6 - protein, 9 aa, synthetic construct
QQHYSPPYT

SEQ ID NO: 7 - protein, 120 aa, synthetic construct
EVQLVESGPGLVKPSETLSLTCTVSGFSLTRFGVHWVRQPPGKGLEWLGVIWRGGSTDYNAAFVSRLTISKDNSKNQVSLKLSSVTAADTAVYYCSNHYYGSSDYALDNWGQGTLVTVSS

SEQ ID NO: 8 - protein, 107 aa, synthetic construct
DIQMTQSPSSLSASVGDRVTITCKASQDVRNLVVWFQQKPGKAPKLLIYSGSYRYSGVPSRFSGSGSGTDFTLTISSLQPEDFATYYCQQHYSPPYTFGQGTKVEIK

SEQ ID NO: 9 - protein, 5 aa, synthetic construct
SYYIH

SEQ ID NO: 10 - protein, 17 aa, synthetic construct
WIYPENDNTKYNEKFKD

SEQ ID NO: 11 - protein, 10 aa, synthetic construct
DGYSRYYFDY

SEQ ID NO: 12 - protein, 17 aa, synthetic construct
KSSQSLLNSRTRKNYLA

SEQ ID NO: 13 - protein, 7 aa, synthetic construct
WTSTRKS

SEQ ID NO: 14 - protein, 8 aa, synthetic construct
KQSFILRT

SEQ ID NO: 15 - protein, 119 aa, synthetic construct
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSS

SEQ ID NO: 16 - protein, 112 aa, synthetic construct
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIK

SEQ ID NO: 17 - protein, 30 aa, synthetic construct
EVQLVESGPGLVKPSETLSLTCTVSGFSLT

SEQ ID NO: 18 - protein, 14 aa, synthetic construct
WVRQPPGKGLEWLG

SEQ ID NO: 19 - protein, 32 aa, synthetic construct
RLTISKDNSKNQVSLKLSSVTAADTAVYYCSN

SEQ ID NO: 20 - protein, 11 aa, synthetic construct
WGQGTLVTVSS

SEQ ID NO: 21 - protein, 23 aa, synthetic construct
DIQMTQSPSSLSASVGDRVTITC

SEQ ID NO: 22 - protein, 15 aa, synthetic construct
WFQQKPGKAPKLLIY

SEQ ID NO: 23 - protein, 32 aa, synthetic construct
GVPSRFSGSGSGTDFTLTISSLQPEDFATYYC

SEQ ID NO: 24 - protein, 10 aa, synthetic construct
FGQGTKVEIK

SEQ ID NO: 25 - protein, 30 aa, synthetic construct
EVQLVQSGAEVKKPGASVKVSCKASGFTFT

SEQ ID NO: 26 - protein, 14 aa, synthetic construct
WVRQAPGQGLEWIG

SEQ ID NO: 27 - protein, 32 aa, synthetic construct
RVTITADTSTSTAYLELSSLRSEDTAVYYCAR

SEQ ID NO: 28 - protein, 11 aa, synthetic construct
WGQGTLVTVSS

SEQ ID NO: 29 - protein, 23 aa, synthetic construct
DIVMTQSPDSLAVSLGERATINC

SEQ ID NO: 30 - protein, 15 aa, synthetic construct
WYQQKPGQSPKLLIY

SEQ ID NO: 31 - protein, 32 aa, synthetic construct
GVPDRFSGSGSGTDFTLTISSLQAEDVAVYYC

SEQ ID NO: 32 - protein, 10 aa, synthetic construct
FGQGTKVEIK

SEQ ID NO: 33 - DNA, 5620 bp, Homo sapiens
  source 1..5620: mol_type = genomic DNA
gcagtttcagaacccagccagcctctctcttgctgcctagcctcctgccggcctcatcttcgcccagccaaccccgcctggagccctatggccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctgagccagtcgctgtggttgttttagctccttgactccttgtggtttatgtcatcatacatgactcagcatacctgctggtgcagagctgaagattttggagggtcctccacaataaggtcaatgccagagacggaagcctttttccccaaagtcttaaaataacttatatcatcagcatacctttattgtgatctatcaatagtcaagaaaaattattgtataagattagaatgaaaattgtatgttaagttacttcactttaattctcatgtgatccttttatgttatttatatattggtaacatcctttctattgaaaaatcaccacaccaaacctctcttattagaacaggcaagtgaagaaaagtgaatgctcaagtttttcagaaagcattacatttccaaatgaatgaccttgttgcatgatgtatttttgtacccttcctacagatagtcaaaccataaacttcatggtcatgggtcatgttggtgaaaattattctgtaggatataagctacccacgtacttggtgctttaccccaacccttccaacagtgctgtgaggttggtattatttcattttttagatgagaaaatgggagctcagagaggttatatatttaagttggtgcaaaagtaattgcaagttttgccaccgaaaggaatggcaaaaccacaattatttttgaaccaacctaataatttaccgtaagtcctacatttagtatcaagctagagactgaatttgaactcaactctgtccaactccaaaattcatgtgctttttccttctaggcctttcataccaaactaatagtagtttatattctcttccaacaaatgcatattggattaaattgactagaatggaatctggaatatagttcttctggatggctccaaaacacatgtttttcttcccccgtcttcctcctcctcttcatgctcagtgttttatatatgtagtatacagttaaaatatacttgttgctggtactggcagcttatattttctctcttttttcatggattaaccttgcttgagggctttaacaattgtattactttttcaaagaactaagctttagcttcattgatttttttctatttaattgggttttgctcttctctttagcattggaaacatagaaatgctttctgatttctttgggtagatttacgtattcagcttcttgagatggaagtttagatcactgatccttcagcttgttttcttttttgtatacatagattttaggacgatatattttcccttgagttctgctttagctgcagctcttatgttttgatatgcctctctttattatccttcagttaaaaatatctttcaattcattgttatataaaaatatgtgcctagtttttaacatctggagattttctagttttgaaaaaaacataagccaggcatggtggctcacacctgtatccccagcactttgggaggccgagacgggaggatcgcctgagctcaggagtttttacaccagcctgggaataacagtgagacattatctccaaaaaaattacctgggtatggtgttgtgcacctgtagtcccagctactctggagactgaggtgggaggattgtttgagcttgggaggttgaggctgcagggagctgtgatcacaccactgcactctggcctgagtgacagattgagaccctgtctcaataaaagcaaaaataaagaaaataaaccatatgtgttgaacaaaggattaataaattaatttgagactccttcagggaatgaccacaatttattgaaaatagcctaaatgttggagtcaggcatttctggattcatattttgacatcatgctgtcatcttgaacaaaatgcctaacctttctgaacttcaacttccttgccactcaaataaggattacaaaacttaaaatgtggtaagtactaaagacgacagcaaaaattgagtccagcacagagcttcctaaataagcaagcactcaacagagttggttcctttcttcctcccctgcttgacaatccagtttcccacaggagcctttgtagctgtagccaccatggtcagtccagggattcttcactagccccttctcccctggcagacatccttgtgggagtttagtcttggctcgacatgaggatgggggtttgggaccagttctgagtgagaatcagacttgccccaagttgccattagctccccctgcagaatgtcttcagaatcggggcccggtcagtctcctgggtgacctgctgttttcctcttaagatcctttccactttggttgctgctttcgggactcatcgagtccttgctcaacaggataccccttgaagtggctgcctgggccacatccccttccaaacaagaaatcaaaatattagaaatcaatttttgaaatttcccctaggaagactcatttgagtgttcaagttcagagccagtggagaccttaggggagggtggtcacaaggattttgcacagtgctttagagggtcccagggagccacagaggtggtgaggggctgggtgctcttttctccgtgcatgaccttgtgtgtctatcttcattaccacaatgcctcatctctacctcctttccccctgtagttccaacgtgggtatctttgccatctctggcccgaaggactttctgacctacatgtataaataccccctcacaatatatattacttttcctataagtgacttctctactggattactggttgctcatacacctcatattttactcgtaaatctactactccctgtctgcctactccattctcatttgctgtagaaaattctcttaccatcccaactttcacccaccatcatgcttacccaaaggctgtgggaatgacctgggccctaatgccccttttctaaattcctaaggctcaccattttcctattgtaatggttcttgaccttataatgtttgaggcaccttttcaaatatagtcctttgatttcagactgaatacttgaaaggacacacacacacatacgtaagtgcatatgactgcatacacccacacacacacacgtgcctgtatacagtcatatgatacatacacaaacacacgcacacaagcctgcatacatcatatgccaacagtggggatatgttctgagaaatgcatcattagatgattttgtcattgtgtgaacatcatagagtgtacttacactaacctagatggtctaacctactacacacccaggctacatggtatcacctattcctcctaggctacaagcctgtacagcgtgtgtctgtactaaatgctgtgggcaattttaacctgatggtaaatgtttgtgtatctaaacatatctaaacatagaaaaggtacagtaaacatgcagtattataatcttatgagaccgtcatcatatatgtggtccactgtttgggccatcattggctgaaaagtggttatgcgacacatgactgtatatatactttcctgttacaacaacagtgtctctcaatccacagtaattgcagcatccagtaggtcttactttagccctgagtcaccatttgtgtcaacgtgtttagtgccatgtccacgtctctcatgtaactggcagagctatcaaatattttggcaaaacacattgtttctttggctttgccttggtaactttctgtgccttttgtagctcttgtttggaagaagctcaacccatgtctgcacactgtgatacaagggggacagcatcgacatcgacttacttcttggtgccttattcctccttagaacaattcctaaatctgtaacttaagtttctcaggaagattccatactgcacagaaaactgcttttgtgggtttttaaaaggcaagttgttatatgtgctggatagtttttaagtatgacataaaaattgtataaagtaaaatattaaaatacacctagaatactgtataactttaagtcattttatcaacacattgctaatccagatattttcccgcagtttttctttgaataacagagcaattaatttacttttactatgaagagtcatcattttagtatgtattttaagcaatccaccaagaactcagtaggcagctgagaggtgctgcccagagaagtggtgattagcttggccttagctcacccacacaaagcacaacaggctttgaactattccctaacggggcatttattcttttttttttttttttttgggagacggagtctcgctgtcgcccaggctagagtgcagtggcgcgatctcggctcactgcaggctccaccccctggggttcacgccattctcctgcctcagcctcccaagtagctgggactgcaggcgcccgccatctcgcccggctaattttttgtatttttagtagagacggggtttcaccgtgttagccaggatagggcatttattcttgaacttgattcagagaggcacacattaccattctctaatcagaatgcaagtagcgcaaggcggtggaaactatggaattcggaggcaggtgatgcattgggcgagtttattaacatctgtgactctctagtttgaaatttatttgtaacagacaaaaatgaattaaacaaacaataaaagtataataaagaa

SEQ ID NO: 34 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 35 - protein, 450 aa, synthetic construct
EVQLVESGPGLVKPSETLSLTCTVSGFSLTRFGVHWVRQPPGKGLEWLGVIWRGGSTDYNAAFVSRLTISKDNSKNQVSLKLSSVTAADTAVYYCSNHYYGSSDYALDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 214 aa, synthetic construct
DIQMTQSPSSLSASVGDRVTITCKASQDVRNLVVWFQQKPGKAPKLLIYSGSYRYSGVPSRFSGSGSGTDFTLTISSLQPEDFATYYCQQHYSPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 449 aa, synthetic construct
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 38 - protein, 219 aa, synthetic construct
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0179188 A1
(43) Pub. Date: Jun. 5, 2025

(54) DOSING FOR TREATMENT WITH ANTI-FCRH5/ANTI-CD3 BISPECIFIC ANTIBODIES

(71) Applicant: Genentech, Inc., South San Francisco, CA (US)

(72) Inventors: Bernard Martin Fine, Menlo Park, CA (US); Teiko Sumiyoshi, San Francisco, CA (US); Mengsong Li, Castro Valley, CA (US); James Niall Cooper, Missoula, MT (US)

(21) Appl. No.: 18/941,183

(22) Filed: Nov. 8, 2024

Related U.S. Application Data
(63) Continuation of application No. PCT/US2022/028770, filed on May 11, 2022.

(51) Int. Cl. C07K 16/28; A61K 39/00; A61K 39/395

(52) U.S. Cl. CPC C07K 16/283; A61K 39/39558; C07K 16/2809; A61K 2039/505; A61K 2039/54; A61K 2039/545; C07K 2317/31

(57) ABSTRACT

The invention provides methods of dosing for the treatment of cancers, such as multiple myelomas, with anti-fragment crystallizable receptor-like 5 (FcRH5)/anti-cluster of differentiation 3 (CD3) bispecific antibodies.

### SEQUENCE LISTING

The instant application contains a Sequence Listing which has been submitted electronically in XML format and is hereby incorporated by reference in its entirety. Said XML copy, created on Nov. 4, 2024, is named “50474-278002_Sequence_Listing_114_24” and is 41,569 bytes in size.

### FIELD OF THE INVENTION

The present invention relates to the treatment of cancers, such as B cell proliferative disorders. More specifically, the invention concerns the treatment of human patients having multiple myeloma (MM) using anti-fragment crystallizable receptor-like 5 (FcRH5)/anti-cluster of differentiation 3 (CD3) bispecific antibodies.

### BACKGROUND

Cancer remains one of the most deadly threats to human health. In the U.S., cancer affects more than 1.7 million new patients each year and is the second leading cause of death after heart disease, accounting for approximately one in four deaths.

Hematologic cancers, in particular, are the second leading cause of cancer-related deaths. Hematologic cancers include multiple myeloma (MM), a neoplasm characterized by the proliferation and accumulation of malignant plasma cells. Worldwide, approximately 160,000 people are diagnosed with MM annually. MM remains incurable despite advances in treatment, with an estimated median survival of 8-10 years for standard-risk myeloma and 2-3 years for high-risk disease, despite receipt of an autologous stem cell transplant. Despite the significant improvement in patient survival over the past 20 years, only 10-15% of patients achieve or exceed expected survival compared with the matched general population. Increased survival has been achieved with the introduction of proteasome inhibitors, immunomodulatory drugs (IMiDs), and monoclonal antibodies. Nevertheless, most patients (if not all) eventually relapse, and the outcome of patients with MM after they become refractory, or ineligible to receive a proteasome inhibitor or an IMiD, is quite poor, with survival less than 1 year.

Therefore, relapsed or refractory (R/R) MM, in particular, continues to constitute a significant unmet medical need, and novel therapeutic agents and treatments are needed.

### SUMMARY OF THE INVENTION

Provided herein are, inter alia, methods of treating a cancer (e.g., a B cell proliferative disorder, such as MM), and related compositions for use, uses, and articles of manufacture.

In one aspect, the invention features a method of treating a subject having a multiple myeloma (MM), the method comprising subcutaneously administering to the subject a bispecific antibody that binds to Fc receptor-homolog 5 (FcRH5) and cluster of differentiation 3 (CD3) in a dosing regimen comprising: (i) a first phase comprising one or more dosing cycles, wherein the first phase comprises administering the bispecific antibody to the subject every week (QW); (ii) a second phase comprising one or more dosing cycles, wherein the second phase comprises administering the bispecific antibody to the subject every two weeks (Q2W); and (iii) a third phase comprising one or more dosing cycles, wherein the third phase comprises administering the bispecific antibody to the subject every four weeks (Q4W).

In some aspects, each dosing cycle is a 28-day dosing cycle.

In some aspects, the first phase comprises a first dosing cycle (C1).

In some aspects, the first phase consists of a C1.

In some aspects, the first phase comprises administration of the bispecific antibody to the subject on Days 1, 8, and 15 of the C1.

In some aspects, a target dose of the bispecific antibody is administered to the subject for each administration during the first phase.

In some aspects, the first phase comprises administration of a first step-up dose of the bispecific antibody to the subject.

In some aspects, the first step-up dose is administered to the subject on Day 1 of C1.

In some aspects, the target dose is administered to the subject on Days 8 and 15 of C1.

In some aspects, the first step-up dose is about 1% to 30% of the target dose.

In some aspects, the first step-up dose is about 5% to 25% of the target dose.

In some aspects, the first step-up dose is 5% of the target dose.

In some aspects, the first step-up dose is 25% of the target dose.

In some aspects, the first step-up dose is 2 mg.

In some aspects, the first step-up dose is 10 mg.

In some aspects, first phase comprises administration of a first step-up dose and a second step-up dose of the bispecific antibody to the subject.

In some aspects, the first step-up dose is administered to the subject on Day 1 of C1 and the second step-up dose is administered to the subject on Day 8 of C1.

In some aspects, the target dose is administered to the subject on Days 15 of C1.

In some aspects: (i) the first step-up dose is 1% to 10% of the target dose; and (ii) the second step-up dose is 15% to 45% of the target dose.

In some aspects: (i) the first step-up dose is 5% of the target dose; and (ii) the second step-up dose is 25% of the target dose.

In some aspects, the first step-up dose is 2 mg and the second step-up dose is 10 mg.

In some aspects, the bispecific antibody is not administered to the subject on Day 22 of the C1.

In some aspects, the bispecific antibody is administered to the subject a total of three times during the C1.

In some aspects, the bispecific antibody is administered to the subject on Day 22 of the C1.

In some aspects, the second phase comprises at least two dosing cycles, at least three dosing cycles, at least four dosing cycles, or at least five dosing cycles.

In some aspects, the second phase comprises a first dosing cycle (C1), a second dosing cycle (C2), a third dosing cycle (C3), a fourth dosing cycle (C4), and a fifth dosing cycle (C5).

In some aspects, the second phase consists of a C1, a C2, a C3, a C4, and a C5.

In some aspects, the second phase comprises administration of the bispecific antibody to the subject on Days 1 and 15 of the C1, C2, C3, C4, and/or C5.

In some aspects, a target dose of the bispecific antibody is administered to the subject for each administration during the second phase.

In some aspects, the third phase comprises at least two dosing cycles, at least three dosing cycles, at least four dosing cycles, at least five dosing cycles, at least six dosing cycles, or at least seven dosing cycles.

In some aspects, the third phase comprises a first dosing cycle (C1), a second dosing cycle (C2), a third dosing cycle (C3), a fourth dosing cycle (C4), a fifth dosing cycle (C5), a sixth dosing cycle (C6), and a seventh dosing cycle (C7).

In some aspects, the third phase consists of a C1, a C2, a C3, a C4, a C5, a C6, and a C7.

In some aspects, the third phase comprises administration of the bispecific antibody to the subject on Day 1 of the C1, C2, C3, C4, C5, C6, and/or C7.

In some aspects, a target dose of the bispecific antibody is administered to the subject for each administration during the third phase.

In some aspects, the dosing regimen further comprises a fourth phase comprising one or more dosing cycles.

In some aspects, the fourth phase comprises administering the bispecific antibody to the subject subcutaneously every week (QW), every two weeks (Q2W), every three weeks (Q3W), or every four weeks (Q4W).

In some aspects, a target dose of the bispecific antibody is administered to the subject for each administration during the fourth phase.

In some aspects, the fourth phase comprises administering the bispecific antibody to the subject until disease progression.

In some aspects, the target dose is 40 mg.

In some aspects, the target dose is 120 mg.

In some aspects, the bispecific antibody is administered to the subject as a monotherapy.

In another aspect, the invention features a method of treating a subject having an MM, the method comprising subcutaneously administering to the subject a bispecific antibody that binds to FcRH5 and CD3 in a dosing regimen comprising: (i) a first dose of the bispecific antibody of between 0.1 mg to 10 mg; (ii) a second dose of the bispecific antibody of between 1 mg to 50 mg; and (iii) a third dose of the bispecific antibody of between 10 mg to 200 mg.

In some aspects: (i) the first dose of the bispecific antibody is between 1 mg to 3 mg; (ii) the second dose of the bispecific antibody is between 8 mg to 12 mg; and (iii) the third dose of the bispecific antibody is between 35 mg to 45 mg.

In some aspects: (i) the first dose of the bispecific antibody is 2 mg; (ii) the second dose of the bispecific antibody is 10 mg; and (iii) the third dose of the bispecific antibody is 40 mg.

In some aspects: (i) the first dose of the bispecific antibody is 2 mg; (ii) the second dose of the bispecific antibody is 10 mg; and (iii) the third dose of the bispecific antibody is 120 mg.

In some aspects, the bispecific antibody is administered to the subcutaneous tissue of the abdomen.

In some aspects, the subject's abdomen comprises four quadrants, and the bispecific antibody is administered to one of the four quadrants.

In some aspects, each sequential dose of the bispecific antibody is administered to a different member of the four quadrants on a rotating basis.

In some aspects, the bispecific antibody is administered to the subject's thigh.

In some aspects, the bispecific antibody is administered subcutaneously by injection or by infusion.

In some aspects, the bispecific antibody is administered subcutaneously by injection.

In some aspects, the bispecific antibody is administered with an injection speed of about 0.25 mL/min to about 4 mL/min.

In some aspects, the bispecific antibody is administered with an injection speed of about 1 mL/min.

In some aspects, the bispecific antibody is administered by a syringe.

In some aspects, the syringe is a pre-filled syringe.

In some aspects, the bispecific antibody is administered by a pump.

In some aspects, the pump comprises a patch pump, a syringe pump, or an infusion pump.

In some aspects, the pump is a wearable pump.

In some aspects, the bispecific antibody comprises an anti-FcRH5 arm comprising a first binding domain comprising the following six hypervariable regions (HVRs): (i) an HVR-H1 comprising the amino acid sequence of RFGVH (SEQ ID NO: 1); (ii) an HVR-H2 comprising the amino acid sequence of VIWRGGSTDYNAAFVS (SEQ ID NO: 2); (iii) an HVR-H3 comprising the amino acid sequence of HYYGSSDYALDN (SEQ ID NO:3); (iv) an HVR-L1 comprising the amino acid sequence of KASQDVRNLVV (SEQ ID NO: 4); (v) an HVR-L2 comprising the amino acid sequence of SGSYRYS (SEQ ID NO: 5); and (vi) an HVR-L3 comprising the amino acid sequence of QQHYSPPYT (SEQ ID NO: 6).

In some aspects, the bispecific antibody comprises an anti-FcRH5 arm comprising a first binding domain comprising (a) a heavy chain variable (VH) domain comprising an amino acid sequence having at least 95% sequence identity to the amino acid sequence of SEQ ID NO: 7; (b) a light chain variable (VL) domain comprising an amino acid sequence having at least 95% sequence identity to the amino acid sequence of SEQ ID NO: 8; or (c) a VH domain as in (a) and a VL domain as in (b).

In some aspects, the first binding domain comprises a VH domain comprising an amino acid sequence of SEQ ID NO: 7 and a VL domain comprising an amino acid sequence of SEQ ID NO: 8.

In some aspects, the bispecific antibody comprises an anti-CD3 arm comprising a second binding domain comprising the following six HVRs: (i) an HVR-H1 comprising the amino acid sequence of SYYIH (SEQ ID NO: 9); (ii) an HVR-H2 comprising the amino acid sequence of WIYPENDNTKYNEKFKD (SEQ ID NO: 10); (iii) an HVR-H3 comprising the amino acid sequence of DGYSRYYFDY (SEQ ID NO: 11); (iv) an HVR-L1 comprising the amino acid sequence of KSSQSLLNSRTRKNYLA (SEQ ID NO: 12); (v) an HVR-L2 comprising the amino acid sequence of WTSTRKS (SEQ ID NO: 13); and (vi) an HVR-L3 comprising the amino acid sequence of KQSFILRT (SEQ ID NO: 14).

In some aspects, the bispecific antibody comprises an anti-CD3 arm comprising a second binding domain comprising (a) a VH domain comprising an amino acid sequence having at least 95% sequence identity to the amino acid sequence of SEQ ID NO: 15; (b) a VL domain comprising an amino acid sequence having at least 95% sequence identity to the amino acid sequence of SEQ ID NO: 16; or (c) a VH domain as in (a) and a VL domain as in (b).

In some aspects, the second binding domain comprises a VH domain comprising an amino acid sequence of SEQ ID NO: 15 and a VL domain comprising an amino acid sequence of SEQ ID NO: 16.

In some aspects, the bispecific antibody comprises an anti-FcRH5 arm comprising a heavy chain polypeptide (H1) and a light chain polypeptide (L1) and an anti-CD3 arm comprising a heavy chain polypeptide (H2) and a light chain polypeptide (L2), and wherein: (i) H1 comprises the amino acid sequence of SEQ ID NO: 35; (ii) L1 comprises the amino acid sequence of SEQ ID NO: 36; (iii) H2 comprises the amino acid sequence of SEQ ID NO: 37; and (iv) L2 comprises the amino acid sequence of SEQ ID NO: 38.

In some aspects, the bispecific antibody comprises an aglycosylation site mutation.

In some aspects, the aglycosylation site mutation reduces effector function of the bispecific antibody.

In some aspects, the aglycosylation site mutation is a substitution mutation.

In some aspects, the bispecific antibody comprises a substitution mutation in the Fc region that reduces effector function.

In some aspects, the bispecific antibody is a monoclonal antibody.

In some aspects, the bispecific antibody is a humanized antibody.

In some aspects, the bispecific antibody is a chimeric antibody.

In some aspects, the bispecific antibody is an antibody fragment that binds FcRH5 and CD3.

In some aspects, the antibody fragment is selected from the group consisting of Fab, Fab′-SH, Fv, scFv, and (Fab′)₂ fragments.

In some aspects, the bispecific antibody is a full-length antibody.

In some aspects, the bispecific antibody is an IgG antibody.

In some aspects, the IgG antibody is an IgG₁ antibody.

In some aspects, the bispecific antibody comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, second CH2 (CH2₂) domain, and a second CH3 (CH3₂) domain.

In some aspects, at least one of the one or more heavy chain constant domains is paired with another heavy chain constant domain.

In some aspects, the CH3₁ and CH3₂ domains each comprise a protuberance or cavity, and wherein the protuberance or cavity in the CH3₁ domain is positionable in the cavity or protuberance, respectively, in the CH3₂ domain.

In some aspects, the CH3₁ and CH3₂ domains meet at an interface between the protuberance and cavity.

In some aspects, the CH2₁ and CH2₂ domains each comprise a protuberance or cavity, and wherein the protuberance or cavity in the CH2₁ domain is positionable in the cavity or protuberance, respectively, in the CH2₂ domain.

In some aspects, the CH2₁ and CH2₂ domains meet at an interface between said protuberance and cavity.

In some aspects, the anti-FcRH5 arm comprises the protuberance and the anti-CD3 arm comprises the cavity.

In some aspects, the CH3 domain of the anti-FcRH5 arm comprises a protuberance comprising a T366W amino acid substitution mutation (EU numbering) and the CH3 domain of the anti-CD3 arm comprises a cavity comprising T366S, L368A, and Y407V amino acid substitution mutations (EU numbering).

In some aspects, the bispecific antibody is cevostamab.

In some aspects, the bispecific antibody is administered to the subject concurrently with one or more additional therapeutic agents.

In some aspects, the bispecific antibody is administered to the subject prior to the administration of one or more additional therapeutic agents.

In some aspects, the bispecific antibody is administered to the subject subsequent to the administration of one or more additional therapeutic agents.

In some aspects, the one or more additional therapeutic agents comprise an effective amount of tocilizumab.

In some aspects, the subject has a cytokine release syndrome (CRS) event, and the method further comprises treating the symptoms of the CRS event while suspending treatment with the bispecific antibody.

In some aspects, the method further comprises administering to the subject an effective amount of tocilizumab to treat the CRS event.

In some aspects, tocilizumab is administered to the subject by intravenous infusion.

In some aspects: (i) the subject weighs >30 kg, and tocilizumab is administered to the subject at a dose of 8 mg/kg; (ii) the subject weighs <30 kg, and tocilizumab is administered to the subject at a dose of 12 mg/kg; or (iii) the final dose administered does not excess 800 mg.

In some aspects, if the CRS event does not resolve or worsens within 8 hours of treating the symptoms of the CRS event, then the method further comprises administering to the subject one or more additional doses of tocilizumab to manage the CRS event.

In some aspects, the one or more additional therapeutic agents comprise an effective amount of a corticosteroid.

In some aspects, the corticosteroid is administered intravenously to the subject.

In some aspects, the corticosteroid is methylprednisolone.

In some aspects, methylprednisolone is administered at a dose of 80 mg.

In some aspects, the corticosteroid is dexamethasone.

In some aspects, dexamethasone is administered at a dose of 20 mg.

In some aspects, the corticosteroid is administered to the subject 45 min to 75 min prior to administration of the bispecific antibody.

In some aspects, the corticosteroid is administered to the subject 60 min prior to administration of the bispecific antibody to the subject.

In some aspects, the corticosteroid is administered to the subject prior to administration of the bispecific antibody if the subject experienced CRS with a prior administration of the bispecific antibody to the subject.

In some aspects, the one or more additional therapeutic agents comprise an effective amount of acetaminophen or paracetamol.

In some aspects, acetaminophen or paracetamol is administered at a dose of between 500 mg to 1000 mg.

In some aspects, acetaminophen or paracetamol is administered orally to the subject.

In some aspects, acetaminophen or paracetamol is administered to the subject prior to administration of the bispecific antibody to the subject.

In some aspects, the one or more additional therapeutic agents comprise an effective amount of diphenhydramine.

In some aspects, diphenhydramine is administered at a dose of between 25 mg to 50 mg.

In some aspects, diphenhydramine is administered orally to the subject.

In some aspects, diphenhydramine is administered to the subject prior to administration of the bispecific antibody to the subject.

In some aspects, the one or more additional therapeutic agents comprise an effective amount of an immunomodulator (IMiD), a cluster of differentiation 38 (CD38)-directed therapy, or a B-cell maturation antigen (BCMA)-directed therapy.

In some aspects, the IMiD is pomalidomide.

In some aspects, the CD38-directed therapy is an anti-CD38 antibody.

In some aspects, the anti-CD38 antibody is daratumumab, MOR202, or isatuximab.

In some aspects, the anti-CD38 antibody is daratumumab.

In some aspects, the BCMA-directed therapy is an antibody-drug conjugate targeting BCMA.

In some aspects, the MM is a relapsed or refractory (R/R) MM.

In some aspects, the subject has a diagnosis of R/R MM for which no established therapy for MM is appropriate and available, or intolerance to established therapies.

In some aspects, the subject has measurable disease, defined as at least one of the following: (i) serum M-protein ≥0.5 g/dL; (ii) urine M-protein ≥200 mg/24 h; or (iii) serum free light chain (SLFC) assay: involved SFLCs ≥10 mg/dL and an abnormal SFLC ratio (<0.26 or >1.65).

In another aspect, the invention features a method of treating a subject having an R/R MM, the method comprising subcutaneously administering cevostamab to the subject in a dosing regimen comprising: (i) a first phase comprising a first dosing cycle (C1), wherein the C1 is a 28-day dosing cycle, wherein the first phase comprises administering the cevostamab to the subject as a first step-up dose on Day 1 of the C1, as a second step-up dose on Day 8 of the C1, and at a target dose on Day 15 of the C1, and wherein the first step-up dose is 2 mg, the second step-up dose is 10 mg, and the target dose is 40 mg; (ii) a second phase comprising a first dosing cycle (C1), a second dosing cycle (C2), a third dosing cycle (C3), a fourth dosing cycle (C4), and a fifth dosing cycle (C5), wherein each dosing cycle of the second phase is a 28-day dosing cycle, and wherein the second phase comprises administering the cevostamab to the subject on Day 1 and Day 15 of the C1, C2, C3, C4, and C5 at a target dose of 40 mg; and (iii) a third phase comprising a first dosing cycle (C1), a second dosing cycle (C2), a third dosing cycle (C3), a fourth dosing cycle (C4), a fifth dosing cycle (C5), a sixth dosing cycle (C6), and a seventh dosing cycle (C7), wherein each dosing cycle of the third phase is a 28-day dosing cycle, and wherein the third phase comprises administering the cevostamab on Day 1 of the C1, C2, C3, C4, C5, C6, and C7 at a target dose of 40 mg.

In another aspect, the invention features a subcutaneous administration device comprising a bispecific antibody that binds to FcRH5 and CD3, wherein the subcutaneous administration device comprises: (i) a first dose of the bispecific antibody of between 0.5 mg to 8 mg; (ii) a second dose of the bispecific antibody of between 2 mg to 40 mg; and/or (iii) a third dose of the bispecific antibody of between 10 mg to 160 mg.

In some aspects: (i) the first dose of the bispecific antibody is between 1 mg to 3 mg; (ii) the second dose of the bispecific antibody is between 8 mg to 12 mg; and/or (iii) the third dose of the bispecific antibody is between 35 mg to 45 mg.

In some aspects: (i) the first dose of the bispecific antibody is 2 mg; (ii) the second dose of the bispecific antibody is 10 mg; and/or (iii) the third dose of the bispecific antibody is 40 mg.

In some aspects: (i) the first dose of the bispecific antibody is 2 mg; (ii) the second dose of the bispecific antibody is 10 mg; and (iii) the third dose of the bispecific antibody is 120 mg.

In some aspects, the subcutaneous administration device is a syringe.

In some aspects, the subcutaneous administration device is a pre-filled syringe.

In some aspects, the subcutaneous administration device is a pump.

In some aspects, the pump comprises a patch pump, a syringe pump, or an infusion pump.

In some aspects, the pump is a wearable pump.

In some aspects, the subcutaneous administration device is for use in the treatment of MM.

In some aspects, the MM is R/R MM.

In another aspect, the invention features a bispecific antibody that binds to FcRH5 and CD3 for use in treatment of a subject having an MM, wherein the treatment comprises subcutaneous administration of the bispecific antibody to the subject in a dosing regimen comprising: (i) a first phase comprising one or more dosing cycles, wherein the first phase comprises administration of the bispecific antibody to the subject every week (QW); (ii) a second phase comprising one or more dosing cycles, wherein the second phase comprises administration of the bispecific antibody to the subject every two weeks (Q2W); and (iii) a third phase comprising one or more dosing cycles, wherein the third phase comprises administration of the bispecific antibody to the subject every four weeks (Q4W).

In another aspect, the invention features a bispecific antibody that binds to FcRH5 and CD3 for use in treatment of a subject having an MM, wherein the treatment comprises subcutaneous administration of the bispecific antibody to the subject in a dosing regimen comprising: (i) a first dose of the bispecific antibody of between 0.5 mg to 8 mg; (ii) a second dose of the bispecific antibody of between 2 mg to 40 mg; and (iii) a third dose of the bispecific antibody of between 10 mg to 160 mg.

In another aspect, the invention features cevostamab for use in treatment of a subject having an R/R MM, wherein the treatment comprises subcutaneous administration of cevostamab to the subject in a dosing regimen comprising: (i) a first phase comprising a first dosing cycle (C1), wherein the C1 is a 28-day dosing cycle, wherein the first phase comprises administering the cevostamab to the subject as a first step-up dose on Day 1 of the C1, as a second step-up dose on Day 8 of the C1, and at a target dose on Day 15 of the C1, and wherein the first step-up dose is 2 mg, the second step-up dose is 10 mg, and the target dose is 40 mg; (ii) a second phase comprising a first dosing cycle (C1), a second dosing cycle (C2), a third dosing cycle (C3), a fourth dosing cycle (C4), and a fifth dosing cycle (C5), wherein each dosing cycle of the second phase is a 28-day dosing cycle, and wherein the second phase comprises administering the cevostamab to the subject on Day 1 and Day 15 of the C1, C2, C3, C4, and C5 at a target dose of 40 mg; and (iii) a third phase comprising a first dosing cycle (C1), a second dosing cycle (C2), a third dosing cycle (C3), a fourth dosing cycle (C4), a fifth dosing cycle (C5), a sixth dosing cycle (C6), and a seventh dosing cycle (C7), wherein each dosing cycle of the third phase is a 28-day dosing cycle, and wherein the third phase comprises administering the cevostamab on Day 1 of the C1, C2, C3, C4, C5, C6, and C7 at a target dose of 40 mg.

### BRIEF DESCRIPTION OF THE DRAWINGS

FIG. 1 shows a schematic of a 28-day dosing cycle schedule for the subcutaneous administration of cevostamab. In brief, in Cycle 1, cevostamab is administered starting with a step-up dose on Days 1 and 8, followed by the target dose on Day 15. In Cycles 2-6, cevostamab is administered Q2W at the target dose. In Cycles 7-13, cevostamab is administered Q4W at the target dose.

FIG. 2 shows a schematic of abdominal injection sites and rotation of injection sites. Cevostamab may be administered to patients subcutaneously into the subcutaneous tissue of the abdomen. The abdomen can be divided into 4 quadrants, and injection sites can be rotated as shown.

### DETAILED DESCRIPTION OF THE INVENTION

### I. Definitions

The term “about” as used herein refers to the usual error range for the respective value readily known to the skilled person in this technical field. Reference to “about” a value or parameter herein includes (and describes) aspects that are directed to that value or parameter per se.

It is understood that aspects of the invention described herein include “comprising,” “consisting,” and “consisting essentially of” aspects.

The term “FcRH5” or “fragment crystallizable receptor-like 5,” as used herein, refers to any native FcRH5 from any vertebrate source, including mammals such as primates (e.g., humans) and rodents (e.g., mice and rats), unless otherwise indicated, and encompasses “full-length,” unprocessed FcRH5, as well as any form of FcRH5 that results from processing in the cell. The term also encompasses naturally occurring variants of FcRH5, including, for example, splice variants or allelic variants. FcRH5 includes, for example, human FcRH5 protein (UniProtKB/Swiss-Prot ID: Q96RD9.3), which is 977 amino acids in length.

The terms “anti-FcRH5 antibody” and “an antibody that binds to FcRH5” refer to an antibody that is capable of binding FcRH5 with sufficient affinity such that the antibody is useful as a diagnostic and/or therapeutic agent in targeting FcRH5. In one embodiment, the extent of binding of an anti-FcRH5 antibody to an unrelated, non-FcRH5 protein is less than about 10% of the binding of the antibody to FcRH5 as measured, e.g., by a radioimmunoassay (RIA). In certain embodiments, an antibody that binds to FcRH5 has a dissociation constant (K_{D}) of ≤1 μM, ≤250 nM, ≤100 nM, ≤15 nM, ≤10 nM, ≤6 nM, ≤4 nM, ≤2 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-FcRH5 antibody binds to an epitope of FcRH5 that is conserved among FcRH5 from different species.

The term “cluster of differentiation 3” or “CD3,” as used herein, refers to any native CD3 from any vertebrate source, including mammals such as primates (e.g., humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP_000724), which is 207 amino acids in length, and human CD3γ protein (NCBI RefSeq No. NP_000064), which is 182 amino acids in length.

The terms “anti-CD3 antibody” and “an antibody that binds to CD3” refer to an antibody that is capable of binding CD3 with sufficient affinity such that the antibody is useful as a diagnostic and/or therapeutic agent in targeting CD3. In one embodiment, the extent of binding of an anti-CD3 antibody to an unrelated, non-CD3 protein is less than about 10% of the binding of the antibody to CD3 as measured, e.g., by a radioimmunoassay (RIA). In certain embodiments, an antibody that binds to CD3 has a dissociation constant (K_{D}) of ≤1 μM, ≤250 nM, ≤100 nM, ≤15 nM, ≤10 nM, ≤5 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-CD3 antibody binds to an epitope of CD3 that is conserved among CD3 from different species.

For the purposes herein, “cevostamab,” also referred to as BFCR4350A or R071 87797, is an Fc-engineered, humanized, full-length non-glycosylated IgG1 kappa T-cell-dependent bispecific antibody (TDB) that binds FcRH5 and CD3 and comprises an anti-FcRH5 arm comprising the heavy chain polypeptide sequence of SEQ ID NO: 35 and the light chain polypeptide sequence of SEQ ID NO: 36 and an anti-CD3 arm comprising the heavy chain polypeptide sequence of SEQ ID NO: 37 and the light chain polypeptide sequence of SEQ ID NO: 38. Cevostamab comprises a threonine to tryptophan amino acid substitution at position 366 on the heavy chain of the anti-FcRH5 arm (T366W) using EU numbering of Fc region amino acid residues and three amino acid substitutions (tyrosine to valine at position 407, threonine to serine at position 366, and leucine to alanine at position 368) on the heavy chain of the anti-CD3 arm (Y407V, T366S, and L368A) using EU numbering of Fc region amino acid residues to drive heterodimerization of the two arms (half-antibodies). Cevostamab also comprises an amino acid substitution (asparagine to glycine) at position 297 on each heavy chain (N297G) using EU numbering of Fc region amino acid residues, which results in a non-glycosylated antibody that has minimal binding to Fc (Fcγ) receptors and, consequently, prevents Fc-effector function. Cevostamab is also described in WHO Drug Information (International Nonproprietary Names for Pharmaceutical Substances), Recommended INN: List 84, Vol. 34, No. 3, published 2020 (see page 701).

The term “antibody” herein is used in the broadest sense and encompasses various antibody structures, including but not limited to monoclonal antibodies, polyclonal antibodies, multispecific antibodies (e.g., bispecific antibodies), and antibody fragments (e.g., bis-Fabs) so long as they exhibit the desired antigen-binding activity.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary aspects for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “full-length antibody,” “intact antibody,” and “whole antibody” are used herein interchangeably to refer to an antibody having a structure substantially similar to a native antibody structure or having heavy chains that contain an Fc region as defined herein.

An “antibody fragment” refers to a molecule other than an intact antibody that comprises a portion of an intact antibody that binds the antigen to which the intact antibody binds. Examples of antibody fragments include but are not limited to bis-Fabs; Fv; Fab; Fab′-SH; F(ab′)2; diabodies; linear antibodies; single-chain antibody molecules (e.g., scFv, ScFab); and multispecific antibodies formed from antibody fragments.

A “single-domain antibody” refers to an antibody fragment comprising all or a portion of the heavy chain variable domain or all or a portion of the light chain variable domain of an antibody. In certain aspects, a single-domain antibody is a human single-domain antibody (see, e.g., U.S. Pat. No. 6,248,516 B1). Examples of single-domain antibodies include but are not limited to a VHH.

A “Fab” fragment is an antigen-binding fragment generated by papain digestion of antibodies and consists of an entire L chain along with the variable region domain of the H chain (VH), and the first constant domain of one heavy chain (CH1). Papain digestion of antibodies produces two identical Fab fragments. Pepsin treatment of an antibody yields a single large F(ab′)2 fragment which roughly corresponds to two disulfide linked Fab fragments having divalent antigen-binding activity and is still capable of cross-linking antigen. Fab′ fragments differ from Fab fragments by having an additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all Lys447 residues removed, antibody populations with no Lys447 residues removed, and antibody populations having a mixture of antibodies with and without the Lys447 residue.

A “functional Fc region” possesses an “effector function” of a native sequence Fc region. Exemplary “effector functions” include C1q binding; complement-dependent cytotoxicity (CDC); Fc receptor binding; antibody-dependent cell-mediated cytotoxicity (ADCC); phagocytosis; down regulation of cell surface receptors (e.g., B cell receptor; BCR); B cell activation, etc. Such effector functions generally require the Fc region to be combined with a binding domain (e.g., an antibody variable domain) and can be assessed using various assays as disclosed, for example, in definitions herein.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region, as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, preferably at least about 90% homology therewith, or preferably at least about 95% homology therewith.

“Fc complex” as used herein refers to CH3 domains of two Fc regions interacting together to form a dimer or, as in certain aspects, two Fc regions interact to form a dimer, wherein the cysteine residues in the hinge regions and/or the CH3 domains interact through bonds and/or forces (e.g., Van der Waals, hydrophobic forces, hydrogen bonds, electrostatic forces, or disulfide bonds).

The term “FcRH5-positive cancer” refers to a cancer comprising cells that express FcRH5 on their surface. For the purposes of determining whether a cell expresses FcRH5 on the surface, FcRH5 mRNA expression is considered to correlate to FcRH5 expression on the cell surface. In some embodiments, expression of FcRH5 mRNA is determined by a method selected from in situ hybridization and RT-PCR (including quantitative RT-PCR). Alternatively, expression of FcRH5 on the cell surface can be determined, for example, using antibodies to FcRH5 in a method such as immunohistochemistry, FACS, etc. In some embodiments, FcRH5 is one or more of FcRH5a, FcRH5b, FcRH5c, UniProt Identifier Q96RD9-2, and/or FcRH5d. In some embodiments, the FcRH5 is FcRH5c.

“Hinge region” is generally defined as stretching from about residue 216 to 230 of an IgG (EU numbering), from about residue 226 to 243 of an IgG (Kabat numbering), or from about residue 1 to 15 of an IgG (IMGT unique numbering).

The “lower hinge region” of an Fc region is normally defined as the stretch of residues immediately C-terminal to the hinge region, i.e., residues 233 to 239 of the Fc region (EU numbering).

“Fc receptor” or “FcR” describes a receptor that binds to the Fc region of an antibody. A preferred FcR is a native sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Dasron, Annu. Rev. Immunol. 15:203-234 (1997)). FcRs are reviewed in Ravetch and Kinet, Annu. Rev. Immunol. 9:457-492 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The term “knob-into-hole” or “KnH” technology as mentioned herein refers to the technology directing the pairing of two polypeptides together in vitro or in vivo by introducing a protuberance (knob) into one polypeptide and a cavity (hole) into the other polypeptide at an interface in which they interact. For example, KnHs have been introduced in the Fc:Fc interaction interfaces, CL:CH1 interfaces or VH/VL interfaces of antibodies (e.g., US2007/0178552, WO 96/027011, WO 98/050431 and Zhu et al., (1997) Protein Science 6:781-788). This is especially useful in driving the pairing of two different heavy chains together during the manufacture of multispecific antibodies. For example, multispecific antibodies having KnH in their Fc regions can further comprise single variable domains linked to each Fc region, or further comprise different heavy chain variable domains that pair with identical, similar, or different light chain variable domains. KnH technology can also be used to pair two different receptor extracellular domains together or any other polypeptide sequences that comprise different target recognition sequences.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The “CH1 region” or “CH1 domain” comprises the stretch of residues from about residue 118 to residue 215 of an IgG (EU numbering), from about residue 114 to 223 of an IgG (Kabat numbering), or from about residue 1.4 to residue 121 of an IgG (IMGT unique numbering) (Lefranc et al., IMGT®, the international ImMunoGeneTics information system® 25 years on. Nucleic Acids Res. 2015 January; 43(Database issue):D413-22).

The “CH2 domain” of a human IgG Fc region usually extends from about residues 244 to about 360 of an IgG (Kabat numbering), from about residues 231 to about 340 of an IgG (EU numbering), or from about residues 1.6 to about 125 of an IgG (IGMT unique numbering). The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22:161-206 (1985).

The “CH3 domain” comprises the stretch of residues C-terminal to a CH2 domain in an Fc region (i.e., from about amino acid residue 361 to about amino acid residue 478 of an IgG (Kabat numbering), from about amino acid residue 341 to about amino acid residue 447 of an IgG (EU numbering), or from about amino acid residue 1.4 to about amino acid residue 130 of an IgG (IGMT unique numbering)).

The “CL domain” or “constant light domain” comprises the stretch of residues C-terminal to a light-chain variable domain (VL). The light chain (LC) of an antibody may be a kappa (κ) (“Cκ”) or lambda (λ) (“Cλ”) light chain region. The Cκ region generally extends from about residue 108 to residue 214 of an IgG (Kabat or EU numbering) or from about residue 1.4 to residue 126 of an IgG (IMGT unique numbering). The CA residue generally extends from about residue 107a to residue 215 (Kabat numbering) or from about residue 1.5 to residue 127 (IMGT unique numbering) (Lefranc et al., supra).

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries. Hoogenboom and Winter, J. Mol. Biol. 227:381,1991; Marks et al., J. Mol. Biol. 222:581, 1991. Also available for the preparation of human monoclonal antibodies are methods described in Cole et al., Monoclonal Antibodies and Cancer Therapy, Alan R. Liss, p. 77 (1985); Boerner et al., J. Immunol., 147 (1): 86-95, 1991. See also van Dijk and van de Winkel, Curr. Opin. Pharmacol. 5:368-74, 2001. Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA. 103:3557-3562, 2006, regarding human antibodies generated via a human B-cell hybridoma technology.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al. Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al. supra. In one aspect, for the VH, the subgroup is subgroup III as in Kabat et al. supra.

A “humanized” antibody refers to a chimeric antibody comprising amino acid residues from non-human HVRs and amino acid residues from human FRs. In certain aspects, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. In certain aspects in which all or substantially all of the FRs of a humanized antibody correspond to those of a human antibody, any of the FRs of the humanized antibody may contain one or more amino acid residues (e.g., one or more Vernier position residues of FRs) from non-human FR(s). A humanized antibody optionally may comprise at least a portion of an antibody constant region derived from a human antibody. A “humanized form” of an antibody, e.g., a non-human antibody, refers to an antibody that has undergone humanization.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al., Kuby Immunology, 6th ed. W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887, 1993; Clarkson et al. Nature 352:624-628, 1991.

The term “hypervariable region” or “HVR” as used herein refers to each of the regions of an antibody variable domain which are hypervariable in sequence (“complementarity determining regions” or “CDRs”). Generally, antibodies comprise six CDRs: three in the VH (CDR-H1, CDR-H2, CDR-H3), and three in the VL (CDR-L1, CDR-L2, CDR-L3). Exemplary CDRs herein include:
(a) CDRs occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917, 1987);
(b) CDRs occurring at amino acid residues 24-34 (L1), 50-56 (L2), 89-97 (L3), 31-35b (H1), 50-65 (H2), and 95-102 (H3) (Kabat et al. Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745, 1996).

Unless otherwise indicated, HVR residues and other residues in the variable domain (e.g., FR residues) are numbered herein according to Kabat et al. supra.

“Single-chain Fv” also abbreviated as “sFv” or “scFv” are antibody fragments that comprise the VH and VL antibody domains connected into a single polypeptide chain. Preferably, the scFv polypeptide further comprises a polypeptide linker between the VH and VL domains, which enables the scFv to form the desired structure for antigen binding. For a review of scFv, see Pluckthun, The Pharmacology of Monoclonal Antibodies, vol. 113, Rosenburg and Moore eds., Springer-Verlag, New York, pp. 269-315 (1994); Malmborg et al., J. Immunol. Methods 183:7-13, 1995.

By “targeting domain” is meant a part of a compound or a molecule that specifically binds to a target epitope, antigen, ligand, or receptor. Targeting domains include but are not limited to antibodies (e.g., monoclonal, polyclonal, recombinant, humanized, and chimeric antibodies), antibody fragments or portions thereof (e.g., bis-Fab fragments, Fab fragments, F(ab′)₂, scFab, scFv antibodies, SMIP, single-domain antibodies, diabodies, minibodies, scFv-Fc, affibodies, nanobodies, and VH and/or VL domains of antibodies), receptors, ligands, aptamers, peptide targeting domains (e.g., cysteine knot proteins (CKP)), and other molecules having an identified binding partner. A targeting domain may target, block, agonize, or antagonize the antigen to which it binds.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “multispecific antibody” is used in the broadest sense and specifically covers an antibody that has polyepitopic specificity. In one aspect, the multispecific antibody binds to two different targets (e.g., bispecific antibody). Such multispecific antibodies include, but are not limited to, an antibody comprising a heavy chain variable domain (VH) and a light chain variable domain (VL), where the VH/VL unit has polyepitopic specificity, antibodies having two or more VL and VH domains with each VH/VL unit binding to a different epitope, antibodies having two or more single variable domains with each single variable domain binding to a different epitope, full-length antibodies, antibody fragments such as Fab, Fv, dsFv, scFv, diabodies, bispecific diabodies and triabodies, antibody fragments that have been linked covalently or non-covalently. “Polyepitopic specificity” refers to the ability to specifically bind to two or more different epitopes on the same or different target(s). “Monospecific” refers to the ability to bind only one antigen. In one aspect, the monospecific biepitopic antibody binds two different epitopes on the same target/antigen. In one aspect, the monospecific polyepitopic antibody binds to multiple different epitopes of the same target/antigen. According to one aspect, the multispecific antibody is an IgG antibody that binds to each epitope with an affinity of 5 μM to 0.001 μM, 3 μM to 0.001 μM, 1 μM to 0.001 μM, 0.5 μM to 0.001 μM, or 0.1 μM to 0.001 μM.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N to C-terminus, each heavy chain has a variable region (VH), also called a variable heavy domain or a heavy chain variable domain, followed by three constant domains (CH1, CH2, and CH3). Similarly, from N to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

As used herein, the term “immunoadhesin” designates molecules which combine the binding specificity of a heterologous protein (an “adhesin”) with the effector functions of immunoglobulin constant domains. Structurally, the immunoadhesins comprise a fusion of an amino acid sequence with a desired binding specificity, which amino acid sequence is other than the antigen recognition and binding site of an antibody (i.e., is “heterologous” compared to a constant region of an antibody), and an immunoglobulin constant domain sequence (e.g., CH2 and/or CH3 sequence of an IgG). The adhesin and immunoglobulin constant domains may optionally be separated by an amino acid spacer. Exemplary adhesin sequences include contiguous amino acid sequences that comprise a portion of a receptor or a ligand that binds to a protein of interest. Adhesin sequences can also be sequences that bind a protein of interest, but are not receptor or ligand sequences (e.g., adhesin sequences in peptibodies). Such polypeptide sequences can be selected or identified by various methods, include phage display techniques and high throughput sorting methods. The immunoglobulin constant domain sequence in the immunoadhesin can be obtained from any immunoglobulin, such as IgG1, IgG2, IgG3, or IgG4 subtypes, IgA (including IgA1 and IgA2), IgE, IgD, or IgM.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitinib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, rapamycin (Sirolimus, RAPAMUNE®, Wyeth), Lapatinib (TYKERB®, GSK572016, Glaxo Smith Kline), lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin y11 and calicheamicin ω11 (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, III.), and TAXOTERER® (docetaxel, doxetaxel; Sanofi-Aventis); chlorambucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4 (5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTING, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see U.S. Pat. No. 4,943,533) and variants thereof, such as chimerized 225 (C225 or Cetuximab; ERBUTIX®) and reshaped human 225 (H225) (see WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al., Eur. J. Cancer 32A: 636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279 (29): 30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl) propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy) quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl) amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl) methoxy]phenyl]-6 [5 [2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis(4-fluoroanilino) phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacizumab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), interleukin 1 (IL-1) blockers such as anakinra (Kineret), T cell costimulation blockers such as abatacept (Orencia), interleukin 6 (IL-6) blockers such as tocilizumab (ACTEMRA®); interleukin 13 (IL-13) blockers such as lebrikizumab; interferon alpha (IFN) blockers such as Rontalizumab; beta 7 integrin blockers such as rhuMAb Beta7; IgE pathway blockers such as Anti-M1 prime; Secreted homotrimeric LTa3 and membrane bound heterotrimer LTa1/β2 blockers such as anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹², and radioactive isotopes of Lu); miscellaneous investigational agents such as thioplatin, PS-341, phenylbutyrate, ET-18—OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhea, metastatic bone pain, headache and migraine, postoperative pain, mild-to-moderate pain due to inflammation and tissue injury, pyrexia, ileus, and renal colic.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹², and radioactive isotopes of Lu); chemotherapeutic agents or drugs (e.g., methotrexate, doxorubicin (ADRIAMYCIN®), vinca alkaloids (vincristine, vinblastine, etoposide), melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

A “disorder” is any condition that would benefit from treatment including, but not limited to, chronic and acute disorders or diseases including those pathological conditions which predispose a mammal to the disorder in question. In one aspect, the disorder is a cancer, e.g., a B cell proliferative disorder such as an MM, e.g., relapsed or refractory MM.

The terms “cell proliferative disorder” and “proliferative disorder” refer to disorders that are associated with some degree of abnormal cell proliferation. In one aspect, the cell proliferative disorder is cancer. In one aspect, the cell proliferative disorder is a tumor.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The terms “cancer” and “cancerous” refer to or describe the physiological condition in mammals that is typically characterized by unregulated cell growth/proliferation. Aspects of cancer include solid tumor cancers and non-solid tumor cancers. Examples of cancer include, but are not limited to, B cell proliferative disorders, such as MM, which may be relapsed or refractory MM. The MM may be, e.g., typical MM (e.g., immunoglobulin G (IgG) MM, IgA MM, IgD MM, IgE MM, or IgM MM), light chain MM (LCMM) (e.g., lambda light chain MM or kappa light chain MM), or non-secretory MM. The MM may have one or more cytogenetic features (e.g., high-risk cytogenic features), e.g., t (4; 14), t (11; 14), t (14; 16), and/or del (17p), as described in Table 1 and in the International Myeloma Working Group (IMWG) criteria provided in Sonneveld et al., Blood, 127 (24): 2955-2962, 2016, and/or 1q21, as described in Chang et al., Bone Marrow Transplantation, 45:117-121, 2010. Cytogenic features may be detected, e.g., using fluorescent in situ hybridization (FISH). Examples of solid tumors include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, as well as abnormal vascular proliferation associated with phakomatoses, edema (such as that associated with brain tumors), Meigs' syndrome, brain, as well as head and neck cancer, and associated metastases. In certain embodiments, cancers that are amenable to treatment by the antibodies of the invention include breast cancer, colorectal cancer, rectal cancer, non-small cell lung cancer, glioblastoma, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, ovarian cancer, and mesothelioma.

TABLE 1
Cytogenic features of MM

| Primary genetic events | | Secondary genetic events | |
|---|---|---|---|
| IgH translocation | Gene(s) | Deletion | Gene(s) |
| t(4; 14) | FGFR3/ | 1p | CDKN2C, FAF1, |
| | MMSET | | FAM46C |
| t(6; 14) | CCND3 | 6q | |
| t(11; 14) | CCND1 | 8p | |
| t(14; 16) | MAF | 13 | RB1, DIS3 |
| t(14; 20) | MAFB | 11q | BIRC2/BIRC3 |
| | | 14q | TRAF3 |
| | | 16q | WWOX, CYLD |
| | | 17p | TP53 |
| Hyperdiploidy | | Gain | |
| Trisomies of chromosomes | | 1q | CKS1B, ANP32E |
| 3, 5, 7, 9, 11, 15, 19, 21 | | | |

Lee 2014 criteria: Lee et al., Blood, 124: 188-195, 2014.
ASTCT consensus grading: Lee et al., Biol Blood Marrow Transplant, 25(4): 625-638, 2019.
^{a} Low-dose vasopressor: single vasopressor at doses below that shown in Table 6.
^{b} High-dose vasopressor: as defined in Table 6.
*Fever is defined as temperature ≥38° C. not attributable to any other cause. In patients who have CRS then receive antipyretic or anticytokine therapy such as tocilizumab or steroids, fever is no longer required to grade subsequent CRS severity. In this case, CRS grading is driven by hypotension and/or hypoxia.
^{†}CRS grade is determined by the more severe event: hypotension or hypoxia not attributable to any other cause. For example, a patient with temperature of 39.5° C., hypotension requiring 1 vasopressor, and hypoxia requiring low-flow nasal cannula is classified as grade 3 CRS.
^{‡}Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

The term “B cell proliferative disorder” or “B cell malignancy” refers to a disorder that is associated with some degree of abnormal B cell proliferation and includes, for example, a lymphoma, leukemia, myeloma, and myelodysplastic syndrome. In one embodiment, the B cell proliferative disorder is a lymphoma, such as non-Hodgkin's lymphoma (NHL), including, for example, diffuse large B cell lymphoma (DLBCL) (e.g., relapsed or refractory DLBCL). In another embodiment, the B cell proliferative disorder is a leukemia, such as chronic lymphocytic leukemia (CLL). Other specific examples of cancer also include germinal-center B cell-like (GCB) diffuse large B cell lymphoma (DLBCL), activated B cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B cell prolymphocytic leukemia, splenic marginal zone lymphoma, hairy cell leukemia, splenic lymphoma/leukemia, unclassifiable, splenic diffuse red pulp small B cell lymphoma, hairy cell leukemia variant, heavy chain diseases, a heavy chain disease, γ heavy chain disease, u heavy chain disease, plasma cell myeloma, solitary plasmacytoma of bone, extraosseous plasmacytoma, extranodal marginal zone lymphoma of mucosa-associated lymphoid tissue (MALT lymphoma), nodal marginal zone lymphoma, pediatric nodal marginal zone lymphoma, pediatric follicular lymphoma, primary cutaneous follicle center lymphoma, T cell/histiocyte rich large B cell lymphoma, primary DLBCL of the CNS, primary cutaneous DLBCL, leg type, EBV-positive DLBCL of the elderly, DLBCL associated with chronic inflammation, lymphomatoid granulomatosis, primary mediastinal (thymic) large B cell lymphoma, intravascular large B cell lymphoma, ALK-positive large B cell lymphoma, plasmablastic lymphoma, large B cell lymphoma arising in HHV8-associated multicentric Castleman disease, primary effusion lymphoma: B cell lymphoma, unclassifiable, with features intermediate between DLBCL and Burkitt lymphoma, and B cell lymphoma, unclassifiable, with features intermediate between DLBCL and classical Hodgkin's lymphoma. Further examples of cancer include, but are not limited to, carcinoma, lymphoma, blastoma, sarcoma, and leukemia or lymphoid malignancies, including B cell lymphomas. More particular examples of such cancers include, but are not limited to, low grade/follicular NHL; small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; AIDS-related lymphoma; and acute lymphoblastic leukemia (ALL); chronic myeloblastic leukemia; and post-transplant lymphoproliferative disorder (PTLD).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) that are bound to their cognate antigen. To assess complement activation, a CDC assay, e.g., as described in Gazzano-Santoro et al., J. Immunol. Methods 202:163 (1996), can be performed.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxic agents. The antibodies “arm” the cytotoxic cells and are absolutely required for such killing. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet. Annu. Rev. Immunol. 9:457-92, 1991. To assess ADCC activity of a molecule of interest, an in vitro ADCC assay, such as that described in U.S. Pat. No. 5,500,362 or 5,821,337 can be performed. Useful effector cells for such assays include peripheral blood mononuclear cells (PBMC) and Natural Killer (NK) cells. Alternatively, or additionally, ADCC activity of the molecule of interest can be assessed in vivo, e.g., in an animal model such as that disclosed in Clynes et al., Proc. Natl. Acad. Sci. USA. 95:652-656, 1998.

“Complex” or “complexed” as used herein refers to the association of two or more molecules that interact with each other through bonds and/or forces (e.g., Van der Waals, hydrophobic, hydrophilic forces) that are not peptide bonds. In one aspect, the complex is heteromultimeric. It should be understood that the term “protein complex” or “polypeptide complex” as used herein includes complexes that have a non-protein entity conjugated to a protein in the protein complex (e.g., including, but not limited to, chemical molecules such as a toxin or a detection agent).

As used herein, “delaying progression” of a disorder or disease means to defer, hinder, slow, retard, stabilize, and/or postpone development of the disease or disorder (e.g., a cell proliferative disorder, e.g., cancer (e.g., MM)). This delay can be of varying lengths of time, depending on the history of the disease and/or individual being treated. As is evident to one skilled in the art, a sufficient or significant delay can, in effect, encompass prevention, in that the individual does not develop the disease. For example, a late-stage cancer, such as development of metastasis, may be delayed.

An “effective amount” of a compound, for example, an anti-FcRH5/anti-CD3 T-cell-dependent bispecific antibody (TDB) disclosed herein or a composition (e.g., pharmaceutical composition) thereof, is at least the minimum amount required to achieve the desired therapeutic or prophylactic result, such as a measurable improvement or prevention of a particular disorder (e.g., a cell proliferative disorder, e.g., cancer). An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “overall survival” or “OS” refers to the percentage of individuals in a group who are likely to be alive after a particular duration of time.

As used herein, “objective response rate” (ORR) refers to the sum of stringent complete response (sCR), complete response (CR), very good partial response (VGPR), and partial response (PR) rates as determined using the International Myeloma Working Group response criteria (e.g., see Table 8A and 8B in Example 1).

The term “epitope” refers to the particular site on an antigen molecule to which an antibody binds. In some aspects, the particular site on an antigen molecule to which an antibody binds is determined by hydroxyl radical footprinting. In some aspects, the particular site on an antigen molecule to which an antibody binds is determined by crystallography.

A “growth inhibitory agent” when used herein refers to a compound or composition which inhibits growth of a cell either in vitro or in vivo. In one aspect, growth inhibitory agent is growth inhibitory antibody that prevents or reduces proliferation of a cell expressing an antigen to which the antibody binds. In another aspect, the growth inhibitory agent may be one which significantly reduces the percentage of cells in S phase. Aspects of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

An “immunoconjugate” is an antibody conjugated to one or more heterologous molecule(s), including but not limited to a cytotoxic agent.

The term “immunomodulatory agent” or “IMID” refers to a class of molecules that modifies the immune system response or the functioning of the immune system. Immunomodulatory agents include, but are not limited to, POMALYST® (pomalidomide), thalidomide (α-N-phthalimido-glutarimide) and its analogues, OTEZLA® (apremilast), REVLIMID® (lenalidomide) and PD-1 axis binding antagonists and pharmaceutically acceptable salts or acids thereof.

A “subject” or an “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the subject or individual is a human. The subject may be a patient.

An “isolated” protein or peptide is one which has been separated from a component of its natural environment. In some aspects, a protein or peptide is purified to greater than 95% or 99% purity as determined by, for example, electrophoresis (e.g., sodium dodecyl sulfate-polyacrylamide gel electrophoresis (SDS-PAGE), isoelectric focusing (IEF), capillary electrophoresis) or chromatography (e.g., ion exchange or reverse phase HPLC).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L2 binding antagonist. In some instances, the PD-1 axis binding antagonist includes a PD-L1 binding antagonist or a PD-1 binding antagonist. In a preferred aspect, the PD-1 axis binding antagonist is a PD-L1 binding antagonist.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some aspects, the anti-PD-L1 antibody is atezolizumab, MDX-1105, MEDI4736 (durvalumab), or MSB0010718C (avelumab). In one specific aspect, the PD-L1 binding antagonist is MDX-1105. In another specific aspect, the PD-L1 binding antagonist is MEDI4736 (durvalumab). In another specific aspect, the PD-L1 binding antagonist is MSB0010718C (avelumab). In other aspects, the PD-L1 binding antagonist may be a small molecule, e.g., GS-4224, INCB086550, MAX-10181, INCB090244, CA-170, or ABSK041, which in some instances may be administered orally. Other exemplary PD-L1 binding antagonists include AVA-004, MT-6035, VXM10, LYN192, GB7003, and JS-003. In a preferred aspect, the PD-L1 binding antagonist is atezolizumab. Atezolizumab is also described in WHO Drug Information (International Nonproprietary Names for Pharmaceutical Substances (proposed INN)) List 112, Vol. 28, No. 4, 2014, p. 488.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. In some instances, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate, or interfere with signal transduction resulting from the interaction of PD-1 with PD-L1 and/or PD-L2. In one instance, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some instances, the PD-1 binding antagonist binds to PD-1. In some instances, the PD-1 binding antagonist is an anti-PD-1 antibody (e.g., an anti-PD-1 antagonist antibody). Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is a PD-L2 Fc fusion protein, e.g., AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In another specific aspect, a PD-1 binding antagonist is prolgolimab. In another specific aspect, a PD-1 binding antagonist is camrelizumab. In another specific aspect, a PD-1 binding antagonist is sintilimab. In another specific aspect, a PD-1 binding antagonist is tislelizumab. In another specific aspect, a PD-1 binding antagonist is toripalimab. Other additional exemplary PD-1 binding antagonists include BION-004, CB201, AUNP-012, ADG104, and LBL-006.

The term “PD-L2 binding antagonist” refers to a molecule that decreases, blocks, inhibits, abrogates or interferes with signal transduction resulting from the interaction of PD-L2 with either one or more of its binding partners, such as PD-1. PD-L2 (programmed death ligand 2) is also referred to in the art as “programmed cell death 1 ligand 2,” “PDCD1LG2,” “CD273,” “B7-DC,” “Btdc,” and “PDL2.” An exemplary human PD-L2 is shown in UniProtKB/Swiss-Prot Accession No. Q9BQ51. In some instances, a PD-L2 binding antagonist is a molecule that inhibits the binding of PD-L2 to one or more of its binding partners. In a specific aspect, the PD-L2 binding antagonist inhibits binding of PD-L2 to PD-1. Exemplary PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some aspects, the PD-L2 binding antagonist binds to PD-L2. In some aspects, a PD-L2 binding antagonist is an immunoadhesin. In other aspects, a PD-L2 binding antagonist is an anti-PD-L2 antagonist antibody.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein as well as any form of the protein that results from processing in the cell. The term also encompasses naturally occurring variants of the protein, e.g., splice variants or allelic variants.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein: protein) program and default options (BLOSUM50; open: −10; ext: −2; Ktup=2) to ensure a global, rather than local, alignment is performed. Percent amino acid identity is given in the output alignment header.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies disclosed herein (e.g., anti-FcRH5/anti-CD3 TDBs disclosed herein) are used to delay development of a disease or to slow the progression of a disease.

By “reduce” or “inhibit” is meant the ability to cause an overall decrease, for example, of 20% or greater, of 50% or greater, or of 75%, 85%, 90%, 95%, or greater. In certain aspects, reduce or inhibit can refer to the effector function of an antibody that is mediated by the antibody Fc region, such effector functions specifically including CDC, ADCC, and ADCP.

According to the invention, the term “vaccine” relates to a pharmaceutical preparation (pharmaceutical composition) or product that upon administration induces an immune response, in particular a cellular immune response, which recognizes and attacks a pathogen or a diseased cell such as a cancer cell. A vaccine may be used for the prevention or treatment of a disease. A vaccine may be a cancer vaccine. A “cancer vaccine” as used herein is a composition that stimulates an immune response in a subject against a cancer. Cancer vaccines typically consist of a source of cancer-associated material or cells (antigen) that may be autologous (from self) or allogenic (from others) to the subject, along with other components (e.g., adjuvants) to further stimulate and boost the immune response against the antigen. Cancer vaccines can result in stimulating the immune system of the subject to produce antibodies to one or several specific antigens, and/or to produce killer T cells to attack cancer cells that have those antigens.

As used herein, “administering” is meant a method of giving a dosage of a compound (e.g., an anti-FcRH5/anti-CD3 TDB such as cevostamab) to a subject. In some aspects, the compositions utilized in the methods herein are administered intravenously. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

“CD38” as used herein refers to a glycoprotein found on the surface of many immune cells, including CD4+, CD8+, B lymphocytes, and natural killer (NK) cells, and includes any native CD38 from any vertebrate source, including mammals such as primates (e.g., humans) and rodents (e.g., mice and rats), unless otherwise indicated. CD38 is typically expressed at a higher level and more uniformly on myeloma cells as compared to normal lymphoid and myeloid cells. The term encompasses “full-length,” unprocessed CD38, as well as any form of CD38 that results from processing in the cell. The term also encompasses naturally occurring variants of CD38, e.g., splice variants or allelic variants. CD38 is also referred to in the art as cluster of differentiation 38, ADP-ribosyl cyclase 1, cADPr hydrolase 1, and cyclic ADP-ribose hydrolase 1. CD38 is encoded by the CD38 gene. The nucleic acid sequence of an exemplary human CD38 is shown under NCBI Reference Sequence: NM_001775.4 or in SEQ ID NO: 33. The amino acid sequence of an exemplary human CD38 protein encoded by CD38 is shown under UniProt Accession No. P28907 or in SEQ ID NO: 34.

The term “anti-CD38 antibody” encompasses all antibodies that bind CD38 with sufficient affinity such that the antibody is useful as a therapeutic agent in targeting a cell expressing the antigen, and does not significantly cross-react with other proteins such as a negative control protein in the assays described below. For example, an anti-CD38 antibody may bind to CD38 on the surface of a MM cell and mediate cell lysis through the activation of complement-dependent cytotoxicity, ADCC, antibody-dependent cellular phagocytosis (ADCP), and apoptosis mediated by Fc cross-linking, leading to the depletion of malignant cells and reduction of the overall cancer burden. An anti-CD38 antibody may also modulate CD38 enzyme activity through inhibition of ribosyl cyclase enzyme activity and stimulation of the cyclic adenosine diphosphate ribose (cADPR) hydrolase activity of CD38. In certain aspects, an anti-CD38 antibody that binds to CD38 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain aspects, the anti-CD38 antibody may bind to both human CD38 and chimpanzee CD38. Anti-CD38 antibodies also include anti-CD38 antagonist antibodies. Bispecific antibodies wherein one arm of the antibody binds CD38 are also contemplated. Also encompassed by this definition of anti-CD38 antibody are functional fragments of the preceding antibodies. Examples of antibodies which bind CD38 include: daratumumab (DARZALEX®) (U.S. Pat. No. 7,829,673 and U.S. Pub. No: 20160067205 A1); “MOR202” (U.S. Pat. No. 8,263,746); and isatuximab (SAR-650984).

A “subcutaneous administration device” refers to a device which is adapted or designed to administer a drug, for example a therapeutic antibody (e.g., an anti-FcRH5/anti-CD3 bispecific antibody (e.g., cevostamab)), or pharmaceutical formulation by the subcutaneous route. Exemplary subcutaneous administration devices include, but are not limited to, a syringe, including a pre-filled syringe, an injection device, infusion pump, injector pen, needleless device, and patch delivery system. A subcutaneous administration device may administer a particular volume of the pharmaceutical formulation, for example about 1.0 mL, about 1.25 mL, about 1.5 mL, about 1.75 mL, about 2.0 mL, about 2.5 mL, about 3 mL, about 3.5 mL, about 4 mL, about 5 mL, or more.

### II. Therapeutic Methods

The invention is based, in part, on methods of treating a subject having cancer (e.g., multiple myeloma (MM)) using dosing regimens, including fractionated, dose-escalation dosing regimens with anti-fragment crystallizable receptor-like 5 (FcRH5)/anti-cluster of differentiation 3 (CD3) bispecific antibodies. The invention provides a cevostamab monotherapy dosing regimen in which cevostamab is administered to the subject subcutaneously. An exemplary dosing regimen described herein is of cevostamab as a single agent in a subcutaneous dosing regimen, in which cevostamab is administered in 28-day cycles where cevostamab is administered subcutaneously Q1W for the first cycle (C1), subcutaneously Q2W for Cycles 2 to 6, and subcutaneously Q4W for cycles 7 to 13. The methods disclosed herein may, e.g., facilitate alignment with the dosing schedules of combination therapy partners. Without wishing to be bound by theory, subcutaneous dosing may, for example, reduce the Cₘₐₓ compared to IV dosing and/or delay the time to Cₘₐₓ (tₘₐₓ) compared to IV dosing. The methods are expected to reduce or inhibit unwanted treatment effects, which include cytokine-driven toxicities (e.g., cytokine release syndrome (CRS)), infusion-related reactions (IRRs), macrophage activation syndrome (MAS), neurologic toxicities, severe tumor lysis syndrome (TLS), neutropenia, thrombocytopenia, and/or elevated liver enzymes. Therefore, the methods are useful for treating the subject while achieving a more favorable benefit-risk profile.

The invention provides methods useful for treating a subject having a cancer (e.g., multiple myeloma) that include subcutaneously administering to the subject a bispecific antibody that binds to FcRH5 and CD3 (i.e., an anti-FcRH5/anti-CD3 antibody), e.g., in a fractionated, dose-escalation dosing regimen, either as a monotherapy or in combination with one or more additional therapeutic agents (e.g., an IMiD (e.g., pomalidomide), an anti-CD38 antibody (e.g., daratumumab, MOR202, or isatuximab), a corticosteroid (e.g., dexamethasone or methylprednisolone), acetaminophen, paracetamol, diphenhydramine, or a combination thereof).

### A. Dosing Regimens: No Step-Up, Single Step-Up, and Double Step-Up Dosages

i. No Step-Up Dosing Regimens

In some aspects, the invention provides methods of treating a subject having a cancer (e.g., a multiple myeloma (MM)) comprising administering to the subject a bispecific antibody that binds to FcRH5 and CD3 in a dosing regimen that does not involve any step-up dosing.

In some aspects, the invention provides a method of treating a subject having an MM comprising subcutaneously administering to the subject a bispecific antibody that binds to FcRH5 and CD3 in a dosing regimen comprising at least a first dosing cycle, wherein the first dosing cycle comprises a first dose (C1D1) of the bispecific antibody, wherein the C1D1 is between about 10 mg to about 1000 mg (e.g., between about 10 mg to about 70 mg, about 15 mg to about 65 mg, about 20 mg to about 60 mg, about 25 mg to about 55 mg, about 30 mg to about 50 mg, about 35 mg to about 45 mg, about 15 mg to about 225 mg, about 20 mg to about 220 mg, about 25 mg to about 215 mg, about 30 mg to about 210 mg, about 35 mg to about 205 mg, about 40 mg to about 200 mg, about 45 mg to about 195 mg, about 50 mg to about 190 mg, about 55 mg to about 185 mg, about 60 mg to about 180 mg, about 65 mg to about 175 mg, about 70 mg to about 170 mg, about 75 mg to about 165 mg, about 80 mg to about 160 mg, about 85 mg to about 155 mg, about 90 mg to about 150 mg, about 95 mg to about 145 mg, about 100 mg to about 140 mg, about 105 mg to about 135 mg, about 110 mg to about 130 mg, about 115 mg to about 125 mg, about 10 mg to about 50 mg, about 25 mg to about 75 mg, about 50 mg to about 100 mg, about 75 mg to about 125 mg, about 100 mg to about 150 mg, about 125 mg to about 175 mg, about 150 mg to about 200 mg, about 175 mg to about 225 mg, about 200 mg to about 250 mg, about 225 mg to about 275 mg, about 250 mg to about 300 mg, about 275 mg to about 325 mg, about 300 mg to about 350 mg, about 325 mg to about 375 mg, about 350 mg to about 400 mg, about 375 mg to about 425 mg, about 400 mg to about 450 mg, about 425 mg to about 475 mg, about 450 mg to about 500 mg, about 475 mg to about 525 mg, about 500 mg to about 550 mg, about 525 mg to about 575 mg, about 550 mg to about 600 mg, about 575 mg to about 625 mg, about 600 mg to about 650 mg, about 625 mg to about 675 mg, about 650 mg to about 700 mg, about 675 mg to about 725 mg, about 700 mg to about 750 mg, about 725 mg to about 775 mg, about 750 mg to about 800 mg, about 775 mg to about 825 mg, about 800 mg to about 850 mg, about 825 mg to about 875 mg, about 850 mg to about 900 mg, about 875 mg to about 925 mg, about 900 mg to about 950 mg, about 925 mg to about 975 mg, or about 950 mg to about 1000 mg).

In some aspects, the C1D1 is between about 10 mg to about 200 mg (e.g., between about 10 mg to about 170 mg, about 11 mg to about 165 mg, about 12 mg to about 160 mg, about 13 mg to about 155 mg, about 14 mg to about 150 mg, about 15 mg to about 145 mg, about 16 mg to about 140 mg, about 17 mg to about 135 mg, about 18 mg to about 130 mg, about 19 mg to about 125 mg, about 20 mg to about 120 mg, about 21 mg to about 115 mg, about 22 mg to about 110 mg, about 23 mg to about 105 mg, about 24 mg to about 100 mg, about 25 mg to about 95 mg, about 26 mg to about 90 mg, about 27 mg to about 85 mg, about 28 mg to about 80 mg, about 29 mg to about 75 mg, about 30 mg to about 70 mg, about 31 mg to about 65 mg, about 32 mg to about 60 mg, about 33 mg to about 55 mg, about 34 mg to about 50 mg, about 35 mg to about 45 mg, about 40 mg to about 200 mg, about 45 mg to about 195 mg, about 50 mg to about 190 mg, about 55 mg to about 185 mg, about 60 mg to about 180 mg, about 65 mg to about 175 mg, about 70 mg to about 170 mg, about 75 mg to about 165 mg, about 80 mg to about 160 mg, about 85 mg to about 155 mg, about 90 mg to about 150 mg, about 95 mg to about 145 mg, about 100 mg to about 140 mg, about 105 mg to about 135 mg, about 110 mg to about 130 mg, about 115 mg to about 125 mg, about 10 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, about 90 mg to about 100 mg, about 100 mg to about 110 mg, about 110 mg to about 120 mg, about 120 mg to about 130 mg, about 130 mg to about 140 mg, about 140 mg to about 150 mg, about 150 mg to about 160 mg, about 160 mg to about 170 mg, about 170 mg to about 180 mg, about 180 mg to about 190 mg, or about 190 mg to about 200 mg).

In some aspects, the C1D1 is between 10 mg to 1000 mg (e.g., between 10 mg to 70 mg, 15 mg to 65 mg, 20 mg to 60 mg, 25 mg to 55 mg, 30 mg to 50 mg, 35 mg to 45 mg, 15 mg to 225 mg, 20 mg to 220 mg, 25 mg to 215 mg, 30 mg to 210 mg, 35 mg to 205 mg, 40 mg to 200 mg, 45 mg to 195 mg, 50 mg to 190 mg, 55 mg to 185 mg, 60 mg to 180 mg, 65 mg to 175 mg, 70 mg and 170 mg, 75 mg and 165 mg, 80 mg and 160 mg, 85 mg and 155 mg, 90 mg and 150 mg, 95 mg and 145 mg, 100 mg and 140 mg, 105 mg and 135 mg, 110 mg and 130 mg, 115 mg and 125 mg, 10 mg to 50 mg, 25 mg to 75 mg, 50 mg to 100 mg, 75 mg to 125 mg, 100 mg to 150 mg, 125 mg to 175 mg, 150 mg to 200 mg, 175 mg to 225 mg, 200 mg to 250 mg, 225 mg to 275 mg, 250 mg to 300 mg, 275 mg to 325 mg, 300 mg to 350 mg, 325 mg to 375 mg, 350 mg to 400 mg, 375 mg to 425 mg, 400 mg to 450 mg, 425 mg to 475 mg, 450 mg to 500 mg, 475 mg to 525 mg, 500 mg to 550 mg, 525 mg to 575 mg, 550 mg to 600 mg, 575 mg to 625 mg, 600 mg to 650 mg, 625 mg to 675 mg, 650 mg to 700 mg, 675 mg to 725 mg, 700 mg to 750 mg, 725 mg to 775 mg, 750 mg to 800 mg, 775 mg to 825 mg, 800 mg to 850 mg, 825 mg to 875 mg, 850 mg to 900 mg, 875 mg to 925 mg, 900 mg to 950 mg, 925 mg to 975 mg, or 950 mg to 1000 mg).

In some aspects, the C1D1 is between 10 mg to 200 mg (e.g., between 10 mg to 170 mg, 11 mg to 165 mg, 12 mg to 160 mg, 13 mg to 155 mg, 14 mg to 150 mg, 15 mg to 145 mg, 16 mg to 140 mg, 17 mg to 135 mg, 18 mg to 130 mg, 19 mg to 125 mg, 20 mg to 120 mg, 21 mg to 115 mg, 22 mg to 110 mg, 23 mg to 105 mg, 24 mg to 100 mg, 25 mg to 95 mg, 26 mg to 90 mg, 27 mg to 85 mg, 28 mg to 80 mg, 29 mg to 75 mg, 30 mg to 70 mg, 31 mg to 65 mg, 32 mg to 60 mg, 33 mg to 55 mg, 34 mg to 50 mg, 35 mg to 45 mg, 40 mg to 200 mg, 45 mg to 195 mg, 50 mg to 190 mg, 55 mg to 185 mg, 60 mg to 180 mg, 65 mg to 175 mg, 70 mg to 170 mg, 75 mg to 165 mg, 80 mg to 160 mg, 85 mg to 155 mg, 90 mg to 150 mg, 95 mg to 145 mg, 100 mg to 140 mg, 105 mg to 135 mg, 110 mg to 130 mg, 115 mg to 125 mg, 10 mg to 20 mg, 20 mg to 30 mg, 30 mg to 40 mg, 40 mg to 50 mg, 50 mg to 60 mg, 60 mg to 70 mg, 70 mg to 80 mg, 80 mg to 90 mg, 90 mg to 100 mg, 100 mg to 110 mg, 110 mg to 120 mg, 120 mg to 130 mg, 130 mg to 140 mg, 140 mg to 150 mg, 150 mg to 160 mg, 160 mg to 170 mg, 170 mg to 180 mg, 180 mg to 190 mg, or 190 mg to 200 mg).

In some aspects, the invention provides a method of treating a subject having a cancer (e.g., an MM) comprising subcutaneously administering to the subject a bispecific antibody that binds to FcRH5 and CD3 in a dosing regimen comprising at least a first dosing cycle, wherein the first dosing cycle comprises a first dose (C1D1) of the bispecific antibody between about 10 mg to about 1000 mg (e.g., between about 10 mg to about 70 mg, about 15 mg to about 65 mg, about 20 mg to about 60 mg, about 25 mg to about 55 mg, about 30 mg to about 50 mg, about 35 mg to about 45 mg, about 15 mg to about 225 mg, about 20 mg to about 220 mg, about 25 mg to about 215 mg, about 30 mg to about 210 mg, about 35 mg to about 205 mg, about 40 mg to about 200 mg, about 45 mg to about 195 mg, about 50 mg to about 190 mg, about 55 mg to about 185 mg, about 60 mg to about 180 mg, about 65 mg to about 175 mg, about 70 mg to about 170 mg, about 75 mg to about 165 mg, about 80 mg to about 160 mg, about 85 mg to about 155 mg, about 90 mg to about 150 mg, about 95 mg to about 145 mg, about 100 mg to about 140 mg, about 105 mg to about 135 mg, about 110 mg to about 130 mg, about 115 mg to about 125 mg, about 10 mg to about 50 mg, about 25 mg to about 75 mg, about 50 mg to about 100 mg, about 75 mg to about 125 mg, about 100 mg to about 150 mg, about 125 mg to about 175 mg, about 150 mg to about 200 mg, about 175 mg to about 225 mg, about 200 mg to about 250 mg, about 225 mg to about 275 mg, about 250 mg to about 300 mg, about 275 mg to about 325 mg, about 300 mg to about 350 mg, about 325 mg to about 375 mg, about 350 mg to about 400 mg, about 375 mg to about 425 mg, about 400 mg to about 450 mg, about 425 mg to about 475 mg, about 450 mg to about 500 mg, about 475 mg to about 525 mg, about 500 mg to about 550 mg, about 525 mg to about 575 mg, about 550 mg to about 600 mg, about 575 mg to about 625 mg, about 600 mg to about 650 mg, about 625 mg to about 675 mg, about 650 mg to about 700 mg, about 675 mg to about 725 mg, about 700 mg to about 750 mg, about 725 mg to about 775 mg, about 750 mg to about 800 mg, about 775 mg to about 825 mg, about 800 mg to about 850 mg, about 825 mg to about 875 mg, about 850 mg to about 900 mg, about 875 mg to about 925 mg, about 900 mg to about 950 mg, about 925 mg to about 975 mg, or about 950 mg to about 1000 mg).

In some aspects, the C1D1 is between about 10 mg to about 200 mg (e.g., between about 10 mg to about 200 mg (e.g., between about 10 mg to about 170 mg, about 11 mg to about 165 mg, about 12 mg to about 160 mg, about 13 mg to about 155 mg, about 14 mg to about 150 mg, about 15 mg to about 145 mg, about 16 mg to about 140 mg, about 17 mg to about 135 mg, about 18 mg to about 130 mg, about 19 mg to about 125 mg, about 20 mg to about 120 mg, about 21 mg to about 115 mg, about 22 mg to about 110 mg, about 23 mg to about 105 mg, about 24 mg to about 100 mg, about 25 mg to about 95 mg, about 26 mg to about 90 mg, about 27 mg to about 85 mg, about 28 mg to about 80 mg, about 29 mg to about 75 mg, about 30 mg to about 70 mg, about 31 mg to about 65 mg, about 32 mg to about 60 mg, about 33 mg to about 55 mg, about 34 mg to about 50 mg, about 35 mg to about 45 mg, about 40 mg to about 200 mg, about 45 mg to about 195 mg, about 50 mg to about 190 mg, about 55 mg to about 185 mg, about 60 mg to about 180 mg, about 65 mg to about 175 mg, about 70 mg to about 170 mg, about 75 mg to about 165 mg, about 80 mg to about 160 mg, about 85 mg to about 155 mg, about 90 mg to about 150 mg, about 95 mg to about 145 mg, about 100 mg to about 140 mg, about 105 mg to about 135 mg, about 110 mg to about 130 mg, about 115 mg to about 125 mg, about 10 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, about 90 mg to about 100 mg, about 100 mg to about 110 mg, about 110 mg to about 120 mg, about 120 mg to about 130 mg, about 130 mg to about 140 mg, about 140 mg to about 150 mg, about 150 mg to about 160 mg, about 160 mg to about 170 mg, about 170 mg to about 180 mg, about 180 mg to about 190 mg, or about 190 mg to about 200 mg).

In some aspects, the C1D1 is between 10 mg to 1000 mg (e.g., between 10 mg to 70 mg, 15 mg to 65 mg, 20 mg to 60 mg, 25 mg to 55 mg, 30 mg to 50 mg, 35 mg to 45 mg, 15 mg to 225 mg, 20 mg to 220 mg, 25 mg to 215 mg, 30 mg to 210 mg, 35 mg to 205 mg, 40 mg to 200 mg, 45 mg to 195 mg, 50 mg to 190 mg, 55 mg to 185 mg, 60 mg to 180 mg, 65 mg to 175 mg, 70 mg and 170 mg, 75 mg and 165 mg, 80 mg and 160 mg, 85 mg and 155 mg, 90 mg and 150 mg, 95 mg and 145 mg, 100 mg and 140 mg, 105 mg and 135 mg, 110 mg and 130 mg, 115 mg and 125 mg, 10 mg to 50 mg, 25 mg to 75 mg, 50 mg to 100 mg, 75 mg to 125 mg, 100 mg to 150 mg, 125 mg to 175 mg, 150 mg to 200 mg, 175 mg to 225 mg, 200 mg to 250 mg, 225 mg to 275 mg, 250 mg to 300 mg, 275 mg to 325 mg, 300 mg to 350 mg, 325 mg to 375 mg, 350 mg to 400 mg, 375 mg to 425 mg, 400 mg to 450 mg, 425 mg to 475 mg, 450 mg to 500 mg, 475 mg to 525 mg, 500 mg to 550 mg, 525 mg to 575 mg, 550 mg to 600 mg, 575 mg to 625 mg, 600 mg to 650 mg, 625 mg to 675 mg, 650 mg to 700 mg, 675 mg to 725 mg, 700 mg to 750 mg, 725 mg to 775 mg, 750 mg to 800 mg, 775 mg to 825 mg, 800 mg to 850 mg, 825 mg to 875 mg, 850 mg to 900 mg, 875 mg to 925 mg, 900 mg to 950 mg, 925 mg to 975 mg, or 950 mg to 1000 mg).

In some aspects, the C1D1 is between 10 mg to 200 mg (e.g., between 10 mg to 170 mg, 11 mg to 165 mg, 12 mg to 160 mg, 13 mg to 155 mg, 14 mg to 150 mg, 15 mg to 145 mg, 16 mg to 140 mg, 17 mg to 135 mg, 18 mg to 130 mg, 19 mg to 125 mg, 20 mg to 120 mg, 21 mg to 115 mg, 22 mg to 110 mg, 23 mg to 105 mg, 24 mg to 100 mg, 25 mg to 95 mg, 26 mg to 90 mg, 27 mg to 85 mg, 28 mg to 80 mg, 29 mg to 75 mg, 30 mg to 70 mg, 31 mg to 65 mg, 32 mg to 60 mg, 33 mg to 55 mg, 34 mg to 50 mg, 35 mg to 45 mg, 40 mg to 200 mg, 45 mg to 195 mg, 50 mg to 190 mg, 55 mg to 185 mg, 60 mg to 180 mg, 65 mg to 175 mg, 70 mg to 170 mg, 75 mg to 165 mg, 80 mg to 160 mg, 85 mg to 155 mg, 90 mg to 150 mg, 95 mg to 145 mg, 100 mg to 140 mg, 105 mg to 135 mg, 110 mg to 130 mg, 115 mg to 125 mg, 10 mg to 20 mg, 20 mg to 30 mg, 30 mg to 40 mg, 40 mg to 50 mg, 50 mg to 60 mg, 60 mg to 70 mg, 70 mg to 80 mg, 80 mg to 90 mg, 90 mg to 100 mg, 100 mg to 110 mg, 110 mg to 120 mg, 120 mg to 130 mg, 130 mg to 140 mg, 140 mg to 150 mg, 150 mg to 160 mg, 160 mg to 170 mg, 170 mg to 180 mg, 180 mg to 190 mg, or 190 mg to 200 mg).

In some aspects, the C1D1 is about 10 mg. In some aspects, the C1D1 is about 15 mg. In some aspects, the C1D1 is about 20 mg. In some aspects, the C1D1 is about 25 mg. In some aspects, the C1D1 is about 30 mg. In some aspects, the C1D1 is about 35 mg. In some aspects, the C1D1 is about 40 mg. In some aspects, the C1D1 is about 45 mg. In some aspects, the C1D1 is about 50 mg. In some aspects, the C1D1 is about 55 mg. In some aspects, the C1D1 is about 60 mg. In some aspects, the C1D1 is about 65 mg. In some aspects, the C1D1 is about 70 mg. In some aspects, the C1D1 is about 75 mg. In some aspects, the C1D1 is about 80 mg. In some aspects, the C1D1 is about 85 mg. In some aspects, the C1D1 is about 90 mg. In some aspects, the C1D1 is about 95 mg. In some aspects, the C1D1 is about 100 mg. In some aspects, the C1D1 is about 105 mg. In some aspects, the C1D1 is about 110 mg. In some aspects, the C1D1 is about 115 mg. In some aspects, the C1D1 is about 120 mg. In some aspects, the C1D1 is about 125 mg. In some aspects, the C1D1 is about 130 mg. In some aspects, the C1D1 is about 135 mg. In some aspects, the C1D1 is about 140 mg. In some aspects, the C1D1 is about 145 mg. In some aspects, the C1D1 is about 150 mg. In some aspects, the C1D1 is about 155 mg. In some aspects, the C1D1 is about 160 mg. In some aspects, the C1D1 is about 165 mg. In some aspects, the C1D1 is about 170 mg. In some aspects, the C1D1 is about 175 mg. In some aspects, the C1D1 is about 180 mg. In some aspects, the C1D1 is about 185 mg. In some aspects, the C1D1 is about 190 mg. In some aspects, the C1D1 is about 195 mg. In some aspects, the C1D1 is about 200 mg. In some aspects, the C1D1 is about 205 mg. In some aspects, the C1D1 is about 210 mg. In some aspects, the C1D1 is about 215 mg. In some aspects, the C1D1 is about 220 mg. In some aspects, the C1D1 is about 225 mg. In some aspects, the C1D1 is about 230 mg. In some aspects, the C1D1 is about 235 mg. In some aspects, the C1D1 is about 240 mg. In some aspects, the C1D1 is about 245 mg. In some aspects, the C1D1 is about 250 mg. In some aspects, the C1D1 is about 255 mg. In some aspects, the C1D1 is about 260 mg. In some aspects, the C1D1 is about 265 mg. In some aspects, the C1D1 is about 270 mg. In some aspects, the C1D1 is about 275 mg. In some aspects, the C1D1 is about 280 mg. In some aspects, the C1D1 is about 285 mg. In some aspects, the C1D1 is about 290 mg. In some aspects, the C1D1 is about 295 mg. In some aspects, the C1D1 is about 300 mg. In some aspects, the C1D1 is about 305 mg. In some aspects, the C1D1 is about 310 mg. In some aspects, the C1D1 is about 315 mg. In some aspects, the C1D1 is about 320 mg. In some aspects, the C1D1 is about 325 mg. In some aspects, the C1D1 is about 330 mg. In some aspects, the C1D1 is about 335 mg. In some aspects, the C1D1 is about 340 mg. In some aspects, the C1D1 is about 345 mg. In some aspects, the C1D1 is about 350 mg. In some aspects, the C1D1 is about 355 mg. In some aspects, the C1D1 is about 360 mg. In some aspects, the C1D1 is about 365 mg. In some aspects, the C1D1 is about 370 mg. In some aspects, the C1D1 is about 375 mg. In some aspects, the C1D1 is about 380 mg. In some aspects, the C1D1 is about 385 mg. In some aspects, the C1D1 is about 390 mg. In some aspects, the C1D1 is about 395 mg. In some aspects, the C1D1 is about 400 mg. In some aspects, the C1D1 is about 405 mg. In some aspects, the C1D1 is about 410 mg. In some aspects, the C1D1 is about 415 mg. In some aspects, the C1D1 is about 420 mg. In some aspects, the C1D1 is about 425 mg. In some aspects, the C1D1 is about 430 mg. In some aspects, the C1D1 is about 435 mg. In some aspects, the C1D1 is about 440 mg. In some aspects, the C1D1 is about 445 mg. In some aspects, the C1D1 is about 450 mg. In some aspects, the C1D1 is about 455 mg. In some aspects, the C1D1 is about 460 mg. In some aspects, the C1D1 is about 465 mg. In some aspects, the C1D1 is about 470 mg. In some aspects, the C1D1 is about 475 mg. In some aspects, the C1D1 is about 480 mg. In some aspects, the C1D1 is about 485 mg. In some aspects, the C1D1 is about 490 mg. In some aspects, the C1D1 is about 495 mg. In some aspects, the C1D1 is about 500 mg. In some aspects, the C1D1 is about 505 mg. In some aspects, the C1D1 is about 510 mg. In some aspects, the C1D1 is about 515 mg. In some aspects, the C1D1 is about 520 mg. In some aspects, the C1D1 is about 525 mg. In some aspects, the C1D1 is about 530 mg. In some aspects, the C1D1 is about 535 mg. In some aspects, the C1D1 is about 540 mg. In some aspects, the C1D1 is about 545 mg. In some aspects, the C1D1 is about 550 mg. In some aspects, the C1D1 is about 555 mg. In some aspects, the C1D1 is about 560 mg. In some aspects, the C1D1 is about 565 mg. In some aspects, the C1D1 is about 570 mg. In some aspects, the C1D1 is about 575 mg. In some aspects, the C1D1 is about 580 mg. In some aspects, the C1D1 is about 585 mg. In some aspects, the C1D1 is about 590 mg. In some aspects, the C1D1 is about 595 mg. In some aspects, the C1D1 is about 600 mg. In some aspects, the C1D1 is about 605 mg. In some aspects, the C1D1 is about 610 mg. In some aspects, the C1D1 is about 615 mg. In some aspects, the C1D1 is about 620 mg. In some aspects, the C1D1 is about 625 mg. In some aspects, the C1D1 is about 630 mg. In some aspects, the C1D1 is about 635 mg. In some aspects, the C1D1 is about 640 mg. In some aspects, the C1D1 is about 645 mg. In some aspects, the C1D1 is about 650 mg. In some aspects, the C1D1 is about 655 mg. In some aspects, the C1D1 is about 660 mg. In some aspects, the C1D1 is about 665 mg. In some aspects, the C1D1 is about 670 mg. In some aspects, the C1D1 is about 675 mg. In some aspects, the C1D1 is about 680 mg. In some aspects, the C1D1 is about 685 mg. In some aspects, the C1D1 is about 690 mg. In some aspects, the C1D1 is about 695 mg. In some aspects, the C1D1 is about 700 mg. In some aspects, the C1D1 is about 705 mg. In some aspects, the C1D1 is about 710 mg. In some aspects, the C1D1 is about 715 mg. In some aspects, the C1D1 is about 720 mg. In some aspects, the C1D1 is about 725 mg. In some aspects, the C1D1 is about 730 mg. In some aspects, the C1D1 is about 735 mg. In some aspects, the C1D1 is about 740 mg. In some aspects, the C1D1 is about 745 mg. In some aspects, the C1D1 is about 750 mg. In some aspects, the C1D1 is about 755 mg. In some aspects, the C1D1 is about 760 mg. In some aspects, the C1D1 is about 765 mg. In some aspects, the C1D1 is about 770 mg. In some aspects, the C1D1 is about 775 mg. In some aspects, the C1D1 is about 780 mg. In some aspects, the C1D1 is about 785 mg. In some aspects, the C1D1 is about 790 mg. In some aspects, the C1D1 is about 795 mg. In some aspects, the C1D1 is about 800 mg. In some aspects, the C1D1 is about 805 mg. In some aspects, the C1D1 is about 810 mg. In some aspects, the C1D1 is about 815 mg. In some aspects, the C1D1 is about 820 mg. In some aspects, the C1D1 is about 825 mg. In some aspects, the C1D1 is about 830 mg. In some aspects, the C1D1 is about 835 mg. In some aspects, the C1D1 is about 840 mg. In some aspects, the C1D1 is about 845 mg. In some aspects, the C1D1 is about 850 mg. In some aspects, the C1D1 is about 855 mg. In some aspects, the C1D1 is about 860 mg. In some aspects, the C1D1 is about 865 mg. In some aspects, the C1D1 is about 870 mg. In some aspects, the C1D1 is about 875 mg. In some aspects, the C1D1 is about 880 mg. In some aspects, the C1D1 is about 885 mg. In some aspects, the C1D1 is about 890 mg. In some aspects, the C1D1 is about 895 mg. In some aspects, the C1D1 is about 900 mg. In some aspects, the C1D1 is about 905 mg. In some aspects, the C1D1 is about 910 mg. In some aspects, the C1D1 is about 915 mg. In some aspects, the C1D1 is about 920 mg. In some aspects, the C1D1 is about 925 mg. In some aspects, the C1D1 is about 930 mg. In some aspects, the C1D1 is about 935 mg. In some aspects, the C1D1 is about 940 mg. In some aspects, the C1D1 is about 945 mg. In some aspects, the C1D1 is about 950 mg. In some aspects, the C1D1 is about 955 mg. In some aspects, the C1D1 is about 960 mg. In some aspects, the C1D1 is about 965 mg. In some aspects, the C1D1 is about 970 mg. In some aspects, the C1D1 is about 975 mg. In some aspects, the C1D1 is about 980 mg. In some aspects, the C1D1 is about 985 mg. In some aspects, the C1D1 is about 990 mg. In some aspects, the C1D1 is about 995 mg. In some aspects, the C1D1 is about 1000 mg.

In some aspects, the C1D1 is 10 mg. In some aspects, the C1D1 is 15 mg. In some aspects, the C1D1 is 20 mg. In some aspects, the C1D1 is 25 mg. In some aspects, the C1D1 is 30 mg. In some aspects, the C1D1 is 35 mg. In some aspects, the C1D1 is 40 mg. In some aspects, the C1D1 is 45 mg. In some aspects, the C1D1 is 50 mg. In some aspects, the C1D1 is 55 mg. In some aspects, the C1D1 is 60 mg. In some aspects, the C1D1 is 65 mg. In some aspects, the C1D1 is 70 mg. In some aspects, the C1D1 is 75 mg. In some aspects, the C1D1 is 80 mg. In some aspects, the C1D1 is 85 mg. In some aspects, the C1D1 is 90 mg. In some aspects, the C1D1 is 95 mg. In some aspects, the C1D1 is 100 mg. In some aspects, the C1D1 is 105 mg. In some aspects, the C1D1 is 110 mg. In some aspects, the C1D1 is 115 mg. In some aspects, the C1D1 is 120 mg. In some aspects, the C1D1 is 125 mg. In some aspects, the C1D1 is 130 mg. In some aspects, the C1D1 is 135 mg. In some aspects, the C1D1 is 140 mg. In some aspects, the C1D1 is 145 mg. In some aspects, the C1D1 is 150 mg. In some aspects, the C1D1 is 155 mg. In some aspects, the C1D1 is 160 mg. In some aspects, the C1D1 is 165 mg. In some aspects, the C1D1 is 170 mg. In some aspects, the C1D1 is 175 mg. In some aspects, the C1D1 is 180 mg. In some aspects, the C1D1 is 185 mg. In some aspects, the C1D1 is 190 mg. In some aspects, the C1D1 is 195 mg. In some aspects, the C1D1 is 200 mg. In some aspects, the C1D1 is 205 mg. In some aspects, the C1D1 is 210 mg. In some aspects, the C1D1 is 215 mg. In some aspects, the C1D1 is 220 mg. In some aspects, the C1D1 is 225 mg. In some aspects, the C1D1 is 230 mg. In some aspects, the C1D1 is 235 mg. In some aspects, the C1D1 is 240 mg. In some aspects, the C1D1 is 245 mg. In some aspects, the C1D1 is 250 mg. In some aspects, the C1D1 is 255 mg. In some aspects, the C1D1 is 260 mg. In some aspects, the C1D1 is 265 mg. In some aspects, the C1D1 is 270 mg. In some aspects, the C1D1 is 275 mg. In some aspects, the C1D1 is 280 mg. In some aspects, the C1D1 is 285 mg. In some aspects, the C1D1 is 290 mg. In some aspects, the C1D1 is 295 mg. In some aspects, the C1D1 is 300 mg. In some aspects, the C1D1 is 305 mg. In some aspects, the C1D1 is 310 mg. In some aspects, the C1D1 is 315 mg. In some aspects, the C1D1 is 320 mg. In some aspects, the C1D1 is 325 mg. In some aspects, the C1D1 is 330 mg. In some aspects, the C1D1 is 335 mg. In some aspects, the C1D1 is 340 mg. In some aspects, the C1D1 is 345 mg. In some aspects, the C1D1 is 350 mg. In some aspects, the C1D1 is 355 mg. In some aspects, the C1D1 is 360 mg. In some aspects, the C1D1 is 365 mg. In some aspects, the C1D1 is 370 mg. In some aspects, the C1D1 is 375 mg. In some aspects, the C1D1 is 380 mg. In some aspects, the C1D1 is 385 mg. In some aspects, the C1D1 is 390 mg. In some aspects, the C1D1 is 395 mg. In some aspects, the C1D1 is 400 mg. In some aspects, the C1D1 is 405 mg. In some aspects, the C1D1 is 410 mg. In some aspects, the C1D1 is 415 mg. In some aspects, the C1D1 is 420 mg. In some aspects, the C1D1 is 425 mg. In some aspects, the C1D1 is 430 mg. In some aspects, the C1D1 is 435 mg. In some aspects, the C1D1 is 440 mg. In some aspects, the C1D1 is 445 mg. In some aspects, the C1D1 is 450 mg. In some aspects, the C1D1 is 455 mg. In some aspects, the C1D1 is 460 mg. In some aspects, the C1D1 is 465 mg. In some aspects, the C1D1 is 470 mg. In some aspects, the C1D1 is 475 mg. In some aspects, the C1D1 is 480 mg. In some aspects, the C1D1 is 485 mg. In some aspects, the C1D1 is 490 mg. In some aspects, the C1D1 is 495 mg. In some aspects, the C1D1 is 500 mg. In some aspects, the C1D1 is 505 mg. In some aspects, the C1D1 is 510 mg. In some aspects, the C1D1 is 515 mg. In some aspects, the C1D1 is 520 mg. In some aspects, the C1D1 is 525 mg. In some aspects, the C1D1 is 530 mg. In some aspects, the C1D1 is 535 mg. In some aspects, the C1D1 is 540 mg. In some aspects, the C1D1 is 545 mg. In some aspects, the C1D1 is 550 mg. In some aspects, the C1D1 is 555 mg. In some aspects, the C1D1 is 560 mg. In some aspects, the C1D1 is 565 mg. In some aspects, the C1D1 is 570 mg. In some aspects, the C1D1 is 575 mg. In some aspects, the C1D1 is 580 mg. In some aspects, the C1D1 is 585 mg. In some aspects, the C1D1 is 590 mg. In some aspects, the C1D1 is 595 mg. In some aspects, the C1D1 is 600 mg. In some aspects, the C1D1 is 605 mg. In some aspects, the C1D1 is 610 mg. In some aspects, the C1D1 is 615 mg. In some aspects, the C1D1 is 620 mg. In some aspects, the C1D1 is 625 mg. In some aspects, the C1D1 is 630 mg. In some aspects, the C1D1 is 635 mg. In some aspects, the C1D1 is 640 mg. In some aspects, the C1D1 is 645 mg. In some aspects, the C1D1 is 650 mg. In some aspects, the C1D1 is 655 mg. In some aspects, the C1D1 is 660 mg. In some aspects, the C1D1 is 665 mg. In some aspects, the C1D1 is 670 mg. In some aspects, the C1D1 is 675 mg. In some aspects, the C1D1 is 680 mg. In some aspects, the C1D1 is 685 mg. In some aspects, the C1D1 is 690 mg. In some aspects, the C1D1 is 695 mg. In some aspects, the C1D1 is 700 mg. In some aspects, the C1D1 is 705 mg. In some aspects, the C1D1 is 710 mg. In some aspects, the C1D1 is 715 mg. In some aspects, the C1D1 is 720 mg. In some aspects, the C1D1 is 725 mg. In some aspects, the C1D1 is 730 mg. In some aspects, the C1D1 is 735 mg. In some aspects, the C1D1 is 740 mg. In some aspects, the C1D1 is 745 mg. In some aspects, the C1D1 is 750 mg. In some aspects, the C1D1 is 755 mg. In some aspects, the C1D1 is 760 mg. In some aspects, the C1D1 is 765 mg. In some aspects, the C1D1 is 770 mg. In some aspects, the C1D1 is 775 mg. In some aspects, the C1D1 is 780 mg. In some aspects, the C1D1 is 785 mg. In some aspects, the C1D1 is 790 mg. In some aspects, the C1D1 is 795 mg. In some aspects, the C1D1 is 800 mg. In some aspects, the C1D1 is 805 mg. In some aspects, the C1D1 is 810 mg. In some aspects, the C1D1 is 815 mg. In some aspects, the C1D1 is 820 mg. In some aspects, the C1D1 is 825 mg. In some aspects, the C1D1 is 830 mg. In some aspects, the C1D1 is 835 mg. In some aspects, the C1D1 is 840 mg. In some aspects, the C1D1 is 845 mg. In some aspects, the C1D1 is 850 mg. In some aspects, the C1D1 is 855 mg. In some aspects, the C1D1 is 860 mg. In some aspects, the C1D1 is 865 mg. In some aspects, the C1D1 is 870 mg. In some aspects, the C1D1 is 875 mg. In some aspects, the C1D1 is 880 mg. In some aspects, the C1D1 is 885 mg. In some aspects, the C1D1 is 890 mg. In some aspects, the C1D1 is 895 mg. In some aspects, the C1D1 is 900 mg. In some aspects, the C1D1 is 905 mg. In some aspects, the C1D1 is 910 mg. In some aspects, the C1D1 is 915 mg. In some aspects, the C1D1 is 920 mg. In some aspects, the C1D1 is 925 mg. In some aspects, the C1D1 is 930 mg. In some aspects, the C1D1 is 935 mg. In some aspects, the C1D1 is 940 mg. In some aspects, the C1D1 is 945 mg. In some aspects, the C1D1 is 950 mg. In some aspects, the C1D1 is 955 mg. In some aspects, the C1D1 is 960 mg. In some aspects, the C1D1 is 965 mg. In some aspects, the C1D1 is 970 mg. In some aspects, the C1D1 is 975 mg. In some aspects, the C1D1 is 980 mg. In some aspects, the C1D1 is 985 mg. In some aspects, the C1D1 is 990 mg. In some aspects, the C1D1 is 995 mg. In some aspects, the C1D1 is 1000 mg.

ii. Single Step-Up Dosing Regimens

In some aspects, the invention provides methods of treating a subject having a cancer (e.g., an MM) comprising subcutaneously administering to the subject a bispecific antibody that binds to FcRH5 and CD3 in a single step-up dosing regimen.

In some aspects, the invention provides a method of treating a subject having an MM comprising subcutaneously administering to the subject a bispecific antibody that binds to FcRH5 and CD3 in a dosing regimen comprising at least a first dosing cycle, wherein the first dosing cycle comprises a first dose (C1D1) of the bispecific antibody and a second dose (C1D2) of the bispecific antibody, wherein the C1D1 is between about 0.1 mg to about 50 mg (e.g., between about 0.1 mg to about 9.5 mg, about 0.2 mg to about 9 mg, about 0.3 mg to about 8.5 mg, about 0.4 mg to about 8 mg, about 0.5 mg to about 7.5 mg, about 0.6 mg to about 7 mg, about 0.7 mg to about 6.5 mg, about 0.8 mg to about 6 mg, about 0.9 mg to about 5.5 mg, about 1 mg to about 5 mg, about 1.1 mg to about 4.5 mg, about 1.2 mg to about 4 mg, about 1.3 mg to about 3.5 mg, about 1.4 mg to about 3 mg, about 1.5 mg to about 2.5 mg, about 1 mg to about 3 mg, about 0.2 mg to about 35 mg, about 0.3 mg to about 30 mg, about 0.4 mg to about 29 mg, about 0.5 mg to about 28 mg, about 1 mg to about 27 mg, about 1.5 mg to about 26 mg, about 2 mg to about 25 mg, about 2.5 mg to about 24 mg, about 3 mg to about 23 mg, about 3.5 mg to about 22 mg, about 4 mg to about 21 mg, about 4.5 mg to about 20 mg, about 5 mg to about 19 mg, about 5.5 mg to about 18 mg, about 6 mg to about 17 mg, about 6.5 mg to about 16 mg, about 7 mg to about 15 mg, about 7.5 mg to about 14 mg, about 8 mg to about 13 mg, about 8 mg to about 12 mg, about 8.5 mg to about 12 mg, about 9 mg to about 11 mg, about 0.1 mg to about 1 mg, about 0.2 mg to about 1.5 mg, about 0.3 mg to about 2 mg, about 0.4 mg to about 2.5 mg, about 0.5 mg to about 3 mg, about 0.6 mg to about 3.5 mg, about 0.7 mg to about 4 mg, about 0.8 mg to about 4.5 mg, about 0.9 mg to about 5 mg, about 1 mg to about 6 mg, about 1.5 mg to about 6.5 mg, about 2 mg to about 7 mg, about 2.5 mg to about 7.5 mg, about 3 mg to about 8 mg, about 3.5 mg to about 8.5 mg, about 4 mg to about 9 mg, about 4.5 mg to about 9.5 mg, about 5 mg to about 10 mg, about 5.5 mg to about 10.5 mg, about 6 mg to about 11 mg, about 6.5 mg to about 11.5 mg, about 7 mg to about 12 mg, about 7.5 mg to about 12.5 mg, about 8 mg to about 13 mg, about 8.5 mg to about 13.5 mg, about 9 mg to about 14 mg, about 9.5 mg to about 14.5 mg, about 10 mg to about 15 mg, about 11 mg to about 16 mg, about 12 mg to about 17 mg, about 13 mg to about 18 mg, about 14 mg to about 19 mg, about 15 mg to about 20 mg, about 16 mg to about 21 mg, about 17 mg to about 22 mg, about 18 mg to about 23 mg, about 19 mg to about 24 mg, about 20 mg to about 25 mg, about 21 mg to about 26 mg, about 22 mg to about 27 mg, about 23 mg to about 28 mg, about 24 mg to about 29 mg, about 25 mg to about 30 mg, about 26 mg to about 31 mg, about 27 mg to about 32 mg, about 28 mg to about 33 mg, about 29 mg to about 34 mg, about 30 mg to about 35 mg, about 31 mg to about 36 mg, about 32 mg to about 37 mg, about 33 mg to about 38 mg, about 34 mg to about 39 mg, about 35 mg to about 40 mg, about 36 mg to about 41 mg, about 37 mg to about 42 mg, about 38 mg to about 43 mg, about 39 mg to about 44 mg, about 40 mg to about 45 mg, about 41 mg to about 46 mg, about 42 mg to about 47 mg, about 43 mg to about 48 mg, about 44 mg to about 49 mg, about 45 mg to about 50 mg) and the C1D2 is between about 10 mg to about 1000 mg (e.g., between about 10 mg to about 70 mg, about 15 mg to about 65 mg, about 20 mg to about 60 mg, about 25 mg to about 55 mg, about 30 mg to about 50 mg, about 35 mg to about 45 mg, about 15 mg to about 225 mg, about 20 mg to about 220 mg, about 25 mg to about 215 mg, about 30 mg to about 210 mg, about 35 mg to about 205 mg, about 40 mg to about 200 mg, about 45 mg to about 195 mg, about 50 mg to about 190 mg, about 55 mg to about 185 mg, about 60 mg to about 180 mg, about 65 mg to about 175 mg, about 70 mg to about 170 mg, about 75 mg to about 165 mg, about 80 mg to about 160 mg, about 85 mg to about 155 mg, about 90 mg to about 150 mg, about 95 mg to about 145 mg, about 100 mg to about 140 mg, about 105 mg to about 135 mg, about 110 mg to about 130 mg, about 115 mg to about 125 mg, about 10 mg to about 50 mg, about 25 mg to about 75 mg, about 50 mg to about 100 mg, about 75 mg to about 125 mg, about 100 mg to about 150 mg, about 125 mg to about 175 mg, about 150 mg to about 200 mg, about 175 mg to about 225 mg, about 200 mg to about 250 mg, about 225 mg to about 275 mg, about 250 mg to about 300 mg, about 275 mg to about 325 mg, about 300 mg to about 350 mg, about 325 mg to about 375 mg, about 350 mg to about 400 mg, about 375 mg to about 425 mg, about 400 mg to about 450 mg, about 425 mg to about 475 mg, about 450 mg to about 500 mg, about 475 mg to about 525 mg, about 500 mg to about 550 mg, about 525 mg to about 575 mg, about 550 mg to about 600 mg, about 575 mg to about 625 mg, about 600 mg to about 650 mg, about 625 mg to about 675 mg, about 650 mg to about 700 mg, about 675 mg to about 725 mg, about 700 mg to about 750 mg, about 725 mg to about 775 mg, about 750 mg to about 800 mg, about 775 mg to about 825 mg, about 800 mg to about 850 mg, about 825 mg to about 875 mg, about 850 mg to about 900 mg, about 875 mg to about 925 mg, about 900 mg to about 950 mg, about 925 mg to about 975 mg, or about 950 mg to about 1000 mg).

In some aspects, the C1D2 is between about 10 mg to 200 mg (e.g., between 10 mg to about 170 mg, about 11 mg to about 165 mg, about 12 mg to about 160 mg, about 13 mg to about 155 mg, about 14 mg to about 150 mg, about 15 mg to about 145 mg, about 16 mg to about 140 mg, about 17 mg to about 135 mg, about 18 mg to about 130 mg, about 19 mg to about 125 mg, about 20 mg to about 120 mg, about 21 mg to about 115 mg, about 22 mg to about 110 mg, about 23 mg to about 105 mg, about 24 mg to about 100 mg, about 25 mg to about 95 mg, about 26 mg to about 90 mg, about 27 mg to about 85 mg, about 28 mg to about 80 mg, about 29 mg to about 75 mg, about 30 mg to about 70 mg, about 31 mg to about 65 mg, about 32 mg to about 60 mg, about 33 mg to about 55 mg, about 34 mg to about 50 mg, about 35 mg to about 45 mg, about 40 mg to about 200 mg, about 45 mg to about 195 mg, about 50 mg to about 190 mg, about 55 mg to about 185 mg, about 60 mg to about 180 mg, about 65 mg to about 175 mg, about 70 mg to about 170 mg, about 75 mg to about 165 mg, about 80 mg to about 160 mg, about 85 mg to about 155 mg, about 90 mg to about 150 mg, about 95 mg to about 145 mg, about 100 mg to about 140 mg, about 105 mg to about 135 mg, about 110 mg to about 130 mg, about 115 mg to about 125 mg, about 10 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, about 90 mg to about 100 mg, about 100 mg to about 110 mg, about 110 mg to about 120 mg, about 120 mg to about 130 mg, about 130 mg to about 140 mg, about 140 mg to about 150 mg, about 150 mg to about 160 mg, about 160 mg to about 170 mg, about 170 mg to about 180 mg, about 180 mg to about 190 mg, or about 190 mg to about 200 mg).

In some aspects, the C1D1 is between 0.1 mg to 50 mg (e.g., between 0.1 mg to 9.5 mg, 0.2 mg to 9 mg, 0.3 mg to 8.5 mg, 0.4 mg to 8 mg, 0.5 mg to 7.5 mg, 0.6 mg to 7 mg, 0.7 mg to 6.5 mg, 0.8 mg to 6 mg, 0.9 mg to 5.5 mg, 1 mg to 5 mg, 1.1 mg to 4.5 mg, 1.2 mg to 4 mg, 1.3 mg to 3.5 mg, 1.4 mg to 3 mg, 1.5 mg to 2.5 mg, 1 mg to 3 mg, 0.2 mg to 35 mg, 0.3 mg to 30 mg, 0.4 mg to 29 mg, 0.5 mg to 28 mg, 1 mg to 27 mg, 1.5 mg to 26 mg, 2 mg to 25 mg, 2.5 mg to 24 mg, 3 mg to 23 mg, 3.5 mg to 22 mg, 4 mg to 21 mg, 4.5 mg to 20 mg, 5 mg to 19 mg, 5.5 mg to 18 mg, 6 mg to 17 mg, 6.5 mg to 16 mg, 7 mg to 15 mg, 7.5 mg to 14 mg, 8 mg to 13 mg, 8 mg to 12 mg, 8.5 mg to 12 mg, 9 mg to 11 mg, 0.1 mg to 1 mg, 0.2 mg to 1.5 mg, 0.3 mg to 2 mg, 0.4 mg to 2.5 mg, 0.5 mg to 3 mg, 0.6 mg to 3.5 mg, 0.7 mg to 4 mg, 0.8 mg to 4.5 mg, 0.9 mg to 5 mg, 1 mg to 6 mg, 1.5 mg to 6.5 mg, 2 mg to 7 mg, 2.5 mg to 7.5 mg, 3 mg to 8 mg, 3.5 mg to 8.5 mg, 4 mg to 9 mg, 4.5 mg to 9.5 mg, 5 mg to 10 mg, 5.5 mg to 10.5 mg, 6 mg to 11 mg, 6.5 mg to 11.5 mg, 7 mg to 12 mg, 7.5 mg to 12.5 mg, 8 mg to 13 mg, 8.5 mg to 13.5 mg, 9 mg to 14 mg, 9.5 mg to 14.5 mg, 10 mg to 15 mg, 11 mg to 16 mg, 12 mg to 17 mg, 13 mg to 18 mg, 14 mg to 19 mg, 15 mg to 20 mg, 16 mg to 21 mg, 17 mg to 22 mg, 18 mg to 23 mg, 19 mg to 24 mg, 20 mg to 25 mg, 21 mg to 26 mg, 22 mg to 27 mg, 23 mg to 28 mg, 24 mg to 29 mg, 25 mg to 30 mg, 26 mg to 31 mg, 27 mg to 32 mg, 28 mg to 33 mg, 29 mg to 34 mg, 30 mg to 35 mg, 31 mg to 36 mg, 32 mg to 37 mg, 33 mg to 38 mg, 34 mg to 39 mg, 35 mg to 40 mg, 36 mg to 41 mg, 37 mg to 42 mg, 38 mg to 43 mg, 39 mg to 44 mg, 40 mg to 45 mg, 41 mg to 46 mg, 42 mg to 47 mg, 43 mg to 48 mg, 44 mg to 49 mg, 45 mg to 50 mg) and the C1D2 is between 10 mg to 1000 mg (e.g., between 10 mg to 70 mg, 15 mg to 65 mg, 20 mg to 60 mg, 25 mg to 55 mg, 30 mg to 50 mg, 35 mg to 45 mg, 15 mg to 225 mg, 20 mg to 220 mg, 25 mg to 215 mg, 30 mg to 210 mg, 35 mg to 205 mg, 40 mg to 200 mg, 45 mg to 195 mg, 50 mg to 190 mg, 55 mg to 185 mg, 60 mg to 180 mg, 65 mg to 175 mg, 70 mg and 170 mg, 75 mg and 165 mg, 80 mg and 160 mg, 85 mg and 155 mg, 90 mg and 150 mg, 95 mg and 145 mg, 100 mg and 140 mg, 105 mg and 135 mg, 110 mg and 130 mg, 115 mg and 125 mg, 10 mg to 50 mg, 25 mg to 75 mg, 50 mg to 100 mg, 75 mg to 125 mg, 100 mg to 150 mg, 125 mg to 175 mg, 150 mg to 200 mg, 175 mg to 225 mg, 200 mg to 250 mg, 225 mg to 275 mg, 250 mg to 300 mg, 275 mg to 325 mg, 300 mg to 350 mg, 325 mg to 375 mg, 350 mg to 400 mg, 375 mg to 425 mg, 400 mg to 450 mg, 425 mg to 475 mg, 450 mg to 500 mg, 475 mg to 525 mg, 500 mg to 550 mg, 525 mg to 575 mg, 550 mg to 600 mg, 575 mg to 625 mg, 600 mg to 650 mg, 625 mg to 675 mg, 650 mg to 700 mg, 675 mg to 725 mg, 700 mg to 750 mg, 725 mg to 775 mg, 750 mg to 800 mg, 775 mg to 825 mg, 800 mg to 850 mg, 825 mg to 875 mg, 850 mg to 900 mg, 875 mg to 925 mg, 900 mg to 950 mg, 925 mg to 975 mg, or 950 mg to 1000 mg).

In some aspects, the C1D2 is between 10 mg to 200 mg (e.g., between 10 mg to 170 mg, 11 mg to 165 mg, 12 mg to 160 mg, 13 mg to 155 mg, 14 mg to 150 mg, 15 mg to 145 mg, 16 mg to 140 mg, 17 mg to 135 mg, 18 mg to 130 mg, 19 mg to 125 mg, 20 mg to 120 mg, 21 mg to 115 mg, 22 mg to 110 mg, 23 mg to 105 mg, 24 mg to 100 mg, 25 mg to 95 mg, 26 mg to 90 mg, 27 mg to 85 mg, 28 mg to 80 mg, 29 mg to 75 mg, 30 mg to 70 mg, 31 mg to 65 mg, 32 mg to 60 mg, 33 mg to 55 mg, 34 mg to 50 mg, 35 mg to 45 mg, 40 mg to 200 mg, 45 mg to 195 mg, 50 mg to 190 mg, 55 mg to 185 mg, 60 mg to 180 mg, 65 mg to 175 mg, 70 mg to 170 mg, 75 mg to 165 mg, 80 mg to 160 mg, 85 mg to 155 mg, 90 mg to 150 mg, 95 mg to 145 mg, 100 mg to 140 mg, 105 mg to 135 mg, 110 mg to 130 mg, 115 mg to 125 mg, 10 mg to 20 mg, 20 mg to 30 mg, 30 mg to 40 mg, 40 mg to 50 mg, 50 mg to 60 mg, 60 mg to 70 mg, 70 mg to 80 mg, 80 mg to 90 mg, 90 mg to 100 mg, 100 mg to 110 mg, 110 mg to 120 mg, 120 mg to 130 mg, 130 mg to 140 mg, 140 mg to 150 mg, 150 mg to 160 mg, 160 mg to 170 mg, 170 mg to 180 mg, 180 mg to 190 mg, or 190 mg to 200 mg).

In some aspects, the invention provides a method of treating a subject having a cancer (e.g., an MM) comprising subcutaneously administering to the subject a bispecific antibody that binds to FcRH5 and CD3 in a dosing regimen comprising at least a first dosing cycle and a second dosing cycle, wherein the first dosing cycle comprises a first dose (C1D1; cycle 1, dose 1) of the bispecific antibody and a second dose (C1D2; cycle 1, dose, 2) of the bispecific antibody, wherein the C1D1 is less than the C1D2, and wherein the C1D1 is between about 0.1 mg to about 50 mg (e.g., between about 0.1 mg to about 9.5 mg, about 0.2 mg to about 9 mg, about 0.3 mg to about 8.5 mg, about 0.4 mg to about 8 mg, about 0.5 mg to about 7.5 mg, about 0.6 mg to about 7 mg, about 0.7 mg to about 6.5 mg, about 0.8 mg to about 6 mg, about 0.9 mg to about 5.5 mg, about 1 mg to about 5 mg, about 1.1 mg to about 4.5 mg, about 1.2 mg to about 4 mg, about 1.3 mg to about 3.5 mg, about 1.4 mg to about 3 mg, about 1.5 mg to about 2.5 mg, about 1 mg to about 3 mg, about 0.2 mg to about 35 mg, about 0.3 mg to about 30 mg, about 0.4 mg to about 29 mg, about 0.5 mg to about 28 mg, about 1 mg to about 27 mg, about 1.5 mg to about 26 mg, about 2 mg to about 25 mg, about 2.5 mg to about 24 mg, about 3 mg to about 23 mg, about 3.5 mg to about 22 mg, about 4 mg to about 21 mg, about 4.5 mg to about 20 mg, about 5 mg to about 19 mg, about 5.5 mg to about 18 mg, about 6 mg to about 17 mg, about 6.5 mg to about 16 mg, about 7 mg to about 15 mg, about 7.5 mg to about 14 mg, about 8 mg to about 13 mg, about 8 mg to about 12 mg, about 8.5 mg to about 12 mg, about 9 mg to about 11 mg, about 0.1 mg to about 1 mg, about 0.2 mg to about 1.5 mg, about 0.3 mg to about 2 mg, about 0.4 mg to about 2.5 mg, about 0.5 mg to about 3 mg, about 0.6 mg to about 3.5 mg, about 0.7 mg to about 4 mg, about 0.8 mg to about 4.5 mg, about 0.9 mg to about 5 mg, about 1 mg to about 6 mg, about 1.5 mg to about 6.5 mg, about 2 mg to about 7 mg, about 2.5 mg to about 7.5 mg, about 3 mg to about 8 mg, about 3.5 mg to about 8.5 mg, about 4 mg to about 9 mg, about 4.5 mg to about 9.5 mg, about 5 mg to about 10 mg, about 5.5 mg to about 10.5 mg, about 6 mg to about 11 mg, about 6.5 mg to about 11.5 mg, about 7 mg to about 12 mg, about 7.5 mg to about 12.5 mg, about 8 mg to about 13 mg, about 8.5 mg to about 13.5 mg, about 9 mg to about 14 mg, about 9.5 mg to about 14.5 mg, about 10 mg to about 15 mg, about 11 mg to about 16 mg, about 12 mg to about 17 mg, about 13 mg to about 18 mg, about 14 mg to about 19 mg, about 15 mg to about 20 mg, about 16 mg to about 21 mg, about 17 mg to about 22 mg, about 18 mg to about 23 mg, about 19 mg to about 24 mg, about 20 mg to about 25 mg, about 21 mg to about 26 mg, about 22 mg to about 27 mg, about 23 mg to about 28 mg, about 24 mg to about 29 mg, about 25 mg to about 30 mg, about 26 mg to about 31 mg, about 27 mg to about 32 mg, about 28 mg to about 33 mg, about 29 mg to about 34 mg, about 30 mg to about 35 mg, about 31 mg to about 36 mg, about 32 mg to about 37 mg, about 33 mg to about 38 mg, about 34 mg to about 39 mg, about 35 mg to about 40 mg, about 36 mg to about 41 mg, about 37 mg to about 42 mg, about 38 mg to about 43 mg, about 39 mg to about 44 mg, about 40 mg to about 45 mg, about 41 mg to about 46 mg, about 42 mg to about 47 mg, about 43 mg to about 48 mg, about 44 mg to about 49 mg, about 45 mg to about 50 mg) and the C1D2 is between about 10 mg to about 1000 mg (e.g., between about 10 mg to about 70 mg, about 15 mg to about 65 mg, about 20 mg to about 60 mg, about 25 mg to about 55 mg, about 30 mg to about 50 mg, about 35 mg to about 45 mg, about 15 mg to about 225 mg, about 20 mg to about 220 mg, about 25 mg to about 215 mg, about 30 mg to about 210 mg, about 35 mg to about 205 mg, about 40 mg to about 200 mg, about 45 mg to about 195 mg, about 50 mg to about 190 mg, about 55 mg to about 185 mg, about 60 mg to about 180 mg, about 65 mg to about 175 mg, about 70 mg to about 170 mg, about 75 mg to about 165 mg, about 80 mg to about 160 mg, about 85 mg to about 155 mg, about 90 mg to about 150 mg, about 95 mg to about 145 mg, about 100 mg to about 140 mg, about 105 mg to about 135 mg, about 110 mg to about 130 mg, about 115 mg to about 125 mg, about 10 mg to about 50 mg, about 25 mg to about 75 mg, about 50 mg to about 100 mg, about 75 mg to about 125 mg, about 100 mg to about 150 mg, about 125 mg to about 175 mg, about 150 mg to about 200 mg, about 175 mg to about 225 mg, about 200 mg to about 250 mg, about 225 mg to about 275 mg, about 250 mg to about 300 mg, about 275 mg to about 325 mg, about 300 mg to about 350 mg, about 325 mg to about 375 mg, about 350 mg to about 400 mg, about 375 mg to about 425 mg, about 400 mg to about 450 mg, about 425 mg to about 475 mg, about 450 mg to about 500 mg, about 475 mg to about 525 mg, about 500 mg to about 550 mg, about 525 mg to about 575 mg, about 550 mg to about 600 mg, about 575 mg to about 625 mg, about 600 mg to about 650 mg, about 625 mg to about 675 mg, about 650 mg to about 700 mg, about 675 mg to about 725 mg, about 700 mg to about 750 mg, about 725 mg to about 775 mg, about 750 mg to about 800 mg, about 775 mg to about 825 mg, about 800 mg to about 850 mg, about 825 mg to about 875 mg, about 850 mg to about 900 mg, about 875 mg to about 925 mg, about 900 mg to about 950 mg, about 925 mg to about 975 mg, or about 950 mg to about 1000 mg).

In some aspects, the C1D2 is between about 10 mg to 200 mg (e.g., between 10 mg to about 170 mg, about 11 mg to about 165 mg, about 12 mg to about 160 mg, about 13 mg to about 155 mg, about 14 mg to about 150 mg, about 15 mg to about 145 mg, about 16 mg to about 140 mg, about 17 mg to about 135 mg, about 18 mg to about 130 mg, about 19 mg to about 125 mg, about 20 mg to about 120 mg, about 21 mg to about 115 mg, about 22 mg to about 110 mg, about 23 mg to about 105 mg, about 24 mg to about 100 mg, about 25 mg to about 95 mg, about 26 mg to about 90 mg, about 27 mg to about 85 mg, about 28 mg to about 80 mg, about 29 mg to about 75 mg, about 30 mg to about 70 mg, about 31 mg to about 65 mg, about 32 mg to about 60 mg, about 33 mg to about 55 mg, about 34 mg to about 50 mg, about 35 mg to about 45 mg, about 40 mg to about 200 mg, about 45 mg to about 195 mg, about 50 mg to about 190 mg, about 55 mg to about 185 mg, about 60 mg to about 180 mg, about 65 mg to about 175 mg, about 70 mg to about 170 mg, about 75 mg to about 165 mg, about 80 mg to about 160 mg, about 85 mg to about 155 mg, about 90 mg to about 150 mg, about 95 mg to about 145 mg, about 100 mg to about 140 mg, about 105 mg to about 135 mg, about 110 mg to about 130 mg, about 115 mg to about 125 mg, about 10 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, about 90 mg to about 100 mg, about 100 mg to about 110 mg, about 110 mg to about 120 mg, about 120 mg to about 130 mg, about 130 mg to about 140 mg, about 140 mg to about 150 mg, about 150 mg to about 160 mg, about 160 mg to about 170 mg, about 170 mg to about 180 mg, about 180 mg to about 190 mg, or about 190 mg to about 200 mg).

In some aspects, the C1D1 is between 0.1 mg to 50 mg (e.g., between 0.1 mg to 9.5 mg, 0.2 mg to 9 mg, 0.3 mg to 8.5 mg, 0.4 mg to 8 mg, 0.5 mg to 7.5 mg, 0.6 mg to 7 mg, 0.7 mg to 6.5 mg, 0.8 mg to 6 mg, 0.9 mg to 5.5 mg, 1 mg to 5 mg, 1.1 mg to 4.5 mg, 1.2 mg to 4 mg, 1.3 mg to 3.5 mg, 1.4 mg to 3 mg, 1.5 mg to 2.5 mg, 1 mg to 3 mg, 0.2 mg to 35 mg, 0.3 mg to 30 mg, 0.4 mg to 29 mg, 0.5 mg to 28 mg, 1 mg to 27 mg, 1.5 mg to 26 mg, 2 mg to 25 mg, 2.5 mg to 24 mg, 3 mg to 23 mg, 3.5 mg to 22 mg, 4 mg to 21 mg, 4.5 mg to 20 mg, 5 mg to 19 mg, 5.5 mg to 18 mg, 6 mg to 17 mg, 6.5 mg to 16 mg, 7 mg to 15 mg, 7.5 mg to 14 mg, 8 mg to 13 mg, 8.5 mg to 12 mg, 9 mg to 11 mg, 0.1 mg to 1 mg, 0.2 mg to 1.5 mg, 0.3 mg to 2 mg, 0.4 mg to 2.5 mg, 0.5 mg to 3 mg, 0.6 mg to 3.5 mg, 0.7 mg to 4 mg, 0.8 mg to 4.5 mg, 0.9 mg to 5 mg, 1 mg to 6 mg, 1.5 mg to 6.5 mg, 2 mg to 7 mg, 2.5 mg to 7.5 mg, 3 mg to 8 mg, 3.5 mg to 8.5 mg, 4 mg to 9 mg, 4.5 mg to 9.5 mg, 5 mg to 10 mg, 5.5 mg to 10.5 mg, 6 mg to 11 mg, 6.5 mg to 11.5 mg, 7 mg to 12 mg, 7.5 mg to 12.5 mg, 8 mg to 13 mg, 8 mg to 12 mg, 8.5 mg to 13.5 mg, 9 mg to 14 mg, 9.5 mg to 14.5 mg, 10 mg to 15 mg, 11 mg to 16 mg, 12 mg to 17 mg, 13 mg to 18 mg, 14 mg to 19 mg, 15 mg to 20 mg, 16 mg to 21 mg, 17 mg to 22 mg, 18 mg to 23 mg, 19 mg to 24 mg, 20 mg to 25 mg, 21 mg to 26 mg, 22 mg to 27 mg, 23 mg to 28 mg, 24 mg to 29 mg, 25 mg to 30 mg, 26 mg to 31 mg, 27 mg to 32 mg, 28 mg to 33 mg, 29 mg to 34 mg, 30 mg to 35 mg, 31 mg to 36 mg, 32 mg to 37 mg, 33 mg to 38 mg, 34 mg to 39 mg, 35 mg to 40 mg, 36 mg to 41 mg, 37 mg to 42 mg, 38 mg to 43 mg, 39 mg to 44 mg, 40 mg to 45 mg, 41 mg to 46 mg, 42 mg to 47 mg, 43 mg to 48 mg, 44 mg to 49 mg, 45 mg to 50 mg) and the C1D2 is between 10 mg to 1000 mg (e.g., between 10 mg to 70 mg, 15 mg to 65 mg, 20 mg to 60 mg, 25 mg to 55 mg, 30 mg to 50 mg, 35 mg to 45 mg, 15 mg to 225 mg, 20 mg to 220 mg, 25 mg to 215 mg, 30 mg to 210 mg, 35 mg to 205 mg, 40 mg to 200 mg, 45 mg to 195 mg, 50 mg to 190 mg, 55 mg to 185 mg, 60 mg to 180 mg, 65 mg to 175 mg, 70 mg and 170 mg, 75 mg and 165 mg, 80 mg and 160 mg, 85 mg and 155 mg, 90 mg and 150 mg, 95 mg and 145 mg, 100 mg and 140 mg, 105 mg and 135 mg, 110 mg and 130 mg, 115 mg and 125 mg, 10 mg to 50 mg, 25 mg to 75 mg, 50 mg to 100 mg, 75 mg to 125 mg, 100 mg to 150 mg, 125 mg to 175 mg, 150 mg to 200 mg, 175 mg to 225 mg, 200 mg to 250 mg, 225 mg to 275 mg, 250 mg to 300 mg, 275 mg to 325 mg, 300 mg to 350 mg, 325 mg to 375 mg, 350 mg to 400 mg, 375 mg to 425 mg, 400 mg to 450 mg, 425 mg to 475 mg, 450 mg to 500 mg, 475 mg to 525 mg, 500 mg to 550 mg, 525 mg to 575 mg, 550 mg to 600 mg, 575 mg to 625 mg, 600 mg to 650 mg, 625 mg to 675 mg, 650 mg to 700 mg, 675 mg to 725 mg, 700 mg to 750 mg, 725 mg to 775 mg, 750 mg to 800 mg, 775 mg to 825 mg, 800 mg to 850 mg, 825 mg to 875 mg, 850 mg to 900 mg, 875 mg to 925 mg, 900 mg to 950 mg, 925 mg to 975 mg, or 950 mg to 1000 mg).

In some aspects, the C1D2 is between 10 mg to 200 mg (e.g., between 10 mg to 170 mg, 11 mg to 165 mg, 12 mg to 160 mg, 13 mg to 155 mg, 14 mg to 150 mg, 15 mg to 145 mg, 16 mg to 140 mg, 17 mg to 135 mg, 18 mg to 130 mg, 19 mg to 125 mg, 20 mg to 120 mg, 21 mg to 115 mg, 22 mg to 110 mg, 23 mg to 105 mg, 24 mg to 100 mg, 25 mg to 95 mg, 26 mg to 90 mg, 27 mg to 85 mg, 28 mg to 80 mg, 29 mg to 75 mg, 30 mg to 70 mg, 31 mg to 65 mg, 32 mg to 60 mg, 33 mg to 55 mg, 34 mg to 50 mg, 35 mg to 45 mg, 40 mg to 200 mg, 45 mg to 195 mg, 50 mg to 190 mg, 55 mg to 185 mg, 60 mg to 180 mg, 65 mg to 175 mg, 70 mg to 170 mg, 75 mg to 165 mg, 80 mg to 160 mg, 85 mg to 155 mg, 90 mg to 150 mg, 95 mg to 145 mg, 100 mg to 140 mg, 105 mg to 135 mg, 110 mg to 130 mg, 115 mg to 125 mg, 10 mg to 20 mg, 20 mg to 30 mg, 30 mg to 40 mg, 40 mg to 50 mg, 50 mg to 60 mg, 60 mg to 70 mg, 70 mg to 80 mg, 80 mg to 90 mg, 90 mg to 100 mg, 100 mg to 110 mg, 110 mg to 120 mg, 120 mg to 130 mg, 130 mg to 140 mg, 140 mg to 150 mg, 150 mg to 160 mg, 160 mg to 170 mg, 170 mg to 180 mg, 180 mg to 190 mg, or 190 mg to 200 mg).

In some aspects, the C1D1 is about 1 mg. In some aspects, the C1D1 is about 2 mg. In some aspects, the C1D1 is about 3 mg. In some aspects, the C1D1 is about 4 mg. In some aspects, the C1D1 is about 5 mg. In some aspects, the C1D1 is about 6 mg. In some aspects, the C1D1 is about 7 mg. In some aspects, the C1D1 is about 8 mg. In some aspects, the C1D1 is about 9 mg. In some aspects, the C1D1 is about 10 mg. In some aspects, the C1D1 is about 11 mg. In some aspects, the C1D1 is about 12 mg. In some aspects, the C1D1 is about 13 mg. In some aspects, the C1D1 is about 14 mg. In some aspects, the C1D1 is about 15 mg. In some aspects, the C1D1 is about 16 mg. In some aspects, the C1D1 is about 17 mg. In some aspects, the C1D1 is about 18 mg. In some aspects, the C1D1 is about 19 mg. In some aspects, the C1D1 is about 20 mg. In some aspects, the C1D1 is about 21 mg. In some aspects, the C1D1 is about 22 mg. In some aspects, the C1D1 is about 23 mg. In some aspects, the C1D1 is about 24 mg. In some aspects, the C1D1 is about 25 mg. In some aspects, the C1D1 is about 26 mg. In some aspects, the C1D1 is about 27 mg. In some aspects, the C1D1 is about 28 mg. In some aspects, the C1D1 is about 29 mg. In some aspects, the C1D1 is about 30 mg. In some aspects, the C1D1 is about 31 mg. In some aspects, the C1D1 is about 32 mg. In some aspects, the C1D1 is about 33 mg. In some aspects, the C1D1 is about 34 mg. In some aspects, the C1D1 is about 35 mg. In some aspects, the C1D1 is about 36 mg. In some aspects, the C1D1 is about 37 mg. In some aspects, the C1D1 is about 38 mg. In some aspects, the C1D1 is about 39 mg. In some aspects, the C1D1 is about 40 mg. In some aspects, the C1D1 is about 41 mg. In some aspects, the C1D1 is about 42 mg. In some aspects, the C1D1 is about 43 mg. In some aspects, the C1D1 is about 44 mg. In some aspects, the C1D1 is about 45 mg. In some aspects, the C1D1 is about 46 mg. In some aspects, the C1D1 is about 47 mg. In some aspects, the C1D1 is about 48 mg. In some aspects, the C1D1 is about 49 mg. In some aspects, the C1D1 is about 50 mg.

In some aspects, the C1D2 is about 10 mg. In some aspects, the C1D2 is about 15 mg. In some aspects, the C1D2 is about 20 mg. In some aspects, the C1D2 is about 25 mg. In some aspects, the C1D2 is about 30 mg. In some aspects, the C1D2 is about 35 mg. In some aspects, the C1D2 is about 40 mg. In some aspects, the C1D2 is about 45 mg. In some aspects, the C1D2 is about 50 mg. In some aspects, the C1D2 is about 55 mg. In some aspects, the C1D2 is about 60 mg. In some aspects, the C1D2 is about 65 mg. In some aspects, the C1D2 is about 70 mg. In some aspects, the C1D2 is about 75 mg. In some aspects, the C1D2 is about 80 mg. In some aspects, the C1D2 is about 85 mg. In some aspects, the C1D2 is about 90 mg. In some aspects, the C1D2 is about 95 mg. In some aspects, the C1D2 is about 100 mg. In some aspects, the C1D2 is about 105 mg. In some aspects, the C1D2 is about 110 mg. In some aspects, the C1D2 is about 115 mg. In some aspects, the C1D2 is about 120 mg. In some aspects, the C1D2 is about 125 mg. In some aspects, the C1D2 is about 130 mg. In some aspects, the C1D2 is about 135 mg. In some aspects, the C1D2 is about 140 mg. In some aspects, the C1D2 is about 145 mg. In some aspects, the C1D2 is about 150 mg. In some aspects, the C1D2 is about 155 mg. In some aspects, the C1D2 is about 160 mg. In some aspects, the C1D2 is about 165 mg. In some aspects, the C1D2 is about 170 mg. In some aspects, the C1D2 is about 175 mg. In some aspects, the C1D2 is about 180 mg. In some aspects, the C1D2 is about 185 mg. In some aspects, the C1D2 is about 190 mg. In some aspects, the C1D2 is about 195 mg. In some aspects, the C1D2 is about 200 mg. In some aspects, the C1D2 is about 205 mg. In some aspects, the C1D2 is about 210 mg. In some aspects, the C1D2 is about 215 mg. In some aspects, the C1D2 is about 220 mg. In some aspects, the C1D2 is about 225 mg. In some aspects, the C1D2 is about 230 mg. In some aspects, the C1D2 is about 235 mg. In some aspects, the C1D2 is about 240 mg. In some aspects, the C1D2 is about 245 mg. In some aspects, the C1D2 is about 250 mg. In some aspects, the C1D2 is about 255 mg. In some aspects, the C1D2 is about 260 mg. In some aspects, the C1D2 is about 265 mg. In some aspects, the C1D2 is about 270 mg. In some aspects, the C1D2 is about 275 mg. In some aspects, the C1D2 is about 280 mg. In some aspects, the C1D2 is about 285 mg. In some aspects, the C1D2 is about 290 mg. In some aspects, the C1D2 is about 295 mg. In some aspects, the C1D2 is about 300 mg. In some aspects, the C1D2 is about 305 mg. In some aspects, the C1D2 is about 310 mg. In some aspects, the C1D2 is about 315 mg. In some aspects, the C1D2 is about 320 mg. In some aspects, the C1D2 is about 325 mg. In some aspects, the C1D2 is about 330 mg. In some aspects, the C1D2 is about 335 mg. In some aspects, the C1D2 is about 340 mg. In some aspects, the C1D2 is about 345 mg. In some aspects, the C1D2 is about 350 mg. In some aspects, the C1D2 is about 355 mg. In some aspects, the C1D2 is about 360 mg. In some aspects, the C1D2 is about 365 mg. In some aspects, the C1D2 is about 370 mg. In some aspects, the C1D2 is about 375 mg. In some aspects, the C1D2 is about 380 mg. In some aspects, the C1D2 is about 385 mg. In some aspects, the C1D2 is about 390 mg. In some aspects, the C1D2 is about 395 mg. In some aspects, the C1D2 is about 400 mg. In some aspects, the C1D2 is about 405 mg. In some aspects, the C1D2 is about 410 mg. In some aspects, the C1D2 is about 415 mg. In some aspects, the C1D2 is about 420 mg. In some aspects, the C1D2 is about 425 mg. In some aspects, the C1D2 is about 430 mg. In some aspects, the C1D2 is about 435 mg. In some aspects, the C1D2 is about 440 mg. In some aspects, the C1D2 is about 445 mg. In some aspects, the C1D2 is about 450 mg. In some aspects, the C1D2 is about 455 mg. In some aspects, the C1D2 is about 460 mg. In some aspects, the C1D2 is about 465 mg. In some aspects, the C1D2 is about 470 mg. In some aspects, the C1D2 is about 475 mg. In some aspects, the C1D2 is about 480 mg. In some aspects, the C1D2 is about 485 mg. In some aspects, the C1D2 is about 490 mg. In some aspects, the C1D2 is about 495 mg. In some aspects, the C1D2 is about 500 mg. In some aspects, the C1D2 is about 505 mg. In some aspects, the C1D2 is about 510 mg. In some aspects, the C1D2 is about 515 mg. In some aspects, the C1D2 is about 520 mg. In some aspects, the C1D2 is about 525 mg. In some aspects, the C1D2 is about 530 mg. In some aspects, the C1D2 is about 535 mg. In some aspects, the C1D2 is about 540 mg. In some aspects, the C1D2 is about 545 mg. In some aspects, the C1D2 is about 550 mg. In some aspects, the C1D2 is about 555 mg. In some aspects, the C1D2 is about 560 mg. In some aspects, the C1D2 is about 565 mg. In some aspects, the C1D2 is about 570 mg. In some aspects, the C1D2 is about 575 mg. In some aspects, the C1D2 is about 580 mg. In some aspects, the C1D2 is about 585 mg. In some aspects, the C1D2 is about 590 mg. In some aspects, the C1D2 is about 595 mg. In some aspects, the C1D2 is about 600 mg. In some aspects, the C1D2 is about 605 mg. In some aspects, the C1D2 is about 610 mg. In some aspects, the C1D2 is about 615 mg. In some aspects, the C1D2 is about 620 mg. In some aspects, the C1D2 is about 625 mg. In some aspects, the C1D2 is about 630 mg. In some aspects, the C1D2 is about 635 mg. In some aspects, the C1D2 is about 640 mg. In some aspects, the C1D2 is about 645 mg. In some aspects, the C1D2 is about 650 mg. In some aspects, the C1D2 is about 655 mg. In some aspects, the C1D2 is about 660 mg. In some aspects, the C1D2 is about 665 mg. In some aspects, the C1D2 is about 670 mg. In some aspects, the C1D2 is about 675 mg. In some aspects, the C1D2 is about 680 mg. In some aspects, the C1D2 is about 685 mg. In some aspects, the C1D2 is about 690 mg. In some aspects, the C1D2 is about 695 mg. In some aspects, the C1D2 is about 700 mg. In some aspects, the C1D2 is about 705 mg. In some aspects, the C1D2 is about 710 mg. In some aspects, the C1D2 is about 715 mg. In some aspects, the C1D2 is about 720 mg. In some aspects, the C1D2 is about 725 mg. In some aspects, the C1D2 is about 730 mg. In some aspects, the C1D2 is about 735 mg. In some aspects, the C1D2 is about 740 mg. In some aspects, the C1D2 is about 745 mg. In some aspects, the C1D2 is about 750 mg. In some aspects, the C1D2 is about 755 mg. In some aspects, the C1D2 is about 760 mg. In some aspects, the C1D2 is about 765 mg. In some aspects, the C1D2 is about 770 mg. In some aspects, the C1D2 is about 775 mg. In some aspects, the C1D2 is about 780 mg. In some aspects, the C1D2 is about 785 mg. In some aspects, the C1D2 is about 790 mg. In some aspects, the C1D2 is about 795 mg. In some aspects, the C1D2 is about 800 mg. In some aspects, the C1D2 is about 805 mg. In some aspects, the C1D2 is about 810 mg. In some aspects, the C1D2 is about 815 mg. In some aspects, the C1D2 is about 820 mg. In some aspects, the C1D2 is about 825 mg. In some aspects, the C1D2 is about 830 mg. In some aspects, the C1D2 is about 835 mg. In some aspects, the C1D2 is about 840 mg. In some aspects, the C1D2 is about 845 mg. In some aspects, the C1D2 is about 850 mg. In some aspects, the C1D2 is about 855 mg. In some aspects, the C1D2 is about 860 mg. In some aspects, the C1D2 is about 865 mg. In some aspects, the C1D2 is about 870 mg. In some aspects, the C1D2 is about 875 mg. In some aspects, the C1D2 is about 880 mg. In some aspects, the C1D2 is about 885 mg. In some aspects, the C1D2 is about 890 mg. In some aspects, the C1D2 is about 895 mg. In some aspects, the C1D2 is about 900 mg. In some aspects, the C1D2 is about 905 mg. In some aspects, the C1D2 is about 910 mg. In some aspects, the C1D2 is about 915 mg. In some aspects, the C1D2 is about 920 mg. In some aspects, the C1D2 is about 925 mg. In some aspects, the C1D2 is about 930 mg. In some aspects, the C1D2 is about 935 mg. In some aspects, the C1D2 is about 940 mg. In some aspects, the C1D2 is about 945 mg. In some aspects, the C1D2 is about 950 mg. In some aspects, the C1D2 is about 955 mg. In some aspects, the C1D2 is about 960 mg. In some aspects, the C1D2 is about 965 mg. In some aspects, the C1D2 is about 970 mg. In some aspects, the C1D2 is about 975 mg. In some aspects, the C1D2 is about 980 mg. In some aspects, the C1D2 is about 985 mg. In some aspects, the C1D2 is about 990 mg. In some aspects, the C1D2 is about 995 mg. In some aspects, the C1D2 is about 1000 mg.

In some aspects, the C1D1 is 1 mg. In some aspects, the C1D1 is 2 mg. In some aspects, the C1D1 is 3 mg. In some aspects, the C1D1 is 4 mg. In some aspects, the C1D1 is 5 mg. In some aspects, the C1D1 is 6 mg. In some aspects, the C1D1 is 7 mg. In some aspects, the C1D1 is 8 mg. In some aspects, the C1D1 is 9 mg. In some aspects, the C1D1 is 10 mg. In some aspects, the C1D1 is 11 mg. In some aspects, the C1D1 is 12 mg. In some aspects, the C1D1 is 13 mg. In some aspects, the C1D1 is 14 mg. In some aspects, the C1D1 is 15 mg. In some aspects, the C1D1 is 16 mg. In some aspects, the C1D1 is 17 mg. In some aspects, the C1D1 is 18 mg. In some aspects, the C1D1 is 19 mg. In some aspects, the C1D1 is 20 mg. In some aspects, the C1D1 is 21 mg. In some aspects, the C1D1 is 22 mg. In some aspects, the C1D1 is 23 mg. In some aspects, the C1D1 is 24 mg. In some aspects, the C1D1 is 25 mg. In some aspects, the C1D1 is 26 mg. In some aspects, the C1D1 is 27 mg. In some aspects, the C1D1 is 28 mg. In some aspects, the C1D1 is 29 mg. In some aspects, the C1D1 is 30 mg. In some aspects, the C1D1 is 31 mg. In some aspects, the C1D1 is 32 mg. In some aspects, the C1D1 is 33 mg. In some aspects, the C1D1 is 34 mg. In some aspects, the C1D1 is 35 mg. In some aspects, the C1D1 is 36 mg. In some aspects, the C1D1 is 37 mg. In some aspects, the C1D1 is 38 mg. In some aspects, the C1D1 is 39 mg. In some aspects, the C1D1 is 40 mg. In some aspects, the C1D1 is 41 mg. In some aspects, the C1D1 is 42 mg. In some aspects, the C1D1 is 43 mg. In some aspects, the C1D1 is 44 mg. In some aspects, the C1D1 is 45 mg. In some aspects, the C1D1 is 46 mg. In some aspects, the C1D1 is 47 mg. In some aspects, the C1D1 is 48 mg. In some aspects, the C1D1 is 49 mg. In some aspects, the C1D1 is 50 mg.

In some aspects, the C1D2 is 10 mg. In some aspects, the C1D2 is 15 mg. In some aspects, the C1D2 is 20 mg. In some aspects, the C1D2 is 25 mg. In some aspects, the C1D2 is 30 mg. In some aspects, the C1D2 is 35 mg. In some aspects, the C1D2 is 40 mg. In some aspects, the C1D2 is 45 mg. In some aspects, the C1D2 is 50 mg. In some aspects, the C1D2 is 55 mg. In some aspects, the C1D2 is 60 mg. In some aspects, the C1D2 is 65 mg. In some aspects, the C1D2 is 70 mg. In some aspects, the C1D2 is 75 mg. In some aspects, the C1D2 is 80 mg. In some aspects, the C1D2 is 85 mg. In some aspects, the C1D2 is 90 mg. In some aspects, the C1D2 is 95 mg. In some aspects, the C1D2 is 100 mg. In some aspects, the C1D2 is 105 mg. In some aspects, the C1D2 is 110 mg. In some aspects, the C1D2 is 115 mg. In some aspects, the C1D2 is 120 mg. In some aspects, the C1D2 is 125 mg. In some aspects, the C1D2 is 130 mg. In some aspects, the C1D2 is 135 mg. In some aspects, the C1D2 is 140 mg. In some aspects, the C1D2 is 145 mg. In some aspects, the C1D2 is 150 mg. In some aspects, the C1D2 is 155 mg. In some aspects, the C1D2 is 160 mg. In some aspects, the C1D2 is 165 mg. In some aspects, the C1D2 is 170 mg. In some aspects, the C1D2 is 175 mg. In some aspects, the C1D2 is 180 mg. In some aspects, the C1D2 is 185 mg. In some aspects, the C1D2 is 190 mg. In some aspects, the C1D2 is 195 mg. In some aspects, the C1D2 is 200 mg. In some aspects, the C1D2 is 205 mg. In some aspects, the C1D2 is 210 mg. In some aspects, the C1D2 is 215 mg. In some aspects, the C1D2 is 220 mg. In some aspects, the C1D2 is 225 mg. In some aspects, the C1D2 is 230 mg. In some aspects, the C1D2 is 235 mg. In some aspects, the C1D2 is 240 mg. In some aspects, the C1D2 is 245 mg. In some aspects, the C1D2 is 250 mg. In some aspects, the C1D2 is 255 mg. In some aspects, the C1D2 is 260 mg. In some aspects, the C1D2 is 265 mg. In some aspects, the C1D2 is 270 mg. In some aspects, the C1D2 is 275 mg. In some aspects, the C1D2 is 280 mg. In some aspects, the C1D2 is 285 mg. In some aspects, the C1D2 is 290 mg. In some aspects, the C1D2 is 295 mg. In some aspects, the C1D2 is 300 mg. In some aspects, the C1D2 is 305 mg. In some aspects, the C1D2 is 310 mg. In some aspects, the C1D2 is 315 mg. In some aspects, the C1D2 is 320 mg. In some aspects, the C1D2 is 325 mg. In some aspects, the C1D2 is 330 mg. In some aspects, the C1D2 is 335 mg. In some aspects, the C1D2 is 340 mg. In some aspects, the C1D2 is 345 mg. In some aspects, the C1D2 is 350 mg. In some aspects, the C1D2 is 355 mg. In some aspects, the C1D2 is 360 mg. In some aspects, the C1D2 is 365 mg. In some aspects, the C1D2 is 370 mg. In some aspects, the C1D2 is 375 mg. In some aspects, the C1D2 is 380 mg. In some aspects, the C1D2 is 385 mg. In some aspects, the C1D2 is 390 mg. In some aspects, the C1D2 is 395 mg. In some aspects, the C1D2 is 400 mg. In some aspects, the C1D2 is 405 mg. In some aspects, the C1D2 is 410 mg. In some aspects, the C1D2 is 415 mg. In some aspects, the C1D2 is 420 mg. In some aspects, the C1D2 is 425 mg. In some aspects, the C1D2 is 430 mg. In some aspects, the C1D2 is 435 mg. In some aspects, the C1D2 is 440 mg. In some aspects, the C1D2 is 445 mg. In some aspects, the C1D2 is 450 mg. In some aspects, the C1D2 is 455 mg. In some aspects, the C1D2 is 460 mg. In some aspects, the C1D2 is 465 mg. In some aspects, the C1D2 is 470 mg. In some aspects, the C1D2 is 475 mg. In some aspects, the C1D2 is 480 mg. In some aspects, the C1D2 is 485 mg. In some aspects, the C1D2 is 490 mg. In some aspects, the C1D2 is 495 mg. In some aspects, the C1D2 is 500 mg. In some aspects, the C1D2 is 505 mg. In some aspects, the C1D2 is 510 mg. In some aspects, the C1D2 is 515 mg. In some aspects, the C1D2 is 520 mg. In some aspects, the C1D2 is 525 mg. In some aspects, the C1D2 is 530 mg. In some aspects, the C1D2 is 535 mg. In some aspects, the C1D2 is 540 mg. In some aspects, the C1D2 is 545 mg. In some aspects, the C1D2 is 550 mg. In some aspects, the C1D2 is 555 mg. In some aspects, the C1D2 is 560 mg. In some aspects, the C1D2 is 565 mg. In some aspects, the C1D2 is 570 mg. In some aspects, the C1D2 is 575 mg. In some aspects, the C1D2 is 580 mg. In some aspects, the C1D2 is 585 mg. In some aspects, the C1D2 is 590 mg. In some aspects, the C1D2 is 595 mg. In some aspects, the C1D2 is 600 mg. In some aspects, the C1D2 is 605 mg. In some aspects, the C1D2 is 610 mg. In some aspects, the C1D2 is 615 mg. In some aspects, the C1D2 is 620 mg. In some aspects, the C1D2 is 625 mg. In some aspects, the C1D2 is 630 mg. In some aspects, the C1D2 is 635 mg. In some aspects, the C1D2 is 640 mg. In some aspects, the C1D2 is 645 mg. In some aspects, the C1D2 is 650 mg. In some aspects, the C1D2 is 655 mg. In some aspects, the C1D2 is 660 mg. In some aspects, the C1D2 is 665 mg. In some aspects, the C1D2 is 670 mg. In some aspects, the C1D2 is 675 mg. In some aspects, the C1D2 is 680 mg. In some aspects, the C1D2 is 685 mg. In some aspects, the C1D2 is 690 mg. In some aspects, the C1D2 is 695 mg. In some aspects, the C1D2 is 700 mg. In some aspects, the C1D2 is 705 mg. In some aspects, the C1D2 is 710 mg. In some aspects, the C1D2 is 715 mg. In some aspects, the C1D2 is 720 mg. In some aspects, the C1D2 is 725 mg. In some aspects, the C1D2 is 730 mg. In some aspects, the C1D2 is 735 mg. In some aspects, the C1D2 is 740 mg. In some aspects, the C1D2 is 745 mg. In some aspects, the C1D2 is 750 mg. In some aspects, the C1D2 is 755 mg. In some aspects, the C1D2 is 760 mg. In some aspects, the C1D2 is 765 mg. In some aspects, the C1D2 is 770 mg. In some aspects, the C1D2 is 775 mg. In some aspects, the C1D2 is 780 mg. In some aspects, the C1D2 is 785 mg. In some aspects, the C1D2 is 790 mg. In some aspects, the C1D2 is 795 mg. In some aspects, the C1D2 is 800 mg. In some aspects, the C1D2 is 805 mg. In some aspects, the C1D2 is 810 mg. In some aspects, the C1D2 is 815 mg. In some aspects, the C1D2 is 820 mg. In some aspects, the C1D2 is 825 mg. In some aspects, the C1D2 is 830 mg. In some aspects, the C1D2 is 835 mg. In some aspects, the C1D2 is 840 mg. In some aspects, the C1D2 is 845 mg. In some aspects, the C1D2 is 850 mg. In some aspects, the C1D2 is 855 mg. In some aspects, the C1D2 is 860 mg. In some aspects, the C1D2 is 865 mg. In some aspects, the C1D2 is 870 mg. In some aspects, the C1D2 is 875 mg. In some aspects, the C1D2 is 880 mg. In some aspects, the C1D2 is 885 mg. In some aspects, the C1D2 is 890 mg. In some aspects, the C1D2 is 895 mg. In some aspects, the C1D2 is 900 mg. In some aspects, the C1D2 is 905 mg. In some aspects, the C1D2 is 910 mg. In some aspects, the C1D2 is 915 mg. In some aspects, the C1D2 is 920 mg. In some aspects, the C1D2 is 925 mg. In some aspects, the C1D2 is 930 mg. In some aspects, the C1D2 is 935 mg. In some aspects, the C1D2 is 940 mg. In some aspects, the C1D2 is 945 mg. In some aspects, the C1D2 is 950 mg. In some aspects, the C1D2 is 955 mg. In some aspects, the C1D2 is 960 mg. In some aspects, the C1D2 is 965 mg. In some aspects, the C1D2 is 970 mg. In some aspects, the C1D2 is 975 mg. In some aspects, the C1D2 is 980 mg. In some aspects, the C1D2 is 985 mg. In some aspects, the C1D2 is 990 mg. In some aspects, the C1D2 is 995 mg. In some aspects, the C1D2 is 1000 mg.

In some instances, the methods described above may include a first dosing cycle of four weeks or 28 days. In some instances, the methods may include administering to the subject the C1D1 and the C1D2 on or about Days 1 and 8, respectively, of the first dosing cycle.

ii. Double Step-Up Dosing Regimens

In other aspects, the invention provides methods of treating a subject having a cancer (e.g., an MM) comprising subcutaneously administering to the subject a bispecific antibody that binds to FcRH5 and CD3 in a double step-up dosing regimen.

In some aspects, the disclosure features a method of treating a subject having a cancer (e.g., an MM) comprising subcutaneously administering to the subject a bispecific antibody that binds to FcRH5 and CD3 in a dosing regimen comprising at least a first dosing cycle, wherein the first dosing cycle comprises a first dose (C1D1) of the bispecific antibody, a second dose (C1D2) of the bispecific antibody, and a third dose (C1D3) of the bispecific antibody, wherein the C1D1 is between about 0.1 mg to about 10 mg (e.g., is between about 0.1 mg to about 2 mg, about 0.2 mg to about 1 mg, or about 0.2 mg to about 0.4 mg, about 0.1 mg to about 9.5 mg, about 0.2 mg to about 9 mg, about 0.3 mg to about 8.5 mg, about 0.4 mg to about 8 mg, about 0.5 mg to about 7.5 mg, about 0.6 mg to about 7 mg, about 0.7 mg to about 6.5 mg, about 0.8 mg to about 6 mg, about 0.9 mg to about 5.5 mg, about 1 mg to about 5 mg, about 1.1 mg to about 4.5 mg, about 1.2 mg to about 4 mg, about 1.3 mg to about 3.5 mg, about 1.4 mg to about 3 mg, about 1.5 mg to about 2.5 mg, about 1 mg to about 3 mg, about 0.1 mg to about 1 mg, about 0.2 mg to about 1.5 mg, about 0.3 mg to about 2 mg, about 0.4 mg to about 2.5 mg, about 0.5 mg to about 3 mg, about 0.6 mg to about 3.5 mg, about 0.7 mg to about 4 mg, about 0.8 mg to about 4.5 mg, about 0.9 mg to about 5 mg, about 1 mg to about 6 mg, about 1.5 mg to about 6.5 mg, about 2 mg to about 7 mg, about 2.5 mg to about 7.5 mg, about 3 mg to about 8 mg, about 3.5 mg to about 8.5 mg, about 4 mg to about 9 mg, about 4.5 mg to about 9.5 mg, or about 5 mg to about 10 mg); and the C1D2 is between about 1 mg to about 50 mg (e.g., between about 3 mg to about 18 mg, between about 3.1 mg to about 15 mg, between about 3.2 mg to about 10 mg, between about 3.3 mg to about 6 mg, between about 3.4 mg to about 4 mg, about 0.2 mg to about 35 mg, about 0.3 mg to about 30 mg, about 0.4 mg to about 29 mg, about 0.5 mg to about 28 mg, about 1 mg to about 27 mg, about 1.5 mg to about 26 mg, about 2 mg to about 25 mg, about 2.5 mg to about 24 mg, about 3 mg to about 23 mg, about 3.5 mg to about 22 mg, about 4 mg to about 21 mg, about 4.5 mg to about 20 mg, about 5 mg to about 19 mg, about 5.5 mg to about 18 mg, about 6 mg to about 17 mg, about 6.5 mg to about 16 mg, about 7 mg to about 15 mg, about 7.5 mg to about 14 mg, about 8 mg to about 13 mg, about 8 mg to about 12 mg, about 8.5 mg to about 12 mg, about 9 mg to about 11 mg, about 1 mg to about 6 mg, about 1.5 mg to about 6.5 mg, about 2 mg to about 7 mg, about 2.5 mg to about 7.5 mg, about 3 mg to about 8 mg, about 3.5 mg to about 8.5 mg, about 4 mg to about 9 mg, about 4.5 mg to about 9.5 mg, about 5 mg to about 10 mg, about 5.5 mg to about 10.5 mg, about 6 mg to about 11 mg, about 6.5 mg to about 11.5 mg, about 7 mg to about 12 mg, about 7.5 mg to about 12.5 mg, about 8 mg to about 13 mg, about 8.5 mg to about 13.5 mg, about 9 mg to about 14 mg, about 9.5 mg to about 14.5 mg, about 10 mg to about 15 mg, about 11 mg to about 16 mg, about 12 mg to about 17 mg, about 13 mg to about 18 mg, about 14 mg to about 19 mg, about 15 mg to about 20 mg, about 16 mg to about 21 mg, about 17 mg to about 22 mg, about 18 mg to about 23 mg, about 19 mg to about 24 mg, about 20 mg to about 25 mg, about 21 mg to about 26 mg, about 22 mg to about 27 mg, about 23 mg to about 28 mg, about 24 mg to about 29 mg, about 25 mg to about 30 mg, about 26 mg to about 31 mg, about 27 mg to about 32 mg, about 28 mg to about 33 mg, about 29 mg to about 34 mg, about 30 mg to about 35 mg, about 31 mg to about 36 mg, about 32 mg to about 37 mg, about 33 mg to about 38 mg, about 34 mg to about 39 mg, about 35 mg to about 40 mg, about 36 mg to about 41 mg, about 37 mg to about 42 mg, about 38 mg to about 43 mg, about 39 mg to about 44 mg, about 40 mg to about 45 mg, about 41 mg to about 46 mg, about 42 mg to about 47 mg, about 43 mg to about 48 mg, about 44 mg to about 49 mg, or about 45 mg to about 50 mg); and the C1D3 is between about 10 mg to about 1000 mg (e.g., between about 10 mg to about 70 mg, about 15 mg to about 65 mg, about 20 mg to about 60 mg, about 25 mg to about 55 mg, about 30 mg to about 50 mg, about 35 mg to about 45 mg, about 15 mg to about 225 mg, about 20 mg to about 220 mg, about 25 mg to about 215 mg, about 30 mg to about 210 mg, about 35 mg to about 205 mg, about 40 mg to about 200 mg, about 45 mg to about 195 mg, about 50 mg to about 190 mg, about 55 mg to about 185 mg, about 60 mg to about 180 mg, about 65 mg to about 175 mg, about 70 mg to about 170 mg, about 75 mg to about 165 mg, about 80 mg to about 160 mg, about 85 mg to about 155 mg, about 90 mg to about 150 mg, about 95 mg to about 145 mg, about 100 mg to about 140 mg, about 105 mg to about 135 mg, about 110 mg to about 130 mg, about 115 mg to about 125 mg, about 10 mg to about 50 mg, about 25 mg to about 75 mg, about 50 mg to about 100 mg, about 75 mg to about 125 mg, about 100 mg to about 150 mg, about 125 mg to about 175 mg, about 150 mg to about 200 mg, about 175 mg to about 225 mg, about 200 mg to about 250 mg, about 225 mg to about 275 mg, about 250 mg to about 300 mg, about 275 mg to about 325 mg, about 300 mg to about 350 mg, about 325 mg to about 375 mg, about 350 mg to about 400 mg, about 375 mg to about 425 mg, about 400 mg to about 450 mg, about 425 mg to about 475 mg, about 450 mg to about 500 mg, about 475 mg to about 525 mg, about 500 mg to about 550 mg, about 525 mg to about 575 mg, about 550 mg to about 600 mg, about 575 mg to about 625 mg, about 600 mg to about 650 mg, about 625 mg to about 675 mg, about 650 mg to about 700 mg, about 675 mg to about 725 mg, about 700 mg to about 750 mg, about 725 mg to about 775 mg, about 750 mg to about 800 mg, about 775 mg to about 825 mg, about 800 mg to about 850 mg, about 825 mg to about 875 mg, about 850 mg to about 900 mg, about 875 mg to about 925 mg, about 900 mg to about 950 mg, about 925 mg to about 975 mg, or about 950 mg to about 1000 mg). In some aspects, the C1D2 is greater than the C1D1 and the C1D3 is greater than the C1D2.

In some aspects, the C1D3 is between about 10 mg to 200 mg (e.g., between 10 mg to about 170 mg, about 11 mg to about 165 mg, about 12 mg to about 160 mg, about 13 mg to about 155 mg, about 14 mg to about 150 mg, about 15 mg to about 145 mg, about 16 mg to about 140 mg, about 17 mg to about 135 mg, about 18 mg to about 130 mg, about 19 mg to about 125 mg, about 20 mg to about 120 mg, about 21 mg to about 115 mg, about 22 mg to about 110 mg, about 23 mg to about 105 mg, about 24 mg to about 100 mg, about 25 mg to about 95 mg, about 26 mg to about 90 mg, about 27 mg to about 85 mg, about 28 mg to about 80 mg, about 29 mg to about 75 mg, about 30 mg to about 70 mg, about 31 mg to about 65 mg, about 32 mg to about 60 mg, about 33 mg to about 55 mg, about 34 mg to about 50 mg, about 35 mg to about 45 mg, about 40 mg to about 200 mg, about 45 mg to about 195 mg, about 50 mg to about 190 mg, about 55 mg to about 185 mg, about 60 mg to about 180 mg, about 65 mg to about 175 mg, about 70 mg to about 170 mg, about 75 mg to about 165 mg, about 80 mg to about 160 mg, about 85 mg to about 155 mg, about 90 mg to about 150 mg, about 95 mg to about 145 mg, about 100 mg to about 140 mg, about 105 mg to about 135 mg, about 110 mg to about 130 mg, about 115 mg to about 125 mg, about 10 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, about 90 mg to about 100 mg, about 100 mg to about 110 mg, about 110 mg to about 120 mg, about 120 mg to about 130 mg, about 130 mg to about 140 mg, about 140 mg to about 150 mg, about 150 mg to about 160 mg, about 160 mg to about 170 mg, about 170 mg to about 180 mg, about 180 mg to about 190 mg, or about 190 mg to about 200 mg).

In some aspects, the C1D1 is between 0.1 mg to 10 mg (e.g., is between 0.2 mg to 0.4 mg, 0.1 mg to 9.5 mg, 0.2 mg to 9 mg, 0.3 mg to 8.5 mg, 0.4 mg to 8 mg, 0.5 mg to 7.5 mg, 0.6 mg to 7 mg, 0.7 mg to 6.5 mg, 0.8 mg to 6 mg, 0.9 mg to 5.5 mg, 1 mg to 5 mg, 1.1 mg to 4.5 mg, 1.2 mg to 4 mg, 1.3 mg to 3.5 mg, 1.4 mg to 3 mg, 1.5 mg to 2.5 mg, 1 mg to 3 mg, 0.1 mg to 1 mg, 0.2 mg to 1.5 mg, 0.3 mg to 2 mg, 0.4 mg to 2.5 mg, 0.5 mg to 3 mg, 0.6 mg to 3.5 mg, 0.7 mg to 4 mg, 0.8 mg to 4.5 mg, 0.9 mg to 5 mg, 1 mg to 6 mg, 1.5 mg to 6.5 mg, 2 mg to 7 mg, 2.5 mg to 7.5 mg, 3 mg to 8 mg, 3.5 mg to 8.5 mg, 4 mg to 9 mg, 4.5 mg to 9.5 mg, or 5 mg to 10 mg).

In some aspects, the C1D2 is between 1 mg to 50 mg (e.g., between 3 mg to 18 mg, between 3.1 mg to 15 mg, between 3.2 mg to 10 mg, between 3.3 mg to 6 mg, between 3.4 mg to 4 mg, 0.2 mg to 35 mg, 0.3 mg to 30 mg, 0.4 mg to 29 mg, 0.5 mg to 28 mg, 1 mg to 27 mg, 1.5 mg to 26 mg, 2 mg to 25 mg, 2.5 mg to 24 mg, 3 mg to 23 mg, 3.5 mg to 22 mg, 4 mg to 21 mg, 4.5 mg to 20 mg, 5 mg to 19 mg, 5.5 mg to 18 mg, 6 mg to 17 mg, 6.5 mg to 16 mg, 7 mg to 15 mg, 7.5 mg to 14 mg, 8 mg to 13 mg, 8 mg to 12 mg, 8.5 mg to 12 mg, 9 mg to 11 mg, 1 mg to 6 mg, 1.5 mg to 6.5 mg, 2 mg to 7 mg, 2.5 mg to 7.5 mg, 3 mg to 8 mg, 3.5 mg to 8.5 mg, 4 mg to 9 mg, 4.5 mg to 9.5 mg, 5 mg to 10 mg, 5.5 mg to 10.5 mg, 6 mg to 11 mg, 6.5 mg to 11.5 mg, 7 mg to 12 mg, 7.5 mg to 12.5 mg, 8 mg to 13 mg, 8.5 mg to 13.5 mg, 9 mg to 14 mg, 9.5 mg to 14.5 mg, 10 mg to 15 mg, 11 mg to 16 mg, 12 mg to 17 mg, 13 mg to 18 mg, 14 mg to 19 mg, 15 mg to 20 mg, 16 mg to 21 mg, 17 mg to 22 mg, 18 mg to 23 mg, 19 mg to 24 mg, 20 mg to 25 mg, 21 mg to 26 mg, 22 mg to 27 mg, 23 mg to 28 mg, 24 mg to 29 mg, 25 mg to 30 mg, 26 mg to 31 mg, 27 mg to 32 mg, 28 mg to 33 mg, 29 mg to 34 mg, 30 mg to 35 mg, 31 mg to 36 mg, 32 mg to 37 mg, 33 mg to 38 mg, 34 mg to 39 mg, 35 mg to 40 mg, 36 mg to 41 mg, 37 mg to 42 mg, 38 mg to 43 mg, 39 mg to 44 mg, 40 mg to 45 mg, 41 mg to 46 mg, 42 mg to 47 mg, 43 mg to 48 mg, 44 mg to 49 mg, or 45 mg to 50 mg).

In some aspects the C1D3 is between 10 mg to 1000 mg (e.g., between 10 mg to 70 mg, 15 mg to 65 mg, 20 mg to 60 mg, 25 mg to 55 mg, 30 mg to 50 mg, 35 mg to 45 mg, 15 mg to 225 mg, 20 mg to 220 mg, 25 mg to 215 mg, 30 mg to 210 mg, 35 mg to 205 mg, 40 mg to 200 mg, 45 mg to 195 mg, 50 mg to 190 mg, 55 mg to 185 mg, 60 mg to 180 mg, 65 mg to 175 mg, 70 mg to 170 mg, 75 mg to 165 mg, 80 mg to 160 mg, 85 mg to 155 mg, 90 mg to 150 mg, 95 mg to 145 mg, 100 mg to 140 mg, 105 mg to 135 mg, 110 mg to 130 mg, 115 mg to 125 mg, 10 mg to 50 mg, 25 mg to 75 mg, 50 mg to 100 mg, 75 mg to 125 mg, 100 mg to 150 mg, 125 mg to 175 mg, 150 mg to 200 mg, 175 mg to 225 mg, 200 mg to 250 mg, 225 mg to 275 mg, 250 mg to 300 mg, 275 mg to 325 mg, 300 mg to 350 mg, 325 mg to 375 mg, 350 mg to 400 mg, 375 mg to 425 mg, 400 mg to 450 mg, 425 mg to 475 mg, 450 mg to 500 mg, 475 mg to 525 mg, 500 mg to 550 mg, 525 mg to 575 mg, 550 mg to 600 mg, 575 mg to 625 mg, 600 mg to 650 mg, 625 mg to 675 mg, 650 mg to 700 mg, 675 mg to 725 mg, 700 mg to 750 mg, 725 mg to 775 mg, 750 mg to 800 mg, 775 mg to 825 mg, 800 mg to 850 mg, 825 mg to 875 mg, 850 mg to 900 mg, 875 mg to 925 mg, 900 mg to 950 mg, 925 mg to 975 mg, or 950 mg to 1000 mg).

In some aspects, the C1D3 is between 10 mg to 200 mg (e.g., between 10 mg to 170 mg, 11 mg to 165 mg, 12 mg to 160 mg, 13 mg to 155 mg, 14 mg to 150 mg, 15 mg to 145 mg, 16 mg to 140 mg, 17 mg to 135 mg, 18 mg to 130 mg, 19 mg to 125 mg, 20 mg to 120 mg, 21 mg to 115 mg, 22 mg to 110 mg, 23 mg to 105 mg, 24 mg to 100 mg, 25 mg to 95 mg, 26 mg to 90 mg, 27 mg to 85 mg, 28 mg to 80 mg, 29 mg to 75 mg, 30 mg to 70 mg, 31 mg to 65 mg, 32 mg to 60 mg, 33 mg to 55 mg, 34 mg to 50 mg, 35 mg to 45 mg, 40 mg to 200 mg, 45 mg to 195 mg, 50 mg to 190 mg, 55 mg to 185 mg, 60 mg to 180 mg, 65 mg to 175 mg, 70 mg to 170 mg, 75 mg to 165 mg, 80 mg to 160 mg, 85 mg to 155 mg, 90 mg to 150 mg, 95 mg to 145 mg, 100 mg to 140 mg, 105 mg to 135 mg, 110 mg to 130 mg, 115 mg to 125 mg, 10 mg to 20 mg, 20 mg to 30 mg, 30 mg to 40 mg, 40 mg to 50 mg, 50 mg to 60 mg, 60 mg to 70 mg, 70 mg to 80 mg, 80 mg to 90 mg, 90 mg to 100 mg, 100 mg to 110 mg, 110 mg to 120 mg, 120 mg to 130 mg, 130 mg to 140 mg, 140 mg to 150 mg, 150 mg to 160 mg, 160 mg to 170 mg, 170 mg to 180 mg, 180 mg to 190 mg, or 190 mg to 200 mg).

In some aspects, the C1D1 is about 0.1 mg. In some aspects, the C1D1 is about 0.2 mg. In some aspects, the C1D1 is about 0.3 mg. In some aspects, the C1D1 is about 0.4 mg. In some aspects, the C1D1 is about 0.5 mg. In some aspects, the C1D1 is about 0.6 mg. In some aspects, the C1D1 is about 0.7 mg. In some aspects, the C1D1 is about 0.8 mg. In some aspects, the C1D1 is about 0.9 mg. In some aspects, the C1D1 is about 1 mg. In some aspects, the C1D1 is about 2 mg. In some aspects, the C1D1 is about 3 mg. In some aspects, the C1D1 is about 4 mg. In some aspects, the C1D1 is about 5 mg. In some aspects, the C1D1 is about 6 mg. In some aspects, the C1D1 is about 7 mg. In some aspects, the C1D1 is about 8 mg. In some aspects, the C1D1 is about 9 mg. In some aspects, the C1D1 is about 10 mg.

In some aspects, the C1D2 is about 1 mg. In some aspects, the C1D2 is about 2 mg. In some aspects, the C1D2 is about 3 mg. In some aspects, the C1D2 is about 4 mg. In some aspects, the C1D2 is about 5 mg. In some aspects, the C1D2 is about 6 mg. In some aspects, the C1D2 is about 7 mg. In some aspects, the C1D2 is about 8 mg. In some aspects, the C1D2 is about 9 mg. In some aspects, the C1D2 is about 10 mg. In some aspects, the C1D2 is about 11 mg. In some aspects, the C1D2 is about 12 mg. In some aspects, the C1D2 is about 13 mg. In some aspects, the C1D2 is about 14 mg. In some aspects, the C1D2 is about 15 mg. In some aspects, the C1D2 is about 16 mg. In some aspects, the C1D2 is about 17 mg. In some aspects, the C1D2 is about 18 mg. In some aspects, the C1D2 is about 19 mg. In some aspects, the C1D2 is about 20 mg. In some aspects, the C1D2 is about 21 mg. In some aspects, the C1D2 is about 22 mg. In some aspects, the C1D2 is about 23 mg. In some aspects, the C1D2 is about 24 mg. In some aspects, the C1D2 is about 25 mg. In some aspects, the C1D2 is about 26 mg. In some aspects, the C1D2 is about 27 mg. In some aspects, the C1D2 is about 28 mg. In some aspects, the C1D2 is about 29 mg. In some aspects, the C1D2 is about 30 mg. In some aspects, the C1D2 is about 31 mg. In some aspects, the C1D2 is about 32 mg. In some aspects, the C1D2 is about 33 mg. In some aspects, the C1D2 is about 34 mg. In some aspects, the C1D2 is about 35 mg. In some aspects, the C1D2 is about 36 mg. In some aspects, the C1D2 is about 37 mg. In some aspects, the C1D2 is about 38 mg. In some aspects, the C1D2 is about 39 mg. In some aspects, the C1D2 is about 40 mg. In some aspects, the C1D2 is about 41 mg. In some aspects, the C1D2 is about 42 mg. In some aspects, the C1D2 is about 43 mg. In some aspects, the C1D2 is about 44 mg. In some aspects, the C1D2 is about 45 mg. In some aspects, the C1D2 is about 46 mg. In some aspects, the C1D2 is about 47 mg. In some aspects, the C1D2 is about 48 mg. In some aspects, the C1D2 is about 49 mg. In some aspects, the C1D2 is about 50 mg.

In some aspects, the C1D3 is about 10 mg. In some aspects, the C1D3 is about 15 mg. In some aspects, the C1D3 is about 20 mg. In some aspects, the C1D3 is about 25 mg. In some aspects, the C1D3 is about 30 mg. In some aspects, the C1D3 is about 35 mg. In some aspects, the C1D3 is about 40 mg. In some aspects, the C1D3 is about 45 mg. In some aspects, the C1D3 is about 50 mg. In some aspects, the C1D3 is about 55 mg. In some aspects, the C1D3 is about 60 mg. In some aspects, the C1D3 is about 65 mg. In some aspects, the C1D3 is about 70 mg. In some aspects, the C1D3 is about 75 mg. In some aspects, the C1D3 is about 80 mg. In some aspects, the C1D3 is about 85 mg. In some aspects, the C1D3 is about 90 mg. In some aspects, the C1D3 is about 95 mg. In some aspects, the C1D3 is about 100 mg. In some aspects, the C1D3 is about 105 mg. In some aspects, the C1D3 is about 110 mg. In some aspects, the C1D3 is about 115 mg. In some aspects, the C1D3 is about 120 mg. In some aspects, the C1D3 is about 125 mg. In some aspects, the C1D3 is about 130 mg. In some aspects, the C1D3 is about 135 mg. In some aspects, the C1D3 is about 140 mg. In some aspects, the C1D3 is about 145 mg. In some aspects, the C1D3 is about 150 mg. In some aspects, the C1D3 is about 155 mg. In some aspects, the C1D3 is about 160 mg. In some aspects, the C1D3 is about 165 mg. In some aspects, the C1D3 is about 170 mg. In some aspects, the C1D3 is about 175 mg. In some aspects, the C1D3 is about 180 mg. In some aspects, the C1D3 is about 185 mg. In some aspects, the C1D3 is about 190 mg. In some aspects, the C1D3 is about 195 mg. In some aspects, the C1D3 is about 200 mg. In some aspects, the C1D3 is about 205 mg. In some aspects, the C1D3 is about 210 mg. In some aspects, the C1D3 is about 215 mg. In some aspects, the C1D3 is about 220 mg. In some aspects, the C1D3 is about 225 mg. In some aspects, the C1D3 is about 230 mg. In some aspects, the C1D3 is about 235 mg. In some aspects, the C1D3 is about 240 mg. In some aspects, the C1D3 is about 245 mg. In some aspects, the C1D3 is about 250 mg. In some aspects, the C1D3 is about 255 mg. In some aspects, the C1D3 is about 260 mg. In some aspects, the C1D3 is about 265 mg. In some aspects, the C1D3 is about 270 mg. In some aspects, the C1D3 is about 275 mg. In some aspects, the C1D3 is about 280 mg. In some aspects, the C1D3 is about 285 mg. In some aspects, the C1D3 is about 290 mg. In some aspects, the C1D3 is about 295 mg. In some aspects, the C1D3 is about 300 mg. In some aspects, the C1D3 is about 305 mg. In some aspects, the C1D3 is about 310 mg. In some aspects, the C1D3 is about 315 mg. In some aspects, the C1D3 is about 320 mg. In some aspects, the C1D3 is about 325 mg. In some aspects, the C1D3 is about 330 mg. In some aspects, the C1D3 is about 335 mg. In some aspects, the C1D3 is about 340 mg. In some aspects, the C1D3 is about 345 mg. In some aspects, the C1D3 is about 350 mg. In some aspects, the C1D3 is about 355 mg. In some aspects, the C1D3 is about 360 mg. In some aspects, the C1D3 is about 365 mg. In some aspects, the C1D3 is about 370 mg. In some aspects, the C1D3 is about 375 mg. In some aspects, the C1D3 is about 380 mg. In some aspects, the C1D3 is about 385 mg. In some aspects, the C1D3 is about 390 mg. In some aspects, the C1D3 is about 395 mg. In some aspects, the C1D3 is about 400 mg. In some aspects, the C1D3 is about 405 mg. In some aspects, the C1D3 is about 410 mg. In some aspects, the C1D3 is about 415 mg. In some aspects, the C1D3 is about 420 mg. In some aspects, the C1D3 is about 425 mg. In some aspects, the C1D3 is about 430 mg. In some aspects, the C1D3 is about 435 mg. In some aspects, the C1D3 is about 440 mg. In some aspects, the C1D3 is about 445 mg. In some aspects, the C1D3 is about 450 mg. In some aspects, the C1D3 is about 455 mg. In some aspects, the C1D3 is about 460 mg. In some aspects, the C1D3 is about 465 mg. In some aspects, the C1D3 is about 470 mg. In some aspects, the C1D3 is about 475 mg. In some aspects, the C1D3 is about 480 mg. In some aspects, the C1D3 is about 485 mg. In some aspects, the C1D3 is about 490 mg. In some aspects, the C1D3 is about 495 mg. In some aspects, the C1D3 is about 500 mg. In some aspects, the C1D3 is about 505 mg. In some aspects, the C1D3 is about 510 mg. In some aspects, the C1D3 is about 515 mg. In some aspects, the C1D3 is about 520 mg. In some aspects, the C1D3 is about 525 mg. In some aspects, the C1D3 is about 530 mg. In some aspects, the C1D3 is about 535 mg. In some aspects, the C1D3 is about 540 mg. In some aspects, the C1D3 is about 545 mg. In some aspects, the C1D3 is about 550 mg. In some aspects, the C1D3 is about 555 mg. In some aspects, the C1D3 is about 560 mg. In some aspects, the C1D3 is about 565 mg. In some aspects, the C1D3 is about 570 mg. In some aspects, the C1D3 is about 575 mg. In some aspects, the C1D3 is about 580 mg. In some aspects, the C1D3 is about 585 mg. In some aspects, the C1D3 is about 590 mg. In some aspects, the C1D3 is about 595 mg. In some aspects, the C1D3 is about 600 mg. In some aspects, the C1D3 is about 605 mg. In some aspects, the C1D3 is about 610 mg. In some aspects, the C1D3 is about 615 mg. In some aspects, the C1D3 is about 620 mg. In some aspects, the C1D3 is about 625 mg. In some aspects, the C1D3 is about 630 mg. In some aspects, the C1D3 is about 635 mg. In some aspects, the C1D3 is about 640 mg. In some aspects, the C1D3 is about 645 mg. In some aspects, the C1D3 is about 650 mg. In some aspects, the C1D3 is about 655 mg. In some aspects, the C1D3 is about 660 mg. In some aspects, the C1D3 is about 665 mg. In some aspects, the C1D3 is about 670 mg. In some aspects, the C1D3 is about 675 mg. In some aspects, the C1D3 is about 680 mg. In some aspects, the C1D3 is about 685 mg. In some aspects, the C1D3 is about 690 mg. In some aspects, the C1D3 is about 695 mg. In some aspects, the C1D3 is about 700 mg. In some aspects, the C1D3 is about 705 mg. In some aspects, the C1D3 is about 710 mg. In some aspects, the C1D3 is about 715 mg. In some aspects, the C1D3 is about 720 mg. In some aspects, the C1D3 is about 725 mg. In some aspects, the C1D3 is about 730 mg. In some aspects, the C1D3 is about 735 mg. In some aspects, the C1D3 is about 740 mg. In some aspects, the C1D3 is about 745 mg. In some aspects, the C1D3 is about 750 mg. In some aspects, the C1D3 is about 755 mg. In some aspects, the C1D3 is about 760 mg. In some aspects, the C1D3 is about 765 mg. In some aspects, the C1D3 is about 770 mg. In some aspects, the C1D3 is about 775 mg. In some aspects, the C1D3 is about 780 mg. In some aspects, the C1D3 is about 785 mg. In some aspects, the C1D3 is about 790 mg. In some aspects, the C1D3 is about 795 mg. In some aspects, the C1D3 is about 800 mg. In some aspects, the C1D3 is about 805 mg. In some aspects, the C1D3 is about 810 mg. In some aspects, the C1D3 is about 815 mg. In some aspects, the C1D3 is about 820 mg. In some aspects, the C1D3 is about 825 mg. In some aspects, the C1D3 is about 830 mg. In some aspects, the C1D3 is about 835 mg. In some aspects, the C1D3 is about 840 mg. In some aspects, the C1D3 is about 845 mg. In some aspects, the C1D3 is about 850 mg. In some aspects, the C1D3 is about 855 mg. In some aspects, the C1D3 is about 860 mg. In some aspects, the C1D3 is about 865 mg. In some aspects, the C1D3 is about 870 mg. In some aspects, the C1D3 is about 875 mg. In some aspects, the C1D3 is about 880 mg. In some aspects, the C1D3 is about 885 mg. In some aspects, the C1D3 is about 890 mg. In some aspects, the C1D3 is about 895 mg. In some aspects, the C1D3 is about 900 mg. In some aspects, the C1D3 is about 905 mg. In some aspects, the C1D3 is about 910 mg. In some aspects, the C1D3 is about 915 mg. In some aspects, the C1D3 is about 920 mg. In some aspects, the C1D3 is about 925 mg. In some aspects, the C1D3 is about 930 mg. In some aspects, the C1D3 is about 935 mg. In some aspects, the C1D3 is about 940 mg. In some aspects, the C1D3 is about 945 mg. In some aspects, the C1D3 is about 950 mg. In some aspects, the C1D3 is about 955 mg. In some aspects, the C1D3 is about 960 mg. In some aspects, the C1D3 is about 965 mg. In some aspects, the C1D3 is about 970 mg. In some aspects, the C1D3 is about 975 mg. In some aspects, the C1D3 is about 980 mg. In some aspects, the C1D3 is about 985 mg. In some aspects, the C1D3 is about 990 mg. In some aspects, the C1D3 is about 995 mg. In some aspects, the C1D3 is about 1000 mg.

In some aspects, the C1D1 is 0.1 mg. In some aspects, the C1D1 is 0.2 mg. In some aspects, the C1D1 is 0.3 mg. In some aspects, the C1D1 is 0.4 mg. In some aspects, the C1D1 is 0.5 mg. In some aspects, the C1D1 is 0.6 mg. In some aspects, the C1D1 is 0.7 mg. In some aspects, the C1D1 is 0.8 mg. In some aspects, the C1D1 is 0.9 mg. In some aspects, the C1D1 is 1 mg. In some aspects, the C1D1 is 2 mg. In some aspects, the C1D1 is 3 mg. In some aspects, the C1D1 is 4 mg. In some aspects, the C1D1 is 5 mg. In some aspects, the C1D1 is 6 mg. In some aspects, the C1D1 is 7 mg. In some aspects, the C1D1 is 8 mg. In some aspects, the C1D1 is 9 mg. In some aspects, the C1D1 is 10 mg.

In some aspects, the C1D2 is 1 mg. In some aspects, the C1D2 is 2 mg. In some aspects, the C1D2 is 3 mg. In some aspects, the C1D2 is 4 mg. In some aspects, the C1D2 is 5 mg. In some aspects, the C1D2 is 6 mg. In some aspects, the C1D2 is 7 mg. In some aspects, the C1D2 is 8 mg. In some aspects, the C1D2 is 9 mg. In some aspects, the C1D2 is 10 mg. In some aspects, the C1D2 is 11 mg. In some aspects, the C1D2 is 12 mg. In some aspects, the C1D2 is 13 mg. In some aspects, the C1D2 is 14 mg. In some aspects, the C1D2 is 15 mg. In some aspects, the C1D2 is 16 mg. In some aspects, the C1D2 is 17 mg. In some aspects, the C1D2 is 18 mg. In some aspects, the C1D2 is 19 mg. In some aspects, the C1D2 is 20 mg. In some aspects, the C1D2 is 21 mg. In some aspects, the C1D2 is 22 mg. In some aspects, the C1D2 is 23 mg. In some aspects, the C1D2 is 24 mg. In some aspects, the C1D2 is 25 mg. In some aspects, the C1D2 is 26 mg. In some aspects, the C1D2 is 27 mg. In some aspects, the C1D2 is 28 mg. In some aspects, the C1D2 is 29 mg. In some aspects, the C1D2 is 30 mg. In some aspects, the C1D2 is 31 mg. In some aspects, the C1D2 is 32 mg. In some aspects, the C1D2 is 33 mg. In some aspects, the C1D2 is 34 mg. In some aspects, the C1D2 is 35 mg. In some aspects, the C1D2 is 36 mg. In some aspects, the C1D2 is 37 mg. In some aspects, the C1D2 is 38 mg. In some aspects, the C1D2 is 39 mg. In some aspects, the C1D2 is 40 mg. In some aspects, the C1D2 is 41 mg. In some aspects, the C1D2 is 42 mg. In some aspects, the C1D2 is 43 mg. In some aspects, the C1D2 is 44 mg. In some aspects, the C1D2 is 45 mg. In some aspects, the C1D2 is 46 mg. In some aspects, the C1D2 is 47 mg. In some aspects, the C1D2 is 48 mg. In some aspects, the C1D2 is 49 mg. In some aspects, the C1D2 is 50 mg.

In some aspects, the C1D3 is 10 mg. In some aspects, the C1D3 is 15 mg. In some aspects, the C1D3 is 20 mg. In some aspects, the C1D3 is 25 mg. In some aspects, the C1D3 is 30 mg. In some aspects, the C1D3 is 35 mg. In some aspects, the C1D3 is 40 mg. In some aspects, the C1D3 is 45 mg. In some aspects, the C1D3 is 50 mg. In some aspects, the C1D3 is 55 mg. In some aspects, the C1D3 is 60 mg. In some aspects, the C1D3 is 65 mg. In some aspects, the C1D3 is 70 mg. In some aspects, the C1D3 is 75 mg. In some aspects, the C1D3 is 80 mg. In some aspects, the C1D3 is 85 mg. In some aspects, the C1D3 is 90 mg. In some aspects, the C1D3 is 95 mg. In some aspects, the C1D3 is 100 mg. In some aspects, the C1D3 is 105 mg. In some aspects, the C1D3 is 110 mg. In some aspects, the C1D3 is 115 mg. In some aspects, the C1D3 is 120 mg. In some aspects, the C1D3 is 125 mg. In some aspects, the C1D3 is 130 mg. In some aspects, the C1D3 is 135 mg. In some aspects, the C1D3 is 140 mg. In some aspects, the C1D3 is 145 mg. In some aspects, the C1D3 is 150 mg. In some aspects, the C1D3 is 155 mg. In some aspects, the C1D3 is 160 mg. In some aspects, the C1D3 is 165 mg. In some aspects, the C1D3 is 170 mg. In some aspects, the C1D3 is 175 mg. In some aspects, the C1D3 is 180 mg. In some aspects, the C1D3 is 185 mg. In some aspects, the C1D3 is 190 mg. In some aspects, the C1D3 is 195 mg. In some aspects, the C1D3 is 200 mg. In some aspects, the C1D3 is 205 mg. In some aspects, the C1D3 is 210 mg. In some aspects, the C1D3 is 215 mg. In some aspects, the C1D3 is 220 mg. In some aspects, the C1D3 is 225 mg. In some aspects, the C1D3 is 230 mg. In some aspects, the C1D3 is 235 mg. In some aspects, the C1D3 is 240 mg. In some aspects, the C1D3 is 245 mg. In some aspects, the C1D3 is 250 mg. In some aspects, the C1D3 is 255 mg. In some aspects, the C1D3 is 260 mg. In some aspects, the C1D3 is 265 mg. In some aspects, the C1D3 is 270 mg. In some aspects, the C1D3 is 275 mg. In some aspects, the C1D3 is 280 mg. In some aspects, the C1D3 is 285 mg. In some aspects, the C1D3 is 290 mg. In some aspects, the C1D3 is 295 mg. In some aspects, the C1D3 is 300 mg. In some aspects, the C1D3 is 305 mg. In some aspects, the C1D3 is 310 mg. In some aspects, the C1D3 is 315 mg. In some aspects, the C1D3 is 320 mg. In some aspects, the C1D3 is 325 mg. In some aspects, the C1D3 is 330 mg. In some aspects, the C1D3 is 335 mg. In some aspects, the C1D3 is 340 mg. In some aspects, the C1D3 is 345 mg. In some aspects, the C1D3 is 350 mg. In some aspects, the C1D3 is 355 mg. In some aspects, the C1D3 is 360 mg. In some aspects, the C1D3 is 365 mg. In some aspects, the C1D3 is 370 mg. In some aspects, the C1D3 is 375 mg. In some aspects, the C1D3 is 380 mg. In some aspects, the C1D3 is 385 mg. In some aspects, the C1D3 is 390 mg. In some aspects, the C1D3 is 395 mg. In some aspects, the C1D3 is 400 mg. In some aspects, the C1D3 is 405 mg. In some aspects, the C1D3 is 410 mg. In some aspects, the C1D3 is 415 mg. In some aspects, the C1D3 is 420 mg. In some aspects, the C1D3 is 425 mg. In some aspects, the C1D3 is 430 mg. In some aspects, the C1D3 is 435 mg. In some aspects, the C1D3 is 440 mg. In some aspects, the C1D3 is 445 mg. In some aspects, the C1D3 is 450 mg. In some aspects, the C1D3 is 455 mg. In some aspects, the C1D3 is 460 mg. In some aspects, the C1D3 is 465 mg. In some aspects, the C1D3 is 470 mg. In some aspects, the C1D3 is 475 mg. In some aspects, the C1D3 is 480 mg. In some aspects, the C1D3 is 485 mg. In some aspects, the C1D3 is 490 mg. In some aspects, the C1D3 is 495 mg. In some aspects, the C1D3 is 500 mg. In some aspects, the C1D3 is 505 mg. In some aspects, the C1D3 is 510 mg. In some aspects, the C1D3 is 515 mg. In some aspects, the C1D3 is 520 mg. In some aspects, the C1D3 is 525 mg. In some aspects, the C1D3 is 530 mg. In some aspects, the C1D3 is 535 mg. In some aspects, the C1D3 is 540 mg. In some aspects, the C1D3 is 545 mg. In some aspects, the C1D3 is 550 mg. In some aspects, the C1D3 is 555 mg. In some aspects, the C1D3 is 560 mg. In some aspects, the C1D3 is 565 mg. In some aspects, the C1D3 is 570 mg. In some aspects, the C1D3 is 575 mg. In some aspects, the C1D3 is 580 mg. In some aspects, the C1D3 is 585 mg. In some aspects, the C1D3 is 590 mg. In some aspects, the C1D3 is 595 mg. In some aspects, the C1D3 is 600 mg. In some aspects, the C1D3 is 605 mg. In some aspects, the C1D3 is 610 mg. In some aspects, the C1D3 is 615 mg. In some aspects, the C1D3 is 620 mg. In some aspects, the C1D3 is 625 mg. In some aspects, the C1D3 is 630 mg. In some aspects, the C1D3 is 635 mg. In some aspects, the C1D3 is 640 mg. In some aspects, the C1D3 is 645 mg. In some aspects, the C1D3 is 650 mg. In some aspects, the C1D3 is 655 mg. In some aspects, the C1D3 is 660 mg. In some aspects, the C1D3 is 665 mg. In some aspects, the C1D3 is 670 mg. In some aspects, the C1D3 is 675 mg. In some aspects, the C1D3 is 680 mg. In some aspects, the C1D3 is 685 mg. In some aspects, the C1D3 is 690 mg. In some aspects, the C1D3 is 695 mg. In some aspects, the C1D3 is 700 mg. In some aspects, the C1D3 is 705 mg. In some aspects, the C1D3 is 710 mg. In some aspects, the C1D3 is 715 mg. In some aspects, the C1D3 is 720 mg. In some aspects, the C1D3 is 725 mg. In some aspects, the C1D3 is 730 mg. In some aspects, the C1D3 is 735 mg. In some aspects, the C1D3 is 740 mg. In some aspects, the C1D3 is 745 mg. In some aspects, the C1D3 is 750 mg. In some aspects, the C1D3 is 755 mg. In some aspects, the C1D3 is 760 mg. In some aspects, the C1D3 is 765 mg. In some aspects, the C1D3 is 770 mg. In some aspects, the C1D3 is 775 mg. In some aspects, the C1D3 is 780 mg. In some aspects, the C1D3 is 785 mg. In some aspects, the C1D3 is 790 mg. In some aspects, the C1D3 is 795 mg. In some aspects, the C1D3 is 800 mg. In some aspects, the C1D3 is 805 mg. In some aspects, the C1D3 is 810 mg. In some aspects, the C1D3 is 815 mg. In some aspects, the C1D3 is 820 mg. In some aspects, the C1D3 is 825 mg. In some aspects, the C1D3 is 830 mg. In some aspects, the C1D3 is 835 mg. In some aspects, the C1D3 is 840 mg. In some aspects, the C1D3 is 845 mg. In some aspects, the C1D3 is 850 mg. In some aspects, the C1D3 is 855 mg. In some aspects, the C1D3 is 860 mg. In some aspects, the C1D3 is 865 mg. In some aspects, the C1D3 is 870 mg. In some aspects, the C1D3 is 875 mg. In some aspects, the C1D3 is 880 mg. In some aspects, the C1D3 is 885 mg. In some aspects, the C1D3 is 890 mg. In some aspects, the C1D3 is 895 mg. In some aspects, the C1D3 is 900 mg. In some aspects, the C1D3 is 905 mg. In some aspects, the C1D3 is 910 mg. In some aspects, the C1D3 is 915 mg. In some aspects, the C1D3 is 920 mg. In some aspects, the C1D3 is 925 mg. In some aspects, the C1D3 is 930 mg. In some aspects, the C1D3 is 935 mg. In some aspects, the C1D3 is 940 mg. In some aspects, the C1D3 is 945 mg. In some aspects, the C1D3 is 950 mg. In some aspects, the C1D3 is 955 mg. In some aspects, the C1D3 is 960 mg. In some aspects, the C1D3 is 965 mg. In some aspects, the C1D3 is 970 mg. In some aspects, the C1D3 is 975 mg. In some aspects, the C1D3 is 980 mg. In some aspects, the C1D3 is 985 mg. In some aspects, the C1D3 is 990 mg. In some aspects, the C1D3 is 995 mg. In some aspects, the C1D3 is 1000 mg.

In some instances, the methods described above may include a first dosing cycle of four weeks or 28 days. In some instances, the methods may include administering to the subject the C1D1 and the C1D2 on or about Days 1 and 8, respectively, of the first dosing cycle. In some instances, the methods may include administering to the subject the C1D1, the C1D2, and the C1D3 on or about Days 1, 8, and 15, respectively, of the first dosing cycle.

In some aspects, the method comprises only a single dosing cycle of the bispecific antibody (e.g., a dosing cycle comprising a C1D1, a C1D2, and a C1D3).

iv. Further Dosing Cycles

Any of the methods described herein may include a further dosing cycle. For example, in some instances, the methods described above may include a second dosing cycle of four weeks or 28 days. In some instances, the methods may include administering to the subject the C2D1 on or about Day 1 and Day 15 of the second dosing cycle.

In some instances, in which the methods include at least a second dosing cycle, the methods may include one or more additional dosing cycles. In some instances, the dosing regimen comprises 1 to 17 additional dosing cycles (e.g., 1, 2, 3, 4, 5, 6, 7, 8, 9, 10, 11, 12, 13, 14, 15, 16, or 17 additional dosing cycles, e.g., 1-3 additional dosing cycles, 1-5 additional dosing cycles, 3-8 additional dosing cycles, 5-10 additional dosing cycles, 8-12 additional dosing cycles, 10-15 additional dosing cycles, 12-17 additional dosing cycles, or 15-17 additional dosing cycles, i.e., the dosing regimen includes one or more of additional dosing cycle(s) C3, C4, C5, C6, C7, C8, C9, C10, C11, C12, C13, C14, C15, C16, C17, C18, and C19.

In some embodiments, the length of each of the one or more additional dosing cycles is 7 days, 14 days, 21 days, or 28 days. In some embodiments, the length of each of the one or more additional dosing cycles is between 5 days and 30 days, e.g., between 5 and 9 days, between 7 and 11 days, between 9 and 13 days, between 11 and 15 days, between 13 and 17 days, between 15 and 19 days, between 17 and 21 days, between 19 and 23 days, between 21 and 25 days, between 23 and 27 days, or between 25 and 30 days. In some instances, the length of each of the one or more additional dosing cycles is three weeks or 21 days. In some instances, the length of each of the one or more additional dosing cycles is four weeks or 28 days.

In some instances, each of the one or more additional dosing cycles comprises a single dose of the bispecific antibody. In some aspects, the dose of the bispecific antibody in the one or more additional dosing cycles is equal to the C1D3, e.g., is between about 10 mg to about 1000 mg (e.g., between about 10 mg to about 70 mg, about 15 mg to about 65 mg, about 20 mg to about 60 mg, about 25 mg to about 55 mg, about 30 mg to about 50 mg, about 35 mg to about 45 mg, about 15 mg to about 225 mg, about 20 mg to about 220 mg, about 25 mg to about 215 mg, about 30 mg to about 210 mg, about 35 mg to about 205 mg, about 40 mg to about 200 mg, about 45 mg to about 195 mg, about 50 mg to about 190 mg, about 55 mg to about 185 mg, about 60 mg to about 180 mg, about 65 mg to about 175 mg, about 70 mg to about 170 mg, about 75 mg to about 165 mg, about 80 mg to about 160 mg, about 85 mg to about 155 mg, about 90 mg to about 150 mg, about 95 mg to about 145 mg, about 100 mg to about 140 mg, about 105 mg to about 135 mg, about 110 mg to about 130 mg, about 115 mg to about 125 mg, about 10 mg to about 50 mg, about 25 mg to about 75 mg, about 50 mg to about 100 mg, about 75 mg to about 125 mg, about 100 mg to about 150 mg, about 125 mg to about 175 mg, about 150 mg to about 200 mg, about 175 mg to about 225 mg, about 200 mg to about 250 mg, about 225 mg to about 275 mg, about 250 mg to about 300 mg, about 275 mg to about 325 mg, about 300 mg to about 350 mg, about 325 mg to about 375 mg, about 350 mg to about 400 mg, about 375 mg to about 425 mg, about 400 mg to about 450 mg, about 425 mg to about 475 mg, about 450 mg to about 500 mg, about 475 mg to about 525 mg, about 500 mg to about 550 mg, about 525 mg to about 575 mg, about 550 mg to about 600 mg, about 575 mg to about 625 mg, about 600 mg to about 650 mg, about 625 mg to about 675 mg, about 650 mg to about 700 mg, about 675 mg to about 725 mg, about 700 mg to about 750 mg, about 725 mg to about 775 mg, about 750 mg to about 800 mg, about 775 mg to about 825 mg, about 800 mg to about 850 mg, about 825 mg to about 875 mg, about 850 mg to about 900 mg, about 875 mg to about 925 mg, about 900 mg to about 950 mg, about 925 mg to about 975 mg, or about 950 mg to about 1000 mg). In some aspects, the C1D2 is greater than the C1D1 and the C1D3 is greater than the C1D2.

In some aspects, the dose of the bispecific antibody in the one or more additional dosing cycles is between about 10 mg to about 200 mg (e.g., between about 10 mg to about 170 mg, about 11 mg to about 165 mg, about 12 mg to about 160 mg, about 13 mg to about 155 mg, about 14 mg to about 150 mg, about 15 mg to about 145 mg, about 16 mg to about 140 mg, about 17 mg to about 135 mg, about 18 mg to about 130 mg, about 19 mg to about 125 mg, about 20 mg to about 120 mg, about 21 mg to about 115 mg, about 22 mg to about 110 mg, about 23 mg to about 105 mg, about 24 mg to about 100 mg, about 25 mg to about 95 mg, about 26 mg to about 90 mg, about 27 mg to about 85 mg, about 28 mg to about 80 mg, about 29 mg to about 75 mg, about 30 mg to about 70 mg, about 31 mg to about 65 mg, about 32 mg to about 60 mg, about 33 mg to about 55 mg, about 34 mg to about 50 mg, about 35 mg to about 45 mg, about 40 mg to about 200 mg, about 45 mg to about 195 mg, about 50 mg to about 190 mg, about 55 mg to about 185 mg, about 60 mg to about 180 mg, about 65 mg to about 175 mg, about 70 mg to about 170 mg, about 75 mg to about 165 mg, about 80 mg to about 160 mg, about 85 mg to about 155 mg, about 90 mg to about 150 mg, about 95 mg to about 145 mg, about 100 mg to about 140 mg, about 105 mg to about 135 mg, about 110 mg to about 130 mg, about 115 mg to about 125 mg, about 10 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, about 90 mg to about 100 mg, about 100 mg to about 110 mg, about 110 mg to about 120 mg, about 120 mg to about 130 mg, about 130 mg to about 140 mg, about 140 mg to about 150 mg, about 150 mg to about 160 mg, about 160 mg to about 170 mg, about 170 mg to about 180 mg, about 180 mg to about 190 mg, or about 190 mg to about 200 mg).

In some aspects, the dose of the bispecific antibody in the one or more additional dosing cycles is about 40 mg. In some aspects, the dose of the bispecific antibody in the one or more additional dosing cycles is about 120 mg. In some aspects, the dose of the bispecific antibody in the one or more additional dosing cycles is equal to the C1D3, e.g., is between 10 mg 10 mg to 1000 mg (e.g., between 10 mg to 70 mg, 15 mg to 65 mg, 20 mg to 60 mg, 25 mg to 55 mg, 30 mg to 50 mg, 35 mg to 45 mg, 15 mg to 225 mg, 20 mg to 220 mg, 25 mg to 215 mg, 30 mg to 210 mg, 35 mg to 205 mg, 40 mg to 200 mg, 45 mg to 195 mg, 50 mg to 190 mg, 55 mg to 185 mg, 60 mg to 180 mg, 65 mg to 175 mg, 70 mg to 170 mg, 75 mg to 165 mg, 80 mg to 160 mg, 85 mg to 155 mg, 90 mg to 150 mg, 95 mg to 145 mg, 100 mg to 140 mg, 105 mg to 135 mg, 110 mg to 130 mg, 115 mg to 125 mg, 10 mg to 50 mg, 25 mg to 75 mg, 50 mg to 100 mg, 75 mg to 125 mg, 100 mg to 150 mg, 125 mg to 175 mg, 150 mg to 200 mg, 175 mg to 225 mg, 200 mg to 250 mg, 225 mg to 275 mg, 250 mg to 300 mg, 275 mg to 325 mg, 300 mg to 350 mg, 325 mg to 375 mg, 350 mg to 400 mg, 375 mg to 425 mg, 400 mg to 450 mg, 425 mg to 475 mg, 450 mg to 500 mg, 475 mg to 525 mg, 500 mg to 550 mg, 525 mg to 575 mg, 550 mg to 600 mg, 575 mg to 625 mg, 600 mg to 650 mg, 625 mg to 675 mg, 650 mg to 700 mg, 675 mg to 725 mg, 700 mg to 750 mg, 725 mg to 775 mg, 750 mg to 800 mg, 775 mg to 825 mg, 800 mg to 850 mg, 825 mg to 875 mg, 850 mg to 900 mg, 875 mg to 925 mg, 900 mg to 950 mg, 925 mg to 975 mg, or 950 mg to 1000 mg). In some aspects, the C1D2 is greater than the C1D1 and the C1D3 is greater than the C1D2.

In some aspects, the C1D3 is between 10 mg to 200 mg (e.g., between 10 mg to 170 mg, 11 mg to 165 mg, 12 mg to 160 mg, 13 mg to 155 mg, 14 mg to 150 mg, 15 mg to 145 mg, 16 mg to 140 mg, 17 mg to 135 mg, 18 mg to 130 mg, 19 mg to 125 mg, 20 mg to 120 mg, 21 mg to 115 mg, 22 mg to 110 mg, 23 mg to 105 mg, 24 mg to 100 mg, 25 mg to 95 mg, 26 mg to 90 mg, 27 mg to 85 mg, 28 mg to 80 mg, 29 mg to 75 mg, 30 mg to 70 mg, 31 mg to 65 mg, 32 mg to 60 mg, 33 mg to 55 mg, 34 mg to 50 mg, 35 mg to 45 mg, 40 mg to 200 mg, 45 mg to 195 mg, 50 mg to 190 mg, 55 mg to 185 mg, 60 mg to 180 mg, 65 mg to 175 mg, 70 mg to 170 mg, 75 mg to 165 mg, 80 mg to 160 mg, 85 mg to 155 mg, 90 mg to 150 mg, 95 mg to 145 mg, 100 mg to 140 mg, 105 mg to 135 mg, 110 mg to 130 mg, 115 mg to 125 mg, 10 mg to 20 mg, 20 mg to 30 mg, 30 mg to 40 mg, 40 mg to 50 mg, 50 mg to 60 mg, 60 mg to 70 mg, 70 mg to 80 mg, 80 mg to 90 mg, 90 mg to 100 mg, 100 mg to 110 mg, 110 mg to 120 mg, 120 mg to 130 mg, 130 mg to 140 mg, 140 mg to 150 mg, 150 mg to 160 mg, 160 mg to 170 mg, 170 mg to 180 mg, 180 mg to 190 mg, or 190 mg to 200 mg). In some aspects, the dose of the bispecific antibody in the one or more additional dosing cycles is 40 mg. In some aspects, the dose of the bispecific antibody in the one or more additional dosing cycles is about 120 mg.

In some instances, the method comprises administering subcutaneously to the subject the single dose of the bispecific antibody on or about Day 1 and Day 15 of the one or more additional dosing cycles. In some instances, the method comprises administering to the subject the single dose of the bispecific antibody on or about Day 1 of the one or more additional dosing cycles. In some instances, the method comprises administering to the subject the single dose of the bispecific antibody on or about Day 1, 8, 15, and/or 22 of the one or more additional dosing cycles.

In some aspects, the bispecific antibody is administered subcutaneously to the subject every 7 days (QW) until progressive disease is observed, for up to 18 cycles, or until minimal residual disease (MRD) is observed. In some aspects, the bispecific antibody is administered to the subject every 14 days (Q2W) until progressive disease is observed, for up to 18 cycles, or until minimal residual disease (MRD) is observed. In some aspects, the bispecific antibody is administered to the subject every 21 days (Q3W) until progressive disease is observed, for up to 18 cycles, or until minimal residual disease (MRD) is observed. In some aspects, the bispecific antibody is administered to the subject every 28 days (Q4W) until progressive disease is observed, for up to 18 cycles, or until minimal residual disease (MRD) is observed.

In some aspects, the bispecific antibody is administered subcutaneously to the subject QW until disease progression is observed. In some aspects, the bispecific antibody is administered subcutaneously to the subject Q2W until disease progression is observed. In some aspects, the bispecific antibody is administered subcutaneously to the subject Q3W until disease progression is observed. In some aspects, the bispecific antibody is administered subcutaneously to the subject Q4W until disease progression is observed. In some instances, the bispecific anti-FcRH5/anti-CD3 antibody is administered to the subject as a monotherapy. In some instances, the bispecific anti-FcRH5/anti-CD3 antibody is administered to the subject in combination with another therapeutic agent. In some instances, the bispecific anti-FcRH5/anti-CD3 antibody is administered to the subject in combination with an anti-CD38 antibody. In some instances, the bispecific anti-FcRH5/anti-CD3 antibody is administered to the subject in combination with a corticosteroid. In some instances, the bispecific anti-FcRH5/anti-CD3 antibody is administered to the subject in combination with an immunomodulatory drug (IMiD). In some instances, the bispecific anti-FcRH5/anti-CD3 antibody is administered to the subject in combination with an anti-CD38 antibody and a corticosteroid. In some instances, the bispecific anti-FcRH5/anti-CD3 antibody is administered to the subject in combination with an IMiD and a corticosteroid. Exemplary anti-CD38 antibodies to be used in combination therapy include daratumumab and isatuximab. Exemplary corticosteroids to be used in combination therapy include dexamethasone and methylprednisolone. Exemplary IMiDs to be used in combination therapy include pomalidomide and lenalidomide.

In some instances, the bispecific anti-FcRH5/anti-CD3 antibody is cevostamab. In some instances, cevostamab is administered to the subject as a monotherapy. In some instances, cevostamab is administered to the subject in combination with pomalidomide (P). In some instances, cevostamab is administered to the subject in combination with dexamethasone (d). In some instances, cevostamab is administered to the subject in combination with pomalidomide and dexamethasone (Pd). In some instances, cevostamab is administered to the subject in combination with daratumumab (D). In some instances, cevostamab is administered to the subject in combination with daratumumab and dexamethasone (Dd).

### B. Dosing Regimens: Frequency and Dosing Cycles

The present disclosure describes a method of treating a subject having a cancer (e.g., a multiple myeloma (MM)), the method comprising subcutaneously administering to the subject a bispecific antibody that binds to FcRH5 and CD3 in a dosing regimen described herein. In some examples, the dosing regimen comprises a first phase comprising one or more dosing cycles, a second phase comprising one or more dosing cycles, and a third phase comprising one or more dosing cycles. In some examples, each dosing cycle is a 28-day dosing cycle. The first phase may include administering the bispecific antibody to the subject every week (QW), the second phase may include administering the bispecific antibody to the subject every two weeks (Q2W), and/or the third phase may include administering the bispecific antibody to the subject every four weeks (Q4W).

For example, provided herein is a method of treating a subject having a cancer (e.g., an MM), the method comprising subcutaneously administering to the subject a bispecific antibody that binds to FcRH5 and CD3 in a dosing regimen comprising: (i) a first phase comprising one or more dosing cycles, wherein the first phase comprises administering the bispecific antibody to the subject every week (QW); (ii) a second phase comprising one or more dosing cycles, wherein the second phase comprises administering the bispecific antibody to the subject every two weeks (Q2W); and/or (iii) a third phase comprising one or more dosing cycles, wherein the third phase comprises administering the bispecific antibody to the subject every four weeks (Q4W). In some examples, the dosing regimen includes the first phase. In some examples, the dosing regimen includes the second phase. In some examples, the dosing regimen includes the third phase. In some examples, the dosing regimen includes the first phase and the second phase. In some examples, the dosing regimen includes the first phase and the third phase. In some examples, the dosing regimen includes the second phase and the third phase. In some examples, the dosing regimen includes the first phase, the second phase, and the third phase.

In another example, provided herein is a bispecific antibody that binds to FcRH5 and CD3 for use in treatment of a subject having a cancer (e.g., an MM), the treatment comprising subcutaneously administering the bispecific antibody to the subject in a dosing regimen comprising: (i) a first phase comprising one or more dosing cycles, wherein the first phase comprises administering the bispecific antibody to the subject every week (QW); (ii) a second phase comprising one or more dosing cycles, wherein the second phase comprises administering the bispecific antibody to the subject every two weeks (Q2W); and/or (iii) a third phase comprising one or more dosing cycles, wherein the third phase comprises administering the bispecific antibody to the subject every four weeks (Q4W). In some examples, the dosing regimen includes the first phase. In some examples, the dosing regimen includes the second phase. In some examples, the dosing regimen includes the third phase. In some examples, the dosing regimen includes the first phase and the second phase. In some examples, the dosing regimen includes the first phase and the third phase. In some examples, the dosing regimen includes the second phase and the third phase. In some examples, the dosing regimen includes the first phase, the second phase, and the third phase.

In another example, provided herein is the use of a bispecific antibody that binds to FcRH5 and CD3 in the manufacture of a medicament for treatment of a subject having a cancer (e.g., an MM), the treatment comprising subcutaneous administration of the bispecific antibody to the subject in a dosing regimen comprising: (i) a first phase comprising one or more dosing cycles, wherein the first phase comprises administering the bispecific antibody to the subject every week (QW); (ii) a second phase comprising one or more dosing cycles, wherein the second phase comprises administering the bispecific antibody to the subject every two weeks (Q2W); and/or (iii) a third phase comprising one or more dosing cycles, wherein the third phase comprises administering the bispecific antibody to the subject every four weeks (Q4W). In some examples, the dosing regimen includes the first phase. In some examples, the dosing regimen includes the second phase. In some examples, the dosing regimen includes the third phase. In some examples, the dosing regimen includes the first phase and the second phase. In some examples, the dosing regimen includes the first phase and the third phase. In some examples, the dosing regimen includes the second phase and the third phase. In some examples, the dosing regimen includes the first phase, the second phase, and the third phase.

The first phase may comprise any suitable number of dosing cycles. For example, in some examples, first phase may comprise at least one dosing cycle, at least two dosing cycles, at least three dosing cycles, at least four dosing cycle, at least five dosing cycles, at least six dosing cycles, at least seven, at least eight dosing cycle, at least nine dosing cycle, at least ten dosing cycle, at least eleven dosing cycles, at least twelve dosing cycles, or at least thirteen dosing cycles, or more.

In some examples, first phase comprises a first dosing cycle (C1); a first dosing cycle and a second dosing cycle (C2); a first dosing cycle, a second dosing cycle (C2), and a third dosing cycle (C3); a first dosing cycle (C1), a second dosing cycle (C2), a third dosing cycle (C3), and a fourth dosing cycle (C4); a first dosing cycle (C1), a second dosing cycle (C2), a third dosing cycle (C3), a fourth dosing cycle (C4), and a fifth dosing cycle (C5); a first dosing cycle (C1), a second dosing cycle (C2), a third dosing cycle (C3), a fourth dosing cycle (C4), a fifth dosing cycle (C5), and a sixth dosing cycle (C6); a first dosing cycle (C1), a second dosing cycle (C2), a third dosing cycle (C3), a fourth dosing cycle (C4), a fifth dosing cycle (C5), a sixth dosing cycle (C6), and a seventh dosing cycle (C7); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), and an eighth dosing cycle (C8); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), and a ninth dosing cycle (C9); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), and a tenth dosing cycle (C10); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), and an eleventh dosing cycle (C11); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), and a twelfth dosing cycle (C12); or a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), and a thirteenth dosing cycle (C13).

The bispecific antibody may be administered on any suitable day of a given dosing cycle. For example, for a 28-day dosing cycle, the bispecific antibody may be administered on Day 1, 2, 3, 4, 5, 6, 7, 8, 9, 10, 11, 12, 13, 14, 15, 16, 17, 18, 19, 20, 21, 22, 23, 24, 25, 26, 27, or 28. In another example, for a 21-day dosing cycle, the bispecific antibody may be administered on Day 1, 2, 3, 4, 5, 6, 7, 8, 9, 10, 11, 12, 13, 14, 15, 16, 17, 18, 19, 20, or 21. In another example, for a 14-day dosing cycle, the bispecific antibody may be administered on Day 1, 2, 3, 4, 5, 6, 7, 8, 9, 10, 11, 12, 13, or 14. In another example, for a 7-day dosing cycle, the bispecific antibody may be administered on Day 1, 2, 3, 4, 5, 6, or 7.

In some examples, the first phase comprises administration of the bispecific antibody to the subject on Days 1, 8, and/or 15 of C1. In a further example, the first phase comprises administration of the bispecific antibody to the subject on Days 1, 8, and/or 15 of C2. In a further example, the first phase comprises administration of the bispecific antibody to the subject on Days 1, 8, and/or 15 of C3. In a further example, the first phase comprises administration of the bispecific antibody to the subject on Days 1, 8, and/or 15 of C4. In a further example, the first phase comprises administration of the bispecific antibody to the subject on Days 1, 8, and/or 15 of C5. In a further example, the first phase comprises administration of the bispecific antibody to the subject on Days 1, 8, and/or 15 of C6. In a further example, the first phase comprises administration of the bispecific antibody to the subject on Days 1, 8, and/or 15 of C7. In a further example, the first phase comprises administration of the bispecific antibody to the subject on Days 1, 8, and/or 15 of C8. In a further example, the first phase comprises administration of the bispecific antibody to the subject on Days 1, 8, and/or 15 of C9. In a further example, the first phase comprises administration of the bispecific antibody to the subject on Days 1, 8, and/or 15 of C10. In a further example, the first phase comprises administration of the bispecific antibody to the subject on Days 1, 8, and/or 15 of C11. In a further example, the first phase comprises administration of the bispecific antibody to the subject on Days 1, 8, and/or 15 of C12. In a further example, the first phase comprises administration of the bispecific antibody to the subject on Days 1, 8, and/or 15 of C13.

In some examples, a target dose of the bispecific antibody is administered to the subject for each administration during the first phase.

In some examples, the first phase comprises administration of a target dose of the bispecific antibody to the subject on Days 1, 8, and/or 15 of C1. In a further example, the first phase comprises administration of the target dose of the bispecific antibody to the subject on Days 1, 8, and/or 15 of C2. In a further example, the first phase comprises administration of the target dose of the bispecific antibody to the subject on Days 1, 8, and/or 15 of C3. In a further example, the first phase comprises administration of the target dose of the bispecific antibody to the subject on Days 1, 8, and/or 15 of C4. In a further example, the first phase comprises administration of the target dose of the bispecific antibody to the subject on Days 1, 8, and/or 15 of C5. In a further example, the first phase comprises administration of the target dose of the bispecific antibody to the subject on Days 1, 8, and/or 15 of C6. In a further example, the first phase comprises administration of the target dose of the bispecific antibody to the subject on Days 1, 8, and/or 15 of C7. In a further example, the first phase comprises administration of the target dose of the bispecific antibody to the subject on Days 1, 8, and/or 15 of C8. In a further example, the first phase comprises administration of the target dose of the bispecific antibody to the subject on Days 1, 8, and/or 15 of C9. In a further example, the first phase comprises administration of the target dose of the bispecific antibody to the subject on Days 1, 8, and/or 15 of C10. In a further example, the first phase comprises administration of the target dose of the bispecific antibody to the subject on Days 1, 8, and/or 15 of C11. In a further example, the first phase comprises administration of the target dose of the bispecific antibody to the subject on Days 1, 8, and/or 15 of C12. In a further example, the first phase comprises administration of the target dose of the bispecific antibody to the subject on Days 1, 8, and/or 15 of C13.

In some examples, the first phase comprises administration of a first step-up dose and a target dose of the bispecific antibody to the subject. The first step-up dose may be administered to the subject during the first phase on Day 1 of C1, on Day 2 of C1, on Day 3 of C1, on day 4 of C1, on Day 5 of C1, on Day 6 of C1, or on Day 7 of C1. The target dose may be administered to the subject during the first phase on Days 8 and/or 15 of C1. In a further example, the target dose may be administered to the subject during the first phase on Days 1, 8, and/or 15 of C2. In a further example, the target dose may be administered to the subject during the first phase on Days 1, 8, and/or 15 of C3. In a further example, the target dose may be administered to the subject during the first phase on Days 1, 8, and/or 15 of C4. In a further example, the target dose may be administered to the subject during the first phase on Days 1, 8, and/or 15 of C5. In a further example, the target dose may be administered to the subject during the first phase on Days 1, 8, and/or 15 of C6. In a further example, the target dose may be administered to the subject during the first phase on Days 1, 8, and/or 15 of C7. In a further example, the target dose may be administered to the subject during the first phase on Days 1, 8, and/or 15 of C8. In a further example, the target dose may be administered to the subject during the first phase on Days 1, 8, and/or 15 of C9. In a further example, the target dose may be administered to the subject during the first phase on Days 1, 8, and/or 15 of C10. In a further example, the target dose may be administered to the subject during the first phase on Days 1, 8, and/or 15 of C11. In a further example, the target dose may be administered to the subject during the first phase on Days 1, 8, and/or 15 of C12. In a further example, the target dose may be administered to the subject during the first phase on Days 1, 8, and/or 15 of C13.

In some examples, the first step-up dose is between about 15% to about 45% of the target dose. In some examples, the first step up dose is about 15%, about 16%, about 17%, about 18%, about 19%, about 20%, about 21%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or about 45% of the target dose. In some examples, the first step-up dose is about 25% of the target dose.

In some examples, the first step-up dose is between 15% to 45% of the target dose. In some examples, the first step up dose is 15%, 16%, 17%, 18%, 19%, 20%, 21%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, or 45% of the target dose. In some examples, the first step-up dose is 25% of the target dose.

In some examples, the first step-up dose is between 0.1 mg to about 50 mg (e.g., between about 0.1 mg to about 9.5 mg, about 0.2 mg to about 9 mg, about 0.3 mg to about 8.5 mg, about 0.4 mg to about 8 mg, about 0.5 mg to about 7.5 mg, about 0.6 mg to about 7 mg, about 0.7 mg to about 6.5 mg, about 0.8 mg to about 6 mg, about 0.9 mg to about 5.5 mg, about 1 mg to about 5 mg, about 1.1 mg to about 4.5 mg, about 1.2 mg to about 4 mg, about 1.3 mg to about 3.5 mg, about 1.4 mg to about 3 mg, about 1.5 mg to about 2.5 mg, about 1 mg to about 3 mg, about 0.2 mg to about 35 mg, about 0.3 mg to about 30 mg, about 0.4 mg to about 29 mg, about 0.5 mg to about 28 mg, about 1 mg to about 27 mg, about 1.5 mg to about 26 mg, about 2 mg to about 25 mg, about 2.5 mg to about 24 mg, about 3 mg to about 23 mg, about 3.5 mg to about 22 mg, about 4 mg to about 21 mg, about 4.5 mg to about 20 mg, about 5 mg to about 19 mg, about 5.5 mg to about 18 mg, about 6 mg to about 17 mg, about 6.5 mg to about 16 mg, about 7 mg to about 15 mg, about 7.5 mg to about 14 mg, about 8 mg to about 13 mg, about 8 mg to about 12 mg, about 8.5 mg to about 12 mg, about 9 mg to about 11 mg, about 0.1 mg to about 1 mg, about 0.2 mg to about 1.5 mg, about 0.3 mg to about 2 mg, about 0.4 mg to about 2.5 mg, about 0.5 mg to about 3 mg, about 0.6 mg to about 3.5 mg, about 0.7 mg to about 4 mg, about 0.8 mg to about 4.5 mg, about 0.9 mg to about 5 mg, about 1 mg to about 6 mg, about 1.5 mg to about 6.5 mg, about 2 mg to about 7 mg, about 2.5 mg to about 7.5 mg, about 3 mg to about 8 mg, about 3.5 mg to about 8.5 mg, about 4 mg to about 9 mg, about 4.5 mg to about 9.5 mg, about 5 mg to about 10 mg, about 5.5 mg to about 10.5 mg, about 6 mg to about 11 mg, about 6.5 mg to about 11.5 mg, about 7 mg to about 12 mg, about 7.5 mg to about 12.5 mg, about 8 mg to about 13 mg, about 8.5 mg to about 13.5 mg, about 9 mg to about 14 mg, about 9.5 mg to about 14.5 mg, about 10 mg to about 15 mg, about 11 mg to about 16 mg, about 12 mg to about 17 mg, about 13 mg to about 18 mg, about 14 mg to about 19 mg, about 15 mg to about 20 mg, about 16 mg to about 21 mg, about 17 mg to about 22 mg, about 18 mg to about 23 mg, about 19 mg to about 24 mg, about 20 mg to about 25 mg, about 21 mg to about 26 mg, about 22 mg to about 27 mg, about 23 mg to about 28 mg, about 24 mg to about 29 mg, about 25 mg to about 30 mg, about 26 mg to about 31 mg, about 27 mg to about 32 mg, about 28 mg to about 33 mg, about 29 mg to about 34 mg, about 30 mg to about 35 mg, about 31 mg to about 36 mg, about 32 mg to about 37 mg, about 33 mg to about 38 mg, about 34 mg to about 39 mg, about 35 mg to about 40 mg, about 36 mg to about 41 mg, about 37 mg to about 42 mg, about 38 mg to about 43 mg, about 39 mg to about 44 mg, about 40 mg to about 45 mg, about 41 mg to about 46 mg, about 42 mg to about 47 mg, about 43 mg to about 48 mg, about 44 mg to about 49 mg, about 45 mg to about 50 mg).

In some examples, the first step-up dose is between 0.1 mg to 50 mg (e.g., between 0.1 mg to 9.5 mg, 0.2 mg to 9 mg, 0.3 mg to 8.5 mg, 0.4 mg to 8 mg, 0.5 mg to 7.5 mg, 0.6 mg to 7 mg, 0.7 mg to 6.5 mg, 0.8 mg to 6 mg, 0.9 mg to 5.5 mg, 1 mg to 5 mg, 1.1 mg to 4.5 mg, 1.2 mg to 4 mg, 1.3 mg to 3.5 mg, 1.4 mg to 3 mg, 1.5 mg to 2.5 mg, 1 mg to 3 mg, 0.2 mg to 35 mg, 0.3 mg to 30 mg, 0.4 mg to 29 mg, 0.5 mg to 28 mg, 1 mg to 27 mg, 1.5 mg to 26 mg, 2 mg to 25 mg, 2.5 mg to 24 mg, 3 mg to 23 mg, 3.5 mg to 22 mg, 4 mg to 21 mg, 4.5 mg to 20 mg, 5 mg to 19 mg, 5.5 mg to 18 mg, 6 mg to 17 mg, 6.5 mg to 16 mg, 7 mg to 15 mg, 7.5 mg to 14 mg, 8 mg to 13 mg, 8 mg to 12 mg, 8.5 mg to 12 mg, 9 mg to 11 mg, 0.1 mg to 1 mg, 0.2 mg to 1.5 mg, 0.3 mg to 2 mg, 0.4 mg to 2.5 mg, 0.5 mg to 3 mg, 0.6 mg to 3.5 mg, 0.7 mg to 4 mg, 0.8 mg to 4.5 mg, 0.9 mg to 5 mg, 1 mg to 6 mg, 1.5 mg to 6.5 mg, 2 mg to 7 mg, 2.5 mg to 7.5 mg, 3 mg to 8 mg, 3.5 mg to 8.5 mg, 4 mg to 9 mg, 4.5 mg to 9.5 mg, 5 mg to 10 mg, 5.5 mg to 10.5 mg, 6 mg to 11 mg, 6.5 mg to 11.5 mg, 7 mg to 12 mg, 7.5 mg to 12.5 mg, 8 mg to 13 mg, 8.5 mg to 13.5 mg, 9 mg to 14 mg, 9.5 mg to 14.5 mg, 10 mg to 15 mg, 11 mg to 16 mg, 12 mg to 17 mg, 13 mg to 18 mg, 14 mg to 19 mg, 15 mg to 20 mg, 16 mg to 21 mg, 17 mg to 22 mg, 18 mg to 23 mg, 19 mg to 24 mg, 20 mg to 25 mg, 21 mg to 26 mg, 22 mg to 27 mg, 23 mg to 28 mg, 24 mg to 29 mg, 25 mg to 30 mg, 26 mg to 31 mg, 27 mg to 32 mg, 28 mg to 33 mg, 29 mg to 34 mg, 30 mg to 35 mg, 31 mg to 36 mg, 32 mg to 37 mg, 33 mg to 38 mg, 34 mg to 39 mg, 35 mg to 40 mg, 36 mg to 41 mg, 37 mg to 42 mg, 38 mg to 43 mg, 39 mg to 44 mg, 40 mg to 45 mg, 41 mg to 46 mg, 42 mg to 47 mg, 43 mg to 48 mg, 44 mg to 49 mg, 45 mg to 50 mg).

In some examples, the first step-up dose is about 0.1 mg, about 0.5 mg, about 1 mg, about 2 mg, about 3 mg, about 4 mg, about 5 mg, about 6 mg, about 7 mg, about 8 mg, about 9 mg, about 10, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg.

In some examples, the first step-up dose is 0.1 mg, 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg.

In some examples, the first phase comprises administration of a first step-up dose and a second step-up dose of the bispecific antibody to the subject. In some examples, the first step-up dose is administered to the subject during the first phase on Day 1 of C1 while the second step-up dose is administered on Day 8 of C1. In some examples, the first step-up dose is administered to the subject during the first phase on Day 2 of C1 while the second step-up dose is administered on Day 9 of C1. In some examples, the first step-up dose is administered to the subject during the first phase on Day 3 of C1 while the second step-up dose is administered on Day 10 of C1. In some examples, the first step-up dose is administered to the subject during the first phase on Day 4 of C1 while the second step-up dose is administered on Day 11 of C1. In some examples, the first step-up dose is administered to the subject during the first phase on Day 5 of C1 while the second step-up dose is administered on Day 12 of C1. In some examples, the first step-up dose is administered to the subject during the first phase on Day 6 of C1 while the second step-up dose is administered on Day 13 of C1. In some examples, the first step-up dose is administered to the subject during the first phase on Day 7 of C1 while the second step-up dose is administered on Day 14 of C1. In some examples, the first step-up dose is administered to the subject during the first phase on Day 8 of C1 while the second step-up dose is administered on Day 15 of C1. In some examples, the first step-up dose is administered to the subject during the first phase on Day 9 of C1 while the second step-up dose is administered on Day 16 of C1. In some examples, the first step-up dose is administered to the subject during the first phase on Day 10 of C1 while the second step-up dose is administered on Day 17 of C1. In some examples, the first step-up dose is administered to the subject during the first phase on Day 11 of C1 while the second step-up dose is administered on Day 18 of C1. In some examples, the first step-up dose is administered to the subject during the first phase on Day 12 of C1 while the second step-up dose is administered on Day 19 of C1. In some examples, the first step-up dose is administered to the subject during the first phase on Day 13 of C1 while the second step-up dose is administered on Day 20 of C1. In some examples, the first step-up dose is administered to the subject during the first phase on Day 14 of C1 while the second step-up dose is administered on Day 21 of C1.

In a further example, a target dose is administered to the subject during the first phase following the administration of the second step-up dose. In some examples, the target dose is administered to the subject on Days 15 and/or 22 of C1. In some examples, the target dose is administered to the subject on Days 16 and/or 23 of C1. In some examples, the target dose is administered to the subject on Days 17 and/or 24 of C1. In some examples, the target dose is administered to the subject on Days 18 and/or 25 of C1. In some examples, the target dose is administered to the subject on Days 19 and/or 26 of C1. In some examples, the target dose is administered to the subject on Days 20 and/or 27 of C1. In some examples, the target dose is administered to the subject on Days 21 and/or 28 of C1.

In a further example, the target dose is further administered to the subject during the first phase on Days 1, 8, and/or 15 of C2. In a further example, the target dose is administered to the subject during the first phase on Days 1, 8, and/or 15 of C3. In a further example, the target dose is administered to the subject during the first phase on Days 1, 8, and/or 15 of C4. In a further example, the target dose is administered to the subject during the first phase on Days 1, 8, and/or 15 of C5. In a further example, the target dose is administered to the subject during the first phase on Days 1, 8, and/or 15 of C6. In a further example, the target dose is administered to the subject during the first phase on Days 1, 8, 15, and 22 of C7. In a further example, the target dose is administered to the subject during the first phase on Days 1, 8, and/or 15 of C8. In a further example, the target dose is administered to the subject during the first phase on Days 1, 8, and/or 15 of C9. In a further example, the target dose is administered to the subject during the first phase on Days 1, 8, and/or 15 of C10. In a further example, the target dose is administered to the subject during the first phase on Days 1, 8, and/or 15 of C11. In a further example, the target dose is administered to the subject during the first phase on Days 1, 8, and/or 15 of C12. In a further example, the target dose is administered to the subject during the first phase on Days 1, 8, and/or 15 of C13.

In some examples, the first step-up dose is about 1% to about 10% of the target dose and the second step-up dose is about 15% to about 45% of the target dose. In some examples, the first step-up dose is about 1%, about 1.5%, about 2%, about 3%, about 4%, about 5%, about 6%, about 7%, about 8%, about 9%, or about 10% of the target dose and the second step-up dose is about 15%, about 16%, about 17%, about 18%, about 19%, about 20%, about 21%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or about 45% of the target dose. In some examples, the first step-up dose is about 5% of the target dose and the second step-up dose is about 25% of the target dose.

In some examples, the first step-up dose is 1% to 10% of the target dose and the second step-up dose is 15% to 45% of the target dose. In some examples, the first step-up dose is 1%, 1.5%, 2%, 3%, 4%, 5%, 6%, 7%, 8%, 9%, or 10% of the target dose and the second step-up dose is 15%, 16%, 17%, 18%, 19%, 20%, 21%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, or 45% of the target dose. In some examples, the first step-up dose is 5% of the target dose and the second step-up dose is 25% of the target dose.

In some examples, the first step-up dose is between about 0.1 mg to about 10 mg (e.g., is between about 0.1 mg to about 2 mg, about 0.2 mg to about 1 mg, or about 0.2 mg to about 0.4 mg, about 0.1 mg to about 9.5 mg, about 0.2 mg to about 9 mg, about 0.3 mg to about 8.5 mg, about 0.4 mg to about 8 mg, about 0.5 mg to about 7.5 mg, about 0.6 mg to about 7 mg, about 0.7 mg to about 6.5 mg, about 0.8 mg to about 6 mg, about 0.9 mg to about 5.5 mg, about 1 mg to about 5 mg, about 1.1 mg to about 4.5 mg, about 1.2 mg to about 4 mg, about 1.3 mg to about 3.5 mg, about 1.4 mg to about 3 mg, about 1.5 mg to about 2.5 mg, about 1 mg to about 3 mg, about 0.1 mg to about 1 mg, about 0.2 mg to about 1.5 mg, about 0.3 mg to about 2 mg, about 0.4 mg to about 2.5 mg, about 0.5 mg to about 3 mg, about 0.6 mg to about 3.5 mg, about 0.7 mg to about 4 mg, about 0.8 mg to about 4.5 mg, about 0.9 mg to about 5 mg, about 1 mg to about 6 mg, about 1.5 mg to about 6.5 mg, about 2 mg to about 7 mg, about 2.5 mg to about 7.5 mg, about 3 mg to about 8 mg, about 3.5 mg to about 8.5 mg, about 4 mg to about 9 mg, about 4.5 mg to about 9.5 mg, or about 5 mg to about 10 mg); and the second step-up dose is between about 1 mg to about 50 mg (e.g., between about 3 mg to about 18 mg, between about 3.1 mg to about 15 mg, between about 3.2 mg to about 10 mg, between about 3.3 mg to about 6 mg, between about 3.4 mg to about 4 mg, about 0.2 mg to about 35 mg, about 0.3 mg to about 30 mg, about 0.4 mg to about 29 mg, about 0.5 mg to about 28 mg, about 1 mg to about 27 mg, about 1.5 mg to about 26 mg, about 2 mg to about 25 mg, about 2.5 mg to about 24 mg, about 3 mg to about 23 mg, about 3.5 mg to about 22 mg, about 4 mg to about 21 mg, about 4.5 mg to about 20 mg, about 5 mg to about 19 mg, about 5.5 mg to about 18 mg, about 6 mg to about 17 mg, about 6.5 mg to about 16 mg, about 7 mg to about 15 mg, about 7.5 mg to about 14 mg, about 8 mg to about 13 mg, about 8 mg to about 12 mg, about 8.5 mg to about 12 mg, about 9 mg to about 11 mg, about 1 mg to about 6 mg, about 1.5 mg to about 6.5 mg, about 2 mg to about 7 mg, about 2.5 mg to about 7.5 mg, about 3 mg to about 8 mg, about 3.5 mg to about 8.5 mg, about 4 mg to about 9 mg, about 4.5 mg to about 9.5 mg, about 5 mg to about 10 mg, about 5.5 mg to about 10.5 mg, about 6 mg to about 11 mg, about 6.5 mg to about 11.5 mg, about 7 mg to about 12 mg, about 7.5 mg to about 12.5 mg, about 8 mg to about 13 mg, about 8.5 mg to about 13.5 mg, about 9 mg to about 14 mg, about 9.5 mg to about 14.5 mg, about 10 mg to about 15 mg, about 11 mg to about 16 mg, about 12 mg to about 17 mg, about 13 mg to about 18 mg, about 14 mg to about 19 mg, about 15 mg to about 20 mg, about 16 mg to about 21 mg, about 17 mg to about 22 mg, about 18 mg to about 23 mg, about 19 mg to about 24 mg, about 20 mg to about 25 mg, about 21 mg to about 26 mg, about 22 mg to about 27 mg, about 23 mg to about 28 mg, about 24 mg to about 29 mg, about 25 mg to about 30 mg, about 26 mg to about 31 mg, about 27 mg to about 32 mg, about 28 mg to about 33 mg, about 29 mg to about 34 mg, about 30 mg to about 35 mg, about 31 mg to about 36 mg, about 32 mg to about 37 mg, about 33 mg to about 38 mg, about 34 mg to about 39 mg, about 35 mg to about 40 mg, about 36 mg to about 41 mg, about 37 mg to about 42 mg, about 38 mg to about 43 mg, about 39 mg to about 44 mg, about 40 mg to about 45 mg, about 41 mg to about 46 mg, about 42 mg to about 47 mg, about 43 mg to about 48 mg, about 44 mg to about 49 mg, or about 45 mg to about 50 mg).

In some examples, the first step-up dose is between 0.1 mg to 10 mg (e.g., is between 0.1 mg to 2 mg, 0.2 mg to 1 mg, or 0.2 mg to 0.4 mg, 0.1 mg to 9.5 mg, 0.2 mg to 9 mg, 0.3 mg to 8.5 mg, 0.4 mg to 8 mg, 0.5 mg to 7.5 mg, 0.6 mg to 7 mg, 0.7 mg to 6.5 mg, 0.8 mg to 6 mg, 0.9 mg to 5.5 mg, 1 mg to 5 mg, 1.1 mg to 4.5 mg, 1.2 mg to 4 mg, 1.3 mg to 3.5 mg, 1.4 mg to 3 mg, 1.5 mg to 2.5 mg, 1 mg to 3 mg, 0.1 mg to 1 mg, 0.2 mg to 1.5 mg, 0.3 mg to 2 mg, 0.4 mg to 2.5 mg, 0.5 mg to 3 mg, 0.6 mg to 3.5 mg, 0.7 mg to 4 mg, 0.8 mg to 4.5 mg, 0.9 mg to 5 mg, 1 mg to 6 mg, 1.5 mg to 6.5 mg, 2 mg to 7 mg, 2.5 mg to 7.5 mg, 3 mg to 8 mg, 3.5 mg to 8.5 mg, 4 mg to 9 mg, 4.5 mg to 9.5 mg, or 5 mg to 10 mg); and the second step-up dose is between 1 mg to 50 mg (e.g., between 3 mg to 18 mg, between 3.1 mg to 15 mg, between 3.2 mg to 10 mg, between 3.3 mg to 6 mg, between 3.4 mg to 4 mg, 0.2 mg to 35 mg, 0.3 mg to 30 mg, 0.4 mg to 29 mg, 0.5 mg to 28 mg, 1 mg to 27 mg, 1.5 mg to 26 mg, 2 mg to 25 mg, 2.5 mg to 24 mg, 3 mg to 23 mg, 3.5 mg to 22 mg, 4 mg to 21 mg, 4.5 mg to 20 mg, 5 mg to 19 mg, 5.5 mg to 18 mg, 6 mg to 17 mg, 6.5 mg to 16 mg, 7 mg to 15 mg, 7.5 mg to 14 mg, 8 mg to 13 mg, 8 mg to 12 mg, 8.5 mg to 12 mg, 9 mg to 11 mg, 1 mg to 6 mg, 1.5 mg to 6.5 mg, 2 mg to 7 mg, 2.5 mg to 7.5 mg, 3 mg to 8 mg, 3.5 mg to 8.5 mg, 4 mg to 9 mg, 4.5 mg to 9.5 mg, 5 mg to 10 mg, 5.5 mg to 10.5 mg, 6 mg to 11 mg, 6.5 mg to 11.5 mg, 7 mg to 12 mg, 7.5 mg to 12.5 mg, 8 mg to 13 mg, 8.5 mg to 13.5 mg, 9 mg to 14 mg, 9.5 mg to 14.5 mg, 10 mg to 15 mg, 11 mg to 16 mg, 12 mg to 17 mg, 13 mg to 18 mg, 14 mg to 19 mg, 15 mg to 20 mg, 16 mg to 21 mg, 17 mg to 22 mg, 18 mg to 23 mg, 19 mg to 24 mg, 20 mg to 25 mg, 21 mg to 26 mg, 22 mg to 27 mg, 23 mg to 28 mg, 24 mg to 29 mg, 25 mg to 30 mg, 26 mg to 31 mg, 27 mg to 32 mg, 28 mg to 33 mg, 29 mg to 34 mg, 30 mg to 35 mg, 31 mg to 36 mg, 32 mg to 37 mg, 33 mg to 38 mg, 34 mg to 39 mg, 35 mg to 40 mg, 36 mg to 41 mg, 37 mg to 42 mg, 38 mg to 43 mg, 39 mg to 44 mg, 40 mg to 45 mg, 41 mg to 46 mg, 42 mg to 47 mg, 43 mg to 48 mg, 44 mg to 49 mg, or 45 mg to 50 mg). In some examples, the first step-up dose is about 2 mg and the second step-up dose is about 10 mg. In some examples, the first step-up dose is about 0.1 mg, about 0.5 mg, about 1 mg, about 2 mg, about 3 mg, about 4 mg, about 5 mg, about 6 mg, about 7 mg, about 8 mg, about 9 mg, or about 10. while the second step-up dose is about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg.

In some examples, the first step-up dose is 2 mg and the second step-up dose is 10 mg. In some examples, the first step-up dose is 0.1 mg, 0.5 mg, 1 mg, 2 mg, 3 mg, 4 mg, 5 mg, 6 mg, 7 mg, 8 mg, 9 mg, or 10 mg, while the second step-up dose is 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg.

In any of the foregoing examples, the second phase may comprise at least one dosing cycle, at least two dosing cycles, at least three dosing cycles, or at least four dosing cycles, at least five dosing cycles, at least six dosing cycles, at least seven, at least eight dosing cycle, at least nine dosing cycle, at least ten dosing cycle, at least eleven dosing cycles, at least twelve dosing cycles, or at least thirteen dosing cycles, or more.

The second phase may comprise any suitable number of dosing cycles. For example, in some examples, the second phase may comprises a first dosing cycle (C1); a first dosing cycle and a second dosing cycle (C2); a first dosing cycle, a second dosing cycle (C2), and a third dosing cycle (C3); a first dosing cycle (C1), a second dosing cycle (C2), a third dosing cycle (C3), and a fourth dosing cycle (C4); a first dosing cycle (C1), a second dosing cycle (C2), a third dosing cycle (C3), a fourth dosing cycle (C4), and a fifth dosing cycle (C5); a first dosing cycle (C1), a second dosing cycle (C2), a third dosing cycle (C3), a fourth dosing cycle (C4), a fifth dosing cycle (C5), and a sixth dosing cycle (C6); a first dosing cycle (C1), a second dosing cycle (C2), a third dosing cycle (C3), a fourth dosing cycle (C4), a fifth dosing cycle (C5), a sixth dosing cycle (C6), and a seventh dosing cycle (C7); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), and an eighth dosing cycle (C8); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), and a ninth dosing cycle (C9); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), and a tenth dosing cycle (C10); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), and an eleventh dosing cycle (C11); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), and a twelfth dosing cycle (C12); or a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), and a thirteenth dosing cycle (C13).

In some examples, a target dose of the bispecific antibody may be administered to the subject during the second phase on Days 1 and/or 15 of C1. In a further example, the target dose of the bispecific antibody may be administered to the subject during the second phase on Days 1 and/or 15 of C2. In a further example, the target dose of the bispecific antibody may be administered to the subject during the second phase on Days 1 and/or 15 of C3. In a further example, the target dose of the bispecific antibody may be administered to the subject during the second phase on Days 1 and/or 15 of C4. In a further example, the target dose of the bispecific antibody may be administered to the subject during the second phase on Days 1 and/or 15 of C5. In a further example, the target dose of the bispecific antibody may be administered to the subject during the second phase on Days 1 and/or 15 of C6. In a further example, the target dose of the bispecific antibody may be administered to the subject during the second phase on Days 1 and/or 15 of C7. In a further example, the target dose of the bispecific antibody may be administered to the subject during the second phase on Days 1 and/or 15 of C8. In a further example, the target dose of the bispecific antibody may be administered to the subject during the second phase on Days 1 and/or 15 of C9. In a further example, the target dose of the bispecific antibody may be administered to the subject during the second phase on Days 1 and/or 15 of C10. In a further example, the target dose of the bispecific antibody may be administered to the subject during the second phase on Days 1 and/or 15 of C11. In a further example, the target dose of the bispecific antibody may be administered to the subject during the second phase on Days 1 and/or 15 of C12. In a further example, the target dose of the bispecific antibody may be administered to the subject during the second phase on Days 1 and/or 15 of C13.

In some examples, a target dose of the bispecific antibody is administered to the subject for each administration during the second phase.

The third phase may comprise any suitable number of dosing cycles. For example, in any of the foregoing examples, the third phase may comprise at least one dosing cycle, at least two dosing cycles, at least three dosing cycles, at least four dosing cycles, at least five dosing cycles, at least six dosing cycles, at least seven dosing cycles, at least eight dosing cycle, at least nine dosing cycle, at least ten dosing cycle, at least eleven dosing cycles, at least twelve dosing cycles, or at least thirteen dosing cycles, or more.

In some examples, third phase comprises a first dosing cycle (C1); a first dosing cycle and a second dosing cycle (C2); a first dosing cycle, a second dosing cycle (C2), and a third dosing cycle (C3); a first dosing cycle (C1), a second dosing cycle (C2), a third dosing cycle (C3), and a fourth dosing cycle (C4); a first dosing cycle (C1), a second dosing cycle (C2), a third dosing cycle (C3), a fourth dosing cycle (C4), and a fifth dosing cycle (C5); a first dosing cycle (C1), a second dosing cycle (C2), a third dosing cycle (C3), a fourth dosing cycle (C4), a fifth dosing cycle (C5), and a sixth dosing cycle (C6); a first dosing cycle (C1), a second dosing cycle (C2), a third dosing cycle (C3), a fourth dosing cycle (C4), a fifth dosing cycle (C5), a sixth dosing cycle (C6), and a seventh dosing cycle (C7); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), and an eighth dosing cycle (C8); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), and a ninth dosing cycle (C9); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), and a tenth dosing cycle (C10); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), and an eleventh dosing cycle (C11); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), and a twelfth dosing cycle (C12); or a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), and a thirteenth dosing cycle (C13).

In some examples, the third phase comprises administration of the bispecific antibody to the subject on Day 1 of C1. In a further example, the third phase comprises administration of the bispecific antibody to the subject on Day 1 of C2. In a further example, the third phase comprises administration of the bispecific antibody to the subject on Day 1 of C3. In a further example, the third phase comprises administration of the bispecific antibody to the subject on Day 1 of C4. In a further example, the third phase comprises administration of the bispecific antibody to the subject on Day 1 of C5. In a further example, the third phase comprises administration of the bispecific antibody to the subject on Day 1 of C6. In a further example, the third phase comprises administration of the bispecific antibody to the subject on Day 1 of C7. In a further example, the third phase comprises administration of the bispecific antibody to the subject on Day 1 of C8. In a further example, the third phase comprises administration of the bispecific antibody to the subject on Day 1 of C9. In a further example, the third phase comprises administration of the bispecific antibody to the subject on Day 1 of C10. In a further example, the third phase comprises administration of the bispecific antibody to the subject on Day 1 of C11. In a further example, the third phase comprises administration of the bispecific antibody to the subject on Day 1 of C12. In a further example, the third phase comprises administration of the bispecific antibody to the subject on Day 1 of C13.

In some examples, a target dose of the bispecific antibody is administered to the subject for each administration during the third phase.

In any of the foregoing examples, the target dose may be about 10 mg to about 1000 mg (e.g., between about 10 mg to about 70 mg, about 15 mg to about 65 mg, about 20 mg to about 60 mg, about 25 mg to about 55 mg, about 30 mg to about 50 mg, about 35 mg to about 45 mg, about 15 mg to about 225 mg, about 20 mg to about 220 mg, about 25 mg to about 215 mg, about 30 mg to about 210 mg, about 35 mg to about 205 mg, about 40 mg to about 200 mg, about 45 mg to about 195 mg, about 50 mg to about 190 mg, about 55 mg to about 185 mg, about 60 mg to about 180 mg, about 65 mg to about 175 mg, about 70 mg to about 170 mg, about 75 mg to about 165 mg, about 80 mg to about 160 mg, about 85 mg to about 155 mg, about 90 mg to about 150 mg, about 95 mg to about 145 mg, about 100 mg to about 140 mg, about 105 mg to about 135 mg, about 110 mg to about 130 mg, about 115 mg to about 125 mg, about 10 mg to about 50 mg, about 25 mg to about 75 mg, about 50 mg to about 100 mg, about 75 mg to about 125 mg, about 100 mg to about 150 mg, about 125 mg to about 175 mg, about 150 mg to about 200 mg, about 175 mg to about 225 mg, about 200 mg to about 250 mg, about 225 mg to about 275 mg, about 250 mg to about 300 mg, about 275 mg to about 325 mg, about 300 mg to about 350 mg, about 325 mg to about 375 mg, about 350 mg to about 400 mg, about 375 mg to about 425 mg, about 400 mg to about 450 mg, about 425 mg to about 475 mg, about 450 mg to about 500 mg, about 475 mg to about 525 mg, about 500 mg to about 550 mg, about 525 mg to about 575 mg, about 550 mg to about 600 mg, about 575 mg to about 625 mg, about 600 mg to about 650 mg, about 625 mg to about 675 mg, about 650 mg to about 700 mg, about 675 mg to about 725 mg, about 700 mg to about 750 mg, about 725 mg to about 775 mg, about 750 mg to about 800 mg, about 775 mg to about 825 mg, about 800 mg to about 850 mg, about 825 mg to about 875 mg, about 850 mg to about 900 mg, about 875 mg to about 925 mg, about 900 mg to about 950 mg, about 925 mg to about 975 mg, or about 950 mg to about 1000 mg). In some aspects, the C1D2 is greater than the C1D1 and the C1D3 is greater than the C1D2.

In some aspects, the target dose is between about 10 mg to 200 mg (e.g., between 10 mg to about 170 mg, about 11 mg to about 165 mg, about 12 mg to about 160 mg, about 13 mg to about 155 mg, about 14 mg to about 150 mg, about 15 mg to about 145 mg, about 16 mg to about 140 mg, about 17 mg to about 135 mg, about 18 mg to about 130 mg, about 19 mg to about 125 mg, about 20 mg to about 120 mg, about 21 mg to about 115 mg, about 22 mg to about 110 mg, about 23 mg to about 105 mg, about 24 mg to about 100 mg, about 25 mg to about 95 mg, about 26 mg to about 90 mg, about 27 mg to about 85 mg, about 28 mg to about 80 mg, about 29 mg to about 75 mg, about 30 mg to about 70 mg, about 31 mg to about 65 mg, about 32 mg to about 60 mg, about 33 mg to about 55 mg, about 34 mg to about 50 mg, about 35 mg to about 45 mg, about 40 mg to about 200 mg, about 45 mg to about 195 mg, about 50 mg to about 190 mg, about 55 mg to about 185 mg, about 60 mg to about 180 mg, about 65 mg to about 175 mg, about 70 mg to about 170 mg, about 75 mg to about 165 mg, about 80 mg to about 160 mg, about 85 mg to about 155 mg, about 90 mg to about 150 mg, about 95 mg to about 145 mg, about 100 mg to about 140 mg, about 105 mg to about 135 mg, about 110 mg to about 130 mg, about 115 mg to about 125 mg, about 10 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, about 90 mg to about 100 mg, about 100 mg to about 110 mg, about 110 mg to about 120 mg, about 120 mg to about 130 mg, about 130 mg to about 140 mg, about 140 mg to about 150 mg, about 150 mg to about 160 mg, about 160 mg to about 170 mg, about 170 mg to about 180 mg, about 180 mg to about 190 mg, or about 190 mg to about 200 mg).

In some examples, the target dose is about 10 mg. In some examples, the target dose is about 15 mg. In some examples, the target dose is about 20 mg. In some examples, the target dose is about 25 mg. In some examples, the target dose is about 30 mg. In some examples, the target dose is about 35 mg. In some examples, the target dose is about 40 mg. In some examples, the target dose is about 45 mg. In some examples, the target dose is about 50 mg. In some examples, the target dose is about 55 mg. In some examples, the target dose is about 60 mg. In some examples, the target dose is about 65 mg. In some examples, the target dose is about 70 mg. In some examples, the target dose is about 75 mg. In some examples, the target dose is about 80 mg. In some examples, the target dose is about 85 mg. In some examples, the target dose is about 90 mg. In some examples, the target dose is about 95 mg. In some examples, the target dose is about 100 mg. In some examples, the target dose is about 105 mg. In some examples, the target dose is about 110 mg. In some examples, the target dose is about 115 mg. In some examples, the target dose is about 120 mg. In some examples, the target dose is about 125 mg. In some examples, the target dose is about 130 mg. In some examples, the target dose is about 132 mg. In some examples, the target dose is about 135 mg. In some examples, the target dose is about 140 mg. In some examples, the target dose is about 145 mg. In some examples, the target dose is about 150 mg. In some examples, the target dose is about 155 mg. In some examples, the target dose is about 160 mg. In some examples, the target dose is about 165 mg. In some examples, the target dose is about 170 mg. In some examples, the target dose is about 175 mg. In some examples, the target dose is about 180 mg. In some examples, the target dose is about 185 mg. In some examples, the target dose is about 190 mg. In some examples, the target dose is about 195 mg. In some examples, the target dose is about 200 mg. In some aspects, the C1D1 is about 210 mg. In some aspects, the C1D1 is about 220 mg. In some aspects, the C1D1 is about 230 mg. In some aspects, the C1D1 is about 240 mg. In some aspects, the C1D1 is about 250 mg. In some aspects, the C1D1 is about 260 mg. In some aspects, the C1D1 is about 270 mg. In some aspects, the C1D1 is about 280 mg. In some aspects, the C1D1 is about 290 mg. In some aspects, the C1D1 is about 300 mg. In some aspects, the C1D1 is about 310 mg. In some aspects, the C1D1 is about 320 mg. In some aspects, the C1D1 is about 330 mg. In some aspects, the C1D1 is about 340 mg. In some aspects, the C1D1 is about 350 mg. In some aspects, the C1D1 is about 360 mg. In some aspects, the C1D1 is about 370 mg. In some aspects, the C1D1 is about 380 mg. In some aspects, the C1D1 is about 390 mg. In some aspects, the C1D1 is about 400 mg. In some aspects, the C1D1 is about 410 mg. In some aspects, the C1D1 is about 420 mg. In some aspects, the C1D1 is about 430 mg. In some aspects, the C1D1 is about 440 mg. In some aspects, the C1D1 is about 450 mg. In some aspects, the C1D1 is about 460 mg. In some aspects, the C1D1 is about 470 mg. In some aspects, the C1D1 is about 480 mg. In some aspects, the C1D1 is about 490 mg. In some aspects, the C1D1 is about 500 mg. In some aspects, the C1D1 is about 510 mg. In some aspects, the C1D1 is about 520 mg. In some aspects, the C1D1 is about 530 mg. In some aspects, the C1D1 is about 540 mg. In some aspects, the C1D1 is about 550 mg. In some aspects, the C1D1 is about 560 mg. In some aspects, the C1D1 is about 570 mg. In some aspects, the C1D1 is about 580 mg. In some aspects, the C1D1 is about 590 mg. In some aspects, the C1D1 is about 600 mg. In some aspects, the C1D1 is about 610 mg. In some aspects, the C1D1 is about 620 mg. In some aspects, the C1D1 is about 630 mg. In some aspects, the C1D1 is about 640 mg. In some aspects, the C1D1 is about 650 mg. In some aspects, the C1D1 is about 660 mg. In some aspects, the C1D1 is about 670 mg. In some aspects, the C1D1 is about 680 mg. In some aspects, the C1D1 is about 690 mg. In some aspects, the C1D1 is about 700 mg. In some aspects, the C1D1 is about 710 mg. In some aspects, the C1D1 is about 720 mg. In some aspects, the C1D1 is about 730 mg. In some aspects, the C1D1 is about 740 mg. In some aspects, the C1D1 is about 750 mg. In some aspects, the C1D1 is about 760 mg. In some aspects, the C1D1 is about 770 mg. In some aspects, the C1D1 is about 780 mg. In some aspects, the C1D1 is about 790 mg. In some aspects, the C1D1 is about 800 mg. In some aspects, the C1D1 is about 810 mg. In some aspects, the C1D1 is about 820 mg. In some aspects, the C1D1 is about 830 mg. In some aspects, the C1D1 is about 840 mg. In some aspects, the C1D1 is about 850 mg. In some aspects, the C1D1 is about 860 mg. In some aspects, the C1D1 is about 870 mg. In some aspects, the C1D1 is about 880 mg. In some aspects, the C1D1 is about 890 mg. In some aspects, the C1D1 is about 900 mg. In some aspects, the C1D1 is about 910 mg. In some aspects, the C1D1 is about 920 mg. In some aspects, the C1D1 is about 930 mg. In some aspects, the C1D1 is about 940 mg. In some aspects, the C1D1 is about 950 mg. In some aspects, the C1D1 is about 960 mg. In some aspects, the C1D1 is about 970 mg. In some aspects, the C1D1 is about 980 mg. In some aspects, the C1D1 is about 990 mg. In some aspects, the C1D1 is about 1000 mg.

In some examples, the target dose is 10 mg. In some examples, the target dose is 15 mg. In some examples, the target dose is 20 mg. In some examples, the target dose is 25 mg. In some examples, the target dose is 30 mg. In some examples, the target dose is 35 mg. In some examples, the target dose is 40 mg. In some examples, the target dose is 45 mg. In some examples, the target dose is 50 mg. In some examples, the target dose is 55 mg. In some examples, the target dose is 60 mg. In some examples, the target dose is 65 mg. In some examples, the target dose is 70 mg. In some examples, the target dose is 75 mg. In some examples, the target dose is 80 mg. In some examples, the target dose is 85 mg. In some examples, the target dose is 90 mg. In some examples, the target dose is 95 mg. In some examples, the target dose is 100 mg. In some examples, the target dose is 105 mg. In some examples, the target dose is 110 mg. In some examples, the target dose is 115 mg. In some examples, the target dose is 120 mg. In some examples, the target dose is 125 mg. In some examples, the target dose is 130 mg. In some examples, the target dose is 132 mg. In some examples, the target dose is 135 mg. In some examples, the target dose is 140 mg. In some examples, the target dose is 145 mg. In some examples, the target dose is 150 mg. In some examples, the target dose is 155 mg. In some examples, the target dose is 160 mg. In some examples, the target dose is 165 mg. In some examples, the target dose is 170 mg. In some examples, the target dose is 175 mg. In some examples, the target dose is 180 mg. In some examples, the target dose is 185 mg. In some examples, the target dose is 190 mg. In some examples, the target dose is 195 mg. In some examples, the target dose is 200 mg. In some aspects, the C1D1 is 210 mg. In some aspects, the C1D1 is 220 mg. In some aspects, the C1D1 is 230 mg. In some aspects, the C1D1 is 240 mg. In some aspects, the C1D1 is 250 mg. In some aspects, the C1D1 is 260 mg. In some aspects, the C1D1 is 270 mg. In some aspects, the C1D1 is 280 mg. In some aspects, the C1D1 is 290 mg. In some aspects, the C1D1 is 300 mg. In some aspects, the C1D1 is 310 mg. In some aspects, the C1D1 is 320 mg. In some aspects, the C1D1 is 330 mg. In some aspects, the C1D1 is 340 mg. In some aspects, the C1D1 is 350 mg. In some aspects, the C1D1 is 360 mg. In some aspects, the C1D1 is 370 mg. In some aspects, the C1D1 is 380 mg. In some aspects, the C1D1 is 390 mg. In some aspects, the C1D1 is 400 mg. In some aspects, the C1D1 is 410 mg. In some aspects, the C1D1 is 420 mg. In some aspects, the C1D1 is 430 mg. In some aspects, the C1D1 is 440 mg. In some aspects, the C1D1 is 450 mg. In some aspects, the C1D1 is 460 mg. In some aspects, the C1D1 is 470 mg. In some aspects, the C1D1 is 480 mg. In some aspects, the C1D1 is 490 mg. In some aspects, the C1D1 is 500 mg. In some aspects, the C1D1 is 510 mg. In some aspects, the C1D1 is 520 mg. In some aspects, the C1D1 is 530 mg. In some aspects, the C1D1 is 540 mg. In some aspects, the C1D1 is 550 mg. In some aspects, the C1D1 is 560 mg. In some aspects, the C1D1 is 570 mg. In some aspects, the C1D1 is 580 mg. In some aspects, the C1D1 is 590 mg. In some aspects, the C1D1 is 600 mg. In some aspects, the C1D1 is 610 mg. In some aspects, the C1D1 is 620 mg. In some aspects, the C1D1 is 630 mg. In some aspects, the C1D1 is 640 mg. In some aspects, the C1D1 is 650 mg. In some aspects, the C1D1 is 660 mg. In some aspects, the C1D1 is 670 mg. In some aspects, the C1D1 is 680 mg. In some aspects, the C1D1 is 690 mg. In some aspects, the C1D1 is 700 mg. In some aspects, the C1D1 is 710 mg. In some aspects, the C1D1 is 720 mg. In some aspects, the C1D1 is 730 mg. In some aspects, the C1D1 is 740 mg. In some aspects, the C1D1 is 750 mg. In some aspects, the C1D1 is 760 mg. In some aspects, the C1D1 is 770 mg. In some aspects, the C1D1 is 780 mg. In some aspects, the C1D1 is 790 mg. In some aspects, the C1D1 is 800 mg. In some aspects, the C1D1 is 810 mg. In some aspects, the C1D1 is 820 mg. In some aspects, the C1D1 is 830 mg. In some aspects, the C1D1 is 840 mg. In some aspects, the C1D1 is 850 mg. In some aspects, the C1D1 is 860 mg. In some aspects, the C1D1 is 870 mg. In some aspects, the C1D1 is 880 mg. In some aspects, the C1D1 is 890 mg. In some aspects, the C1D1 is 900 mg. In some aspects, the C1D1 is 910 mg. In some aspects, the C1D1 is 920 mg. In some aspects, the C1D1 is 930 mg. In some aspects, the C1D1 is 940 mg. In some aspects, the C1D1 is 950 mg. In some aspects, the C1D1 is 960 mg. In some aspects, the C1D1 is 970 mg. In some aspects, the C1D1 is 980 mg. In some aspects, the C1D1 is 990 mg. In some aspects, the C1D1 is 1000 mg. In any of the preceding examples, each dose of the bispecific anti-FcRH5/anti-CD3 antibody may be administered subcutaneously. However, it is also contemplated that a subset of doses may be administered using alternative administration routes, e.g., intravenously.

### C. Combination Therapies

In some instances, the bispecific anti-FcRH5/anti-CD3 antibody is administered to the subject in a combination therapy. For example, the bispecific anti-FcRH5/anti-CD3 antibody may be co-administered with one or more additional therapeutic agents described herein.

i. Anti-CD38 Antibodies

In some instances, the bispecific anti-FcRH5/anti-CD3 antibody is administered to the subject in combination with an anti-CD38 antibody. The anti-CD38 antibody may be administered by any suitable administration route, e.g., intravenously (IV) or subcutaneously (SC) to the subject. In some aspects, the anti-CD38 antibody is daratumumab (e.g., daratumumab/rHuPH20). The daratumumab may be administered to the subject at a dose of about 900 mg to about 3600 mg (e.g., about 900 mg, about 950 mg, about 1000 mg, about 1100 mg, about 1200 mg, about 1300 mg, about 1400 mg, about 1500 mg, about 1600 mg, about 1650 mg, about 1700 mg, about 1750 mg, about 1800 mg, about 1850 mg about 1900 mg, about 1950 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, or about 3600 mg). The daratumumab may be administered to the subject at a dose of about 1800 mg. In some aspects, the daratumumab is administered by intravenous infusion (e.g., infusion over 3-5 hours) at a dose of 16 mg/kg once every week, once every two weeks, or once every four weeks. In some aspects, the daratumumab is administered by intravenous infusion (e.g., infusion over 3-5 hours) at a dose of 16 mg/kg. In some aspects, the daratumumab is administered subcutaneously. In other aspects, the anti-CD38 antibody is isatuximab. In some aspects, the anti-CD38 antibody (e.g., daratumumab or isatuxamab) is administered to the subject prior to the administration of the bispecific anti-FcRH5/anti-CD3 antibody, e.g., administered one day prior to the administration of the bispecific anti-FcRH5/anti-CD3 antibody. In some aspects, the anti-CD38 antibody (e.g., daratumumab or isatuxamab) is administered to the subject concurrently with the administration of the bispecific anti-FcRH5/anti-CD3 antibody.

ii. Corticosteroids

In some instances, the bispecific anti-FcRH5/anti-CD3 antibody is administered to the subject in combination with a corticosteroid. The corticosteroid may be administered orally to the subject. The corticosteroid may be administered by any suitable administration route, e.g., intravenously or subcutaneously to the subject. Any suitable corticosteroid may be used, e.g., dexamethasone, methylprednisolone, prednisone, prednisolone, betamethasone, hydrocortisone, and the like. In some aspects, the corticosteroid is methylprednisolone. The methylprednisolone may be administered to the subject at a dose of about 80 mg. In other aspects, the corticosteroid is dexamethasone. The dexamethasone may be administered to the subject at a dose of about 20 mg. In some aspects, the corticosteroid (e.g., methylprednisolone or dexamethasone) is administered to the subject prior to the administration of the bispecific anti-FcRH5/anti-CD3 antibody, e.g., administered one hour prior to the administration of the bispecific anti-FcRH5/anti-CD3 antibody. In some aspects, the corticosteroid (e.g., methylprednisolone or dexamethasone) is administered to the subject about one day prior to the administration of the bispecific anti-FcRH5/anti-CD3 antibody. In some aspects, the corticosteroid (e.g., methylprednisolone or dexamethasone) is administered to the subject concurrently with the administration of the bispecific anti-FcRH5/anti-CD3 antibody.

iii. Immunomodulatory Drugs (IMiD)

In some instances, the bispecific anti-FcRH5/anti-CD3 antibody is administered to the subject in combination with an immunomodulatory drug (IMiD). The IMiD may be administered by any suitable administration route, e.g., orally to the subject. The IMiD may be administered intravenously to the subject. The IMiD may be administered subcutaneously to the subject. In some aspects, the IMID is pomalidomide. The pomalidomide may be administered to the subject at a dose of about 4 mg. In other aspects, the IMiD is lenalidomide. In some aspects, the IMID (e.g., pomalidomide or lenalidomide) is administered to the subject prior to the administration of the bispecific anti-FcRH5/anti-CD3 antibody, e.g., administered one hour prior to the administration of the bispecific anti-FcRH5/anti-CD3 antibody. In some aspects, the IMiD (e.g., pomalidomide or lenalidomide) is administered to the subject concurrently with the administration of the bispecific anti-FcRH5/anti-CD3 antibody. In some aspects, the IMiD (e.g., pomalidomide or lenalidomide) is administered daily between doses of the bispecific anti-FcRH5/anti-CD3 antibody.

iv. Tocilizumab and Treatment of CRS

In one instance, the additional therapeutic agent is an effective amount of tocilizumab (ACTEMRA®). In some instances, the subject has a cytokine release syndrome (CRS) event (e.g., has a CRS event following treatment with the bispecific antibody, e.g., has a CRS event following a C1D1, a C1D2, a C1D3, a C2D1, or an additional dose of the bispecific antibody), and the method further comprises treating the symptoms of the CRS event (e.g., treating the CRS event by administering to the subject an effective amount of tocilizumab) while suspending treatment with the bispecific antibody. In some aspects, tocilizumab is administered intravenously to the subject as a single dose of about 8 mg/kg. In some aspects, the CRS event does not resolve or worsens within 24 hours of treating the symptoms of the CRS event, and the method further comprising administering to the subject one or more additional doses of tocilizumab to manage the CRS event, e.g., administering one or more additional doses of tocilizumab intravenously to the subject at a dose of about 8 mg/kg.

In some aspects, treating the symptoms of the CRS event further comprises treatment with a high-dose vasopressor (e.g., norepinephrine, dopamine, phenylephrine, epinephrine, or vasopressin and norepinephrine), e.g., as described in Tables 2A, 2B, and 6.

In other instances, tocilizumab is administered as a premedication, e.g., is administered to the subject prior to the administration of the bispecific anti-FcRH5/anti-CD3 antibody. In some instances, tocilizumab is administered as a premedication in Cycle 1, e.g., is administered prior to a first dose (C1D1) of the bispecific antibody, a second dose (C1D2) of the bispecific antibody, and/or a third dose (C1D3) of the bispecific anti-FcRH5/anti-CD3 antibody. In some aspects, the tocilizumab is administered intravenously to the subject as a single dose of about 8 mg/kg.

v. CRS Symptoms and Grading

CRS may be graded according to the Modified Cytokine Release Syndrome Grading System established by Lee et al., Blood, 124:188-195, 2014 or Lee et al., Biol Blood Marrow Transplant, 25 (4): 625-638, 2019, as described in Table 2A. In addition to diagnostic criteria, recommendations on management of CRS based on its severity, including early intervention with corticosteroids and/or anti-cytokine therapy, are provided and referenced in Tables 2A and 2B.

TABLE 2A
Cytokine release syndrome grading systems

| | Modified Cytokine Release | |
|---|---|---|
| Grade | Syndrome Grading System | ASTCT Consensus Grading System |
| Grade 1 | Symptoms are not life threatening and | Temperature ≥38° C. |
| | require symptomatic treatment only (e.g., | No hypotension |
| | fever, nausea, fatigue, headache, myalgia, | No hypoxia |
| | malaise) | |
| Grade 2 | Symptoms require and respond to | Temperature ≥38° C.* with |
| | moderate intervention | hypotension not requiring |
| | Oxygen requirement <40%; or | vasopressors and/or^{†} hypoxia |
| | Hypotension responsive to fluids or low | requiring low-flow nasal cannula^{‡} or |
| | dose ^{a} of one vasopressor; or | blow-by |
| | Grade 2 organ toxicity | |
| Grade 3 | Symptoms require and respond to | Temperature ≥38° C.* with |
| | aggressive intervention | hypotension requiring a vasopressor |
| | Oxygen requirement ≥40%; or | with or without vasopressin and/or^{†} |
| | Hypotension requiring high dose ^{b} or | hypoxia requiring high-flow nasal |
| | multiple vasopressors; or | cannula^{‡}, facemask, nonrebreather |
| | Grade 3 organ toxicity or | mask, or Venturi mask |
| | Grade 4 transaminitis | |
| Grade 4 | Life-threatening symptoms | Temperature ≥38° C.* with |
| | Requirement for ventilation support or | hypotension requiring multiple |
| | Grade 4 organ toxicity (excluding | vasopressors (excluding vasopressin) |
| | transaminitis) | and/or^{†} hypoxia requiring positive |
| | | pressure (e.g., CPAP, BiPAP, |
| | | intubation and mechanical ventilation) |
| Grade 5 | Death | Death |

TABLE 2B
High-dose vasopressors
High-Dose Vasopressors (duration ≥3 hours)

| Pressor | Dose | |
|---|---|---|
| Norepinephrine monotherapy | ≥20 | μg/min |
| Dopamine monotherapy | ≥10 | μg/kg/min |
| Phenylephrine monotherapy | ≥200 | μg/min |
| Epinephrine monotherapy | ≥10 | μg/min |

| | |
|---|---|
| If on vasopressin | Vasopressin + norepinephrine |
| | equivalent of ≥10 μg/min ^{a} |
| If on combination or | Norepinephrine equivalent |
| vasopressors (not vasopressin) | of ≥20 μg/min ^{a} |

min = minute; VASST = Vasopressin and Septic Shock Trial.
^{a} VASST vasopressor equivalent equation: norepinephrine equivalent dose = [norepinephrine (μg/min)] + [dopamine (μg/kg/min) ÷ 2] + [epinephrine (μg/min)] + [phenylephrine (μg/min) ÷ 10].

Mild to moderate presentations of CRS and/or infusion-related reaction (IRR) may include symptoms such as fever, headache, and myalgia, and may be treated symptomatically with analgesics, anti-pyretics, and antihistamines as indicated. Severe or life-threatening presentations of CRS and/or IRR, such as hypotension, tachycardia, dyspnea, or chest discomfort should be treated aggressively with supportive and resuscitative measures as indicated, including the use of high-dose corticosteroids, IV fluids, admission to intensive care unit, and other supportive measures. Severe CRS may be associated with other clinical sequelae such as disseminated intravascular coagulation, capillary leak syndrome, or macrophage activation syndrome (MAS). Standard of care for severe or life-threatening CRS resulting from immune-based therapy has not been established; case reports and recommendations using anti-cytokine therapy such as tocilizumab have been published (Teachey et al., Blood, 121:5154-5157, 2013; Lee et al., Blood, 124:188-195, 2014; Maude et al., New Engl J Med, 371:1507-1517, 2014).

As noted in Table 2A, even moderate presentations of CRS in subjects with extensive comorbidities should be monitored closely, with consideration given to intensive care unit admission and tocilizumab administration.

vi. Administration of Tocilizumab as a Premedication

In some aspects, an effective amount of an interleukin-6 receptor (IL-6R) antagonist (e.g., an anti-IL-6R antibody, e.g., tocilizumab (ACTEMRA®/ROACTEMRA®)) is administered as a premedication (prophylaxis), e.g., is administered to the subject prior to the administration of the bispecific antibody (e.g., administered about 2 hours prior to the administration of the bispecific antibody). Administration of tocilizumab as a premedication may reduce the frequency or severity of CRS. In some aspects, tocilizumab is administered as a premedication in Cycle 1, e.g., is administered prior to a first dose (C1D1; cycle 1, dose 1), a second dose (C1D2; cycle 1, dose, 2), and/or a third dose (C1D3; cycle 1, dose 3) of the bispecific antibody. In some aspects, the tocilizumab is administered intravenously to the subject as a single dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, the tocilizumab is administered intravenously to the subject as a single dose of about 8 mg/kg. In some aspects, the tocilizumab is administered intravenously to the subject as a single dose of about 8 mg/kg for patients weighing 30 kg or more (maximum 800 mg) and at a dose of about 12 mg/kg for patients weighing less than 30 kg. Other anti-IL-6R antibodies that could be used in combination with tocilizumab include sarilumab, vobarilizumab (ALX-0061), SA-237, and variants thereof.

For example, in one aspect, the bispecific antibody is co-administered with tocilizumab (ACTEMRA®/ROACTEMRA®), wherein the subject is first administered with tocilizumab (ACTEMRA®/ROACTEMRA®) and then separately administered with the bispecific antibody (e.g., the subject is pre-treated with tocilizumab (ACTEMRA®/ROACTEMRA®)).

In some aspects, the incidence of CRS (e.g., Grade 1 CRS, Grade 2 CRS, and/or Grade 3+ CRS) is reduced in patients who are treated with tocilizumab as a premedication relative to patients who are not treated with tocilizumab as a premedication. In some aspects, less intervention to treat CRS (e.g., less need for additional tocilizumab, IV fluids, steroids, or O₂) is required in patients who are treated with tocilizumab as a premedication relative to patients who are not treated with tocilizumab as a premedication. In some aspects, CRS symptoms have decreased severity (e.g., are limited to fevers and rigors) in patients who are treated with tocilizumab as a premedication relative to patients who are not treated with tocilizumab as a premedication.

vii. Tocilizumab Administered to Treat CRS

In some aspects, the subject experiences a CRS event during treatment with the therapeutic bispecific antibody and an effective amount of an IL-6R antagonist (e.g., an anti-IL-6R antibody, e.g., tocilizumab (ACTEMRA®/ROACTEMRA®)) is administered to manage the CRS event.

In some aspects, the subject has a CRS event (e.g., has a CRS event following treatment with the bispecific antibody, e.g., has a CRS event following a first dose or a subsequent dose of the bispecific antibody), and the method further includes treating the symptoms of the CRS event while suspending treatment with the bispecific antibody.

In some aspects, the subject experiences a CRS event, and the method further includes administering to the subject an effective amount of an interleukin-6 receptor (IL-6R) antagonist (e.g., an anti-IL-6R antibody, e.g., tocilizumab (ACTEMRA®/ROACTEMRA®)) to manage the CRS event while suspending treatment with the bispecific antibody. In some aspects, the IL-6R antagonist (e.g., tocilizumab) is administered intravenously to the subject as a single dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, the tocilizumab is administered intravenously to the subject as a single dose of about 8 mg/kg. Other anti-IL-6R antibodies that could be used in combination with tocilizumab include sarilumab, vobarilizumab (ALX-0061), SA-237, and variants thereof.

In some aspects, the CRS event does not resolve or worsens within 24 hours of treating the symptoms of the CRS event, and the method further includes administering to the subject one or more additional doses of the IL-6R antagonist (e.g., an anti-IL-6R antibody, e.g., tocilizumab) to manage the CRS event, e.g., administering one or more additional doses of tocilizumab intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, the one or more additional doses of tocilizumab are administered intravenously to the subject as a single dose of about 8 mg/kg.

In some aspects, the method further includes administering to the subject an effective amount of a corticosteroid. The corticosteroid may be administered intravenously to the subject. In other examples, the corticosteroid may be administered subcutaneously to the subject. In some aspects, the corticosteroid is methylprednisolone. In some instances, the methylprednisolone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

The subject may be administered a corticosteroid, such as methylprednisolone or dexamethasone, if the CRS event is not managed with administration of the IL-6R antagonist (e.g., tocilizumab) alone. In some aspects, treating the symptoms of the CRS event further includes treatment with a high-dose vasopressor (e.g., norepinephrine, dopamine, phenylephrine, epinephrine, or vasopressin and norepinephrine), e.g., as described in Tables 2A, Table 2B, and Table 6. Tables 3A and 2A provide details about tocilizumab treatment of severe or life-threatening CRS.

viii. Management of CRS Events by Grade

Management of the CRS events may be tailored based on the grade of the CRS (Tables 2A and 3A) and the presence of comorbidities. Table 3A provides recommendations for the management of CRS syndromes by grade. Table 3B provides recommendations for the management of IRR syndromes by grade.

TABLE 3A
Recommendations for management of cytokine release syndrome (CRS)

| Event ^{a, b} | Action to Be Taken ^{b} |
|---|---|
| Grade 1 | Immediate actions: |
| Fever (≥38° C. or 100.4° F. not | If cevostamab infusion is still ongoing, interrupt infusion |
| attributable to any other cause) | immediately. |
| | Call to ensure tocilizumab is readily available if needed. |
| | Provide supportive care for constitutional symptoms. ^{c} |
| | Treat fever and neutropenia if present. |
| | Monitor fluid balance; administer IV fluids as clinically indicated. |
| | Consider hospitalization until symptoms completely resolve. |
| | In case of rapid decline in overall health or prolonged CRS |
| | (>24 hours) or in patients with significant symptoms and/or |
| | comorbidities (per investigator discretion, e.g., impaired |
| | cardiovascular function, reduced pulmonary reserve), consider |
| | administration of IV corticosteroids and tocilizumab or |
| | another anti-cytokine agent based on characterization of CRS. |
| | Restarting infusion: |
| | If cevostamab infusion was interrupted, wait until 30 minutes after |
| | the event has resolved before restarting the infusion at 50% of the |
| | original infusion rate. |
| | If symptoms recur, discontinue infusion for this dose. |
| | Next dose: |
| | Pretreat with antihistamines, antipyretics, and/or analgesics. |
| | Pretreat with IV corticosteroids (dexamethasone 20 mg |
| | (preferred) or methylprednisolone 80 mg). |
| | Consider hospitalization for next dose. |
| | Consider extending infusion time (slower infusion rate) for |
| | subsequent cycles. |
| Grade 2 | Immediate actions: |
| Fever (≥38° C. or 100.4° F. not | Follow all Grade 1 recommendations. |
| attributable to any other cause) | Hold further cevostamab treatment until symptoms completely |
| Hypotension: responds to fluids, | resolved. |
| not requiring vasopressors | Hospitalize patient until complete resolution of signs and |
| Hypoxia: requiring low flow nasal | symptoms. |
| cannula or blow-by | Administer tocilizumab 8 mg/kg IV ^{d} |
| | Consider treatment with IV corticosteroids (such as |
| | methylprednisolone 2 mg/kg/day or, if neurologic symptoms are |
| | present, dexamethasone 10 mg IV every 6 hours if needed). ^{b} |
| | Monitor cardiac and other organ function closely. |
| | Manage constitutional symptoms and organ toxicities as |
| | required. |
| | Provide hemodynamic support with IV fluid bolus 250-500 mL up |
| | to 2000 mL total (avoid fluid overload). |
| | Provide oxygen for hypoxia. |
| | Admit to ICU as appropriate. |
| | If no improvement within 8-12 hours after administration of |
| | tocilizumab, manage as a Grade 3 event: |
| | Initiate workup and assess for signs and symptoms of |
| | MAS/HLH. |
| | Restarting infusion: |
| | Wait until 30 minutes after the event has resolved before |
| | restarting the infusion at up to 25% of the original infusion rate. |
| | If symptoms recur, stop infusion immediately. Cevostamab |
| | should not be restarted. |
| | If hypotension or hypoxia recurs, manage as a Grade 3 event. |
| | Next cycle: |
| | May receive the next dose of cevostamab if symptoms resolve to |
| | Grade ≤1 for 3 consecutive days as follows: |
| | Consider hospitalization for at least 24 hours after the |
| | infusion. |
| | Administer cevostamab at 50% of the initial infusion rate of |
| | the previous cycle if the event occurred during or within 24 |
| | hours of the infusion. ^{e} |
| | Pretreat with antihistamines, antipyretics, and/or |
| | analgesics. |
| | Pretreat with IV corticosteroids (dexamethasone 20 mg |
| | (preferred) or methylprednisolone 80 mg). |
| | Subsequent cycles: |
| | If there is an occurrence of IRR or CRS Grade ≥3 in any of the |
| | subsequent cycles, permanently discontinue cevostamab |
| | regardless of recovery (see Grade 3 management guidelines). |
| | If there is an occurrence of a Grade ≤2 CRS in subsequent |
| | cycles, manage as indicated by severity (see Grade 1 or 2 |
| | management guidelines). |
| Grade 3 | Immediate actions: |
| Fever (≥38° C. or 100.4° F. not | Stop further infusion of cevostamab. |
| attributable to any other cause) | Provide supportive care and treat symptomatically as clinically |
| Hypotension: requires a | indicated. ^{c} |
| vasopressor with or without | Monitor fluid balance; administer IV fluids bolus 250-500 mL up to |
| vasopressin | 2000 mL total (avoid fluid overload); provide vasopressor support |
| Hypoxia: requiring a high-flow nasal | for hypotension with high and repeated doses if required. |
| cannula, face mask, non- rebreather | Hospitalize patient until complete resolution of signs and |
| mask, or Venturi mask | symptoms. |
| | Treat with IV corticosteroids (such as methylprednisolone 2 |
| | mg/kg/day or, if neurologic symptoms are present, |
| | dexamethasone 10 mg IV every 6 hours if needed). ^{b} |
| | Administer tocilizumab 8 mg/kg IV. ^{d} |
| | If there is no improvement after 8 hours, repeat tocilizumab |
| | administration. |
| | If no improvement within 8-12 hours after administering |
| | second dose of tocilizumab, manage as a Grade 4 event. |
| | Initiate workup and assess for signs and symptoms of |
| | MAS/HLH. |
| | Monitoring of cardiopulmonary and organ function in ICU is |
| | recommended. |
| | Provide oxygen for hypoxia. |
| | Restarting infusion: |
| | Do not restart infusion of cevostamab. |
| | Next cycle: |
| | If the patient had a Grade ≥2 IRR or CRS in any previous not |
| | cycle, permanently discontinue cevostamab. |
| | Patients who experience Grade 3 wheezing, |
| | bronchospasm, or generalized urticaria must be |
| | discontinued from cevostamab. |
| | If the patient recovers to Grade ≤1 for 3 consecutive days, |
| | cevostamab can be administered in next cycle as follows: |
| | Hospitalize patient for at least 24 hours after the mask |
| | infusion. |
| | Administer cevostamab at 50% of the initial infusion rate of |
| | the previous cycle if the event occurred during or within 24 |
| | hours of the infusion. ^{e} |
| | Pretreat with antihistamines, antipyretics, and/or analgesics |
| | Pretreat with IV corticosteroids (dexamethasone 20 mg |
| | (preferred) or methylprednisolone 80 mg). |
| | If the patient experienced Grade 3 CRS following the Cycle 1 |
| | Day 1 dose, the step-up dose must be repeated. |
| | Subsequent cycles: |
| | If a Grade ≥3 CRS recurs, permanently discontinue cevostamab. |
| | If there is an occurrence of a Grade ≤2 CRS in subsequent |
| | cycles, manage as indicated by severity (i.e., Grade 1 or 2 |
| | management guidelines). |
| Grade 4 | Stop cevostamab infusion. |
| Fever (≥38° C. or 100.4° F. not | Follow all Grade 3 management guidelines. |
| attributable to any other cause) | Patient requires ICU admission for hemodynamic monitoring, |
| Hypotension: requires multiple | and/or mechanical ventilation, and/or IV fluids and vasopressors |
| vasopressors | as needed. |
| Hypoxia: requiring positive pressure | Administer tocilizumab 8 mg/kg IV (see footnote). ^{d} |
| (e.g., CPAP, BiPAP, intubation, and | Treat with IV corticosteroids (such as methylprednisolone 2 |
| mechanical ventilation) | mg/kg/day or, if neurologic symptoms are present, |
| | dexamethasone 10 mg IV every 6 hours if needed) |
| | For patients who are refractory to tocilizumab therapy, |
| | experimental therapies (e.g., siltuximab, anakinra, emapalumab, |
| | or LCK-inhibitors) ^{b} may be considered based on the discretion |
| | of the investigator and institutional guidelines. |
| | Permanently discontinue study treatment. |

BiPAP = bilevel positive airway pressure; CPAP = continuous positive airway pressure; CRS = cytokine release syndrome; HLH = hemophagocytic lymphohistiocytosis; ICU = intensive care unit; IV = intravenous; MAS = macrophage activation syndrome.
^{a} Refer to Table 2A for the complete description of grading of symptoms.
^{b} Guidance for CRS management based on Lee et al., Biol Blood Marrow Transplant, 25(4); 625-638, (2019) and Riegler et al. (2019).
^{c} Patients should be treated with acetaminophen and an antihistamine (e.g., diphenhydramine) if they have not been administered in the previous 4 hours. For bronchospasm, urticaria, or dyspnea, treat per institutional practice. Treat fever and neutropenia as required; consider broad-spectrum antibiotics and/or G-CSF if indicated.
^{d} Tocilizumab should be administered at dose of 8 mg/kg IV (8 mg/kg for patients ≥30 kg weight only; 12 mg/kg for patients <30 kg weight; doses exceeding 800 mg per infusion are not recommended); repeat every 8 hours as necessary (up to a maximum of 4 doses).
^{e} If the patient does not experience CRS during the next infusion at the 50% reduced rate, the infusion rate can be increased to the initial rate in subsequent cycles. However, if this patient experiences another CRS event, the infusion rate should be reduced by 25%-50% depending on the severity of the event.

TABLE 3B
Recommendations for management of cevostamab
infusion related reactions (IRR)

| Event ^{a} | Action to Be Taken |
|---|---|
| Grade 1-2 | Slow infusion to ≤50% or interrupt infusion. |
| | Give supportive treatment. ^{b} |
| | Upon symptom resolution, may resume infusion (if interrupted) at 50% |
| | starting rate. The infusion must remain at the lower rate, resulting in |
| | symptom resolution for the remainder of the infusion. ^{c} |
| | Premedicate with acetaminophen/paracetamol and an antihistamine such as |
| | diphenhydramine for all subsequent infusions. |
| | Notes: |
| | 1. For Grade 2 wheezing or urticaria, patient must be premedicated prior to |
| | subsequent doses. |
| | 2. If symptoms recur with the same or greater severity following the slower |
| | or interrupted infusion of cevostamab, the infusion must be stopped |
| | immediately. No further cevostamab should be administered for the cycle. |
| Grade 3 | Discontinue cevostamab infusion. Do not re-initiate infusion for the |
| | current cycle. |
| | Give supportive treatment. ^{b} |
| | Admission to ICU is recommended to monitor cardiopulmonary and other |
| | organ functions. |
| | Provide oxygen for hypoxia and/or mechanical ventilation as needed. |
| | Subsequent cycles of cevostamab may be administered with |
| | premedication. |
| | Patients who experience Grade 3 wheezing, bronchospasm, or generalized |
| | urticaria at first occurrence must be discontinued from study treatment. |
| | Notes: |
| | 1. If symptoms recur despite premedications with the same or greater |
| | severity at subsequent cycles, the infusion must be stopped immediately |
| | and patient permanently discontinued from study treatment. |
| Grade 4 | Discontinue infusion immediately. |
| | Give supportive treatment. ^{b} |
| | Admit to ICU to monitor cardiopulmonary and other organ functions. |
| | Provide oxygen for hypoxia and/or mechanical ventilation as needed. |
| | Permanently discontinue study treatment. |

ICU = intensive care unit; NCI CTCAE = National Cancer Institute Common Terminology Criteria for Adverse Events.
^{a} Refer to NCI CTCAE v5.0 for the grading of symptoms.
^{b} Supportive treatment: Patients should be treated with acetaminophen/paracetamol and an antihistamine such as diphenhydramine if they have not been administered in the last 4 hours. Intravenous fluids (e.g., normal saline) may be administered as clinically indicated. For bronchospasm, urticaria, or dyspnea, antihistamines, oxygen, corticosteroids (e.g., 100 mg IV prednisolone or equivalent), and/or bronchodilators may be administered per institutional practice. Provide fluids and vasopressor support for hypotension if required.
^{c} Subsequent infusions of cevostamab may be started at the original rate.

ix. Management of Grade 2 CRS Events

If the subject has a grade 2 CRS event (e.g., a grade 2 CRS event in the absence of comorbidities or in the presence of minimal comorbidities) following administration of the therapeutic bispecific antibody, the method may further include treating the symptoms of the grade 2 CRS event while suspending treatment with the bispecific antibody. If the grade 2 CRS event then resolves to a grade≤1 CRS event for at least three consecutive days, the method may further include resuming treatment with the bispecific antibody without altering the dose. On the other hand, if the grade 2 CRS event does not resolve or worsens to a grade ≥3 CRS event within 24 hours of treating the symptoms of the grade 2 CRS event, the method may further involve administering to the subject an effective amount of an interleukin-6 receptor (IL-6R) antagonist (e.g., an anti-IL-6R antibody, e.g., tocilizumab (ACTEMRA®/ROACTEMRA®)) to manage the grade 2 or grade ≥3 CRS event. In some instances, tocilizumab is administered intravenously to the subject as a single dose of about 8 mg/kg. Other anti-IL-6R antibodies that could be used in combination with tocilizumab include sarilumab, vobarilizumab (ALX-0061), SA-237, and variants thereof.

If the subject has a grade 2 CRS event in the presence of extensive comorbidities following administration of the therapeutic bispecific antibody, the method may further include administering to the subject a first dose of an IL-6R antagonist (e.g., an anti-IL-6R antibody, e.g., tocilizumab (ACTEMRA®/ROACTEMRA®)) to manage the grade 2 CRS event while suspending treatment with the bispecific antibody. In some instances, the first dose of tocilizumab is administered intravenously to the subject at a dose of about 8 mg/kg. Other anti-IL-6R antibodies that could be used in combination with tocilizumab include sarilumab, vobarilizumab (ALX-0061), SA-237, and variants thereof. In some instances, if the grade 2 CRS event resolves to a grade≤1 CRS event within two weeks, the method further includes resuming treatment with the bispecific antibody at a reduced dose. In some instances, the reduced dose is 50% of the initial infusion rate of the previous cycle if the event occurred during or within 24 hours of the infusion. If, on the other hand, the grade 2 CRS event does not resolve or worsens to a grade ≥3 CRS event within 24 hours of treating the symptoms of the grade 2 CRS event, the method may further include administering to the subject one or more (e.g., one, two, three, four, or five or more) additional doses of an IL-6R antagonist (e.g., an anti-IL-6R antibody, e.g., tocilizumab) to manage the grade 2 or grade ≥3 CRS event. In some particular instances, the grade 2 CRS event does not resolve or worsens to a grade ≥3 CRS event within 24 hours of treating the symptoms of the grade 2 CRS event, and the method may further include administering to the subject one or more additional doses of tocilizumab to manage the grade 2 or grade ≥3 CRS event. In some instances, the one or more additional doses of tocilizumab is administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some instances, the method further includes administering to the subject an effective amount of a corticosteroid. The corticosteroid may be administered before, after, or concurrently with the one or more additional doses of tocilizumab or another anti-IL-6R antibody. In some instances, the corticosteroid is administered intravenously to the subject. In some instances, the corticosteroid is methylprednisolone. In some instances, the methylprednisolone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

### X. Management of Grade 3 CRS Events

If the subject has a grade 3 CRS event following administration of the therapeutic bispecific antibody, the method may further include administering to the subject a first dose of an IL-6R antagonist (e.g., an anti-IL-6R antibody, e.g., tocilizumab (ACTEMRA®/ROACTEMRA®)) to manage the grade 3 CRS event while suspending treatment with the bispecific antibody. In some instances, the first dose of tocilizumab is administered intravenously to the subject at a dose of about 8 mg/kg. Other anti-IL-6R antibodies that could be used in combination with tocilizumab include sarilumab, vobarilizumab (ALX-0061), SA-237, and variants thereof. In some instances, the subject recovers (e.g., is afebrile and off vasopressors) within 8 hours following treatment with the bispecific antibody, and the method further includes resuming treatment with the bispecific antibody at a reduced dose. In some instances, the reduced dose is 50% of the initial infusion rate of the previous cycle if the event occurred during or within 24 hours of the infusion. In other instances, if the grade 3 CRS event does not resolve or worsens to a grade 4 CRS event within 24 hours of treating the symptoms of the grade 3 CRS event, the method may further include administering to the subject one or more (e.g., one, two, three, four, or five or more) additional doses of an IL-6R antagonist (e.g., an anti-IL-6R antibody, e.g., tocilizumab) to manage the grade 3 or grade 4 CRS event. In some particular instances, the grade 3 CRS event does not resolve or worsens to a grade 4 CRS event within 24 hours of treating the symptoms of the grade 3 CRS event, and the method further includes administering to the subject one or more additional doses of tocilizumab to manage the grade 3 or grade 4 CRS event. In some instances, the one or more additional doses of tocilizumab is administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some instances, the method further includes administering to the subject an effective amount of a corticosteroid. The corticosteroid may be administered before, after, or concurrently with the one or more additional doses of tocilizumab or another anti-IL-6R antibody. In some instances, the corticosteroid is administered intravenously to the subject. In some instances, the corticosteroid is methylprednisolone. In some instances, the methylprednisolone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

xi. Management of Grade 4 CRS Events

If the subject has a grade 4 CRS event following administration of the therapeutic bispecific antibody, the method may further include administering to the subject a first dose of an IL-6R antagonist (e.g., an anti-IL-6R antibody, e.g., tocilizumab (ACTEMRA®/ROACTEMRA®)) to manage the grade 4 CRS event and permanently discontinuing treatment with the bispecific antibody. In some instances, the first dose of tocilizumab is administered intravenously to the subject at a dose of about 8 mg/kg. Other anti-IL-6R antibodies that could be used in combination with tocilizumab include sarilumab, vobarilizumab (ALX-0061), SA-237, and variants thereof. The grade 4 CRS event may, in some instances, resolve within 24 of treating the symptoms of the grade 4 CRS event. If the grade 4 CRS event does not resolve within 24 hours of treating the symptoms of the grade 4 CRS event, the method may further include administering to the subject one or more additional doses of an IL-6R antagonist (e.g., an anti-IL-6R antibody, e.g., tocilizumab (ACTEMRA®/ROACTEMRA®)) to manage the grade 4 CRS event. In some particular instances, the grade 4 CRS event does not resolve within 24 hours of treating the symptoms of the grade 4 CRS event, and the method further includes administering to the subject one or more (e.g., one, two, three, four, or five or more) additional doses of tocilizumab to manage the grade 4 CRS event. In some instances, the one or more additional doses of tocilizumab is administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some instances, the method further includes administering to the subject an effective amount of a corticosteroid. The corticosteroid may be administered before, after, or concurrently with the one or more additional doses of tocilizumab or another anti-IL-6R antibody. In some instances, the corticosteroid is administered intravenously to the subject. In some instances, the corticosteroid is methylprednisolone. In some instances, the methylprednisolone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

xii. Acetaminophen or Paracetamol

In another instance, the additional therapeutic agent is an effective amount of acetaminophen or paracetamol. The acetaminophen or paracetamol may be administered orally to the subject, e.g., administered orally at a dose of between about 500 mg to about 1000 mg. In some aspects, the acetaminophen or paracetamol is administered to the subject as a premedication, e.g., is administered prior to the administration of the bispecific anti-FcRH5/anti-CD3 antibody.

xiii. Diphenhydramine

In another instance, the additional therapeutic agent is an effective amount of diphenhydramine. The diphenhydramine may be administered orally to the subject, e.g., administered orally at a dose of between about 25 mg to about 50 mg. In some aspects, the diphenhydramine is administered to the subject as a premedication, e.g., is administered prior to the administration of the bispecific anti-FcRH5/anti-CD3 antibody.

xiv. Anti-Myeloma Agents

In another instance, the additional therapeutic agent is an effective amount of an anti-myeloma agent, e.g., an anti-myeloma agent that augments and/or complements T-cell-mediated killing of myeloma cells. The anti-myeloma agent may be, e.g., pomalidomide, daratumumab, and/or a B-cell maturation antigen (BCMA)-directed therapy (e.g., an antibody-drug conjugate targeting BCMA (BCMA-ADC)). In some aspects, the anti-myeloma agent is administered in four-week cycles.

xv. Other Combination Therapies

In some aspects, the one or more additional therapeutic agents comprise a PD-1 axis binding antagonist, an immunomodulatory agent, an anti-neoplastic agent, a chemotherapeutic agent, a growth inhibitory agent, an anti-angiogenic agent, a radiation therapy, a cytotoxic agent, a cell-based therapy, or a combination thereof.

xvi. PD-1 Axis Binding Antagonists

In some aspects, the additional therapeutic agent is a PD-1 axis binding antagonist. PD-1 axis binding antagonists may include PD-L1 binding antagonists, PD-1 binding antagonists, and PD-L2 binding antagonists. Any suitable PD-1 axis binding antagonist may be used.

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. The PD-L1 binding antagonist may be, without limitation, an antibody, an antigen-binding fragment thereof, an immunoadhesin, a fusion protein, an oligopeptide, or a small molecule. In some instances, the PD-L1 binding antagonist is a small molecule that inhibits PD-L1 (e.g., GS-4224, INCB086550, MAX-10181, INCB090244, CA-170, or ABSK041). In some instances, the PD-L1 binding antagonist is a small molecule that inhibits PD-L1 and VISTA. In some instances, the PD-L1 binding antagonist is CA-170 (also known as AUPM-170). In some instances, the PD-L1 binding antagonist is a small molecule that inhibits PD-L1 and TIM3. In some instances, the small molecule is a compound described in WO 2015/033301 and/or WO 2015/033299.

In some instances, the PD-L1 binding antagonist is an anti-PD-L1 antibody. A variety of anti-PD-L1 antibodies are contemplated and described herein. In any of the instances herein, the isolated anti-PD-L1 antibody can bind to a human PD-L1, for example a human PD-L1 as shown in UniProtKB/Swiss-Prot Accession No. Q9NZQ7-1, or a variant thereof. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)₂ fragments. In some instances, the anti-PD-L1 antibody is a humanized antibody. In some instances, the anti-PD-L1 antibody is a human antibody. Exemplary anti-PD-L1 antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some instances, the anti-PD-L1 antibody is atezolizumab. Examples of anti-PD-L1 antibodies useful in the methods of this invention and methods of making them are described in International Patent Application Publication No. WO 2010/077634 and U.S. Pat. No. 8,217,149, each of which is incorporated herein by reference in its entirety.

In some instances, the anti-PD-L1 antibody is avelumab (CAS Registry Number: 1537032-82-8). Avelumab, also known as MSB0010718C, is a human monoclonal IgG1 anti-PD-L1 antibody (Merck KGaA, Pfizer).

In some instances, the anti-PD-L1 antibody is durvalumab (CAS Registry Number: 1428935-60-7). Durvalumab, also known as MEDI4736, is an Fc-optimized human monoclonal IgG1 kappa anti-PD-L1 antibody (MedImmune, AstraZeneca) described in WO 2011/066389 and US 2013/034559.

In some instances, the anti-PD-L1 antibody is MDX-1105 (Bristol Myers Squibb). MDX-1105, also known as BMS-936559, is an anti-PD-L1 antibody described in WO 2007/005874.

In some instances, the anti-PD-L1 antibody is LY3300054 (Eli Lilly).

In some instances, the anti-PD-L1 antibody is STI-A1014 (Sorrento). STI-A1014 is a human anti-PD-L1 antibody.

In some instances, the anti-PD-L1 antibody is KN035 (Suzhou Alphamab). KN035 is single-domain antibody (dAB) generated from a camel phage display library.

In some instances, the anti-PD-L1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some instances, the anti-PD-L1 antibody is CX-072 (CytomX Therapeutics).

In some instances, the anti-PD-L1 antibody comprises the six HVR sequences (e.g., the three heavy chain HVRs and the three light chain HVRs) and/or the heavy chain variable domain and light chain variable domain from an anti-PD-L1 antibody described in US20160108123, WO 2016/000619, WO 2012/145493, U.S. Pat. No. 9,205,148, WO 2013/181634, or WO 2016/061142.

In some instances, the PD-1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. The PD-1 binding antagonist may be, without limitation, an antibody, an antigen-binding fragment thereof, an immunoadhesin, a fusion protein, an oligopeptide, or a small molecule. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). For example, in some instances, the PD-1 binding antagonist is an Fc-fusion protein. In some instances, the PD-1 binding antagonist is AMP-224. AMP-224, also known as B7-DClg, is a PD-L2-Fc fusion soluble receptor described in WO 2010/027827 and WO 2011/066342. In some instances, the PD-1 binding antagonist is a peptide or small molecule compound. In some instances, the PD-1 binding antagonist is AUNP-12 (PierreFabre/Aurigene). See, e.g., WO 2012/168944, WO 2015/036927, WO 2015/044900, WO 2015/033303, WO 2013/144704, WO 2013/132317, and WO 2011/161699. In some instances, the PD-1 binding antagonist is a small molecule that inhibits PD-1.

In some instances, the PD-1 binding antagonist is an anti-PD-1 antibody. A variety of anti-PD-1 antibodies can be utilized in the methods and uses disclosed herein. In any of the instances herein, the PD-1 antibody can bind to a human PD-1 or a variant thereof. In some instances, the anti-PD-1 antibody is a monoclonal antibody. In some instances, the anti-PD-1 antibody is an antibody fragment selected from the group consisting of Fab, Fab′, Fab′-SH, Fv, scFv, and (Fab′)₂ fragments. In some instances, the anti-PD-1 antibody is a humanized antibody. In other instances, the anti-PD-1 antibody is a human antibody. Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21.

In some instances, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO 2006/121168.

In some instances, the anti-PD-1 antibody is pembrolizumab (CAS Registry Number: 1374853-91-4). Pembrolizumab (Merck), also known as MK-3475, Merck 3475, lambrolizumab, SCH-900475, and KEYTRUDA®, is an anti-PD-1 antibody described in WO 2009/114335.

In some instances, the anti-PD-1 antibody is MEDI-0680 (AMP-514; AstraZeneca). MEDI-0680 is a humanized IgG4 anti-PD-1 antibody.

In some instances, the anti-PD-1 antibody is PDR001 (CAS Registry No. 1859072-53-9; Novartis). PDR001 is a humanized IgG4 anti-PD-1 antibody that blocks the binding of PD-L1 and PD-L2 to PD-1.

In some instances, the anti-PD-1 antibody is REGN2810 (Regeneron). REGN2810 is a human anti-PD-1 antibody.

In some instances, the anti-PD-1 antibody is BGB-108 (BeiGene).

In some instances, the anti-PD-1 antibody is BGB-A317 (BeiGene).

In some instances, the anti-PD-1 antibody is JS-001 (Shanghai Junshi). JS-001 is a humanized anti-PD-1 antibody.

In some instances, the anti-PD-1 antibody is STI-A1110 (Sorrento). STI-A1110 is a human anti-PD-1 antibody.

In some instances, the anti-PD-1 antibody is INCSHR-1210 (Incyte). INCSHR-1210 is a human IgG4 anti-PD-1 antibody.

In some instances, the anti-PD-1 antibody is PF-06801591 (Pfizer).

In some instances, the anti-PD-1 antibody is TSR-042 (also known as ANB011; Tesaro/AnaptysBio).

In some instances, the anti-PD-1 antibody is AM0001 (ARMO Biosciences).

In some instances, the anti-PD-1 antibody is ENUM 244C8 (Enumeral Biomedical Holdings). ENUM 244C8 is an anti-PD-1 antibody that inhibits PD-1 function without blocking binding of PD-L1 to PD-1.

In some instances, the anti-PD-1 antibody is ENUM 388D4 (Enumeral Biomedical Holdings). ENUM 388D4 is an anti-PD-1 antibody that competitively inhibits binding of PD-L1 to PD-1.

In some instances, the anti-PD-1 antibody comprises the six HVR sequences (e.g., the three heavy chain HVRs and the three light chain HVRs) and/or the heavy chain variable domain and light chain variable domain from an anti-PD-1 antibody described in WO 2015/112800, WO 2015/112805, WO 2015/112900, US20150210769, WO2016/089873, WO 2015/035606, WO 2015/085847, WO 2014/206107, WO 2012/145493, U.S. Pat. No. 9,205,148, WO 2015/119930, WO 2015/119923, WO 2016/032927, WO 2014/179664, WO 2016/106160, and WO 2014/194302.

In some instances, the PD-1 axis binding antagonist is a PD-L2 binding antagonist. In some instances, the PD-L2 binding antagonist is a molecule that inhibits the binding of PD-L2 to its ligand binding partners. In a specific aspect, the PD-L2 binding ligand partner is PD-1. The PD-L2 binding antagonist may be, without limitation, an antibody, an antigen-binding fragment thereof, an immunoadhesin, a fusion protein, an oligopeptide, or a small molecule.

In some instances, the PD-L2 binding antagonist is an anti-PD-L2 antibody. In any of the instances herein, the anti-PD-L2 antibody can bind to a human PD-L2 or a variant thereof. In some instances, the anti-PD-L2 antibody is a monoclonal antibody. In some instances, the anti-PD-L2 antibody is an antibody fragment selected from the group consisting of Fab, Fab′, Fab′-SH, Fv, scFv, and (Fab′)₂ fragments. In some instances, the anti-PD-L2 antibody is a humanized antibody. In other instances, the anti-PD-L2 antibody is a human antibody. In a still further specific aspect, the anti-PD-L2 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-L2 antibody is aglycosylated.

xvii. Growth Inhibitory Agents

In some aspects, the additional therapeutic agent is a growth inhibitory agent. Exemplary growth inhibitory agents include agents that block cell cycle progression at a place other than S phase, e.g., agents that induce G1 arrest (e.g., DNA alkylating agents such as tamoxifen, prednisone, dacarbazine, mechlorethamine, cisplatin, methotrexate, 5-fluorouracil, or ara-C) or M-phase arrest (e.g., vincristine, vinblastine, taxanes (e.g., paclitaxel and docetaxel), doxorubicin, epirubicin, daunorubicin, etoposide, or bleomycin).

xviii. Radiation Therapies

In some aspects, the additional therapeutic agent is a radiation therapy. Radiation therapies include the use of directed gamma rays or beta rays to induce sufficient damage to a cell so as to limit its ability to function normally or to destroy the cell altogether. Typical treatments are given as a one-time administration and typical dosages range from 10 to 200 units (Grays) per day.

xix. Cytotoxic Agents

In some aspects, the additional therapeutic agent is a cytotoxic agent, e.g., a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹², and radioactive isotopes of Lu); chemotherapeutic agents or drugs (e.g., methotrexate, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and antitumor or anticancer agents.

xx. Anti-Cancer Therapies

In some instances, the methods include administering to the individual an anti-cancer therapy other than, or in addition to, a bispecific anti-FcRH5/anti-CD3 antibody (e.g., an anti-neoplastic agent, a chemotherapeutic agent, a growth inhibitory agent, an anti-angiogenic agent, a radiation therapy, or a cytotoxic agent).

In some instances, the methods further involve administering to the patient an effective amount of an additional therapeutic agent. In some instances, the additional therapeutic agent is selected from the group consisting of an anti-neoplastic agent, a chemotherapeutic agent, a growth inhibitory agent, an anti-angiogenic agent, a radiation therapy, a cytotoxic agent, and combinations thereof. In some instances, a bispecific anti-FcRH5/anti-CD3 antibody may be administered in conjunction with a chemotherapy or chemotherapeutic agent. In some instances, a bispecific anti-FcRH5/anti-CD3 antibody may be administered in conjunction with a radiation therapy agent. In some instances, a bispecific anti-FcRH5/anti-CD3 antibody may be administered in conjunction with a targeted therapy or targeted therapeutic agent. In some instances, a bispecific anti-FcRH5/anti-CD3 antibody may be administered in conjunction with an immunotherapy or immunotherapeutic agent, for example a monoclonal antibody. In some instances, the additional therapeutic agent is an agonist directed against a co-stimulatory molecule. In some instances, the additional therapeutic agent is an antagonist directed against a co-inhibitory molecule.

Without wishing to be bound to theory, it is thought that enhancing T-cell stimulation, by promoting a co-stimulatory molecule or by inhibiting a co-inhibitory molecule, may promote tumor cell death thereby treating or delaying progression of cancer. In some instances, a bispecific anti-FcRH5/anti-CD3 antibody may be administered in conjunction with an agonist directed against a co-stimulatory molecule. In some instances, a co-stimulatory molecule may include CD40, CD226, CD28, OX40, GITR, CD137, CD27, HVEM, or CD127. In some instances, the agonist directed against a co-stimulatory molecule is an agonist antibody that binds to CD40, CD226, CD28, OX40, GITR, CD137, CD27, HVEM, or CD127. In some instances, a bispecific anti-FcRH5/anti-CD3 antibody may be administered in conjunction with an antagonist directed against a co-inhibitory molecule. In some instances, a co-inhibitory molecule may include CTLA-4 (also known as CD152), TIM-3, BTLA, VISTA, LAG-3, B7-H3, B7-H4, IDO, TIGIT, MICA/B, or arginase. In some instances, the antagonist directed against a co-inhibitory molecule is an antagonist antibody that binds to CTLA-4, TIM-3, BTLA, VISTA, LAG-3, B7-H3, B7-H4, IDO, TIGIT, MICA/B, or arginase.

In some instances, a bispecific anti-FcRH5/anti-CD3 antibody may be administered in conjunction with an antagonist directed against CTLA-4 (also known as CD152), e.g., a blocking antibody. In some instances, a bispecific anti-FcRH5/anti-CD3 antibody may be administered in conjunction with ipilimumab (also known as MDX-010, MDX-101, or YERVOY®). In some instances, a bispecific anti-FcRH5/anti-CD3 antibody may be administered in conjunction with tremelimumab (also known as ticilimumab or CP-675,206). In some instances, a bispecific anti-FcRH5/anti-CD3 antibody may be administered in conjunction with an antagonist directed against B7-H3 (also known as CD276), e.g., a blocking antibody. In some instances, a bispecific anti-FcRH5/anti-CD3 antibody may be administered in conjunction with MGA271. In some instances, a bispecific anti-FcRH5/anti-CD3 antibody may be administered in conjunction with an antagonist directed against a TGF-beta, e.g., metelimumab (also known as CAT-192), fresolimumab (also known as GC1008), or LY2157299.

In some instances, a bispecific anti-FcRH5/anti-CD3 antibody may be administered in conjunction with a treatment comprising adoptive transfer of a T-cell (e.g., a cytotoxic T-cell or CTL) expressing a chimeric antigen receptor (CAR). In some instances, bispecific anti-FcRH5/anti-CD3 antibody may be administered in conjunction with a treatment comprising adoptive transfer of a T-cell comprising a dominant-negative TGF beta receptor, e.g., a dominant-negative TGF beta type II receptor. In some instances, a bispecific anti-FcRH5/anti-CD3 antibody may be administered in conjunction with a treatment comprising a HERCREEM protocol (see, e.g., ClinicalTrials.gov Identifier NCT00889954).

In some instances, a bispecific anti-FcRH5/anti-CD3 antibody may be administered in conjunction with an agonist directed against CD137 (also known as TNFRSF9, 4-1BB, or ILA), e.g., an activating antibody. In some instances, a bispecific anti-FcRH5/anti-CD3 antibody may be administered in conjunction with urelumab (also known as BMS-663513). In some instances, a bispecific anti-FcRH5/anti-CD3 antibody may be administered in conjunction with an agonist directed against CD40, e.g., an activating antibody. In some instances, bispecific anti-FcRH5/anti-CD3 antibody may be administered in conjunction with CP-870893. In some instances, bispecific anti-FcRH5/anti-CD3 antibody may be administered in conjunction with an agonist directed against OX40 (also known as CD134), e.g., an activating antibody. In some instances, a bispecific anti-FcRH5/anti-CD3 antibody may be administered in conjunction with an anti-OX40 antibody (e.g., AgonOX). In some instances, a bispecific anti-FcRH5/anti-CD3 antibody may be administered in conjunction with an agonist directed against CD27, e.g., an activating antibody. In some instances, a bispecific anti-FcRH5/anti-CD3 antibody may be administered in conjunction with CDX-1127. In some instances, a bispecific anti-FcRH5/anti-CD3 antibody may be administered in conjunction with an antagonist directed against indoleamine-2,3-dioxygenase (IDO). In some instances, with the IDO antagonist is 1-methyl-D-tryptophan (also known as 1-D-MT).

In some instances, a bispecific anti-FcRH5/anti-CD3 antibody may be administered in conjunction with an antibody-drug conjugate. In some instances, the antibody-drug conjugate comprises mertansine or monomethyl auristatin E (MMAE). In some instances, a bispecific anti-FcRH5/anti-CD3 antibody may be administered in conjunction with an anti-NaPi2b antibody-MMAE conjugate (also known as DNIB0600A or RG7599). In some instances, a bispecific anti-FcRH5/anti-CD3 antibody may be administered in conjunction with trastuzumab emtansine (also known as T-DM1, ado-trastuzumab emtansine, or KADCYLA®, Genentech). In some instances, a bispecific anti-FcRH5/anti-CD3 antibody may be administered in conjunction with DMUC5754A. In some instances, a bispecific anti-FcRH5/anti-CD3 antibody may be administered in conjunction with an antibody-drug conjugate targeting the endothelin B receptor (EDNBR), e.g., an antibody directed against EDNBR conjugated with MMAE.

In some instances, a bispecific anti-FcRH5/anti-CD3 antibody may be administered in conjunction with an anti-angiogenesis agent. In some instances, a bispecific anti-FcRH5/anti-CD3 antibody may be administered in conjunction with an antibody directed against a VEGF, e.g., VEGF-A. In some instances, a bispecific anti-FcRH5/anti-CD3 antibody may be administered in conjunction with bevacizumab (also known as AVASTIN®, Genentech). In some instances, a bispecific anti-FcRH5/anti-CD3 antibody may be administered in conjunction with an antibody directed against angiopoietin 2 (also known as Ang2). In some instances, a bispecific anti-FcRH5/anti-CD3 antibody may be administered in conjunction with MEDI3617.

In some instances, a bispecific anti-FcRH5/anti-CD3 antibody may be administered in conjunction with an antineoplastic agent. In some instances, a bispecific anti-FcRH5/anti-CD3 antibody may be administered in conjunction with an agent targeting CSF-1R (also known as M-CSFR or CD115). In some instances, a bispecific anti-FcRH5/anti-CD3 antibody may be administered in conjunction with anti-CSF-1R (also known as IMC-CS4). In some instances, a bispecific anti-FcRH5/anti-CD3 antibody may be administered in conjunction with an interferon, for example interferon alpha or interferon gamma. In some instances, a bispecific anti-FcRH5/anti-CD3 antibody may be administered in conjunction with Roferon-A (also known as recombinant Interferon alpha-2a). In some instances, a bispecific anti-FcRH5/anti-CD3 antibody may be administered in conjunction with GM-CSF (also known as recombinant human granulocyte macrophage colony stimulating factor, rhu GM-CSF, sargramostim, or LEUKINE®). In some instances, a bispecific anti-FcRH5/anti-CD3 antibody may be administered in conjunction with IL-2 (also known as aldesleukin or PROLEUKIN®). In some instances, a bispecific anti-FcRH5/anti-CD3 antibody may be administered in conjunction with IL-12. In some instances, a bispecific anti-FcRH5/anti-CD3 antibody may be administered in conjunction with an antibody targeting CD20. In some instances, the antibody targeting CD20 is obinutuzumab (also known as GA101 or GAZYVAR) or rituximab. In some instances, a bispecific anti-FcRH5/anti-CD3 antibody may be administered in conjunction with an antibody targeting GITR. In some instances, the antibody targeting GITR is TRX518.

In some instances, a bispecific anti-FcRH5/anti-CD3 antibody may be administered in conjunction with a cancer vaccine. In some instances, the cancer vaccine is a peptide cancer vaccine, which in some instances is a personalized peptide vaccine. In some instances, the peptide cancer vaccine is a multivalent long peptide, a multi-peptide, a peptide cocktail, a hybrid peptide, or a peptide-pulsed dendritic cell vaccine (see, e.g., Yamada et al., Cancer Sci. 104:14-21, 2013). In some instances, a bispecific anti-FcRH5/anti-CD3 antibody may be administered in conjunction with an adjuvant. In some instances, a bispecific anti-FcRH5/anti-CD3 antibody may be administered in conjunction with a treatment comprising a TLR agonist, e.g., Poly-ICLC (also known as HILTONOL®), LPS, MPL, or CpG ODN. In some instances, a bispecific anti-FcRH5/anti-CD3 antibody may be administered in conjunction with tumor necrosis factor (TNF) alpha. In some instances, a bispecific anti-FcRH5/anti-CD3 antibody may be administered in conjunction with IL-1. In some instances, a bispecific anti-FcRH5/anti-CD3 antibody may be administered in conjunction with HMGB1. In some instances, a bispecific anti-FcRH5/anti-CD3 antibody may be administered in conjunction with an IL-10 antagonist. In some instances, a bispecific anti-FcRH5/anti-CD3 antibody may be administered in conjunction with an IL-4 antagonist. In some instances, a bispecific anti-FcRH5/anti-CD3 antibody may be administered in conjunction with an IL-13 antagonist. In some instances, a bispecific anti-FcRH5/anti-CD3 antibody may be administered in conjunction with an HVEM antagonist. In some instances, a bispecific anti-FcRH5/anti-CD3 antibody may be administered in conjunction with an ICOS agonist, e.g., by administration of ICOS-L, or an agonistic antibody directed against ICOS. In some instances, a bispecific anti-FcRH5/anti-CD3 antibody may be administered in conjunction with a treatment targeting CX3CL1. In some instances, a bispecific anti-FcRH5/anti-CD3 antibody may be administered in conjunction with a treatment targeting CXCL9. In some instances, a bispecific anti-FcRH5/anti-CD3 antibody may be administered in conjunction with a treatment targeting CXCL10. In some instances, a bispecific anti-FcRH5/anti-CD3 antibody may be administered in conjunction with a treatment targeting CCL5. In some instances, a bispecific anti-FcRH5/anti-CD3 antibody may be administered in conjunction with an LFA-1 or ICAM1 agonist. In some instances, a bispecific anti-FcRH5/anti-CD3 antibody may be administered in conjunction with a Selectin agonist.

In some instances, a bispecific anti-FcRH5/anti-CD3 antibody may be administered in conjunction with a targeted therapy. In some instances, a bispecific anti-FcRH5/anti-CD3 antibody may be administered in conjunction with an inhibitor of B-Raf. In some instances, a bispecific anti-FcRH5/anti-CD3 antibody may be administered in conjunction with vemurafenib (also known as ZELBORAF®). In some instances, a bispecific anti-FcRH5/anti-CD3 antibody may be administered in conjunction with dabrafenib (also known as TAFINLAR®). In some instances, a bispecific anti-FcRH5/anti-CD3 antibody may be administered in conjunction with erlotinib (also known as TARCEVA®). In some instances, a bispecific anti-FcRH5/anti-CD3 antibody may be administered in conjunction with an inhibitor of a MEK, such as MEK1 (also known as MAP2K1) or MEK2 (also known as MAP2K2). In some instances, a bispecific anti-FcRH5/anti-CD3 antibody may be administered in conjunction with cobimetinib (also known as GDC-0973 or XL-518). In some instances, a bispecific anti-FcRH5/anti-CD3 antibody may be administered in conjunction with trametinib (also known as MEKINIST®). In some instances, a bispecific anti-FcRH5/anti-CD3 antibody may be administered in conjunction with an inhibitor of K-Ras. In some instances, a bispecific anti-FcRH5/anti-CD3 antibody may be administered in conjunction with an inhibitor of c-Met. In some instances, a bispecific anti-FcRH5/anti-CD3 antibody may be administered in conjunction with onartuzumab (also known as MetMAb). In some instances, a bispecific anti-FcRH5/anti-CD3 antibody may be administered in conjunction with an inhibitor of Alk. In some instances, a bispecific anti-FcRH5/anti-CD3 antibody may be administered in conjunction with AF802 (also known as CH5424802 or alectinib). In some instances, a bispecific anti-FcRH5/anti-CD3 antibody may be administered in conjunction with an inhibitor of a phosphatidylinositol 3-kinase (PI3K). In some instances, a bispecific anti-FcRH5/anti-CD3 antibody may be administered in conjunction with BKM120. In some instances, a bispecific anti-FcRH5/anti-CD3 antibody may be administered in conjunction with idelalisib (also known as GS-1101 or CAL-101). In some instances, a bispecific anti-FcRH5/anti-CD3 antibody may be administered in conjunction with perifosine (also known as KRX-0401). In some instances, a bispecific anti-FcRH5/anti-CD3 antibody may be administered in conjunction with an inhibitor of an Akt. In some instances, a bispecific anti-FcRH5/anti-CD3 antibody may be administered in conjunction with MK2206. In some instances, a bispecific anti-FcRH5/anti-CD3 antibody may be administered in conjunction with GSK690693. In some instances, a bispecific anti-FcRH5/anti-CD3 antibody may be administered in conjunction with GDC-0941. In some instances, a bispecific anti-FcRH5/anti-CD3 antibody may be administered in conjunction with an inhibitor of mTOR. In some instances, a bispecific anti-FcRH5/anti-CD3 antibody may be administered in conjunction with sirolimus (also known as rapamycin). In some instances, a bispecific anti-FcRH5/anti-CD3 antibody may be administered in conjunction with temsirolimus (also known as CCI-779 or TORISEL®). In some instances, a bispecific anti-FcRH5/anti-CD3 antibody may be administered in conjunction with everolimus (also known as RAD001). In some instances, a bispecific anti-FcRH5/anti-CD3 antibody may be administered in conjunction with ridaforolimus (also known as AP-23573, MK-8669, or deforolimus). In some instances, a bispecific anti-FcRH5/anti-CD3 antibody may be administered in conjunction with OSI-027. In some instances, a bispecific anti-FcRH5/anti-CD3 antibody may be administered in conjunction with AZD8055. In some instances, a bispecific anti-FcRH5/anti-CD3 antibody may be administered in conjunction with INK128. In some instances, a bispecific anti-FcRH5/anti-CD3 antibody may be administered in conjunction with a dual PI3K/mTOR inhibitor. In some instances, a bispecific anti-FcRH5/anti-CD3 antibody may be administered in conjunction with XL765. In some instances, a bispecific anti-FcRH5/anti-CD3 antibody may be administered in conjunction with GDC-0980. In some instances, a bispecific anti-FcRH5/anti-CD3 antibody may be administered in conjunction with BEZ235 (also known as NVP-BEZ235). In some instances, a bispecific anti-FcRH5/anti-CD3 antibody may be administered in conjunction with BGT226. In some instances, a bispecific anti-FcRH5/anti-CD3 antibody may be administered in conjunction with GSK2126458. In some instances, a bispecific anti-FcRH5/anti-CD3 antibody may be administered in conjunction with PF-04691502. In some instances, a bispecific anti-FcRH5/anti-CD3 antibody may be administered in conjunction with PF-05212384 (also known as PKI-587).

In some instances, a bispecific anti-FcRH5/anti-CD3 antibody may be administered in conjunction with a chemotherapeutic agent. A chemotherapeutic agent is a chemical compound useful in the treatment of cancer. Exemplary chemotherapeutic agents include, but are not limited to erlotinib (TARCEVA®, Genentech/OSI Pharm.), anti-hormonal agents that act to regulate or inhibit hormone action on tumors such as anti-estrogens and selective estrogen receptor modulators (SERMs), antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), or trastuzumab (HERCEPTIN®, Genentech), EGFR inhibitors (EGFR antagonists), tyrosine kinase inhibitors, and chemotherapeutic agents also include non-steroidal anti-inflammatory drugs (NSAIDs) with analgesic, antipyretic and anti-inflammatory effects.

In instances for which the methods described herein involve a combination therapy, such as a particular combination therapy noted above, the combination therapy encompasses the co-administration of the bispecific anti-FcRH5/anti-CD3 antibody with one or more additional therapeutic agents, and such co-administration may be combined administration (where two or more therapeutic agents are included in the same or separate formulations) or separate administration, in which case, administration of the bispecific anti-FcRH5/anti-CD3 antibody can occur prior to, simultaneously, and/or following, administration of the additional therapeutic agent or agents. In one embodiment, administration of the bispecific anti-FcRH5/anti-CD3 antibody and administration of an additional therapeutic agent or exposure to radiotherapy can occur within about one month, or within about one, two or three weeks, or within about one, two, three, four, five, or six days, of each other.

In some aspects, the subject does not have an increased risk of CRS (e.g., has not experienced Grade 3+ CRS during treatment with a bispecific antibody or CAR-T therapy; does not have detectable circulating plasma cells; and/or does not have extensive extramedullary disease).

### D. Cancers

Any of the methods of the invention described herein may be useful for treating cancer, such as a B cell proliferative disorder, including multiple myeloma (MM), which may be relapsed or refractory (R/R) MM. In some aspects, the patient has received at least three prior lines of treatment for the B cell proliferative disorder (e.g., MM), e.g., has received three, four, five, six, or more than six prior lines of treatment. In some aspects, the patient has received at least three prior lines of treatment for the B cell proliferative disorder, wherein the treatment is a 4L₊ treatment. For example, the patient may have been exposed to a proteasome inhibitor (PI), an immunomodulatory drug (IMiD), an autologous stem cell transplant (ASCT), an anti-CD38 therapy (e.g., anti-CD38 antibody therapy, e.g., daratumumab therapy), a CAR-T therapy, or a therapy comprising a bispecific antibody. In some instances, the patient has been exposed to all three of PI, IMID, and anti-CD38 therapy. Other examples of B cell proliferative disorders/malignancies amenable to treatment with a bispecific anti-FcRH5/anti-CD3 antibody in accordance with the methods described herein include, without limitation, non-Hodgkin's lymphoma (NHL), including diffuse large B cell lymphoma (DLBCL), which may be relapsed or refractory DLBCL, as well as other cancers including germinal-center B cell-like (GCB) diffuse large B cell lymphoma (DLBCL), activated B cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B cell prolymphocytic leukemia, splenic marginal zone lymphoma, hairy cell leukemia, splenic lymphoma/leukemia, unclassifiable, splenic diffuse red pulp small B cell lymphoma, hairy cell leukemia variant, Waldenström macroglobulinemia, heavy chain diseases, a heavy chain disease, γ heavy chain disease, u heavy chain disease, plasma cell myeloma, solitary plasmacytoma of bone, extraosseous plasmacytoma, extranodal marginal zone lymphoma of mucosa-associated lymphoid tissue (MALT lymphoma), nodal marginal zone lymphoma, pediatric nodal marginal zone lymphoma, pediatric follicular lymphoma, primary cutaneous follicle centre lymphoma, T cell/histiocyte rich large B cell lymphoma, primary DLBCL of the CNS, primary cutaneous DLBCL, leg type, EBV-positive DLBCL of the elderly, DLBCL associated with chronic inflammation, lymphomatoid granulomatosis, primary mediastinal (thymic) large B cell lymphoma, intravascular large B cell lymphoma, ALK-positive large B cell lymphoma, plasmablastic lymphoma, large B cell lymphoma arising in HHV8-associated multicentric Castleman disease, primary effusion lymphoma: B cell lymphoma, unclassifiable, with features intermediate between DLBCL and Burkitt lymphoma, and B cell lymphoma, unclassifiable, with features intermediate between DLBCL and classical Hodgkin's lymphoma. Further examples of B cell proliferative disorders include, but are not limited to, multiple myeloma (MM); low grade/follicular NHL; small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; AIDS-related lymphoma; and acute lymphoblastic leukemia (ALL); chronic myeloblastic leukemia; and post-transplant lymphoproliferative disorder (PTLD). Further examples of cancer include, but are not limited to, carcinoma, lymphoma, blastoma, sarcoma, and leukemia or lymphoid malignancies, including B cell lymphomas. More particular examples of such cancers include, but are not limited to, low grade/follicular NHL; small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; AIDS-related lymphoma; and acute lymphoblastic leukemia (ALL); chronic myeloblastic leukemia; and post-transplant lymphoproliferative disorder (PTLD).

In some examples, the cancer is an FcRH5-positive cancer. Any suitable FcRH5-positive cancer may be treated using the methods disclosed herein. Examples of FcRH5-positive cancers include, but are not limited to MM), chronic lymphoid leukemia (CLL, mantle cell lymphoma (MCL), diffuse large B-cell lymphoma (DLBCL), follicular lymphoma (FL), acute myeloid leukemia (AML), myelodysplastic syndrome (MDS), chronic myelogenous leukemia (CML), chronic myelomonocytic leukemia, acute promyelocytic leukemia (APL), chronic myeloproliferative disorder, thrombocytic leukemia, precursor B-cell acute lymphoblastic leukemia (pre-B-ALL), precursor T cell acute lymphoblastic leukemia (pre-T-ALL), mast cell disease, mast cell leukemia, mast cell sarcoma, myeloid sarcomas, lymphoid leukemia, and undifferentiated leukemia.

Solid tumors that may by amenable to treatment with a bispecific anti-FcRH5/anti-CD3 antibody in accordance with the methods described herein include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, as well as abnormal vascular proliferation associated with phakomatoses, edema (such as that associated with brain tumors), Meigs' syndrome, brain, as well as head and neck cancer, and associated metastases. In certain embodiments, cancers that are amenable to treatment by the antibodies of the invention include breast cancer, colorectal cancer, rectal cancer, non-small cell lung cancer, glioblastoma, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, ovarian cancer, and mesothelioma.

E. Prior anti-cancer therapy

In some aspects, the subject has previously been treated for the B cell proliferative disorder (e.g., MM). In some aspects, the subject has received at least one, two, three, four, five, six, seven, eight, nine, ten, eleven, twelve, thirteen, fourteen, fifteen, or more than fifteen lines of treatment for the B cell proliferative disorder. In some aspects, the patient has received at least one prior line of treatment for the B cell proliferative disorder, e.g., the treatment is a 2L+, 3L+, 4L+, 5L+, 6L+, 7L+, 8L+, 9L+, 10L+, 11L+, 12L+, 13L+, 14L+, or 15L+ treatment. In some aspects, the subject has received at least three prior lines of treatment for the B cell proliferative disorder (e.g., MM), e.g., the patient has received a 4L+ treatment, e.g., has received three, four, five, six, seven, eight, nine, ten, eleven, twelve, thirteen, fourteen, fifteen, or more than fifteen lines of treatment. In some aspects, the subject has relapsed or refractory (R/R) multiple myeloma (MM), e.g., a patient having an R/R MM who is receiving a 4L+ treatment for R/R MM.

In some aspects, the prior lines of treatment include one or more of a proteasome inhibitor (PI), e.g., bortezomib, carfilzomib, or ixazomib; an immunomodulatory drug (IMiD), e.g., thalidomide, lenalidomide, or pomalidomide; an autologous stem cell transplant (ASCT); an anti-CD38 agent, e.g., daratumumab (DARZALEX®) (U.S. Pat. No. 7,829,673 and U.S. Pub. No: 20160067205 A1), “MOR202” (U.S. Pat. No. 8,263,746), isatuximab (SAR-650984); a CAR-T therapy; a therapy comprising a bispecific antibody; an anti-SLAMF7 therapeutic agent (e.g., an anti-SLAMF7 antibody, e.g., elotuzumab); a nuclear export inhibitor (e.g., selinexor); and a histone deacetylase (HDAC) inhibitor (e.g., panobinostat). In some aspects, the prior lines of treatment include an antibody-drug conjugate (ADC).

In some aspects, the prior lines of treatment include a B-cell maturation antigen (BCMA)-directed therapy, e.g., an antibody-drug conjugate targeting BCMA (BCMA-ADC).

In some aspects, the prior lines of treatment include all three of a proteasome inhibitor (PI), an IMiD, and an anti-CD38 agent (e.g., daratumumab).

In some aspects, the B cell proliferative disorder (e.g., MM) is refractory to the lines of treatment, e.g., is refractory to one or more of daratumumab, a PI, an IMID, an ASCT, an anti-CD38 agent, a CAR-T therapy, a therapy comprising a bispecific antibody, an anti-SLAMF7 therapeutic agent, a nuclear export inhibitor, a HDAC inhibitor, an ADC, or a BCMA-directed therapy. In some aspects, the B cell proliferative disorder (e.g., MM) is refractory to daratumumab.

### F. Risk-Benefit Profile

The methods described herein may result in an improved benefit-risk profile for patients having cancer (e.g., a multiple myeloma (MM), e.g., a relapsed or refractory (R/R) MM), e.g., a patient having an R/R MM who is receiving a 4L+ treatment for R/R MM, being treated with a bispecific anti-FcRH5/anti-CD3 antibody. In some instances, treatment using the methods described herein that result in administering the bispecific anti-FcRH5/anti-CD3 antibody subcutaneously may result in a reduction (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) or complete inhibition (100% reduction) of undesirable events, such as cytokine-driven toxicities (e.g., cytokine release syndrome (CRS)), infusion-related reactions (IRRs), macrophage activation syndrome (MAS), neurologic toxicities, severe tumor lysis syndrome (TLS), neutropenia, thrombocytopenia, elevated liver enzymes, and/or central nervous system (CNS) toxicities, following treatment with a bispecific anti-FcRH5/anti-CD3 antibody using a dosing regimen of the invention relative to treatment with a bispecific anti-FcRH5/anti-CD3 antibody that is not administered subcutaneously.

### G. Safety and Efficacy

i. Safety

In some aspects, less than 15% (e.g., less than 14%, less than 13%, less than 12%, less than 11%, less than 10%, less than 9%, less than 8%, less than 7%, less than 6%, less than 5%, less than 4%, less than 3%, less than 2%, or less than 1%) of patients treated using the methods described herein experience Grade 3 or Grade 4 cytokine release syndrome (CRS). In some aspects, less than 5% of patients treated using the methods described herein experience Grade 3 or Grade 4 CRS.

In some aspects, less than 10% (e.g., less than 9%, less than 8%, less than 7%, less than 6%, less than 5%, less than 4%, less than 3%, less than 2%, or less than 1%) of patients treated using the methods described herein experience Grade 4+ CRS. In some aspects, less than 3% of patients treated using the methods described herein experience Grade 4+ CRS. In some aspects, no patients experience Grade 4+ CRS.

In some aspects, less than 10% (e.g., less than 9%, less than 8%, less than 7%, less than 6%, less than 5%, less than 4%, less than 3%, less than 2%, or less than 1%) of patients treated using the methods described herein experience Grade 3 CRS. In some aspects, less than 5% of patients treated using the methods described herein experience Grade 3 CRS. In some aspects, no patients experience Grade 3 CRS.

In some aspects, Grade 2+ CRS events occur only in the first cycle of treatment. In some aspects, Grade 2 CRS events occur only in the first cycle of treatment. In some aspects, Grade 2 CRS events do not occur.

In some aspects, less than 3% of patients treated using the methods described herein experience Grade 4+ CRS, less than 5% of patients treated using the methods described herein experience Grade 3 CRS, and Grade 2+ CRS events occur only in the first cycle of treatment.

In some aspects, no Grade 3+ CRS events occur and Grade 2 CRS events occur only in the first cycle of treatment.

In some aspects, symptoms of immune effector cell-associated neurotoxicity syndrome (ICANS) are limited to confusion, disorientation, and expressive aphasia and resolve with steroids.

In some aspects, less than 10% (e.g., less than 9%, less than 8%, less than 7%, less than 6%, less than 5%, less than 4%, less than 3%, less than 2%, or less than 1%) of patients treated using the methods described herein experience seizures or other Grade 3+ neurologic adverse events. In some aspects, less than 5% of patients experience seizures or other Grade 3+ neurologic adverse events. In some aspects, no patients experience seizures or other Grade 3+ neurologic adverse events.

In some aspects, all neurological symptoms are either self-limited or resolved with steroids and/or tocilizumab therapy.

ii. Efficacy

In some aspects, the overall response rate (ORR) for patients treated using the methods described herein is at least 25%, e.g., is at least 30%, 35%, 40%, 45%, 50%, 55%, 60%, 65%, 70%, 75%, 80%, 85%, 90%, 95%, 99%, or 100%. In some aspects, the ORR is at least 40%. In some aspects, the ORR is at least 45% (e.g., at least 45%, 45.5%, 46%, 46.5% 47%, 47.5%, 48%, 48.5%, 49%, 49.5%, or 50%) at least 55%, or at least 65%. In some aspects, the ORR is at least 47.2%. In some aspects, the ORR is about 47.2%. In some aspects, the ORR is 75% or greater. In some aspects, at least 1% of patients (e.g., at least 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of patients) have a complete response (CR) or a very good partial response (VGPR). In some aspects, the ORR is 40%-50%, and 10%-20% of patients have a CR or a VGPR. In some aspects, the ORR is at least 40%, and at least 20% of patients have a CR or a VGPR.

In some aspects, the average duration of response (DoR) for patients treated using the methods described herein is at least two months, e.g., at least three months, at least four months, at least five months, at least six months, at least seven months, at least eight months, at least nine months, at least ten months, at least eleven months, at least one year, or more than one year. In some aspects, the average DoR is at least four months. In some aspects, the average DoR is at least five months. In some aspects, the average DoR is at least seven months.

In some aspects, the six month progression-free survival (PFS) rate for patients treated using the methods described herein is at least 10%, e.g., is at least 15%, 20%, 25%, 30%, 35%, 40%, 45%, 50%, 55%, 60%, 65%, 70%, 75%, 80%, 85%, 90%, 95%, 99%, or 100%. In some aspects, the six month PFS rate is at least 25%. In some aspects, the six month PFS rate is at least 40%. In some aspects, the six month PFS rate is at least 55%.

### H. Methods of Administration

The methods may involve administering the bispecific anti-FcRH5/anti-CD3 antibody (and/or any additional therapeutic agent) by any suitable means, including parenteral, intrapulmonary, and intranasal, and, if desired for local treatment, intralesional administration. Parenteral infusions include intravenous, subcutaneous, intramuscular, intraarterial, and intraperitoneal administration routes. In some embodiments, the bispecific anti-FcRH5/anti-CD3 antibody is administered by intravenous infusion. In other instances, the bispecific anti-FcRH5/anti-CD3 antibody is administered subcutaneously.

In some instances, the bispecific anti-FcRH5/anti-CD3 antibody administered by intravenous injection exhibits a less toxic response (i.e., fewer unwanted effects) in a patient than the same bispecific anti-FcRH5/anti-CD3 antibody administered by subcutaneous injection, or vice versa.

In some aspects, the bispecific anti-FcRH5/anti-CD3 antibody is administered intravenously over 4 hours (±15 minutes), e.g., the first dose of the antibody is administered over 4 hours±15 minutes.

In some aspects, the first dose and the second dose of the antibody are administered intravenously with a median infusion time of less than four hours (e.g., less than three hours, less than two hours, or less than one hour) and further doses of the antibody are administered intravenously with a median infusion time of less than 120 minutes (e.g., less than 90 minutes, less than 60 minutes, or less than 30 minutes.

In some aspects, the first dose and the second dose of the antibody are administered intravenously with a median infusion time of less than three hours and further doses of the antibody are administered intravenously with a median infusion time of less than 90 minutes.

In some aspects, the first dose and the second dose of the antibody are administered intravenously with a median infusion time of less than three hours and further doses of the antibody are administered intravenously with a median infusion time of less than 60 minutes. In some aspects, the patient is hospitalized (e.g., hospitalized for 72 hours, 48 hours, 24 hours, or less than 24 hours) during one or more administrations of the anti-FcRH5/anti-CD3 antibody, e.g., hospitalized for the C1D1 (cycle 1, dose 1) or the C1D1 and the C1D2 (cycle 1, dose 2). In some aspects, the patient is hospitalized for 72 hours following administration of the C1D1 and the C1D2. In some aspects, the patient is hospitalized for 24 hours following administration of the C1D1 and the C1D2. In some aspects, the patient is not hospitalized following the administration of any dose of the anti-FcRH5/anti-CD3 antibody.

For all the methods described herein, the bispecific anti-FcRH5/anti-CD3 antibody would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The bispecific anti-FcRH5/anti-CD3 antibody need not be, but is optionally formulated with, one or more agents currently used to prevent or treat the disorder in question. The effective amount of such other agents depends on the amount of the bispecific anti-FcRH5/anti-CD3 antibody present in the formulation, the type of disorder or treatment, and other factors discussed above. The bispecific anti-FcRH5/anti-CD3 antibody may be suitably administered to the patient over a series of treatments.

Any of the doses disclosed herein may be administered SC. SC administration of therapeutic antibodies can offer advantages over intravenous (IV) dosing. Compared with IV administration, SC dosing can be more convenient for patients and can provide improved healthcare utilization, including ease of administration, reduced treatment burden, and reduced hospitalization. The slower absorption rate associated with SC versus IV dosing may also confer an improved CRS profile.

Any suitable approach for SC administration may be used, including injection (e.g., a bolus injection) or infusion. For example, the therapeutic agent (e.g., bispecific anti-FcRH5/anti-CD3 antibody, anti-CD38 antibody (e.g., daratumumab), or IMiD (e.g., pomalidomide) may be administered SC using a pump (e.g., a patch pump, a syringe pump (e.g., a syringe pump with an infusion set), or an infusion pump (e.g., an ambulatory infusion pump or a stationary infusion pump)), a pre-filled syringe, a pen injector, or an autoinjector.

For example, in any of the methods or uses disclosed herein, the therapeutic agent may be administered SC using a pump. In some examples, a pump may be used for patient or health care provider (HCP) convenience, an improved safety profile (e.g., in terms of a drug's mechanism of action or the risk of IV-related infection), and/or for a combination therapy. Any suitable pump may be used, e.g., a patch pump, a syringe pump (e.g., a syringe pump with an infusion set), an infusion pump (e.g., an ambulatory infusion pump or a stationary infusion pump), or an LVP. In particular examples, the therapeutic agent may be administered SC using a patch pump. In some examples, the pump (e.g., the patch pump) may be a wearable or on-body pump (e.g., a wearable or on-body patch pump), for example, an Enable ENFUSE® on-body infusor or a West SMARTDOSER wearable injector (e.g., a West SMARTDOSER 10 wearable injector). In other examples, the therapeutic agent may be administered SC using a syringe pump (e.g., a syringe pump with an infusion set).

Other exemplary devices suitable for SC delivery include: a syringe (including a pre-filled syringe); an injection device (e.g., the INJECT-EASE™ and GENJECT™ device); an infusion pump (such as e.g., Accu-Chek™, CADD-PCAR, Braun Perfusor MR or MER, Braun Perfusor Compact® or LogoMed Pegasus PCA®.); an injector pen (such as the GENPEN™); a needleless device (e.g., MEDDECTOR™ and BIOJECTOR™); an autoinjector, a subcutaneous patch delivery system, etc.

In some embodiments, the disclosure provides a subcutaneous administration device, which delivers to a patient a fixed dose of the bispecific anti-FcRH5/anti-CD3 antibody. Preferably the bispecific anti-FcRH5/anti-CD3 antibody is cevostamab and the fixed dose is any dosage described herein.

In certain embodiments, the subcutaneous administration device is a prefilled syringe comprising a glass barrel, a plunger rod comprising a plunger stopper and a needle. In certain embodiments, the subcutaneous administration device further comprises a needle shield and optionally a needle shield device. In certain embodiments, the volume of formulation contained in the prefilled syringe is 0.3 mL, 1 mL, 1.5 mL, or 2.0 mL, in certain embodiments, the needle is a staked-in needle comprising a 3-bevel tip or a 5-bevel tip. In one embodiment, the subcutaneous administration device comprises a prefilled 1.0 mL low tungsten borosilicate glass (type I) syringe and a stainless steel 5-bevel 27 G ½ inch long thin-wall staked-in needle. In certain embodiments, the plunger rod comprises a rubber plunger stopper. In certain embodiments, the rubber plunger stopper comprises 4023/50 rubber and FLUROTEC® ethylene-tetrafluoroethylene (ETFE) coating. In some embodiments, the width (diameter, in particular outer diameter) of a needle for subcutaneous administration is typically between 25 gauge (G) and 31 G and is between ½ inch, long and 8 inch long. In some particular examples, the diameter, in particular the outer diameter, of a needle for subcutaneous administration is at least 28 G. Even more preferably, the diameter, in particular the outer diameter, of a needle or subcutaneous administration (e.g., injection) is at least 29 G, for example 29 G, 291/2 G, 30 G, 30 5/16 G, or 31 G. In some further particular examples, the diameter, in particular the outer diameter, of a needle for subcutaneous administration is at least 30 G. The use of such needles having very small outer diameters is assumed to further modify the cytokine release, possibly by causing smaller lesions and/or by causing a slower administration (less volume released over the same time). Needle injection typically requires injection by positioning the needle at an angle within the range of 40 to 50°. In certain embodiments, the subcutaneous administration device comprises a rigid needle shield. In certain embodiments, the rigid needle shield comprises a rubber formulation having low zinc content. In one embodiment, the needle shield is rigid and comprises an elastomeric component, FM27/0, and rigid polypropylene shield. In certain embodiments the subcutaneous administration device comprises a needle safety device. Exemplary needle safety devices include, but are not limited to, Ultrasafe Passive® Needle Guard X100L (Safety Syringes, Inc.) and Rexam Safe n Sound™ (Rexam).

In some embodiments, administration with the bispecific anti-FcRH5/anti-CD3 antibody is used with, for example, a self-inject device, autoinjector device, or other device designed for self-administration. In certain embodiments, the bispecific anti-FcRH5/anti-CD3 antibody is administered using a subcutaneous administration device. Various self-inject devices and subcutaneous administration devices, including autoinjector devices, are known in the art and are commercially available. Exemplary devices include, but are not limited to, prefilled syringes (such as BD HYPAK SCF®, READYFILL™, and STERIFILL SCF™ from Becton Dickinson; CLEARSHOT™ copolymer prefilled syringes from Baxter; and Daikyo Seiko CRYSTAL ZENITH® prefilled syringes available from West Pharmaceutical Services); disposable pen injection devices such as BD Pen from Becton Dickinson; ultra-sharp and microneedle devices (such as INJECT-EASE™ and microinfuser devices from Becton Dickinson; and H-PATCH™ available from Valeritas) as well as needle-free injection devices (such as BIOJECTOR® and IJECT® available from Bioject; and SOF-SERTER® and patch devices available from Medtronic). Certain embodiments of subcutaneous administration devices are described further herein. Co-formulations or co-administrations with such self-inject devices or subcutaneous administration devices of the bispecific anti-FcRH5/anti-CD3 antibody with at least a second therapeutic compound are envisioned.

In some embodiments, administration with the bispecific anti-FcRH5/anti-CD3 antibody is in combination with soluble hyaluronidase glycoproteins (sHASEGPs), which has been shown to facilitate the subcutaneous injection of therapeutic antibodies; see WO2006/091871. It has been shown that the addition of such soluble hyaluronidase glycoproteins (either as a combined formulation or by co-administration) may facilitate the administration of therapeutic drug into the hypodermis. By rapidly depolymerizing hyaluronan HA in the extracellular space, sHASEGP can reduce the viscosity of the interstitium, thereby increasing hydraulic conductance and allowing for larger volumes to be administered safely and comfortably into the subcutaneous tissue. The increased hydraulic conductance induced by sHASEGP through reduced interstitial viscosity can allow for greater dispersion, potentially increasing the systemic bioavailability of SC administered therapeutic drug. In some embodiments, a hyaluronidase, such as rHuPH20, is included in the formulation, for example, in an amount from about 1,400 U/mL to about 1,600 U/mL (e.g., about 1,500 U/mL). Optionally, the device delivers 0.9 mL, 1.8 mL, or 3.6 mL of the formulation to a subject.

Hyaluronidase products of animal origin have been used clinically for over 60 years, primarily to increase the dispersion and absorption of other co-administered drugs and for hypodermoclysis (SC injection/infusion of fluid in large volume) (Frost G. I., “Recombinant human hyaluronidase (rHuPH20): an enabling platform for subcutaneous drug and fluid administration”, Expert Opinion on Drug Delivery, 2007; 4:427-440). The details on the mechanism of action of hyaluronidases have been described in detail in the following publications: Duran-Reynolds F., “A spreading factor in certain snake venoms and its relation to their mode of action”, CR Soc Biol Paris, 1938; 69-81; Chain E., “A mucolvtic enzyme in testes extracts”, Nature 1939; 977-978; Weissmann B., “The transglycosylative action of testicular hyaluronidase”, J. Biol. Chem., 1955; 216:783-94; Tammi, R., Saamanen, A. M., Maibach, H. I., Tammi M., “Degradation of newly synthesized high molecular mass hyaluronan in the epidermal and dermal compartments of human skin in organ culture”, J. Invest. Dermatol. 1991:97:126-130; Laurent, U. B. G., Dahl, L. B., Reed, R. K., “Catabolismof hyaluronan in rabbit skin takes place locally, in lymph nodes and liver”. Exp. Physiol. 1991; 76:695-703; Laurent, T. C. and Fraser, J. R. E., “Degradation of Bioactive Substances: Physiology and Pathophysiology”, Henriksen. J. H. (Ed) CRC Press, Boca Raton, Fla.; 1991. pp. 249-265; Hams, E. N., et al., “Endocytic function, glycosaminoglycan specificity, and antibody sensitivity of the recombinant human 190-kDa hyaluronan receptor for endocytosis (HARE)”, J. Biol. Chem. 2004; 279:36201-36209; Frost, G. I., “Recombinant human hyaluronidase (rHuPH20): an enabling platform for subcutaneous drug and fluid administration”. Expert Opinion on Drug Delivery, 2007; 4:427-440.

Cevostamab may be administered to patients subcutaneously into the subcutaneous tissue of the abdomen. The abdomen can be divided into 4 quadrants, and injection sites can be rotated as shown. Other sites for administering the cevostamab subcutaneously may include, but are not limited to, the outer area of the upper arm, the thoracic region, in particular the lower thoracic region, the abdominal wall, above or below the waist, the upper area of the buttock, just behind the hip bone and the thigh, in particular the front of the thigh. Preferred sites for administering the antibody subcutaneously include the abdominal wall, and the lower thoracic region. Within a treatment cycle, each single dose may be administered to essentially the same body site, e.g., the thigh or abdomen. Alternatively, each single dose within a treatment cycle may be administered to different body sites. The target area of administration can be the fat layer located between the dermis and underlying fascia.

### I. Anti-FcRH5/Anti-CD3 Bispecific Antibodies

The methods described herein include administering to a subject having a cancer (e.g., a multiple myeloma, e.g., an R/R multiple myeloma) a bispecific antibody that binds to FcRH5 and CD3 (i.e., a bispecific anti-FcRH5/anti-CD3 antibody).

In some instances, any of the methods described herein may include administering a bispecific antibody that includes an anti-FcRH5 arm having a first binding domain comprising at least one, two, three, four, five, or six hypervariable regions (HVRs) selected from (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO:3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6). In some instances, the bispecific anti-FcRH5/anti-CD3 antibody comprises at least one (e.g., 1, 2, 3, or 4) of the heavy chain framework regions FR-H1, FR-H2, FR-H3, and FR-H4 comprising the sequences of SEQ ID NOs: 17-20, respectively, and/or at least one (e.g., 1, 2, 3, or 4) of the light chain framework regions FR-L1, FR-L2, FR-L3, and FR-L4 comprising the sequences of SEQ ID NOs: 21-24, respectively.

In some instances, any of the methods described herein may include administering a bispecific antibody that includes an anti-FcRH5 arm having a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO:3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6). In some instances, the bispecific anti-FcRH5/anti-CD3 antibody comprises at least one (e.g., 1, 2, 3, or 4) of the heavy chain framework regions FR-H1, FR-H2, FR-H3, and FR-H4 comprising the sequences of SEQ ID NOs: 17-20, respectively, and/or at least one (e.g., 1, 2, 3, or 4) of the light chain framework regions FR-L1, FR-L2, FR-L3, and FR-L4 comprising the sequences of SEQ ID NOs: 21-24, respectively.

In some instances, the bispecific antibody comprises an anti-FcRH5 arm comprising a first binding domain comprising (a) a heavy chain variable (VH) domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 7; (b) a light chain variable (VL) domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 8; or (c) a VH domain as in (a) and a VL domain as in (b). Accordingly, in some instances, the first binding domain comprises a VH domain comprising an amino acid sequence of SEQ ID NO: 7 and a VL domain comprising an amino acid sequence of SEQ ID NO: 8.

In some instances, any of the methods described herein may include administering a bispecific anti-FcRH5/anti-CD3 antibody that includes an anti-CD3 arm having a second binding domain comprising at least one, two, three, four, five, or six HVRs selected from (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14). In some instances, the anti-FcRH5/anti-CD3 bispecific antibody comprises at least one (e.g., 1, 2, 3, or 4) of heavy chain framework regions FR-H1, FR-H2, FR-H3, and FR-H4 comprising the sequences of SEQ ID NOs: 25-28, respectively, and/or at least one (e.g., 1, 2, 3, or 4) of the light chain framework regions FR-L1, FR-L2, FR-L3, and FR-L4 comprising the sequences of SEQ ID NOs: 29-32, respectively.

In some instances, any of the methods described herein may include administering a bispecific anti-FcRH5/anti-CD3 antibody that includes an anti-CD3 arm having a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14). In some instances, the anti-FcRH5/anti-CD3 bispecific antibody comprises at least one (e.g., 1, 2, 3, or 4) of heavy chain framework regions FR-H1, FR-H2, FR-H3, and FR-H4 comprising the sequences of SEQ ID NOs: 25-28, respectively, and/or at least one (e.g., 1, 2, 3, or 4) of the light chain framework regions FR-L1, FR-L2, FR-L3, and FR-L4 comprising the sequences of SEQ ID NOs: 29-32, respectively.

In some instances, the bispecific antibody comprises an anti-CD3 arm comprising a second binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 15; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 16; or (c) a VH domain as in (a) and a VL domain as in (b). Accordingly, in some instances, the second binding domain comprises a VH domain comprising an amino acid sequence of SEQ ID NO: 15 and a VL domain comprising an amino acid sequence of SEQ ID NO: 16.

In some instances, any of the methods described herein may include administering a bispecific antibody that includes (1) an anti-FcRH5 arm having a first binding domain comprising at least one, two, three, four, five, or six HVRs selected from (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO: 3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6) and (2) an anti-CD3 arm having a second binding domain comprising at least one, two, three, four, five, or six HVRs selected from (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14).

In some instances, any of the methods described herein may include administering a bispecific antibody that includes (1) an anti-FcRH5 arm having a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO:3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6) and (2) an anti-CD3 arm having a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14).

In some instances, the anti-FcRH5/anti-CD3 bispecific antibody comprises (1) at least one (e.g., 1, 2, 3, or 4) of heavy chain framework regions FR-H1, FR-H2, FR-H3, and FR-H4 comprising the sequences of SEQ ID NOs: 17-20, respectively, and/or at least one (e.g., 1, 2, 3, or 4) of the light chain framework regions FR-L1, FR-L2, FR-L3, and FR-L4 comprising the sequences of SEQ ID NOs: 21-24, respectively, and (2) at least one (e.g., 1, 2, 3, or 4) of heavy chain framework regions FR-H1, FR-H2, FR-H3, and FR-H4 comprising the sequences of SEQ ID NOs: 25-28, respectively, and/or at least one (e.g., 1, 2, 3, or 4) of the light chain framework regions FR-L1, FR-L2, FR-L3, and FR-L4 comprising the sequences of SEQ ID NOs: 29-32, respectively. In some instances, the anti-FcRH5/anti-CD3 bispecific antibody comprises (1) all four of heavy chain framework regions FR-H1, FR-H2, FR-H3, and FR-H4 comprising the sequences of SEQ ID NOs: 17-20, respectively, and/or all four of the light chain framework regions FR-L1, FR-L2, FR-L3, and FR-L4 comprising the sequences of SEQ ID NOs: 21-24, respectively, and (2) all four of heavy chain framework regions FR-H1, FR-H2, FR-H3, and FR-H4 comprising the sequences of SEQ ID NOs: 25-28, respectively, and/or all four (e.g., 1, 2, 3, or 4) of the light chain framework regions FR-L1, FR-L2, FR-L3, and FR-L4 comprising the sequences of SEQ ID NOs: 29-32, respectively.

In some instances, the anti-FcRH5/anti-CD3 bispecific antibody comprises (1) an anti-FcRH5 arm comprising a first binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 7; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 8; or (c) a VH domain as in (a) and a VL domain as in (b), and (2) an anti-CD3 arm comprising a second binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 15; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 16; or (c) a VH domain as in (a) and a VL domain as in (b). In some instances, the anti-FcRH5/anti-CD3 bispecific antibody comprises (1) a first binding domain comprising a VH domain comprising an amino acid sequence of SEQ ID NO: 7 and a VL domain comprising an amino acid sequence of SEQ ID NO: 8 and (2) a second binding domain comprising a VH domain comprising an amino acid sequence of SEQ ID NO: 15 and a VL domain comprising an amino acid sequence of SEQ ID NO: 16.

In some instances, the anti-FcRH5/anti-CD3 bispecific antibody comprises an anti-FcRH5 arm comprising a heavy chain polypeptide (H1) and a light chain polypeptide (L1), wherein (a) H1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 35 and/or (b) L1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 36.

In some instances, the anti-FcRH5/anti-CD3 bispecific antibody comprises an anti-FcRH5 arm comprising a heavy chain polypeptide (H1) and a light chain polypeptide (L1), wherein (a) H1 comprises the amino acid sequence of SEQ ID NO: 35 and/or (b) L1 comprises the amino acid sequence of SEQ ID NO: 36.

In some instances, the anti-FcRH5/anti-CD3 bispecific antibody comprises an anti-CD3 arm comprising a heavy chain polypeptide (H2) and a light chain polypeptide (L2), wherein (a) H2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 37 and/or (b) L2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 38.

In some instances, the anti-FcRH5/anti-CD3 bispecific antibody comprises an anti-CD3 arm comprising a heavy chain polypeptide (H2) and a light chain polypeptide (L2), wherein (a) H2 comprises the amino acid sequence of SEQ ID NO: 37; and (b) L2 comprises the amino acid sequence of SEQ ID NO: 38.

In some instances, the anti-FcRH5/anti-CD3 bispecific antibody comprises an anti-FcRH5 arm comprising a heavy chain polypeptide (H1) and a light chain polypeptide (L1) and an anti-CD3 arm comprising a heavy chain polypeptide (H2) and a light chain polypeptide (L2), and wherein (a) H1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 35; (b) L1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 36; (c) H2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 37; and (d) L2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 38.

In some instances, the anti-FcRH5/anti-CD3 bispecific antibody comprises an anti-FcRH5 arm comprising a heavy chain polypeptide (H1) and a light chain polypeptide (L1) and an anti-CD3 arm comprising a heavy chain polypeptide (H2) and a light chain polypeptide (L2), and wherein (a) H1 comprises the amino acid sequence of SEQ ID NO: 35; (b) L1 comprises the amino acid sequence of SEQ ID NO: 36; (c) H2 comprises the amino acid sequence of SEQ ID NO: 37; and (d) L2 comprises the amino acid sequence of SEQ ID NO: 38.

In some instances, the anti-FcRH5/anti-CD3 bispecific antibody is cevostamab.

In some instances, the anti-FcRH5/anti-CD3 bispecific antibody according to any of the above embodiments described above may incorporate any of the features, singly or in combination, as described in Sections 1-7 below.

### 1. Antibody Affinity

In certain embodiments, an antibody provided herein has a dissociation constant (K_{D}) of ≤1 μM, ≤250 nM, ≤100 nM, ≤15 nM, ≤10 nM, ≤6 nM, ≤4 nM, ≤2 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

In one embodiment, K_{D} is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 PM [¹²⁵]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20 ™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, K_{D} is measured using a BIACORE® surface plasmon resonance assay. For example, an assay using a BIACORE®-2000 or a BIACORE®-3000 (BIAcore, Inc., Piscataway, NJ) is performed at 37° C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 37° C. at a flow rate of approximately 25 μl/min. Association rates (kₒₙ, or kₐ) and dissociation rates (K_{off}, or k_{d}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio K_{off}/kₒₙ. See, for example, Chen et al., J. Mol. Biol. 293:865-881 (1999). If the on-rate exceeds 10⁶M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 37° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

### 2. Antibody Fragments

In certain embodiments, an antibody provided herein (e.g., an anti-FcRH5/anti-CD3 TDB) is an antibody fragment that binds FcRH5 and CD3. Antibody fragments include, but are not limited to, Fab, Fab′, Fab′-SH, F (ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134 (2003); and Hollinger et al. Proc. Natl. Acad. Sci. USA 90:6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

### 3. Chimeric and Humanized Antibodies

In certain embodiments, an antibody provided herein (e.g., an anti-FcRH5/anti-CD3 TDB) is a chimeric antibody. Certain chimeric antibodies are described, e.g., in U.S. Pat. No. 4,816,567; and Morrison et al. Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs (or portions thereof), for example, are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein (e.g., an anti-FcRH5/anti-CD3 TDB) is a human antibody. Human antibodies can be produced using various techniques known in the art. Human antibodies are described generally in van Dijk and van de Winkel, Curr. Opin. Pharmacol. 5:368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26 (4): 265-268 (2006) (describing human-human hybridomas). Human hybridoma technology (Trioma technology) is also described in Vollmers and Brandlein, Histology and Histopathology, 20 (3): 927-937 (2005) and Vollmers and Brandlein, Methods and Findings in Experimental and Clinical Pharmacology, 27 (3): 185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Multispecific Antibodies

In any one of the above aspects, an anti-FcRH5/anti-CD3 antibody provided herein is a multispecific antibody, for example, a bispecific antibody. Multispecific antibodies are antibodies (e.g., monoclonal antibodies) that have binding specificities for at least two different sites, e.g., antibodies having binding specificities for an immune effector cell and for a cell surface antigen (e.g., a tumor antigen, e.g., FcRH5) on a target cell other than an immune effector cell. In some aspects, one of the binding specificities is for FcRH5 and the other is for CD3.

In some aspects, the cell surface antigen may be expressed in low copy number on the target cell. For example, in some aspects, the cell surface antigen is expressed or present at less than 35,000 copies per target cell. In some embodiments, the low copy number cell surface antigen is present between 100 and 35,000 copies per target cell; between 100 and 30,000 copies per target cell; between 100 and 25,000 copies per target cell; between 100 and 20,000 copies per target cell; between 100 and 15,000 copies per target cell; between 100 and 10,000 copies per target cell; between 100 and 5,000 copies per target cell; between 100 and 2,000 copies per target cell; between 100 and 1,000 copies per target cell; or between 100 and 500 copies per target cell. Copy number of the cell surface antigen can be determined, for example, using a standard Scatchard plot.

In some embodiments, a bispecific antibody may be used to localize a cytotoxic agent to a cell that expresses a tumor antigen, e.g., FcRH5. Bispecific antibodies may be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305:537 (1983)), WO 93/08829, and Traunecker et al., EMBO J. 10:3655 (1991)), and “knob-in-hole” engineering (see, e.g., U.S. Pat. No. 5,731,168). “Knob-in-hole” engineering of multispecific antibodies may be utilized to generate a first arm containing a knob and a second arm containing the hole into which the knob of the first arm may bind. The knob of the multispecific antibodies of the invention may be an anti-CD3 arm in one embodiment. Alternatively, the knob of the multispecific antibodies of the invention may be an anti-target/antigen arm in one embodiment. The hole of the multispecific antibodies of the invention may be an anti-CD3 arm in one embodiment. Alternatively, the hole of the multispecific antibodies of the invention may be an anti-target/antigen arm in one embodiment.

Multispecific antibodies may also be engineered using immunoglobulin crossover (also known as Fab domain exchange or CrossMab format) technology (see, e.g., WO2009/080253; Schaefer et al., Proc. Natl. Acad. Sci. USA, 108:11187-11192 (2011)). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229:81 (1985)); using leucine zippers to produce bi-specific antibodies (see, e.g., Kostelny et al., J. Immunol., 148 (5): 1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147:60 (1991).

Engineered antibodies with three or more functional antigen binding sites, including “Octopus antibodies,” are also included herein (see, e.g., US 2006/0025576A1).

The antibodies, or antibody fragments thereof, may also include a “Dual Acting FAb” or “DAF” comprising an antigen binding site that binds to CD3 as well as another, different antigen (e.g., a second biological molecule) (see, e.g., US 2008/0069820).

### 6. Antibody Variants

In some aspects, amino acid sequence variants of the antibodies described herein, e.g., bispecific anti-FcRH5/anti-CD3 antibodies, are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

a. Substitution, Insertion, and Deletion Variants

In certain embodiments, antibody variants having one or more amino acid substitutions are provided. Sites of interest for substitutional mutagenesis include the CDRs and FRs. Conservative substitutions are shown in Table 4 under the heading of “preferred substitutions.” More substantial changes are provided in Table 4 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 4
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or residues that contact an antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

b. Glycosylation Variants

In certain embodiments, antibodies disclosed herein, e.g., bispecific anti-FcRH5/anti-CD3 antibodies, can be altered to increase or decrease the extent to which the antibody is glycosylated. Addition or deletion of glycosylation sites to anti-FcRH5 antibody of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants, e.g., bispecific anti-FcRH5/anti-CD3 antibody variants, are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87:614 (2004); Kanda, Y. et al., Biotechnol. Bioeng., 94 (4): 680-688 (2006); and WO2003/085107).

Antibody variants, e.g., bispecific anti-FcRH5/anti-CD3 antibody variants, are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### C. Fc Region Variants

In certain embodiments, one or more amino acid modifications may be introduced into the Fc region of an antibody disclosed herein, e.g., a bispecific anti-FcRH5/anti-CD3 antibody, thereby generating an Fc region variant (see e.g., US 2012/0251531). The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the invention contemplates an antibody variant, e.g., a bispecific anti-FcRH5/anti-CD3 antibody variant, that possesses some but not all effector functions, which make it a desirable candidate for applications in which the half-life of the antibody in vivo is important, yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc (RIII only, whereas monocytes express Fc (RI, Fc (RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and Cyto Tox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol. Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half-life determinations can also be performed using methods known in the art (see, e.g., Petkova, S. B. et al. Int'l. Immunol. 18 (12): 1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain embodiments, the proline at position 329 of a wild-type human Fc region in the antibody is substituted with glycine or arginine or an amino acid residue large enough to destroy the proline sandwich within the Fc/Fcγ receptor interface that is formed between the proline 329 of the Fc and tryptophan residues Trp 87 and Trp 110 of FcgRIII (Sondermann et al. Nature. 406, 267-273, 2000). In certain embodiments, the antibody comprises at least one further amino acid substitution. In one embodiment, the further amino acid substitution is S228P, E233P, L234A, L235A, L235E, N297A, N297D, or P331S, and still in another embodiment the at least one further amino acid substitution is L234A and L235A of the human IgG1 Fc region or S228P and L235E of the human IgG4 Fc region (see e.g., US 2012/0251531), and still in another embodiment the at least one further amino acid substitution is L234A and L235A and P329G of the human IgG1 Fc region.

Certain antibody variants with improved or diminished binding to FcRs are described. (See, e.g., U.S. Pat. No. 6,737,056; WO 2004/056312, and Shields et al., J. Biol. Chem. 9 (2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

In some aspects, the antibody, e.g., the anti-FcRH5 and/or anti-CD3 antibody (e.g., bispecific anti-FcRH5 antibody) comprises an Fc region comprising an N297G mutation (EU numbering). In some aspects, the anti-FcRH5 arm of the bispecific anti-FcRH5 antibody comprises a N297G mutation and/or the anti-CD3 arm of the bispecific anti-FcRH5 antibody comprises an Fc region comprising an N297G mutation.

In some embodiments, the anti-FcRH5 antibody comprising the N297G mutation comprises an anti-FcRH5 arm comprising a first binding domain comprising the following six HVRs (a) an HVR-H1 comprising the amino acid sequence of SEQ ID NO: 1; (b) an HVR-H2 comprising the amino acid sequence of SEQ ID NO: 2; (c) an HVR-H3 comprising the amino acid sequence of SEQ ID NO: 3; (d) an HVR-L1 comprising the amino acid sequence of SEQ ID NO: 4; (e) an HVR-L2 comprising the amino acid sequence of SEQ ID NO: 5; and (f) an HVR-L3 comprising the amino acid sequence of SEQ ID NO: 6; and an anti-CD3 arm comprising an N297G mutation. In some embodiments, the anti-CD3 arm comprising the N297G mutation comprises the following six HVRs: (a) an HVR-H1 comprising the amino acid sequence of SEQ ID NO: 9; (b) an HVR-H2 comprising the amino acid sequence of SEQ ID NO: 10; (c) an HVR-H3 comprising the amino acid sequence of SEQ ID NO: 11; (d) an HVR-L1 comprising the amino acid sequence of SEQ ID NO: 12; (e) an HVR-L2 comprising the amino acid sequence of SEQ ID NO: 13; and (f) an HVR-L3 comprising the amino acid sequence of SEQ ID NO: 14.

In some embodiments, the anti-FcRH5 antibody comprising the N297G mutation comprises an anti-FcRH5 arm comprising a first binding domain comprising (a) a VH domain comprising an amino acid sequence of SEQ ID NO: 7 and (b) a VL domain comprising an amino acid sequence of SEQ ID NO: 8, and an anti-CD3 arm comprising an N297G mutation. In some embodiments, the anti-CD3 arm comprising the N297G mutation comprises comprising (a) a VH domain comprising an amino acid sequence of SEQ ID NO: 15 and (b) a VL domain comprising an amino acid sequence of SEQ ID NO: 16.

In some embodiments, the anti-FcRH5 antibody comprising the N297G mutation comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, second CH2 (CH2₂) domain, and a second CH3 (CH3₂) domain. In some aspects, at least one of the one or more heavy chain constant domains is paired with another heavy chain constant domain. In some aspects, the CH3₁ and CH3₂ domains each comprise a protuberance or cavity, and wherein the protuberance or cavity in the CH3₁ domain is positionable in the cavity or protuberance, respectively, in the CH3₂ domain. In some aspects, the CH3₁ and CH3₂ domains meet at an interface between said protuberance and cavity. In some aspects, the CH2₁ and CH2₂ domains each comprise a protuberance or cavity, and wherein the protuberance or cavity in the CH2₁ domain is positionable in the cavity or protuberance, respectively, in the CH2₂ domain. In other instances, the CH2₁ and CH2₂ domains meet at an interface between said protuberance and cavity. In some aspects, the anti-FcRH5 antibody is an IgG1 antibody.

In some embodiments, the anti-FcRH5 antibody comprising the N297G mutation comprises an anti-FcRH5 arm comprising a first binding domain comprising (a) a VH domain comprising the amino acid sequence of SEQ ID NO: 7 and (b) a VL domain comprising the amino acid sequence of SEQ ID NO: 8, and an anti-CD3 arm, wherein (a) the anti-FcRH5 arm comprises T366S, L368A, Y407V, and N297G amino acid substitution mutations (EU numbering) and (b) the anti-CD3 arm comprises T366W and N297G substitution mutations (EU numbering). In some embodiments, the anti-CD3 arm comprising the T366W and N297G mutations comprises comprising (a) a VH domain comprising an amino acid sequence of SEQ ID NO: 15 and (b) a VL domain comprising an amino acid sequence of SEQ ID NO: 16.

In other embodiments, the anti-FcRH5 antibody comprising the N297G mutation comprises an anti-FcRH5 arm comprising a first binding domain comprising (a) a VH domain comprising an amino acid sequence of SEQ ID NO: 7 and (b) a VL domain comprising an amino acid sequence of SEQ ID NO: 8, and an anti-CD3 arm, wherein (a) the anti-FcRH5 arm comprises T366W and N297G substitution mutations (EU numbering) and (b) the anti-CD3 arm comprises T366S, L368A, Y407V, and N297G mutations (EU numbering). In some embodiments, the anti-CD3 arm comprising the N297G mutation comprises comprising (a) a VH domain comprising an amino acid sequence of SEQ ID NO: 15 and (b) a VL domain comprising an amino acid sequence of SEQ ID NO: 16.

d. Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, for example, in U.S. Pat. No. 7,521,541.

e. Antibody Derivatives

In certain embodiments, an antibody provided herein, e.g., a bispecific anti-FcRH5/anti-CD3 antibody provided herein, may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly (n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102:11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### 7. Charged Regions

In some aspects, the binding domain that binds FcRH5 or CD3 comprises a VH1 comprising a charged region (CR₁) and a VL1 comprising a charged region (CR₂), wherein the CR; in the VH1 forms a charge pair with the CR₂ in the VL1. In some aspects, the CRI comprises a basic amino acid residue and the CR₂ comprises an acidic amino acid residue. In some aspects, the CRI comprises a Q39K substitution mutation (Kabat numbering). In some aspects, the CR 1 consists of the Q39K substitution mutation. In some aspects, the CR₂ comprises a Q38E substitution mutation (Kabat numbering). In some aspects, the CR₂ consists of the Q38E substitution mutation. In some aspects, the second binding domain that binds CD3 comprises a VH2 comprising a charged region (CR₃) and a VL2 comprising a charged region (CR₄), wherein the CR₄ in the VL2 forms a charge pair with the CR₃ in the VH2. In some aspects, the CR₄ comprises a basic amino acid residue and the CR₃ comprises an acidic amino acid residue. In some aspects, the CR₄ comprises a Q38K substitution mutation (Kabat numbering). In some aspects, the CR₄ consists of the Q38K substitution mutation. In some aspects, the CR₃ comprises a Q39E substitution mutation (Kabat numbering). In some aspects, the CR₃ consists of the Q39E substitution mutation. In some aspects, the VL1 domain is linked to a light chain constant domain (CL1) domain and the VH1 is linked to a first heavy chain constant domain (CH1), wherein the CL1 comprises a charged region (CR₅) and the CH1 comprises a charged region (CR₆), and wherein the CR₅ in the CL1 forms a charge pair with the CR₆ in the CH11. In some aspects, the CR₅ comprises a basic amino acid residue and the CR₆ comprises an acidic residue. In some aspects, the CR₅ comprises a V133K substitution mutation (EU numbering). In some aspects, the CR₅ consists of the V133K substitution mutation. In some aspects, the CR₆ comprises a S183E substitution mutation (EU numbering). In some aspects, the CR₆ consists of the S183E substitution mutation.

In other aspects, the VL2 domain is linked to a CL domain (CL2) and the VH2 is linked to a CH1 domain (CH1₂), wherein the CL2 comprises a charged region (CR₇) and the CH1₂ comprises a charged region (CR₈), and wherein the CR; in the CH1₂ forms a charge pair with the CR₇ in the CL2. In some aspects, the CR₈ comprises a basic amino acid residue and the CR₇ comprises an acidic amino acid residue. In some aspects, the CR₈ comprises a S183K substitution mutation (EU numbering). In some aspects, the CR₈ consists of the S183K substitution mutation. In some aspects, the CR₇ comprises a V133E substitution mutation (EU numbering). In some aspects, the CR₇ consists of the V133E substitution mutation.

In other aspects, the VL2 domain is linked to a CL domain (CL2) and the VH2 is linked to a CH1 domain (CH1₂), wherein (a) the CL2 comprises one or more mutations at amino acid residues F116, L135, S174, S176, and/or T178 (EU numbering) and (b) the CH12 comprises one or more mutations at amino acid residues A141, F170, S181, S183, and/or V185 (EU numbering). In some aspects, the CL2 comprises one or more of the following substitution mutations: F116A, L135V, S174A, S176F, and/or T178V. In some aspects, the CL2 comprises the following substitution mutations: F116A, L135V, S174A, S176F, and T178V. In some aspects, the CH12 comprises one or more of the following substitution mutations: A1411, F170S, S181M, S183A, and/or V185A. In some aspects, the CH1₂ comprises the following substitution mutations: A1411, F170S, S181M, S183A, and V185A.

In other aspects, the binding domain that binds FcRH5 or CD3 comprises a VH domain (VH1) comprising a charged region (CR₁) and a VL domain (VL1) comprising a charged region (CR₂), wherein the CR₂ in the VL₁ forms a charge pair with the CR₁ in the VH1. In some aspects, the CR₂ comprises a basic amino acid residue and the CR₁ comprises an acidic amino acid residue. In some aspects, the CR₂ comprises a Q38K substitution mutation (Kabat numbering). In some aspects, the CR₂ consists of the Q38K substitution mutation. In some aspects, the CR; comprises a Q39E substitution mutation (Kabat numbering). In some aspects, the CR₁ consists of the Q39E substitution mutation. In some aspects, the second binding domain that binds CD3 comprises a VH domain (VH2) comprising a charged region (CR₃) and a VL domain (VL2) comprising a charged region (CR₄), wherein the CR₃ in the VH2 forms a charge pair with the CR₄ in the VL2. In some aspects, the CR₃ comprises a basic amino acid residue and the CR₄ comprises an acidic amino acid residue. In some aspects, the CR₃ comprises a Q39K substitution mutation (Kabat numbering). In some aspects, the CR₃ consists of the Q39K substitution mutation. In some aspects, the CR₄ comprises a Q38E substitution mutation (Kabat numbering). In some aspects, the CR₄ consists of the Q38E substitution mutation. In some aspects, the VL1 domain is linked to a light chain constant domain (CL1) and the VH1 is linked to a first heavy chain constant domain (CH1₁), wherein the CL1 comprises a charged region (CR₅) and the CH1₁ comprises a charged region (CR₆), and wherein the CR₆ in the CH1₁ forms a charge pair with the CR₅ in the CL1. In some aspects, the CR₆ comprises a basic amino acid residue and the CR₅ comprises an acidic amino acid residue. In some aspects, the CR₆ comprises a S183K substitution mutation (EU numbering). In some aspects, the CR₆ consists of the S183K substitution mutation. In some aspects, the CR₅ comprises a V133E substitution mutation (EU numbering). In some aspects, the CR₅ consists of the V133E substitution mutation.

In other aspects, the VL2 domain is linked to a CL domain (CL2) and the VH2 is linked to a CH1 domain (CH1₂), wherein the CL2 comprises a charged region (CR₇) and the CH12 comprises a charged region (CR₈), and wherein the CR₇ in the CL2 forms a charged pair with the CR₈ in the CH1₂. In some aspects, the CR₇ comprises a basic amino acid residue and the CR₈ comprises an acidic residue. In some aspects, the CR₇ comprises a V133K substitution mutation (EU numbering). In some aspects, the CR₇ consists of the V133K substitution mutation. In some aspects, the CR: comprises a S183E substitution mutation (EU numbering). In some aspects, the CR: consists of the S183E substitution mutation.

In other aspects, the VL2 domain is linked to a CL domain (CL2) and the VH2 is linked to a CH1 domain (CH1₂), wherein (a) the CL2 comprises one or more mutations at amino acid residues F116, L135, S174, S176, and/or T178 (EU numbering) and (b) the CH12 comprises one or more mutations at amino acid residues A141, F170, S181, S183, and/or V185 (EU numbering). In some aspects, the CL2 comprises one or more of the following substitution mutations: F116A, L135V, S174A, S176F, and/or T178V. In some aspects, the CL2 comprises the following substitution mutations: F116A, L135V, S174A, S176F, and T178V. In some aspects, the CH12 comprises one or more of the following substitution mutations: A1411, F170S, S181M, S183A, and/or V185A. In some aspects, the CH12 comprises the following substitution mutations: A1411, F170S, S181M, S183A, and V185A. In some aspects, the anti-FcRH5 antibody comprises one or more heavy chain constant domains, wherein the one or more heavy chain constant domains are selected from a first CH2 domain (CH2₁), a first CH3 domain (CH3₁), a second CH2 domain (CH2₂), and a second CH3 domain (CH3₂). In some aspects, at least one of the one or more heavy chain constant domains is paired with another heavy chain constant domain. In some aspects, the CH3₁ and the CH3₂ each comprise a protuberance (P1) or a cavity (C1), and wherein the P1 or the C₁ in the CH3₁ is positionable in the C₁ or the P₁, respectively, in the CH3₂. In some aspects, the CH3₁ and the CH3₂ meet at an interface between the P₁ and the C₁. In some aspects, the CH2₁ and the CH2₂ each comprise (P₂) or a cavity (C₂), and wherein the P₂ or the C₂ in the CH2₁ is positionable in the C₂ or the P₂, respectively, in the CH2₂. In some aspects, the CH2₁ and the CH2₂ meet at an interface between the P₂ and the C₂.

### J. Recombinant Methods and Compositions

Antibodies disclosed herein, e.g., bispecific anti-FcRH5/anti-CD3 antibodies as disclosed herein, may be produced using recombinant methods and compositions, for example, as described in U.S. Pat. No. 4,816,567. In one embodiment, an isolated nucleic acid encoding an antibody, e.g., anti-FcRH5 antibody (e.g., a bispecific anti-FcRH5/anti-CD3 antibody) described herein is provided. Such nucleic acid may encode an amino acid sequence comprising the VL and/or an amino acid sequence comprising the VH of the antibody (e.g., the light and/or heavy chains of the antibody). In another embodiment, an isolated nucleic acid encoding an anti-CD3 antibody described herein is provided. Such a nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such a nucleic acid are provided. In a further embodiment, a host cell comprising such a nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NSO, Sp20 cell). In one embodiment, a method of making an antibody, e.g., an bispecific anti-FcRH5/anti-CD3 antibody, is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, e.g., a bispecific anti-FcRH5/anti-CD3 antibody, a nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

### 1. Two-Cell Methods for Manufacturing Bispecific Antibodies

In some aspects, an antibody as disclosed herein (e.g., a bispecific anti-FcRH5/anti-CD3 antibody) is manufactured using a method comprising two host cell lines. In some aspects, a first arm of the antibody (e.g., a first arm comprising a hole (cavity) region) is produced in a first host cell line, and a second arm of the antibody (e.g., a second arm comprising a knob (protuberance) region) is produced in a second host cell line. The arms of the antibody are purified from the host cell lines and assembled in vitro.

### 2. One-Cell Methods for Manufacturing Bispecific Antibodies

In some aspects, an antibody as disclosed herein (e.g., a bispecific anti-FcRH5/anti-CD3 antibody) is manufactured using a method comprising a single host cell line. In some aspects, a first arm of the antibody (e.g., a first arm comprising a hole (cavity) region) and a second arm of the antibody (e.g., a second arm comprising a knob (protuberance) region) are produced in and purified from a single host cell line. Preferably, the first arm and the second arm are expressed at comparable levels in the host cell, e.g., are both expressed at a high level in the host cell. Similar levels of expression increase the likelihood of efficient TDB production and decrease the likelihood of light chain (LC) mispairing of TDB components. The first arm and second arm of the antibody may each further comprise amino acid substitution mutations introducing charge pairs, as described in Section II (I) (7) herein. The charge pairs promote the pairing of heavy and light chain cognate pairs of each arm of the bispecific antibody, thereby minimizing mispairing.

### 3. Host Cells

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NSO and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, NJ), pp. 255-268 (2003).

### K. Immunoconjugates

The disclosure also provides immunoconjugates comprising an antibody as disclosed herein, e.g., a bispecific anti-FcRH5/anti-CD3 antibody disclosed herein, conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as disclosed herein, e.g., a bispecific anti-FcRH5/anti-CD3 antibody as described herein, conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another embodiment, an immunoconjugate comprises an antibody as disclosed herein, e.g., a bispecific anti-FcRH5/anti-CD3 antibody described herein, conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese, or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### L. Pharmaceutical Compositions and Formulations

Pharmaceutical compositions and formulations of the therapeutic agents described herein (e.g., anti-FcRH5/anti-CD3 bispecific antibodies, anti-CD38 antibodies (e.g., daratumumab), IMiDs (e.g., pomalidomide, and corticosteroids (e.g., dexamethasone or methylprednisolone)) can be prepared by mixing such therapeutic agents having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as L-Histidine/glacial acetic acid (e.g., at pH 5.8), phosphate, citrate, and other organic acids; tonicity agents, such as sucrose; stabilizers, such as L-methionine; antioxidants including N-acetyl-DL-tryptophan, ascorbic acid, and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polysorbate 20 or polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles, and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, for example, films, or microcapsules.

The formulations to be used for in vivo administration are generally sterile. Sterility may be readily accomplished, e.g., by filtration through sterile filtration membranes.

### III. Articles of Manufacture

In another aspect of the invention, an article of manufacture containing materials useful for the treatment, prevention, and/or diagnosis of the disorders described above is provided. For example, an article of manufacture for use in any of the methods disclosed herein is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition may be an anti-FcRH5/anti-CD3 bispecific antibody described herein. In some examples, at least one active agent in the composition may be an anti-CD38 antibody (e.g., daratumumab), an IMiD (e.g., pomalidomide), a corticosteroid (e.g., dexamethasone or methylprednisolone), or a combination thereof.

In some aspects, the article of manufacture comprises at least two containers (e.g., vials), a first container holding an amount of the composition suitable for a C1D1 (cycle 1, dose 1) and a second container holding an amount of the composition suitable for a C1D2 (cycle 1, dose 2). In some aspects, the article of manufacture comprises at least three containers (e.g., vials), a first container holding an amount of the composition suitable for a C1D1, a second container holding an amount of the composition suitable for a C1D2, and a third container holding an amount of the composition suitable for a C1D3. In some aspects, the containers (e.g., vials) may be different sizes, e.g., may have sizes proportional to the amount of the composition they contain. Articles of manufacture comprising containers (e.g., vials) proportional to the intended doses may, e.g., increase convenience, minimize waste, and/or increase cost-effectiveness. The label or package insert indicates that the composition is used for treating the condition of choice (e.g., a multiple myeloma (MM), e.g., relapsed or refractory MM, e.g., 4L+ treatment for R/R MM) and further includes information related to at least one of the dosing regimens described herein. Moreover, the article of manufacture may comprise (a) a first container with a composition contained therein, wherein the composition comprises an anti-FcRH5/anti-CD3 bispecific antibody described herein; and (b) a second container with a composition contained therein, wherein the composition comprises a further cytotoxic or otherwise therapeutic agent. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In some aspects, the article of manufacture comprises a subcutaneous administration device comprising a bispecific antibody that binds to FcRH5 and CD3 (e.g., cevostamab). The antibody may be administered by the administration device at an amount suitable for C1D1, C1D2, and/or C1D3, as described above. The subcutaneous administration device may include any subcutaneous administration device described herein or known in the art. For example, the subcutaneous administration device may be a syringe (e.g., a pre-filled syringe), a pump (e.g., a patch pump, a syringe pump, or an infusion pump), or a wearable pump. Administration of the bispecific antibody (e.g., cevostamab) by the subcutaneous administration device may be used for the treatment of cancer (e.g., R/R MM).

In some examples, the subcutaneous administration device comprises a first dose, a second dose, and/or a third dose of the bispecific antibody. For example, in some instances, the subcutaneous administration device comprises: (i) a first dose of the bispecific antibody of between 0.5 mg to 8 mg; (ii) a second dose of the bispecific antibody of between 2 mg to 40 mg; and/or (iii) a third dose of the bispecific antibody of between 10 mg to 160 mg.

In some aspects: (i) the first dose of the bispecific antibody is between 1 mg to 3 mg; (ii) the second dose of the bispecific antibody is between 8 mg to 12 mg; and/or (iii) the third dose of the bispecific antibody is between 35 mg to 45 mg.

In some aspects: (i) the first dose of the bispecific antibody is 2 mg; (ii) the second dose of the bispecific antibody is 10 mg; and/or (iii) the third dose of the bispecific antibody is 40 mg.

In some aspects: (i) the first dose of the bispecific antibody is 2 mg; (ii) the second dose of the bispecific antibody is 10 mg; and (iii) the third dose of the bispecific antibody is 120 mg.

### IV. Example

The following is an example of the methods of the invention. It is understood that various other embodiments may be practiced, given the general description provided above, and the example is not intended to limit the scope of the claims.

### Example 1. An Open-Label, Multicenter, Phase Lb Trial Evaluating the Safety, Pharmacokinetics, and Activity of Subcutaneous (SC) Cevostamab in Patients with Relapsed or Refractory Multiple Myeloma

This example describes GO43227 (International Standard Randomized Controlled Trial Number (ISRCTN) identifier: 26168155) a Phase Ib, multicenter, open-label study designed to evaluate the safety, tolerability, pharmacokinetics (PK), and pharmacodynamics of single-agent cevostamab administered subcutaneously (SC) in patients with Relapsed or Refractory (R/R) Multiple Myeloma (MM). The study consists of a dose-escalation phase followed by an expansion phase. The dose escalation phase will employ a 3+3 design to evaluate the safety and determine the recommended Phase II dose (RP2D) and schedule of SC cevostamab. This will be followed by an expansion phase to further characterize the safety, pharmacokinetics, and activity of cevostamab at the RP2D. Patients will undergo a screening period, a treatment period, and follow-up. The study will enroll approximately 60 patients at approximately 6-15 sites globally.

### A. Objectives and Endpoints

The primary objective of this study is to evaluate the safety, tolerability, and pharmacokinetics of cevostamab administered subcutaneously in patients with R/R MM.

In this protocol, “study treatment” refers to cevostamab. Specific objectives and corresponding endpoints for the study are outlined below.

| | Corresponding Endpoints |
|---|---|
| Safety Objectives (Primary Study Objective) | |
| To evaluate the safety and tolerability of SC | Incidence and severity of adverse events, with severity |
| cevostamab, including estimation of the | determined according to NCI CTCAE v5.0 and ASTCT |
| maximum tolerated dose (MTD) and | Consensus Grading for Cytokine Release Syndrome |
| characterization of Dose Limiting Toxicities | (Lee et al., Biol Blood Marrow Transplant, 25(4): 625- |
| (DLTs). | 638, 2019). |
| To identify a recommended Phase II dose | Relationship between cevostamab dose and safety, PK, |
| (RP2D) for SC cevostamab. | and immunogenicity endpoints. |
| Activity Objective | |
| To make a preliminary assessment of the | Note: International Myeloma Working Group (IMWG) |
| activity of SC cevostamab. | Criteria (Table 8A) will be used for the following endpoints. |
| | Objective response rate (ORR) as determined by the |
| | investigator. |
| | ORR is defined as the proportion of patients |
| | with a stringent complete response (sCR), |
| | complete response (CR), very good partial |
| | response (VGPR), or partial response (PR) on |
| | two consecutive occasions. |
| | Rate of CR/sCR as determined by the investigator |
| | CR/sCR rate is defined as proportion of |
| | patients achieving a CR or sCR on two |
| | consecutive occasions. |
| | Rate of VGPR or better as determined by the |
| | investigator. |
| | Rate of VGPR or better is defined as the |
| | proportion of patients achieving a VGPR or |
| | better on two consecutive occasions. |
| | Progression-free survival (PFS) as determined by the |
| | investigator. |
| | PFS is defined as the time from enrollment to |
| | the first occurrence of disease progression or |
| | death from any cause, whichever occurs first. |
| | DOR as determined by the investigator. |
| | DOR is defined as the number of days from the |
| | date of first documented response of PR or |
| | better until date of disease progression or death |
| | from any cause, whichever occurs first. |
| | Time to first response (for patients who achieve a |
| | response of PR or better). |
| | Time to first response is defined as time from |
| | initiation of study treatment to achieving a |
| | confirmed PR or better. |
| | Time to best response (for patients who achieve a |
| | response of PR or better). |
| | Time to best response is defined as time from |
| | initiation of study treatment to achieving the |
| | deepest response. |
| | Minimal residual disease (MRD) negativity as defined by |
| | next generation sequencing (NGS) (<10-5) on bone |
| | marrow aspirate. |
| | Overall Survival (OS) |
| | OS is defined as the time from initiation of study |
| | treatment to death from any cause. |
| Pharmacokinetic Objective | |
| To characterize the pharmacokinetics of SC | Serum concentration of cevostamab at specified timepoints. |
| cevostamab. | PK parameters of cevostamab. |
| Exploratory Pharmacokinetic Objectives | |
| To evaluate potential relationships between | Relationship between selected covariates and serum |
| selected covariates and exposure to | concentration or PK parameters for cevostamab. |
| cevostamab. | |

| | Corresponding Endpoint |
|---|---|
| Immunogenicity Objective | |
| To evaluate the immune response to SC | Prevalence of anti-drug antibodies (ADAs) against |
| cevostamab. | cevostamab at baseline and incidence of ADAs against |
| | cevostamab during the study. |
| Exploratory Immunogenicity Objective | |
| To evaluate potential effects of ADAs against | Relationship between status of ADA against cevostamab |
| cevostamab. | and safety, PK, or activity endpoints. |
| Additional Exploratory Objective | |
| To make a preliminary assessment of the | Outcome of CRS following the administration of tocilizumab. |
| activity of tocilizumab (e.g., | |
| Actemra ®/RoActemra ®) in ameliorating the | |
| symptoms of CRS following treatment with | |
| cevostamab. | |

ADA = anti-drug antibody; ASTCT = American Society of Transplantation and Cellular Therapy CR = complete response; CRS = cytokine-release syndrome; DLT = Dose Limiting Toxicity; DOR = duration of response; IMWG = International Myeloma Working Group; MRD = minimal residual disease; MTD = maximum tolerated dose; NCI CTCAE v5.0 = National Cancer Institute Common Terminology Criteria for Adverse Events, Version 5.0; NGS = next-generation sequencing; ORR = objective response rate; PD = pharmacodynamic; PFS = progression-free survival; PK = pharmacokinetic; PR = partial response; RP2D = recommended Phase II dose; sCR = stringent complete response; VGPR = very good partial response.

### B. Study Design

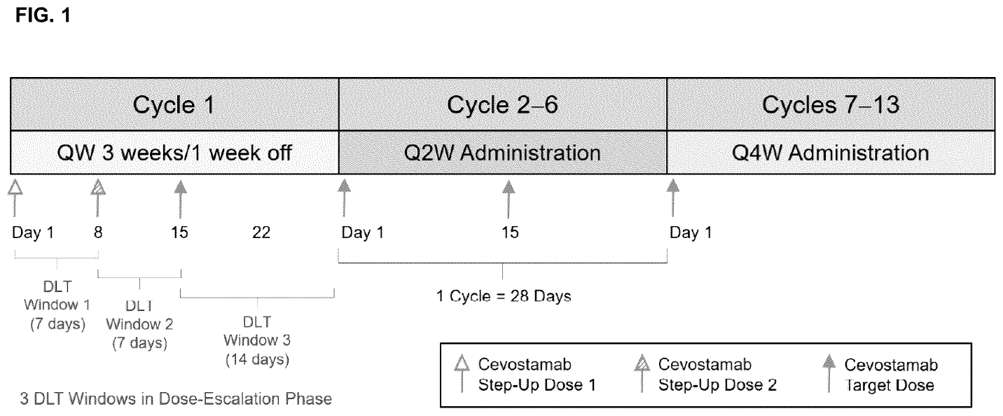

Cevostamab will be administered subcutaneously in 28-day cycles, as illustrated in FIG. 1 and as follows:
In Cycle 1, cevostamab will be administered weekly (QW) starting with a step-up dose on Days 1 and 8, followed by the target dose on Day 15.
In Cycles 2-6, cevostamab will be administered every two weeks (Q2W) at the target dose.
In Cycles 7-13, cevostamab will be administered every four weeks (Q4W) at the target dose.

Patients with acceptable toxicity and evidence of clinical benefit as described herein, may continue to receive cevostamab up to a maximum of 13 cycles, or until disease progression (as determined by the investigator according to International Myeloma Working Group (IMWG) criteria; Table 8A) or unacceptable toxicity, whichever occurs first. An exception will be made for patients who undergo intrapatient dose escalation; these patients may continue to receive cevostamab up to a maximum of 13 cycles at the new, increased dose, or until disease progression or unacceptable toxicity, whichever occurs first.

Patients who complete 13 cycles of study treatment will continue to undergo follow-up tumor and additional assessments until disease progression, start of new anti-cancer therapy, or withdrawal from study participation, whichever occurs first.

Patients who have completed 13 cycles of treatment may be eligible for cevostamab re-treatment.

Tocilizumab will be administered for the management of treatment-emergent CRS when necessary.

i. Screening

The screening period will last up to 28 days for both the escalation and expansion stages. Patients who do not meet the criteria for participation in this study (screen failure) may qualify for two re-screening opportunities (maximum three screenings per participant) at the investigator's discretion. In the absence of new information that may relate to the patient's willingness to participate (e.g., additional procedures, new or updated risk information), patients are not required to re-sign the consent form. The investigator will record reasons for screen failure in the screening log.

ii. Safety Monitoring

All patients will be closely monitored for adverse events throughout the study and for at least 30 days after the last dose of study treatment on an ongoing basis. Adverse events will be graded according to the National Cancer Institute Common Terminology Criteria for Adverse Events, Version 5.0 (NCI CTCAE v5.0), with the exception of cytokine-release syndrome (CRS), which will be graded according to American Society of Transplantation and Cellular Therapy (ASTCT) Consensus Grading for Cytokine Release Syndrome (Lee et al., Biol Blood Marrow Transplant, 25 (4): 625-638, 2019).

iii. Dose-Escalation Stage

Dose escalation will use a standard 3+3 design. Cohorts of approximately 3-6 patients each will be treated at escalating doses of SC cevostamab administered on Days 1, 8, and 15 of Cycle 1.

For each dose-escalation cohort, treatment with the first dose of cevostamab will be staggered such that the second patient enrolled in the cohort will receive cevostamab at least 72 hours after the first enrolled patient receives the first dose of cevostamab in order to assess for any severe and unexpected acute drug or injection-related toxicities; dosing in subsequent patients in each cohort will be staggered by at least 24 hours from the end of the prior patients' administration. Staggered patient enrollment will not be required for enrollment of additional patients to acquire additional safety and pharmacodynamic data at a dose level that has been shown to not exceed the MTD.

The initial step-up dose given on cycle (C) 1, day (D) 1 (C1D1) will be less than the C1D8 dose and the target dose administered on C1D15. Patients will be hospitalized during Cycle 1 according to the safety rules described herein.

Thereafter, cevostamab will be administered on Days 1 and 15 of Cycles 2-6 and on Day 1 of Cycles 7-13, as described herein. The C2D1 dose and all subsequent doses will be equal to the Cycle 1 target dose unless a dose modification is required, or intrapatient dose escalation occurs. Doses may be delayed to allow patients to recover from toxicity or for other selected reasons.

iv. Dose Limiting Toxicity (DLT) Assessment Windows

In Cycle 1, three consecutive DLT assessment windows will be utilized as follows:
1. The first DLT assessment window (Window 1) will be defined as the period between the C1D1 dose and the next cevostamab dose administration.
2. The second DLT assessment window (Window 2) will be defined as the period between the second Cycle 1 cevostamab dose and the administration of the third Cycle 1 cevostamab dose.
3. The third DLT assessment window (Window 3) will be defined as the period between the administration of the third Cycle 1 cevostamab dose and the administration of the first Cycle 2 dose.
   v. Definition of Dose-Limiting Toxicity (DLT)

All adverse events (including neurological events) will be reported according to the instructions described herein, and graded according to the NCI CTCAE v5.0, with the exception of CRS, which will be graded according to the ASTCT Consensus Grading for Cytokine-Release Syndrome (Lee et al., Biol Blood Marrow Transplant, 25 (4): 625-638, 2019; see Table 2A).

DLTs will be treated according to clinical practice and will be monitored through their resolution. All adverse events should be considered related to study drug unless such events are clearly attributed by the investigator to another clearly identifiable cause (e.g., documented disease progression, concomitant medication, or preexisting medical condition). Decreases in B cells, lymphopenia, and/or leukopenia due to decreases in B cells or T cells will not be considered DLTs, as they are expected pharmacodynamic (PD) outcomes of cevostamab treatment based on nonclinical and clinical testing of this molecule.

A DLT will be defined as any of the following adverse events occurring during the DLT assessment periods as defined above, in Cycle 1:
Any Grade 4 or 5 adverse event not considered by the investigator to be attributable to another clearly identifiable cause, with the following exceptions:
   Grade 4 lymphopenia, which is an expected outcome of therapy.
   Grade 4 neutropenia that is not accompanied by temperature elevation (oral or tympanic temperature of ≥100.4° F. (38° C.)) and improves to Grade≤2 (or to ≥80% of the baseline ANC, whichever is lower) within 1 week with or without G-CSF.
   Grade 4 thrombocytopenia that improves to Grade≤2 (or to ≥80% of the baseline platelet count, whichever is lower) within 1 week, does not require platelet transfusion, or is not associated with bleeding.
Any Grade 3 CRS (per ASTCT Consensus Grading; see Table 2A).
Any Grade 3 hematologic adverse event not considered to be attributable to another clearly identifiable cause, with the following exceptions:
   Grade 3 lymphopenia, which is an expected outcome of therapy.
   Grade 3 neutropenia that is not accompanied by temperature elevation (oral or tympanic temperature of ≥100.4° F. (38° C.)) and improves to Grade≤2 (or to ≥80% of the baseline ANC, whichever is lower) within 1 week with or without G-CSF.
   Grade 3 thrombocytopenia that improves to Grade≤2 (or to ≥80% of the baseline platelet count, whichever is lower) within 1 week, is not associated with bleeding, and does not require a transfusion.
   Grade 3 anemia that improves to Grade≤2 (or to ≥80% of the baseline hemoglobin, whichever is lower) within 1 week with or without a transfusion.
Any Grade 3 non-hematologic adverse event not considered to be attributable to another clearly identifiable cause, with the following exceptions:
   Grade 3 nausea or vomiting in the absence of premedication or that can be managed with resulting resolution to Grade≤2 with oral or IV anti-emetics within 24 hours (Grade 3 nausea or vomiting that requires total parenteral nutrition or hospitalization are not excluded and should be considered a DLT).
   Grade 3 fatigue lasting≤3 days.
   Grade 3 laboratory abnormalities that are asymptomatic and considered not to be clinically significant.
   Grade 3 diarrhea responding to standard of care within 72 hours.
   Grade 3 infections responding to standard intervention within 1 week.
Any hepatic function abnormality as defined by the following:
   Any case of aspartate aminotransferase (AST) or alanine aminotransferase (ALT)>3× Upper limit of normal (ULN) (if baseline was within normal limits) or baseline (if baseline was >ULN) and total bilirubin >2×ULN, with the following exception:
      AST or ALT >3×ULN and total bilirubin >2×ULN where no individual laboratory value exceeds Grade 3 that occurs in the context of CRS and resolves to Grade≤1 within <7 days.
   Any Grade 3 AST or ALT elevation with the following exception:
      Any Grade 3 AST or ALT elevation that occurs in the context of Grade ≤ 2 CRS and resolves to Grade≤1 within ≤7 days will not be considered a DLT.
Any Grade 2 neurologic toxicity mapping to a Medical Dictionary for Regulatory Activities (MedDRA) High-Level Group Term listed in Table 5 that is not considered by the investigator to be attributable to another clearly identifiable cause and that does not resolve to baseline within 72 hours.
Grade 1 depressed level of consciousness or Grade 1 dysarthria that is not considered by the investigator to be attributable to another clearly identifiable cause and that does not resolve to baseline within 72 hours.
Any grade seizure that is not considered by the investigator to be attributable to another clearly identifiable cause.

TABLE 5
Clinically Relevant Neurologic Toxicities
Defined by MedDRA High-Level Group Terms

Cranial nerve disorders (excluding neoplasms)
Demyelinating disorders
Encephalopathies
Mental impairment disorders
Movement disorders (including parkinsonism)
Neurological disorders NEC (not elsewhere classified)
Seizures (including subtypes)
Cognitive and attention disorders and disturbances
Communication disorders and disturbances
Delirium (including confusion)
Dementia and amnestic conditions

vi. Dose-Escalation Rules and Determination of the Maximum Tolerated Dose

Dose escalation will occur in accordance with the rules listed below. However, the totality of available safety data will be considered when making dose-escalation decisions for each dose and may decide to make smaller dose increases or to stop dose escalation for the step-up doses or target dose before it is required by the dose-escalation rules.

Relevant demographic, adverse event, laboratory, dose administration, and available PK and PD data (e.g., serum cytokines and markers of T-cell activation) will be reviewed prior to each dose escalation decision

The step-up and target doses may be increased up to a maximum of 3-fold of the preceding dose levels for each successive cohort until a safety threshold (defined as the observation of a Grade ≥2 adverse event not considered by the investigator to be attributable to another clearly identifiable cause in more than one-third of patients) is met. Once this safety threshold has been met during a DLT window of a given cohort, the corresponding dose may be increased up to a maximum of 2-fold of the preceding dose for subsequent cohorts. Following the observation of one DLT out of 6 patients during a DLT window of a given cohort, the corresponding dose may be increased up to a maximum of 1.5-fold of the preceding dose for subsequent cohorts.

Rules for dose escalation of the step-up doses are as follows:
If none of the first 3 DLT-evaluable patients in a given cohort experiences a DLT during the step-up dose DLT windows (DLT Windows 1 and 2), the step-up dose(s) may be escalated in the next cohort, as described above.
If 1 of the first 3 DLT-evaluable patients experiences a DLT during the step-up dose DLT windows (DLT Windows 1 and 2), the cohort will be expanded to 6 patients. If there are no further DLTs in the 6 DLT-evaluable patients during the step-up dose DLT windows, the step-up dose(s) may be increased up to a maximum of 1.5-fold of the preceding C1D1/C1D8 dose(s) in subsequent cohorts.
If 2 or more of the first 3 DLT-evaluable patients or at least one-third of 4 or more DLT-evaluable patients in a given cohort experience a DLT during the step-up dose DLT windows (DLT Windows 1 and 2), the corresponding step-up MTD will have been exceeded and escalation at that step-up dose will stop. An additional 3 patients will be evaluated for DLTs using a dosing scheme consisting of the preceding step-up dose level and the highest cleared target dose level, unless 6 patients have already been evaluated at that level.
If the step-up dose level at which the step-up MTD is exceeded is >25% higher than the preceding tested step-up dose, additional dose cohorts of at least 6 patients may be evaluated at intermediate step-up doses for evaluation as the step-up MTD.

Rules for dose escalation of the target dose are as follows:
If none of the first 3 DLT-evaluable patients in a given cohort experiences a DLT during the target-dose DLT window (DLT Window 3), enrollment of the next cohort at the next highest dose level for the target dose DLT window may proceed as described above.
If 1 of the first 3 DLT-evaluable patients experiences a DLT during the target-dose DLT window, the cohort will be expanded to 6 patients at the same dose level. (Note: if the step-up dose at a given level has been shown to exceed the step-up dose MTD, the additional patients enrolled in the cohort will be enrolled at a lower, previously cleared step-up dose.) If there are no further DLTs in 6 DLT-evaluable patients during the target dose DLT window, enrollment of the next cohort may proceed with the target dose being increased up to a maximum of 1.5-fold of the preceding target dose.
If 2 or more of the first 3 DLT-evaluable patients or at least one-third of 4 or more DLT-evaluable patients in a cohort experience a DLT during the target dose DLT window, the target dose MTD will have been exceeded and escalation of the target dose will stop, with the following exception:
   If all DLTs experienced at a given target dose were reported as CRS or its symptoms, an additional 3 patients may be evaluated for DLTs by dose-escalating the step-up dose(s) and using a lower, previously cleared target dose. If all 3 patients do not experience CRS or its symptoms in the new regimen, then the previously tested target dose can be re-tested using a higher step-up regimen. Escalation of target dosing may continue in this case in accordance with dose escalation rules above as the MTD in this case will not have been reached.
If the target dose MTD has been exceeded and no escalation of the step-up dose is planned, the following rules will apply:
   An additional 3 patients may be evaluated for DLTs using a dosing scheme consisting of the highest cleared step-up dose levels and the highest cleared target dose level, unless 6 patients have already been evaluated at that level.
   If the target dose MTD is exceeded at any dose level, the highest target dose at which fewer than 2 of 6 DLT-evaluable patients (i.e., <17%) experience a DLT will be declared the target dose MTD.
   If the target dose level at which the target dose MTD is exceeded is ≥25% higher than the preceding tested target dose, additional dose cohorts of at least 6 patients may be evaluated at intermediate target dose(s) for evaluation as the MTD.
Additional dose cohorts that assess intermediate dose levels between two dose levels that have been demonstrated to not exceed the MTD may be evaluated to further characterize dose-dependent toxicities.
   Enrollment of cohorts to evaluate intermediate dose levels may occur concurrently with enrollment of dose-escalation cohorts to identify the MTD.

If the target dose MTD is not exceeded at any dose level, the highest doses administered in this study for step-up and target dose in a single cohort will be declared the MTDs.

More than one dose-escalation cohort may be open in parallel as long as the MTD has not been exceeded.

When supported by clinical data, the second step-up dose can be escalated to match the target dose as long as the target dose is not exceeding the MTD. In such a case, the double step dosing will become a single step dosing in subsequent cohorts.

vii. Intrapatient Dose Escalation

To maximize the collection of information at relevant doses and minimize the exposure of patients to sub-optimal doses of cevostamab, intrapatient dose escalation may be permitted. Within each assigned dose-escalation cohort, the dose of cevostamab for an individual patient may be increased to the highest cleared dose level that is tolerated by completed cohorts through at least one cycle of cevostamab administration. Patients will be able to undergo intrapatient dose escalation after completing at least two cycles at their originally assigned dose level. Once the MTD is declared and the RP2D is determined, intrapatient dose escalation directly to the RP2D is permitted for patients who remain on study and continue to tolerate cevostamab.

viii. Dose-Expansion Stage

The dose-expansion stage for this study is designed to further characterize the safety, tolerability, pharmacokinetics, and clinical activity of cevostamab.

One or more expansion cohorts may be opened in order to, for example, test a combination of step up versus target doses. Not all expansion cohorts need to be opened at the same time.

Approximately 30 patients will be enrolled into any one expansion cohort. Patients will be treated at a previously cleared dose (at or below the MTD).

At no time will a cevostamab dose level studied in the expansion stage exceed the highest dose level tested and cleared in the dose-escalation stage. Additionally, for the expansion cohort, interim analyses will be conducted to guide potential early stopping of enrollment in the event of unacceptable toxicity/tolerability or lower than expected response rate.

If the frequency of Grade 3 or 4 toxicities or other unacceptable toxicities at the initial expansion-stage dose level suggests that the MTD has been exceeded, accrual at that dose level will be halted. Consideration will then be given to enrolling patients in an expansion cohort at a lower dose level.

ix. Rules for Continued Dosing Beyond Cycle 1

Patients in the dose-escalation and the dose-expansion stages will be eligible to receive ongoing cycles of study treatment provided the following criteria are met:
Ongoing clinical benefit: Patients must have no clinical signs or symptoms of progressive disease; patients will be clinically assessed for disease progression on Day 1 of each cycle). Patients will also be assessed at the beginning of each cycle for progression based on the IMWG criteria (Tables 8A and 8B). Patients with solely biochemical disease progression (defined as an increase of monoclonal paraprotein in absence of organ dysfunction and clinical symptoms) and who qualify for intrapatient dose escalation may receive additional study treatment. For determining disease progression according to IMWG criteria after a patient has undergone intrapatient dose escalation, baseline will be re-established at each new dose level assessed for a patient.
Acceptable toxicity: Patients who experience Grade 4 non-hematologic adverse events with the possible exception of Grade 4 tumor lysis syndrome (TLS) should discontinue study treatment and may not be re-treated. Patients who experience Grade 4 TLS may be considered for continued study treatment.
All other study treatment-related adverse events from prior study treatment injections must have decreased to Grade≤1 or baseline grade by the next injection. Exceptions on the basis of ongoing overall clinical benefit may be allowed after a careful assessment. Dose reductions of cevostamab may be allowed if it is determined that clinical benefit may be maintained according to the rules outlined further.

Patients who experienced a DLT in Cycle 1 may be allowed to continue treatment based on clinical benefit on a case-by-case basis.

### X. Cevostamab Re-Treatment

Patients who initially respond to cevostamab, but subsequently develop recurrent or progressive disease either after the completion of therapy (13 cycles of treatment) or after a dose delay of more than 28 days due to a non-treatment-related event (on treatment, before completion of 13 cycles of therapy), may benefit from additional cycles of cevostamab treatment. Patients will be eligible for cevostamab re-treatment as described below. Patients who enroll in re-treatment will begin treatment on Cycle 1 per the protocol-specified schedule and may continue treatment until disease progression (according to IMWG criteria) or unacceptable toxicity, whichever occurs first, provided the following criteria are met:
Pertinent eligibility criteria are met at the time that cevostamab treatment is re-initiated, with the following exceptions:
   Prior therapy with cevostamab is allowed.
   Patients who do not have measurable disease are allowed if they have clear indicators of recurrent disease (i.e., new lesion or plasmacytoma).
   The investigator and patient would like to pursue cevostamab re-treatment, despite the appropriateness and availability of any new therapies for the treatment of MM.
   Serology tests to demonstrate HIV, hepatitis C virus (HCV), and hepatitis B virus (HBV) status do not need to be repeated unless clinically indicated.
   Epstein-Barr virus (EBV), cytomegalovirus (CMV), and human herpes virus 6 (HHV-6) PCR must be repeated.
   Manageable and reversible immune-related adverse events with initial cevostamab treatment are allowed and do not constitute an exclusionary history of autoimmune disease.
   Radiotherapy is allowed within 4 weeks of cevostamab re-treatment.
Patients must have had documented objective response (complete response (CR), very good partial response (VGPR), or partial response (PR)) per IMWG criteria at the end of initial cevostamab treatment and for at least one post-treatment tumor assessment after the end of treatment.
Patients without biochemical disease progression (defined as an increase of monoclonal paraprotein in absence of organ dysfunction and clinical symptoms) but who have clear indication of recurrent disease (i.e., development of new bone lesions or soft tissue plasmacytomas or an increase in size of existing bone lesions or soft tissue plasmacytomas) are allowed.
Patients who experienced Grade 2 or Grade 3 adverse events considered to be related to treatment during initial treatment must have resolved these toxicities to Grade≤1.
Patients will be required to follow hospitalization instructions for Cycle 1 following the first re-treatment with cevostamab.
No intervening systemic anti-cancer therapy was administered between the completion of initial cevostamab treatment and re-initiation of cevostamab treatment.

A repeat bone marrow biopsy and aspirate to assess FcRH5 expression status must be obtained prior to cevostamab re-treatment.

The above rules for re-treatment apply both to patients in the dose-escalation stage and dose-expansion stage.

xi. End of Study and Length of Study

The end of this study is defined as the date when the last patient, last visit (LPLV) occurs or the date at which the last data point required for statistical analysis or protocol defined safety monitoring is received from the last patient, whichever occurs later.

xii. Rationale for Patient Population

In Study GO39775, cevostamab has demonstrated preliminary activity in patients with R/R MM, the same patient population selected for this Example (GO43227). This study will enroll patients with a history of R/R MM who meet the inclusion and exclusion criteria as described herein.

Confirmation of FcRH5 expression will not be required during eligibility screening prior to enrollment, but it will be evaluated retrospectively, based on the following rationale:
FcRH5 is a cell-surface antigen whose expression is restricted to cells of the B linage, including plasma cells. It is expressed with 100% prevalence on MM samples tested to date (Elkins et al., Mol Cancer Ther, 11:2222-32, 2012; Li et al., Cancer Cell, 31:383-95, 2017).
Nonclinical studies have demonstrated that cevostamab is broadly active in cell killing in multiple human MM cell lines and primary human MM plasma cells with a wide range of FcRH5 expression levels, including cells with minimal FcRH5 expression, suggesting that even very low levels of FcRH5 expression may be sufficient for clinical activity (Li et al., Cancer Cell, 31:383-95, 2017).
Preliminary findings from Study GO39775 suggest that patients are able to achieve an objective response to cevostamab treatment regardless of baseline FcRH5 expression. Clinical response has been observed in patients with the lowest FcRH5 expression.
   xiii. Rationale for Dosing Schedule for Cevostamab

The primary objective of this study is to evaluate the safety and tolerability of SC cevostamab monotherapy.

In Study GO39775, IV cevostamab is being evaluated in a Q3W dosing schedule; this schedule does not align with the dosing schedules of other MM therapies such as pomalidomide or daratumumab, which are administered in 4-week cycles. Combination of SC cevostamab with other anti-myeloma agents that augment and/or complement T-cell-mediated killing of myeloma cells may in the future provide enhanced clinical benefit to patients. Therefore, the proposed schedule for monotherapy SC cevostamab in this study (GO43227) will be 28-day cycles, with SC cevostamab administered QW for the first 3 weeks followed by 1 week off (Cycle 1), followed by Q2W administration for 20 weeks (Cycles 2-6) and Q4W administration for 28 weeks (Cycles 7-13).

xiv. Rationale for Cevostamab Dose

The starting doses for SC cevostamab are based on safety and PK data from the ongoing first-in-human (FIH) Study GO39775 and on available nonclinical data, as described below. Initially, SC cevostamab will be delivered as follows: 2 mg (C1D1 dose), 10 mg (C1D8 dose), and 40 mg (C1D15 target dose) in Cycle 1.

a. Nonclinical Data on SC Cevostamab

Nonclinical testing in cynomolgus monkeys assessed the safety, tolerability, and pharmacodynamic effects of 4 mg/kg cevostamab delivered intravenously and 4 mg/kg cevostamab delivered subcutaneously. All animals tolerated SC cevostamab well over the whole sampling interval; no localized skin reactions were observed following SC administration. With SC dosing of cevostamab, the Cₘₐₓ was reduced by 85% compared with the 1-hour IV infusion. The time to Cₘₐₓ (tₘₐₓ) was delayed to 6 hours following SC cevostamab dosing as compared with IV dosing, where tₘₐₓ was 1.2 hours.

b. Clinical Data on IV Cevostamab

Step-Up Dosing: In Study GO39775, as of the CCOD of 16 Nov. 2020, approximately 115 R/R MM patients have received IV cevostamab, either with single or double step-up dosing in Cycle 1. Single step-up doses from 0.05 mg up to 3.6 mg have been evaluated. While the MTD for single step-up dosing for C1D1 was not reached, the 3.6 mg IV step-up dose was demonstrated to be tolerable and was able to mitigate the frequency of CRS observed at the subsequent target dose, beginning at 10.8 mg and ranging up to 160 mg. As noted with double step-up dosing in Arm B, a first step-up dose of 0.3 mg was associated with a reduced number of Grade 2 CRS events associated with the C1D1 infusion (versus 0.6 mg and 1.2 mg), and the subsequent 3.6-mg second step-up dose mitigated the frequency of CRS after the C1D15 target dose.

Furthermore, results of the preliminary exposure-safety analysis from Study GO39775 indicate that the incidence of CRS events was likely associated with the maximum concentrations (Cₘₐₓ) following the step-up dose of cevostamab (0.05-3.6 mg), a noted class effect of the bispecific T-cell engager antibodies (Nagorsen et al., Pharmacol Ther, 136:334-42, 2012; Staflin et al., JCI Insight, 5: e133757, 2020).

Target Dosing: As of the clinical cut-off date (CCOD) of 16 Nov. 2020, in Study GO39775, the MTD of the target dose has not been reached. With a C1D1 fixed step-up dose of 3.6 mg, the target dose has been escalated from 10.8 mg to 160 mg with no apparent increase in the frequency or severity of CRS. Similarly, the frequency of non-CRS adverse events did not appear to increase with escalation of the target dose. Anti-myeloma activity was observed with IV cevostamab doses of 20 mg and higher and associated with exposure metrics such as AUC and Cₘᵢₙ.

c. Population Pharmacokinetic Simulations to Support the Starting Dose for SC Cevostamab

To further support the starting dose schedule for SC cevostamab, a previously developed population pharmacokinetic (popPK) model was implemented to simulate the Cₘₐₓ equivalent to ˜0.3-0.6 mg IV doses on C1D1, the Cₘₐₓ equivalent to ˜3.6 mg IV dose on C1D8, and the AUC equivalent to IV doses in the active dose range of ˜20-40 mg on C1D15. Given the uncertainty in PK behavior following SC administration for cevostamab, the SC starting doses were selected to attain systemic exposures equivalent to the aforementioned IV doses based on the popPK simulations performed using the variability in bioavailability (F range: 60%-99%) and absorption rate constants (Ka range: 0.13-0.5/day) for typical monoclonal antibodies published in the literature.

Based on the above data, the estimates of 2 mg (Day 1; first step-up dose), 10 mg (Day 8; second step-up dose), and 40 mg (target dose) were selected as the starting doses for cevostamab SC administration in Cycle 1.

Given that the peak serum concentrations (Cₘₐₓ) of cevostamab following SC dosing in cynomolgus monkeys were lower than for IV dosing when tested at the same dose levels, and the incidence of CRS events was likely associated with the maximum concentrations (Cₘₐₓ) following the step-up doses of IV cevostamab (0.05-3.6 mg) in Study GO39775, an initial dose of 2 mg is expected to provide an acceptable margin of safety for the initial SC dosing cohort.

It is therefore expected that an initial C1D1 SC cevostamab dose of 2 mg will be associated with a low number of CRS events, given the Cₘₐₓ equivalent for IV doses of 0.3-0.6 mg. Similarly, the initial C1D8 dose of 10 mg was selected as the Cₘₐₓ equivalent of the 3.6-mg IV dose and is expected to mitigate CRS at the subsequent C1D15 target dose. The initial target dose of 40 mg was selected to provide an AUC equivalent of the 20-40 mg IV dose, thereby allowing delivery of a clinically active dose for the initial dosing cohort.

### C. MATERIALS AND METHODS

i. Patients

Approximately 60 patients with R/R MM will be enrolled in this study.

ii. Inclusion Criteria

Patients must meet the following criteria for study entry:
Signed Informed Consent Form(s).
Age ≥18 years at time of signing Informed Consent Form.
Ability to comply with the study protocol, in the investigator's judgment.
Eastern Cooperative Oncology Group (ECOG) Performance Status of 0 or 1.
Life expectancy of at least 12 weeks.
Diagnosis of R/R MM for which no established therapy for MM is appropriate and available, or intolerance to those established therapies.
Agreement to provide bone marrow biopsy and aspirate samples.
Resolution of adverse events from prior anti-cancer therapy to Grade≤1, with the following exceptions:
   Any grade alopecia is allowed.
   Peripheral sensory or motor neuropathy must have resolved to Grade≤2.
Measurable disease, defined as at least one of the following:
   Serum M-protein ≥0.5 g/dl (≥5 g/L).
   Urine M-protein ≥200 mg/24 hr.
   Serum free light chain (SFLC) assay: Involved SFLCs ≥10 mg/dl (≥100 mg/L) and an abnormal SFLC ratio (<0.26 or >1.65).
Laboratory values as follows:
   Hepatic function-
      AST and ALT≤3×ULN.
      Total bilirubin≤1.5×ULN; patients with a documented history of Gilbert syndrome and in whom total bilirubin elevations are accompanied by elevated indirect bilirubin are eligible.
   Hematologic function (requirement prior to first dose of cevostamab)
      Platelet count >50,000/mm³ without transfusion within 7 days prior to first dose.
      ANC ≥1000/mm³.
      Total hemoglobin ≥8 g/dL.
(Note: Patients may receive supportive care (e.g., transfusion, G-CSF, etc.) to meet hematologic function eligibility criteria.)
Patients who do not meet criteria for hematologic function because of MM-related cytopenias (e.g., due to extensive marrow involvement by MM) may be enrolled into the study.
   Creatinine≤2.0 mg/dl and creatinine clearance (CrCl) ≥30 mL/min (either calculated using modified Cockcroft-Gault equation or per 24-hr urine collection).
   Serum calcium (corrected for albumin) level≤11.5 mg/dl (treatment of hypercalcemia is allowed and patient may enroll if hypercalcemia returns to Grade≤ 1 with standard treatment).
      iii. Exclusion Criteria

Patients who meet any of the following criteria will be excluded from study entry:
Prior treatment with cevostamab or another agent targeting FcRH5.
Prior use of any monoclonal antibody, radioimmunoconjugate, or antibody-drug conjugate as anti-cancer therapy within 4 weeks prior to first study treatment, except for the use of non-myeloma therapy (e.g., denosumab for hypercalcemia is allowed).
Prior treatment with systemic checkpoint inhibitors, including, but not limited to anti-CTLA4, anti-PD-1, and anti-PD-L1 therapeutic antibodies within 12 weeks or 5 half-lives of the drug, whichever is shorter, prior to first study treatment.
Prior treatment with allogeneic or autologous chimeric antigen receptor (CAR) T-cell therapy within 12 weeks prior to first study treatment.
Known treatment-related, immune-mediated adverse events associated with prior checkpoint inhibitors as follows:
   Prior PD-L1/PD-1 or CTLA-4 inhibitor: Grade ≥3 adverse events with the exception of Grade 3 endocrinopathy managed with replacement therapy
   Grade 1-2 adverse events that did not resolve to baseline after treatment discontinuation.
Treatment with any chemotherapeutic agent or other anti-cancer agent (investigational or otherwise) within 4 weeks or 5 half-lives of the drug, whichever is shorter, prior to first study treatment
Treatment with radiotherapy within 4 weeks (systemic radiation) or 14 days (focal radiation) prior to first study treatment.
Autologous SCT within 100 days prior to first study treatment.
Prior allogeneic SCT
   Prior allogeneic CAR T-cell therapy is allowed as long as treatment was completed >12 weeks prior to first study treatment.
      iv. Tocilizumab

Tocilizumab will be administered as a rescue investigational medicinal product (IMP) when necessary to patients who experience a CRS event (e.g., see Table 6). It will be formulated, prepared, and handled according to standard institutional practices.

TABLE 6
Tocilizumab treatment of cytokine release syndrome (CRS)

| | Pre-TCZ Tx | | | | | | |
|---|---|---|---|---|---|---|---|
| | (at CRS | TCZ | Post-TCZ Treatment ^{b, c} | | | | |
| Assessment/Procedure ^{a} | onset) | Admin | 6 hours | 1 day | 2 days | 3 days | 8 days |

| | |
|---|---|
| TCZ administration ^{d} | x |

| | | |
|---|---|---|
| Vital signs ^{e} | x ^{f} | Measure at least every 6 hours until resolution to |
| | | baseline, then every 12 hours until end of |
| | | hospitalization ^{f} |
| Pressor documentation ^{g} | x ^{f} | Record at least every 6 hours until pressors are |
| | | discontinued ^{f} |
| FiO2 | x ^{f} | Record at least every 6 hours until patient on room |
| | | air ^{f} |
| Pulse oximetry, resting | x ^{f} | Measure at least every 6 hours until resolution to |
| | | baseline, then every 12 hours until end of |
| | | hospitalization ^{f} |

Local Laboratory Assessments

| | | | | | | |
|---|---|---|---|---|---|---|
| Hematology | x | x | x | x | x | x |
| Liver function tests | x | x | x | x | x | x |
| (AST, ALT, total | | | | | | |
| bilirubin) | | | | | | |
| Serum chemistry and | x | x | x | x | x | x |
| creatinine ^{h} | | | | | | |
| C-reactive protein | x | x | x | x | x | x |
| (CRP), lactate | | | | | | |
| dehydrogenase (LDH), | | | | | | |
| and serum ferritin | | | | | | |
| Coagulation (activated | x | x | x | x | x | x |
| partial thromboplastin | | | | | | |
| time (aPTT), | | | | | | |
| prothrombin time | | | | | | |
| (PT)/international | | | | | | |
| normalized ratio (INR), | | | | | | |
| fibrinogen) | | | | | | |

Admin. = administration; aPTT = activated partial thromboplastin time; CRP = C-reactive protein; CRS = cytokine release syndrome; FiO₂ = fraction of inhaled oxygen; INR = international normalized ratio; IL-6 = interleukin 6; LDH = lactate dehydrogenase; PT = prothrombin time; TCZ = tocilizumab; Tx = treatment.
^{a} Any assessments/procedures in Table 6 may be waived by the if the patient is hospitalized at a facility that does not have the capacity to perform such study assessments. Hospitalization should not be prolonged to perform study assessments.
^{b} If the TCZ dose is repeated, follow Table 6 following the second TCZ dose.
^{c} For post-TCZ treatment timepoints, the windows are as follows: 6 hours (±30 minutes), 1 day (24 ± 4 hours), 2 days (48 ± 4 hours), 3 days (72 ± 4 hours), and 8 days (192 ± 48 hours) after completion of TCZ infusion, respectively.
^{d} TCZ dosing: 8 mg/kg IV for patients at or above 30 kg weight; 12 mg/kg IV for patients less than 30 kg weight; repeat every 8 hours as necessary (up to a maximum of 4 doses). Doses exceeding 800 mg per infusion are not recommended.
^{e} Includes respiratory rate, heart rate, and systolic and diastolic blood pressure while the patient is in a seated or supine position, and temperature.
^{f} The maximum and minimum values for any 24-hour period should be recorded.
^{g} Document vasopressor type and dose.
^{h} Includes sodium, potassium, chloride, bicarbonate, glucose, and blood urea nitrogen (BUN).

v. Cevostamab

Flat dosing independent of body weight may be used for cevostamab.

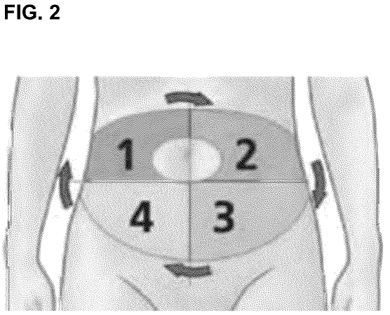

Cevostamab will be administered to patients subcutaneously, preferentially into the subcutaneous tissue of the abdomen, with an injection speed of approximately 1 mL/min. The abdomen may be divided into 4 quadrants, and injection sites will be rotated as shown in FIG. 2.

Alternatively, if injections cannot be performed in any of the abdominal quadrants, they may be administered in the thighs.

The Drug Product may be delivered by medical syringe, with the final cevostamab volume determined by the dose.

Cevostamab will be administered in a setting with immediate access to trained critical care personnel and facilities equipped to respond to and manage medical emergencies. Cevostamab dosing will occur only if a patient's clinical assessment and laboratory test values are acceptable.

All patients must have IV access in place prior to cevostamab SC administration for at least the first cycle. Placement of IV access may be optional from Cycle 2 onwards.

All cevostamab doses will be administered to well-hydrated patients. Premedication(s) must be administered prior to the administration of cevostamab as indicated in Table 7.

TABLE 7
Premedication(s) Required Prior to Cevostamab Administration

| | Dose and | |
|---|---|---|
| Premedication | Route | Timepoints |
| Dexamethasone ^{a} | 20 | Cycles 1-2: 1 hour (±15 minutes) prior |
| | mg IV | to each cevostamab injection |
| | | Cycles 3 and beyond: 1 hour (±15 |
| | | minutes) prior to cevostamab |
| | | injection if patient experienced |
| | | CRS with the prior injection |
| Acetaminophen/ | 500-1000 | Prior to each cevostamab injection (all |
| paracetamol | mg PO | cycles) |
| Diphenhydramine ^{b} | 25-50 | Prior to each cevostamab injection (all |
| | mg PO | cycles) |

^{a} Dexamethasone 20 mg IV preferred; alternative corticosteroid equivalent such as methylprednisolone 80 mg IV is also acceptable.
^{b} For sites that do not have access to diphenhydramine, an equivalent medication may be substituted per local practice.

Patients will be observed for at least 90 minutes for fever, chills, rigors, hypotension, nausea, or other signs and symptoms following each subsequent cevostamab administration.

Patients who receive less than 80% of a cevostamab step-up dose should repeat the step-up dose (if the patient meets all the dosing requirements) prior to receiving the higher target dose. In Cycle 1, a repeat step-up dose will be allowed if a patient experiences an adverse event during step-up dosing, that the investigator determines to be clinically significant and warrants a repeat step-up dose at the next dosing. The step-up dose must be repeated for any patient that experiences a Grade ≥3 CRS event following a step-up dose prior to receiving the first target dose.

For management of adverse events, including infusion-related reactions (IRRs), CRS, and injection-site reactions (ISRs), and guidelines for dosage and schedule modification and treatment interruption or discontinuation are provided further.

vi. Permitted Therapy

Patients who use oral contraceptives, hormone-replacement therapy, or other maintenance therapy should continue their use.

Concomitant use of hematopoietic growth factors such as erythropoietin, granulocyte colony-stimulating factor (filgrastim, pegfilgrastim), granulocyte/macrophage colony-stimulating factor (sargramostim), or thrombopoietin (oprelvekin, eltrombopag) is allowed in accordance with instructions provided in the package inserts, institutional practice and/or published guidelines. For patients with Grade 3 or 4 neutropenia, growth factor support is encouraged and should be administered per institution guidelines. Platelet transfusions for patients with thrombocytopenia are permitted and should be instituted per institutional guidelines.

Anti-infective prophylaxis for viral, fungal, bacterial, or pneumocystis infections is encouraged for patients at high risk of infections and should be instituted per institutional practice. The use of IV immunoglobulin (Ig) replacement therapy is permitted to reduce the risk of recurrent infection due to hypogammaglobulinemia per institutional guidelines.

Bisphosphonates or denosumab, as indicated for hypercalcemia or prevention of skeletal-related events, may be continued if the patient is already on either therapy prior to starting the study; additionally, initiation of these therapies during screening, pre-phase, or Cycle 1 is permitted. After Cycle 1, bisphosphonates may be prescribed so long as there is no sign of disease progression.

The use of inhaled corticosteroids and mineralocorticoids (e.g., fludrocortisone) for patients with orthostatic hypotension or adrenocortical insufficiency is allowed.

Physiologic doses of corticosteroids for adrenal insufficiency are allowed. Megestrol administered as an appetite stimulant is acceptable while the patient is enrolled in the study.

Influenza vaccination should be given during influenza season only. Inactivated vaccines are allowed.

In general, investigators should manage a patient's care (including preexisting conditions) with supportive therapies other than those defined as prohibited therapies as clinically indicated, per local standard practice.

Treatment of severe CRS, hemophagocytic lymphohistiocytosis (HLH), or macrophage activation syndrome (MAS) according to published recommendations and/or institutional practice is permitted.

vii. Medications Given with Precaution Due to Effects Related to CYP Enzymes

Given the expected pharmacology of cevostamab, the transient release of cytokines may suppress CYP450 enzymes and cause drug-drug interactions. Based on clinical data, cytokine levels (IL-6 and IFN-γ are the highest during the first 24 hours of the first cycle. During subsequent cycles, cytokine levels are substantially reduced. Patients who may be at risk of a drug-drug interaction are those receiving concomitant medications that are CYP450 substrates and have a narrow therapeutic index. Such concomitant medications should be monitored for toxicity, and dose adjusted accordingly.

viii. Study Assessments

Screening and pretreatment tests and evaluations will be performed within 14 days preceding the first dose of study treatment with the exception of the positron emission tomography (PET; also referred to as an 18F-fluorodeoxyglucose (FDG)-PET scan)/computed tomography (CT), low dose CT, or MRI scan to evaluate for suspected or known extramedullary disease, which may be performed up to 28 days preceding the first dose of study drug, providing no anti-tumor therapy was administered in this period.

Results of standard-of-care tests or examinations performed prior to obtaining informed consent and within 14 days prior to C1D1 may be used; these tests do not need to be repeated for screening.

ix. Disease-Specific Assessments

Patients will be evaluated for disease response and progression according to the IMWG response criteria as described below (e.g., see Tables 8A and 8B).

TABLE 8A
A International Myeloma Working
Group Uniform Response Criteria (2016)
Adapted from Durie et al. Leukemia 2015; 29: 2416-7
and Kumar et al. Lancet Oncol 2016; 17: e328-46.

| | |
|---|---|
| Response | |
| Subcategory | Response Criteria |

All response categories require two consecutive
assessments made any time before starting any new therapy.

| | |
|---|---|
| sCR | CR as defined below, plus: |
| | Normal free light chain (FLC) ratio and absence of clonal cells in bone marrow (BM) by |
| | immunohistochemistry (kappa/lambda ratio ≤4:1 or ≥1:2 |
| | for kappa and lambda patients, respectively after |
| | counting ≥100 plasma cells) ^{a} |
| CR | No evidence of initial monoclonal protein isotype(s) on |
| | immunofixation of the serum and urine, ^{b} disappearance |
| | of any soft tissue plasmacytomas, and ≤5% plasma cells |
| | in BM. |
| VGPR | Serum and urine M-protein detectable by immunofixation |
| | but not on electrophoresis; or ≥90% reduction in serum |
| | M-protein plus urine M-protein level <100 mg/24 hr ^{c}. |
| PR | ≥50% reduction of serum M-protein and reduction in |
| | 24-hour urine M-protein by ≥90% or to <200 mg/24 hr. |
| | If the serum and urine M-protein are unmeasurable, |
| | a ≥50% decrease in the difference between involved |
| | and uninvolved FLC levels is required in place of |
| | the M-protein criteria. |
| | If serum and urine M-protein are unmeasurable and |
| | serum FLC assay is also unmeasurable, ≥50% reduction |
| | in plasma cells is required in place of M-protein, |
| | provided baseline BM plasma cell percentage was ≥30%. |
| | In addition to the above listed criteria, if present |
| | at baseline, a ≥50% reduction in the size (SPD) ^{d} |
| | of soft tissue plasmacytomas is also required. |
| MR | ≥25% but ≤49% reductions of serum M-protein and |
| | reduction in 24-hour urine M-protein by 50%-89%. |
| | In addition to the above criteria, if present at |
| | baseline, 25%-49% reduction in the size (SPD) ^{d} |
| | of soft tissue plasmacytomas is also required. |
| SD | Not meeting criteria for MR, CR, VGPR, PR, or PD |

TABLE 8B
International Myeloma Working Group Uniform Response Criteria (2016)
Adapted from Durie et al. Leukemia 2015; 29: 2416-7
and Kumar et al. Lancet Oncol 2016; 17: e328-46

| Relapse | |
|---|---|
| Subcategory | Relapse Criteria |
| Pd ^{e, f} | Any one or more of the following criteria: |
| | Increase of ≥25% from lowest response value |
| | in one or more of the following: |
| | Serum M-protein (absolute increase must |
| | be ≥0.5 g/dL). |
| | Serum M-protein increase ≥1 g/dL, if |
| | the lowest M component was ≥5 g/dL. |
| | Urine M-protein (absolute increase must be |
| | ≥200 mg/24 hr). |
| | In patients without measurable serum and urine |
| | M-protein levels: the difference between involved |
| | and uninvolved FLC levels (absolute increase must |
| | be >10 mg/dL). |
| | In patients without measurable serum and urine |
| | M-protein levels and without measurable disease |
| | by FLC: BM plasma cell percentage irrespective |
| | of baseline status (absolute % must be ≥10%) ^{b}. |
| | Appearance of new lesion(s), ≥50% increase from |
| | nadir in SPD of >1 lesion,or ≥50% increase in |
| | the longestdiameter of a previous lesion >1 cm in |
| | short axis. |
| | ≥50% increase in circulating plasma cells (minimum |
| | 200 cells per microliter) if this is the only measure |
| | of disease. |
| Clinical | Requires one or more of the following: |
| relapse | Direct indications of increasing disease and/or end |
| | organ dysfunction (CRAB features) ^{g} related to the |
| | underlying clonal plasma cell proliferative disorder. |
| | It is not used in calculation of time to progression |
| | or PFS but is listed here as something that can be |
| | reported optionally or for use in clinical practice. |
| | Development of new soft tissue plasmacytomas |
| | or bone lesions (osteoporotic fractures do not |
| | constitute progression). |
| | Definite increase in the size of existing |
| | plasmacytomas or bone lesions. A definite increase |
| | is defined as a 50% (and ≥1 cm) increase as |
| | measured serially by the sum of the products of |
| | the cross-diameters of the measurable lesion. |
| | Hypercalcemia >11 mg/dL (2.65 mmol/L). |
| | Decrease in hemoglobin of ≥2 g/dL (1.25 mmol/L) |
| | not related to therapy or other non-myeloma |
| | related conditions. |
| | Rise in serum creatinine by 2 mg/dL or more |
| | (177 μmol/L or more) from the start of therapy |
| | and attributable to myeloma. |
| | Hyperviscosity related to serum paraprotein. |
| Relapse from | Any one or more of the following: |
| CR (to be | Reappearance of serum or urine M-protein by |
| used only if | immunofixation or electrophoresis. |
| the endpoint | Development of ≥5% plasma cells in the BM. |
| studied is | Appearance of any other sign of progression |
| PFS) ^{d} | (i.e., new plasmacytoma, lytic bone lesion, |
| | or hypercalcemia). |

BM=bone marrow; CR=complete response; CT=computed tomography; FLC=free light chain; M-protein=monoclonal protein; MR=minimal response; MRI=magnetic resonance imaging; PD=progressive disease; PET=positron emission tomography; PFS=progression-free survival;PR=partial response; sCR=stringent complete response; SD=stable disease; SPD=sum of the products of diameters; VGPR=very good partial response.
Note: Patients should be categorized as having stable disease until they meet criteria for any response category or have progressive disease. Patients will continue in the last confirmed response category until there is confirmation of progression or improvement to a higher response status;patients cannot move to a lower response category.
^{a} Special attention should be given to the emergence of a different M-protein following treatment, especially in the setting of patients having achieved a conventional CR, often related to oligoclonal reconstitution of the immune system. These bands typically disappear over time, and insome studies, have been associated with a better outcome. Also, appearance of IgGk in patients receiving monoclonal antibodies should be differentiated from the therapeutic antibody.
^{b} In some cases it is possible that the original M-protein light-chain isotype is still detected on immunofixation, but the accompanying heavy-chain component has disappeared; this would not be considered a CR even though the heavy-chain component is not detectable, since it is possible that the clone evolved to one that secreted only light chains. Thus, if a patient has IgA lambda myeloma, then to qualify as a CR there should beno IIgA detectable on serum or urine immunofixation; if free lambda is detected without IgA, then it must be accompanied by a different heavy-chain isotype (IgG, IgM, etc.). Modified from Durie etal. Leukemia; 20:1467-73 2006. Requires two consecutive assessments to be carried out at any time before the institution of any new therapy (Durie et al. Leukemia 2015; 29:2416-7).
^{c F}or patients achieving very good partial response by other criteria, a soft tissue plasmacytoma must decrease by more than 90% in the sum of the maximal perpendicular diameter (SPD) compared with baseline.
^{d} Plasmacytoma measurements should be taken from the CT portion of the PET/CT or MRI scans, or dedicated CT scans where applicable. Forpatients with only skin involvement, the skin lesions should be measured with a ruler. Measurement of tumor size will be determined by the SPD. Any soft tissue plasmacytoma documented at baseline must undergo serial monitoring; otherwise, the patient is classified as not evaluable.
^{e} Positive immunofixation alone in a patient previously classified as achieving a CR will not be considered progression. Criteria for relapse from aCR should be used only when calculating disease-free survival.
^{f} In the case where a value is felt to be a spurious result per investigator discretion (e.g., a possible laboratory error), that value will not beconsidered when determining the lowest value.
^{g} CRAB features=calcium elevation, renal failure, anemia, lytic bone lesions.

A bone marrow biopsy and aspirate may be required prior to initiation of study treatment, within 3 days prior to or on C2D1, and at the time of confirmation of CR or disease progression. The bone marrow sample scheduled prior to initiation of study treatment may be obtained after the patient's other screening procedures have been completed. Patients who are re-screened after an initial screen failure do not need to undergo a repeat bone marrow biopsy and aspirate if these assessments were completed during the initial screening period. For the bone marrow biopsy and aspirate at disease progression, if a bone marrow biopsy and aspirate was done within 28 days of the scheduled timepoint to confirm response, then this assessment may be omitted.

The following myeloma-specific tests will be performed at the beginning of every cycle
Serum protein electrophoresis (SPEP) with serum immunofixation electrophoresis (SIFE).
SFLCs.
Quantitative Ig levels.

The following myeloma-specific tests should be performed at screening and as needed to confirm a response:
A 24-hour urine protein electrophoresis (UPEP) with urine immunofixation electrophoresis (UIFE) for M-protein quantitation.

The following confirmatory assessments are required for all response categories (stringent complete response (sCR), CR, VGPR, PR, and minimal response (MR)):
If extra-medullary disease was previously present, CT scan or MRI with bi-dimensional measurements to confirm reduction in size per IMWG criteria.
If extra-medullary disease was previously present, PET/CT scan, CT scan, or MRI is required to confirm complete resolution.
24-hour UPEP/UIFE is required to confirm VGPR even if a UPEP was not performed at screening.

The following additional samples/assessments are required to confirm a sCR or CR:
SIFE.
SFLC.
24-hour UPEP/UIFE is required to confirm CR/sCR even if a UPEP was not performed at screening.
Bone marrow aspiration and biopsy.
If extra-medullary disease was previously present, PET-CT scan, CT scan, or MRI to confirm complete resolution.

To confirm progressive disease, the following are required:
If progressive disease is suspected by rising M-protein, SPEP, UPEP, or SFLC analysis should be obtained on two consecutive assessments per IMWG criteria (see Tables 8A and 8B).
If progressive disease is suspected on development of new bone lesions or soft tissue plasmacytomas or an increase in size of existing bone lesions or soft tissue plasmacytomas, skeletal survey/CT scan/MRI should be obtained and compared with baseline imaging.
If progressive disease is suspected on hypercalcemia attributed solely to MM, local laboratory results levels of serum calcium should be >11 mg/dl and confirmed on a second assessment.

### X. Skeletal Survey

A skeletal survey will be completed at screening and as clinically indicated. The skeletal survey may be completed up to 28 days prior to C1D1. Plain films and CT scans are both acceptable imaging modalities for assessing skeletal disease. Imaging should include the skull, long bones, chest, and pelvis. If plasmacytomas are seen on skeletal survey, bi-dimensional tumor measurements should be recorded. The skeletal survey may be omitted if a PET/CT scan or a low-dose, whole-body CT, or whole-body MRI is performed as part of screening.

xi. Extramedullary Disease

All patients with clinically suspected extra-medullary disease or known extra-medullary disease at the time of screening must undergo imaging during screening to evaluate for the presence/extent of extramedullary disease. This should be performed using PET/CT, CT scan of the chest, abdomen, and pelvis (preferably with IV contrast if renal function is adequate), or whole body MRI. Patients who are found to have extra-medullary disease will undergo repeat imaging with the same modality used at screening when possible every 12 weeks (+7 days). Imaging should also be performed upon clinical suspicion of progressive disease (the same method as at screening should be used throughout study if possible). Ultrasound of the abdomen/liver/spleen may be substituted for CT, PET/CT, or MRI if, per the investigator's assessment, patients are not able to safely tolerate these imaging modalities, and the anatomic location of the extramedullary disease is compatible with these alternative imaging methods.

xii. Evaluation of Patient/Caregiver Alert Card

A questionnaire to evaluate the utility and adequacy of the patient/caregiver alert card may be administered to patients by study staff prior to Cycle 1 Day 1 (after eligibility is confirmed) and at the end of treatment visit.

xiii. Laboratory and Other Biological Samples

Samples for the following laboratory tests will be analyzed:
Standard-of-care assessments of bone marrow biopsies and aspirate for local clinical pathology assessment, which will include, but are not limited to, multiple myeloma response assessment, cytogenetic analysis by fluorescence in situ hybridization (FISH; including markers such as 1q gain, del17, t (11:14), t (4; 14), t (14; 16)), percent abnormal plasma cells, cytoplasmic kappa: lambda ratio of plasma cells, and immunophenotyping of abnormal plasma cells.
Myeloma-specific tests: quantitative Igs (IgA, IgG, and IgM), SPEP with SIFE, UPEP, SFLC.
Hematology: hemoglobin, hematocrit, red blood cell count, white blood cell count, platelet count, absolute neutrophil count, and percent or absolute differential counts (segmented neutrophils bands, lymphocytes, eosinophils, monocytes, basophils, and other cells).
Coagulation: aPTT, PT, INR, and fibrinogen.
Serum chemistry: sodium, potassium, chloride, bicarbonate, glucose, BUN, creatinine, calcium, magnesium, phosphorous, LDH, and uric acid.
Liver function tests (LFTs): total and direct bilirubin, total protein, albumin, ALT, AST, alkaline phosphatase (ALP), and gamma-glutamyl transpeptidase (GGT).
Serum β-2 microglobulin.
C-reactive protein (CRP).
Serum ferritin.
Viral serology and detection
   Hepatitis B (hepatitis B surface antigen (hBsAg), hepatitis B surface antibody (hBsAb), and hepatitis B core antibody (HBcAb); HBV DNA by PCR if acute or chronic HBV infection cannot be ruled out by serology results (www.cdc.gov/hepatitis/hbv/pdfs/serologicchartv8.pdf)).
   HCV antibody; HCV RNA by PCR if the patient is HCV antibody positive.
   HIV serology.
   EBV, CMV, and HHV-6 quantitative PCR.

Samples for the following laboratory tests will be analyzed:
Myeloma-specific tests: SPEP with SIFE, UPEP, SFLC.
Blood samples for leukocyte immunophenotyping/flow cytometry (fluorescence-activated cell sorting (FACS) lymphocyte subsets) including, but not limited to, enumeration of leukocyte subsets (e.g., T cells (CD3+, CD4+, CD8+), B cells (CD19+), and NK cells (CD16+, CD56+)), and assessment T-cell functional status (using markers including, but not limited to, CD69, CD25, and Ki67) by flow cytometry.
Blood sample for whole genome sequencing (WGS) and human leukocyte antigen (HLA) genotyping, to be collected on C1D1 (or at any time during the study after C1D1).
Plasma and serum for cytokines, including but not limited to IL-6 and IFN-γ.
Analyses of bone marrow aspirate, clot, and biopsy samples may include, but are not limited to:
   Changes in PD biomarkers, cevostamab pharmacokinetics, and MRD status. Samples may be processed to obtain bone marrow mononuclear cells and their derivatives (e.g., RNA and DNA).
   Leukocyte immunophenotyping including, but not limited to, enumeration of leukocyte subsets (e.g., T cells (CD3+, CD4+, CD8+), B cells (CD19+), and NK cells (CD16+, CD56+)), and assessment FcRH5+ target cell depletion, T-cell functional status (using markers including, but not limited to, CD69, CD25, and Ki67).
Fresh bone marrow aspirate will be used to confirm cytogenetic status at a central testing lab (at screening only).
Prior to C1D1 dosing, within 3 days prior to or on C2D1 before study treatment and at the time of confirmation of CR, a bone marrow aspirate and trephine biopsy with an associated pathology report is recommended. For bone marrow aspirate samples, please refer to the central laboratory manual for the volume of aspirate that should be collected. Trephine/core biopsy tissue samples should preferably be a minimum of 1.5 cm in length (≥2 cm is optimal). Bone marrow aspirate clot samples should be submitted only if the aspirate material unexpectedly clots.
   In patients with extramedullary disease, in the rare instance that a bone marrow biopsy is not feasible, tissue obtained from an extramedullary plasmacytoma is acceptable, but should meet the following criteria: if an excisional biopsy is performed, then a formalin-fixed, paraffin-embedded block (preferred) or a minimum of 15 serially sectioned, unstained slides is required. For core needle biopsy tissue specimens, at least three core tissue samples should be submitted for evaluation.
   Tumor tissue should be of good quality based on total and viable tumor content (sites will be informed if the quality of the submitted specimen is inadequate).
   Fine-needle aspiration, brushing, cell pellets from pleural effusion, and lavage samples are not recommended.
   Acceptable samples include core needle biopsy tissue samples for deep tumor tissue or lymph nodes or excisional, incisional, punch, or forceps tissue sample biopsies for cutaneous, subcutaneous, or mucosal lesions.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1-132. (canceled)

133. A method of treating a subject having a multiple myeloma (MM), the method comprising subcutaneously administering to the subject a bispecific antibody that binds to Fc receptor-homolog 5 (FcRH5) and cluster of differentiation 3 (CD3) in a dosing regimen comprising:
(i) a first phase comprising one or more dosing cycles, wherein the first phase comprises administering the bispecific antibody to the subject every week (QW);
(ii) a second phase comprising one or more dosing cycles, wherein the second phase comprises administering the bispecific antibody to the subject every two weeks (Q2W); and
(iii) a third phase comprising one or more dosing cycles, wherein the third phase comprises administering the bispecific antibody to the subject every four weeks (Q4W),
wherein the bispecific antibody that binds to FcRH5 and CD3 comprises an anti-FcRH5 arm comprising a first binding domain comprising the following six hypervariable regions (HVRs):
(a) an HVR-H1 comprising the amino acid sequence of RFGVH (SEQ ID NO: 1);
(b) an HVR-H2 comprising the amino acid sequence of VIWRGGSTDYNAAFVS (SEQ ID NO: 2);
(c) an HVR-H3 comprising the amino acid sequence of HYYGSSDYALDN (SEQ ID NO:3);
(d) an HVR-L1 comprising the amino acid sequence of KASQDVRNLVV (SEQ ID NO: 4);
(e) an HVR-L2 comprising the amino acid sequence of SGSYRYS (SEQ ID NO: 5); and
(f) an HVR-L3 comprising the amino acid sequence of QQHYSPPYT (SEQ ID NO: 6); and
the anti-CD3 arm comprising a second binding domain comprising the following six HVRs:
(a) an HVR-H1 comprising the amino acid sequence of SYYIH (SEQ ID NO: 9);
(b) an HVR-H2 comprising the amino acid sequence of WIYPENDNTKYNEKFKD (SEQ ID NO: 10);
(c) an HVR-H3 comprising the amino acid sequence of DGYSRYYFDY (SEQ ID NO: 11);
(d) an HVR-L1 comprising the amino acid sequence of KSSQSLLNSRTRKNYLA (SEQ ID NO: 12);
(e) an HVR-L2 comprising the amino acid sequence of WTSTRKS (SEQ ID NO: 13); and
(f) an HVR-L3 comprising the amino acid sequence of KQSFILRT (SEQ ID NO: 14).

134. The method of claim 133, wherein each dosing cycle is a 28-day dosing cycle and the first phase comprises or consists of a first dosing cycle (C1).

135. The method of claim 134, wherein the first phase comprises administration of the bispecific antibody to the subject on Days 1, 8, and 15 of the C1.

136. The method of claim 133, wherein:
(a) a target dose of the bispecific antibody is administered to the subject for each administration during the first phase;
(b) the first phase comprises administration of a first step-up dose of the bispecific antibody to the subject; or
(c) the first phase comprises administration of a first step-up dose and a second step-up dose of the bispecific antibody to the subject.

137. The method of claim 136, wherein:
(a) the first phase comprises administration of the first step-up dose of the bispecific antibody to the subject, wherein the first step-up dose is administered to the subject on Day 1 of the C1 and a target dose is administered to the subject on Days 8 and 15 of the C1; or
(b) the first phase comprises administration of the first step-up dose and the second step-up dose of the bispecific antibody to the subject, wherein the first step-up dose is administered to the subject on Day 1 of the C1, the second step-up dose is administered to the subject on Day 8 of the C1, and a target dose is administered to the subject on Day 15 of the C1.

138. The method of claim 137, wherein:
(a) the first step-up dose is 2 mg or 10 mg; or
(b) the first step-up dose is 2 mg and the second step-up dose is 10 mg.

139. The method of claim 134, wherein:
(a) the bispecific antibody
(i) is not administered to the subject on Day 22 of the C1 and is administered to the subject a total of three times during the C1, or
(ii) is administered to the subject on Day 22 of the C1;
(b) the second phase comprises at least two dosing cycles, at least three dosing cycles, at least four dosing cycles, or at least five dosing cycles; and/or
(c) the third phase comprises at least two dosing cycles, at least three dosing cycles, at least four dosing cycles, at least five dosing cycles, at least six dosing cycles, or at least seven dosing cycles.

140. The method of claim 139, wherein:
(a) the second phase comprises or consists of a first dosing cycle (C1), a second dosing cycle (C2), a third dosing cycle (C3), a fourth dosing cycle (C4), and a fifth dosing cycle (C5); and/or
(b) the third phase comprises or consists of a first dosing cycle (C1), a second dosing cycle (C2), a third dosing cycle (C3), a fourth dosing cycle (C4), a fifth dosing cycle (C5), a sixth dosing cycle (C6), and a seventh dosing cycle (C7).

141. The method of claim 140, wherein:
(a) the second phase comprises administration of the bispecific antibody to the subject on Days 1 and 15 of the C1, C2, C3, C4, and/or C5; and/or
(b) the third phase comprises administration of the bispecific antibody to the subject on Day 1 of the C1, C2, C3, C4, C5, C6, and/or C7.

142. The method of claim 141, wherein a target dose of the bispecific antibody is administered to the subject for each administration during the second phase and/or the third phase.

143. The method of claim 133, further comprising a fourth phase comprising one or more dosing cycles, wherein a target dose of the bispecific antibody is subcutaneously administered to the subject QW, Q2W, Q3W, or Q4W for each administration until disease progression.

144. The method of claim 136, wherein the target dose is 40 mg and/or the bispecific antibody is administered to the subject as a monotherapy.

145. A method of treating a subject having an MM, the method comprising subcutaneously administering to the subject a bispecific antibody that binds to FcRH5 and CD3 in a dosing regimen comprising:
(i) a first dose of the bispecific antibody of between 0.1 mg to 10 mg;
(ii) a second dose of the bispecific antibody of between 1 mg to 50 mg; and
(iii) a third dose of the bispecific antibody of between 10 mg to 200 mg,
wherein the bispecific antibody that binds to FcRH5 and CD3 comprises an anti-FcRH5 arm comprising a first binding domain comprising the following six HVRs:
(a) an HVR-H1 comprising the amino acid sequence of RFGVH (SEQ ID NO: 1);
(b) an HVR-H2 comprising the amino acid sequence of VIWRGGSTDYNAAFVS (SEQ ID NO: 2);
(c) an HVR-H3 comprising the amino acid sequence of HYYGSSDYALDN (SEQ ID NO:3);
(d) an HVR-L1 comprising the amino acid sequence of KASQDVRNLVV (SEQ ID NO: 4);
(e) an HVR-L2 comprising the amino acid sequence of SGSYRYS (SEQ ID NO: 5); and
(f) an HVR-L3 comprising the amino acid sequence of QQHYSPPYT (SEQ ID NO: 6); and
(a) an HVR-H1 comprising the amino acid sequence of SYYIH (SEQ ID NO: 9);
(b) an HVR-H2 comprising the amino acid sequence of WIYPENDNTKYNEKFKD (SEQ ID NO: 10);
(c) an HVR-H3 comprising the amino acid sequence of DGYSRYYFDY (SEQ ID NO: 11);
(d) an HVR-L1 comprising the amino acid sequence of KSSQSLLNSRTRKNYLA (SEQ ID NO: 12);
(e) an HVR-L2 comprising the amino acid sequence of WTSTRKS (SEQ ID NO: 13); and
(f) an HVR-L3 comprising the amino acid sequence of KQSFILRT (SEQ ID NO: 14).

146. The method of claim 145, wherein:
(a) the first dose of the bispecific antibody is between 1 mg to 3 mg, the second dose of the bispecific antibody is between 8 mg to 12 mg, and the third dose of the bispecific antibody is between 35 mg to 45 mg;
(b) the first dose of the bispecific antibody is 2 mg, the second dose of the bispecific antibody is 10 mg, and the third dose of the bispecific antibody is 40 mg; or
(c) the first dose of the bispecific antibody is 2 mg, the second dose of the bispecific antibody is 10 mg, and the third dose of the bispecific antibody is 120 mg.

147. The method of claim 145, wherein the bispecific antibody is administered subcutaneously by injection or by infusion to the subcutaneous tissue of the subject's abdomen or thigh.

148. The method of claim 147, wherein:
(a) the subject's abdomen comprises four quadrants, and the bispecific antibody is administered to one of the four quadrants, wherein each sequential dose of the bispecific antibody is administered to a different member of the four quadrants on a rotating basis; and/or
(b) the bispecific antibody is administered subcutaneously by injection, wherein the bispecific antibody is administered with an injection speed of about 0.25 mL/minute to about 4 mL/minute, wherein the bispecific antibody is administered by a syringe or by a pump.

149. The method of claim 148, wherein:
(a) the bispecific antibody is administered with an injection speed of about 1 mL/minute; and or
(b) the syringe is a pre-filled syringe or the pump is a patch pump, a syringe pump, an infusion pump, or a wearable pump.

150. The method of claim 145, wherein:
(I) the first binding domain of the anti-FcRH5 arm comprises:
(a) a heavy chain variable (VH) domain comprising an amino acid sequence having at least 95% sequence identity to the amino acid sequence of SEQ ID NO: 7;
(b) a light chain variable (VL) domain comprising an amino acid sequence having at least 95% sequence identity to the amino acid sequence of SEQ ID NO: 8; or
(c) a VH domain as in (a) and a VL domain as in (b), and/or
(II) the second binding domain of the anti-CD3 arm comprises:
(a) a VH domain comprising an amino acid sequence having at least 95% sequence identity to the amino acid sequence of SEQ ID NO: 15;
(b) a VL domain comprising an amino acid sequence having at least 95% sequence identity to the amino acid sequence of SEQ ID NO: 16; or
(c) a VH domain as in (a) and a VL domain as in (b).

151. The method of claim 150, wherein:
(a) the first binding domain comprises a VH domain comprising an amino acid sequence of SEQ ID NO: 7 and a VL domain comprising an amino acid sequence of SEQ ID NO: 8;
(b) the second binding domain of the anti-CD3 binding arm comprises a VH domain comprising an amino acid sequence of SEQ ID NO: 15 and a VL domain comprising an amino acid sequence of SEQ ID NO: 16;
(c) the bispecific antibody comprises an anti-FcRH5 arm comprising a heavy chain polypeptide (H1) and a light chain polypeptide (L1) and an anti-CD3 arm comprising a heavy chain polypeptide (H2) and a light chain polypeptide (L2), wherein:
(i) H1 comprises the amino acid sequence of SEQ ID NO: 35,
(ii) L1 comprises the amino acid sequence of SEQ ID NO: 36,
(iii) H2 comprises the amino acid sequence of SEQ ID NO: 37, and
(iv) L2 comprises the amino acid sequence of SEQ ID NO: 38; and/or
(d) the bispecific antibody is cevostamab.

152. The method of claim 145, wherein:
(a) the bispecific antibody comprises an aglycosylation site mutation; and/or
(b) the bispecific antibody is a monoclonal antibody, a humanized antibody, a chimeric antibody, or an antibody fragment that binds FcRH5 and CD3.

153. The method of claim 152, wherein:
(a) the aglycosylation site mutation reduces effector function of the bispecific antibody;
(b) the aglycosylation site mutation is a substitution mutation; and/or
(c) the antibody fragment is selected from the group consisting of Fab, Fab′-SH, Fv, scFv, and (Fab′)₂ fragments.

154. The method of claim 145, wherein:
(a) the bispecific antibody is a full-length antibody;
(b) the bispecific antibody is an IgG antibody;
(c) the bispecific antibody comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, second CH2 (CH2₂) domain, and a second CH3 (CH3₂) domain; and/or
(d) the MM is a relapsed or refractory (R/R) MM.

155. The method of claim 154, wherein:
(a) the IgG antibody is an IgG₁ antibody;
(b) at least one of the one or more heavy chain constant domains is paired with another heavy chain constant domain;
(c) the CH3₁ and CH3₂ domains each comprise a protuberance or cavity, and wherein the protuberance or cavity in the CH3₁ domain is positionable in the cavity or protuberance, respectively, in the CH3₂ domain; and/or
(d) the CH2₁ and CH2₂ domains each comprise a protuberance or cavity, and wherein the protuberance or cavity in the CH2₁ domain is positionable in the cavity or protuberance, respectively, in the CH2₂ domain.

156. The method of claim 155, wherein:
(a) the CH3₁ and CH3₂ domains meet at an interface between the protuberance and cavity; and/or
(b) the CH2₁ and CH2₂ domains meet at an interface between said protuberance and cavity, wherein the anti-FcRH5 arm comprises the protuberance and the anti-CD3 arm comprises the cavity.

157. The method of claim 156, wherein a CH3 domain of the anti-FcRH5 arm comprises a protuberance comprising a T366W amino acid substitution mutation (EU numbering) and a CH3 domain of the anti-CD3 arm comprises a cavity comprising T366S, L368A, and Y407V amino acid substitution mutations (EU numbering).

158. The method of claim 145, wherein:
(a) the bispecific antibody is administered to the subject concurrently with one or more additional therapeutic agents;
(b) the bispecific antibody is administered to the subject prior to an administration of one or more additional therapeutic agents; or
(c) the bispecific antibody is administered to the subject subsequent to an administration of one or more additional therapeutic agents.

159. The method of claim 158, wherein:
(I) the one or more additional therapeutic agents comprise:
(a) an effective amount of tocilizumab;
(b) an effective amount of a corticosteroid;
(b) an effective amount of an immunomodulator (IMiD), a cluster of differentiation 38 (CD38)-directed therapy, or a B-cell maturation antigen (BCMA)-directed therapy;
(c) an effective amount of acetaminophen or paracetamol; or
(d) an effective amount of diphenhydramine, or
(II) the subject has a cytokine release syndrome (CRS) event, and the method comprises administering tocilizumab to the subject by intravenous infusion while suspending treatment with the bispecific antibody, wherein:
(a) the subject weighs ≥30 kg, and tocilizumab is administered to the subject at a dose of 8 mg/kg;
(b) the subject weighs <30 kg, and tocilizumab is administered to the subject at a dose of 12 mg/kg; or
(c) the final dose of tocilizumab administered to the subject does not exceed 800 mg, wherein the CRS event does not resolve or worsens within 8 hours of treating the symptoms of the CRS event, and the method further comprises administering to the subject one or more additional doses of tocilizumab to manage the CRS event.

160. The method of claim 159, wherein:
(a) the corticosteroid (i) is administered intravenously to the subject, (ii) is methylprednisolone or dexamethanone, and/or (iii) is administered to the subject 45 minutes to 75 minutes prior to administration of the bispecific antibody;
(b) acetaminophen or paracetamol is administered to the subject orally at a dose of between 500 mg to 1000 mg and/or prior to administration of the bispecific antibody;
(c) diphenhydramine is administered to the subject orally at a dose of between 25 mg to 50 mg and/or prior to administration of the bispecific antibody to the subject; or
(d) the IMiD is pomalidomide, the CD38-directed therapy is an anti-CD38 antibody, or the BCMA-directed therapy is an antibody-drug conjugate targeting BCMA.

161. The method of claim 160, wherein:
(a) methylprednisolone is administered at a dose of 80 mg;
(b) dexamethasone is administered at a dose of 20 mg;
(c) the corticosteroid is administered to the subject 60 minutes prior to administration of the bispecific antibody to the subject;
(d) the corticosteroid is administered to the subject prior to administration of the bispecific antibody if the subject experienced CRS with a prior administration of the bispecific antibody to the subject; or
(e) the anti-CD38 antibody is daratumumab, MOR202, or isatuximab.

162. The method of claim 154, wherein:
(a) the subject has a diagnosis of R/R MM for which no established therapy for MM is appropriate and available, or intolerance to established therapies; and/or
(b) the subject has measurable disease, defined as at least one of the following:
(i) serum M-protein ≥0.5 g/dL;
(ii) urine M-protein ≥200 mg/24 h; or
(iii) serum free light chain (SLFC) assay: involved SFLCs ≥10 mg/dl and an abnormal SFLC ratio (<0.26 or >1.65).

163. A subcutaneous administration device comprising a bispecific antibody that binds to FcRH5 and CD3, wherein the subcutaneous administration device comprises:
(i) a first dose of the bispecific antibody of between 0.5 mg to 8 mg;
(ii) a second dose of the bispecific antibody of between 2 mg to 40 mg; and/or
(iii) a third dose of the bispecific antibody of between 10 mg to 160 mg,
wherein the bispecific antibody that binds to FcRH5 and CD3 comprises an anti-FcRH5 arm comprising a first binding domain comprising the following six hypervariable regions (HVRs):
(a) an HVR-H1 comprising the amino acid sequence of RFGVH (SEQ ID NO: 1);
(b) an HVR-H2 comprising the amino acid sequence of VIWRGGSTDYNAAFVS (SEQ ID NO: 2);
(c) an HVR-H3 comprising the amino acid sequence of HYYGSSDYALDN (SEQ ID NO:3);
(d) an HVR-L1 comprising the amino acid sequence of KASQDVRNLVV (SEQ ID NO: 4);
(e) an HVR-L2 comprising the amino acid sequence of SGSYRYS (SEQ ID NO: 5); and
(f) an HVR-L3 comprising the amino acid sequence of QQHYSPPYT (SEQ ID NO: 6); and
(a) an HVR-H1 comprising the amino acid sequence of SYYIH (SEQ ID NO: 9);
(b) an HVR-H2 comprising the amino acid sequence of WIYPENDNTKYNEKFKD (SEQ ID NO: 10);
(c) an HVR-H3 comprising the amino acid sequence of DGYSRYYFDY (SEQ ID NO: 11);
(d) an HVR-L1 comprising the amino acid sequence of KSSQSLLNSRTRKNYLA (SEQ ID NO: 12);
(e) an HVR-L2 comprising the amino acid sequence of WTSTRKS (SEQ ID NO: 13); and
(f) an HVR-L3 comprising the amino acid sequence of KQSFILRT (SEQ ID NO: 14).
